(19) United States
(12) Patent Application Publication
(10) Pub. No.: US 2024/0266069 A1
(43) Pub. Date: Aug. 8, 2024

(54) COMPUTER ASSISTED METHOD FOR THE EVALUATION OF CARDIAC METABOLISM

(71) Applicant: Charité – Universitätsmedizin Berlin, Berlin (DE)

(72) Inventors: Nikolaus BERNDT, Berlin (DE); Titus KUEHNE, Berlin (DE)

(73) Assignee: Charité – Universitätsmedizin Berlin, Berlin (DE)

(21) Appl. No.: 18/561,751

(22) PCT Filed: May 19, 2022
(86) PCT No.: PCT/EP2022/063620

§ 371 Date: Nov. 17, 2023

(30) Foreign Application Priority Data
May 19, 2021 (EP) 21174633.4
Jun. 25, 2021 (EP) 21181803.4

(51) Int. Cl. G16H 50/50; G01N 33/68; G16B 5/00; G16B 25/10; G16B 40/20

(52) U.S. Cl. CPC G16H 50/50; G01N 33/6842; G16B 5/00; G16B 25/10; G16B 40/20

(57) ABSTRACT

The invention relates to a computation based method for determining an individual cardiac metabolic profile in a subject and related materials, devices and mathematical model usage. The present invention therefore relates to a computation-based method for determining an individual metabolic cardiac profile of a subject comprising provision of a heart tissue sample from said subject, quantifying proteins in said sample from said subject, and applying information about quantities of said proteins to a mathematical model. In some embodiments, individual cardiac parameters and/or the metabolites of the subject are additionally introduced into the mathematical model, wherein individual cardiac parameters are determined for a plurality of cardiac workloads, including rest, stress or cardiac pacing. The invention also relates to the individual cardiac metabolic profile comprising a substrate uptake rate, a myocardial ATP consumption, a myocardial ATP production reserve, a myocardial ATP production at said cardiac workload, and a myocardial ATP production at maximal workload, wherein the myocardial ATP production reserve is calculated as the difference between the myocardial ATP-production at said cardiac workload and the myocardial ATP production at maximal workload. The invention further relates to the medical use and corresponding therapeutic methods based on the individual metabolic cardiac profile of the invention in the treatment, prevention, ascertainment, prognosis, of a medical condition associated with a cardiovascular disorder, in addition to detect a perturbation of a normal biological state of the heart from the subject. The invention further relates to the medical use and corresponding therapeutic methods based on the individual metabolic cardiac profile of the invention for the heart at physiological state and/or at pathological state. In further aspects, the invention relates to a computer program adapted to execute a mathematical modelling algorithm that will be performed by a computing device/module to produce outputs given data provided as inputs according to preceding claims, wherein said computer program, preferably MATLAB, is written in a programming language selected from a group comprising Fortran, C #, C/C++, High Level Shading Language, or Python.

The invention relates to a computation based method for determining an individual cardiac metabolic profile in a subject and related materials, devices and mathematical model usage.

The present invention therefore relates to a computation-based method for determining an individual metabolic cardiac profile of a subject comprising provision of a heart tissue sample from said subject, quantifying proteins in said sample from said subject, and applying information about quantities of said proteins to a mathematical model. In some embodiments, individual cardiac parameters and/or the metabolites of the subject are additionally introduced into the mathematical model, wherein individual cardiac parameters are determined for a plurality of cardiac workloads, including rest, stress or cardiac pacing. The invention also relates to the individual cardiac metabolic profile comprising a substrate uptake rate, a myocardial ATP consumption, a myocardial ATP production reserve, a myocardial ATP production at said cardiac workload, and a myocardial ATP production at maximal workload, wherein the myocardial ATP production reserve is calculated as the difference between the myocardial ATP-production at said cardiac workload and the myocardial ATP production at maximal workload.

The invention further relates to the medical use and corresponding therapeutic methods based on the individual metabolic cardiac profile of the invention in the treatment, prevention, ascertainment, prognosis, of a medical condition associated with a cardiovascular disorder, in addition to detect a perturbation of a normal biological state of the heart from the subject. The invention further relates to the medical use and corresponding therapeutic methods based on the individual metabolic cardiac profile of the invention for the heart at physiological state and/or at pathological state.

In further aspects, the invention relates to a computer program adapted to execute a mathematical modelling algorithm that will be performed by a computing device/module to produce outputs given data provided as inputs according to preceding claims, wherein said computer program, preferably MATLAB, is written in a programming language selected from a group comprising Fortran, C #, C/C++, High Level Shading Language, or Python.

### BACKGROUND OF THE INVENTION

Cardiovascular diseases are the leading cause of death worldwide and are primarily caused by an individual's lifestyle and dietary intake, as well as by inborn, genetic and non-genetic, predispositions. Smoking, high cholesterol, high blood pressure, lack of exercise and diabetes are factors that influence the occurrence of heart diseases. According to the World Health Organization (WHO), heart diseases are responsible for 12% of all deaths.

Heart diseases have assumed epidemic status worldwide, and despite advances in the development of drugs, surgical techniques, and medical practices, there are still a need to provide new and improved means of prevention, early detection, and correct assessment of an individual's heart disease, as well as to identify the appropriate course of treatment and its success. For example, monitoring of an individual's cardiac condition, to date the best means of prevention, can be accomplished through regular visits to the cardiologist, with the electrocardiogram (ECG) being the primary means of detecting changes in an individual's cardiac condition.

In recent years, numerous studies have firmly established metabolic disturbances as a cardinal feature of cardiac disease pathophysiology [1-5]. Although alterations in cardiac metabolism are understood to be an underlying component in almost all cardiac myopathies, the potential contribution of myocardial energy metabolism to the reduction in cardiac output and associated exercise intolerance is not fully understood [6]. In heart failure, gene expression of key proteins involved in cardiac energy metabolism is often downregulated (see, e.g., [8, 9]), as are levels of key metabolic enzymes (e.g., creatine kinase, CK [10]) and cardiac energy-rich phosphates (ATP, CrP). Although these findings suggest a mismatch between ATP demand and ATP supply, they do not allow to assess the degree of the mismatch. There is a high medical need to identify or predict this mismatch to determine risks for heart disease, such as valve disease, and/or heart failure.

The most common types of valve disease are aortic stenosis (AS) and mitral regurgitation (MI), which expose the heart to long-term pressure and volume overload, respectively. Pressure-volume overload triggers cardiac remodeling, which typically results in myocardial hypertrophy. Some patients tolerate this condition well for years, whereas others rapidly progress from compensated to decompensated heart failure despite similar characteristics. Therefore, it is of particular importance in the clinic to be able to predict the risk of transition from compensated to decompensate heart failure as accurately as possible. This would include a better knowledge of the metabolic status of the myocardium in heart disease. In the state of the art, cardiac magnetic resonance imaging and 31P magnetic resonance spectroscopy can previously be used to visualize whether reduced ATP delivery from mitochondria to myosin ATPase through the CK shuttle is associated with an otherwise unexplained reduced LV (left ventricle) ejection fraction in some (but not all) patients with severe AS. However, this does not allow for evidence of a significant difference in CK flow in patients with cardiac dysfunction. In the prior art, network reconstruction of the metabolic network of cardio-myocytes and modeling work on metabolic subsystems are known [15-18] as well as methods of diagnosing metabolic syndromes or chronic rejection of a cardiac allograft using genomic expression profiling, proteomic expression profiling, or a combination of genomic and proteomic expression profiling or a virtual surgical guides as a three-dimensional digital representation of the heart [65-68]. There is still a lack of a method and mathematical model to demonstrate changes in cardiac metabolism that are associated with cardiovascular abnormalities. In particular, there is a lack of a method to show a gradual reduction in myocardial ATP production capacity (MVATP) in cardiovascular tissue, especially in cardiac (dys-)function, e.g., in association with deterioration in LV systolic function.

This addresses an unmet and urgent medical need in the health care of cardiovascular abnormalities.

### SUMMARY OF THE INVENTION

In light of the prior art, the technical problem underlying the invention was the provision of novel means for preventing, prognosis, ascertaining, and treating a cardiovascular related disorder or a pathophysiology state of the heart. Changes is cardiac metabolism, e.g. ATP production capacity, is an underlying component of a cardiovascular related disorder, cardiac morbidity and pathophysiology state of the heart.

One objective was to provide a computation-based model to determine an individual cardiac metabolic profile for preventing, prognosis, ascertaining, and treating a cardiovascular related disorder or a pathophysiology state of the heart. Another objective was to provide a method to process a heart tissue sample from a subject and cardiac parameter for modelling an individualized cardiac metabolic profile using a trained reference data set. Despite the high medical need, the state of the art currently does not provide means to determine the metabolic profile of the heart of a subject and thus to assess the ability of the heart tissue to increase energy supply in response to an increase in energy demand, e.g. ATP demand.

The technical problem can also be seen in the provision of means to assess to energetic capacity of the heart tissue from a subject by combining kinetic modeling with protein abundance data of metabolic enzymes determined in the heart tissue. The technical problem can also be seen in providing the means to generate a complex physiology-based mathematical model of cardiac energy metabolism that includes pathways that use energy-providing substrates. The technical problem can also be seen in providing the means to execute a mathematical modeling algorithm by a computer program.

These problems are solved by the features of the independent claims. Preferred embodiments of the present invention are provided by the dependent claims.

The invention therefore relates to computation-based method for determining an individual metabolic cardiac profile to prevent, to prognose, to ascertain and/or to treat a cardiovascular related disorder or a pathophysiology state of the heart.

The invention therefore relates to a computer-implemented method for determining an individual metabolic cardiac profile of a subject comprising:
a) Providing a heart tissue sample from said subject, and
b) Quantifying proteins in said sample from said subject, and
c) Applying information about quantities of said proteins from step b) to a mathematical model.

The invention also relates to a computer-implemented method for determining an individual cardiac metabolite and proteome profile of a human subject comprising:
a) Providing a heart tissue sample comprising proteins from said subject, and
b) Quantifying a protein expression level of multiple proteins in said sample from said subject using a large-scale protein quantification method, and
c) Applying the protein expression level from step b) to a mathematical model,
d) Calculating the individual cardiac metabolite and proteome profile based on protein expression level from step b) from said subject using the mathematical model in step c).

Access to human cardiac muscle samples is very challenging and thus exceptional and is of particular value to the present invention. To date, the prior art has only been able to detect proteins individually or a small number of proteins from a human heart muscle sample. For example, equal to or less than 100 proteins were detected. However, this information was not sufficient to provide an entire profile for the proteome and inferred metabolism for a human subject. Such a profile has the advantage of looking at the interactions of proteins with each other and thus correctly assessing the metabolic state of the heart and its ATP capacity and identifying pathological conditions. A particular advantage of the invention is that the human subject receives a nutritional or a therapeutic intervention that corresponds to the actual metabolic energy status and biological condition of the heart of said subject without and the treatment is selected on the basis of this condition, minimizing, if not completely avoiding, the risk of misinterpretation or mistreatment by intermediate steps and interference of the measurement results by other organs.

In this regard, the application of the method is not limited to a specific sample from one part of the heart, but rather can be applied to any cardiac muscle sample.

Furthermore, in the prior art, proteins and metabolites are often measured from a blood sample from a human subject and conclusions are drawn about the activity, metabolic status, and possible pathological conditions of the heart. However, the proteins or metabolites measured in the blood need not originate from the heart, or the measured changes in proteins need not be a response to conditions of the heart. This may lead to misinterpretation, resulting in mistreatment, or disease may be overlooked.

The present invention is based on quantified protein expression levels. Protein expression levels have the advantage over measurements of nucleic acids (e.g., with microarrays) that they accurately reflect the metabolic state of the heart, since proteins are the executive elements of metabolism, and therefore avoid misinterpretation due to the step of producing proteins from nucleic acid information.

The method described herein relate to hearts from a human subject, reflecting the autologous, metabolic state of the individual heart from said human subject. Rejection reactions, as they may occur after heart transplantation, are in particular a reaction of the immune system of the recipient of the donor heart. In particular, the immune system response is measurable by the immune cells circulating in the blood and is indicative of a rejection status. The invention provided herein is not intended to be used to determine a rejection reaction or rejection status in a human subject, such as diagnosis of chronic allograft rejection of a cardiac allograft.

In one embodiment, the individual metabolic cardiac profile of a diseased subject (patient, affected) can be compared to the individual metabolic cardiac profile of a non-diseased subject (control, normal).

The individual metabolic cardiac profile, if compared to a non-diseased subject, providing information about cardiac metabolic changes in the heart from the subjects can be used for (i) selecting a nutritional or a therapeutic intervention, and (ii) evaluating or preventing a therapeutic intervention. Cardiovascular related disorders or a perturbation of a normal biological state of the heart are characterized by cardiac metabolic changes.

In cardiovascular related disorders, gene expression of key proteins involved in cardiac energy metabolism as well as important metabolic enzymes (e.g., creatine kinase, CK) and cardiac energy-rich phosphates (ATP, CrP) are often downregulated. These findings suggest a mismatch between cardiac ATP demand and cardiac ATP production, and therefore in the individual metabolic cardiac profile. There is a high medical need to determine this mismatch to identify risks for heart diseases, such as valve disease, and/or heart failure. The individual cardiac metabolic profile comprises a substrate uptake rate, a myocardial ATP consumption, a myocardial ATP production reserve, a myocardial ATP production. It has proven very difficult in the art to determine the cardiac ATP production rate and the degree of reduction of cardiac ATP production in the heart tissue of a subject. The skilled person could not expect that using the protein and metabolic information of the model and the protein data from the subject's heart tissue, the ATP capacity at rest and maximum workload could be calculated. It was surprising that pathological conditions of the heart could not only be determined but also predicted, such as a cardiovascular disease or a perturbation of a normal biological state of a heart in an autologous situation in the human subject. Cardiovascular disorders are a large class of diseases that affect the heart and/or blood vessels (arteries and veins). In one embodiment, a cardiovascular related disorder can be one of, but not limited to, arrhythmias, vascular disease, myocardial infarction, heart failure, myocarditis, atherosclerosis, restenosis, coronary heart disease, coronary artery disease, atherosclerotic cardiovascular disease, arterial hypertension, cardiac fibrosis, stroke, sudden cardiac death syndrome, heart failure, ischemic heart disease, ischemic cardiomyopathy, myocardial infarction, coronary artery calcification. These diseases have similar causes, mechanisms, and treatments. Most cardiovascular disorders have common risk factors, including change cardiac metabolism, inflammation, fibrosis, diabetes, cholesterol, and vascular deposits. In one embodiment, the common risk factor is a change in cardiac metabolism.

In one embodiment, changes in cardiac metabolism comprise changes in cardiac enzyme activity, cardiac gene expression, cardiac substrate uptake rate, cardiac hormone concentration (e.g. insulin, catecholamines), cardiac metabolite concentration, cardiac ATP consumption, cardiac oxygen consumption, cardiac NO, cardiac ion exchange, cardiac energy-rich phosphates, and/or cardiac ATP production capacity. In one embodiment, changes in cardiac metabolism are associated to a pathological state of the heart and may lead to congestive heart failure, compromised cardiac function, cardio-embolism, vascular and cardiac damage, diastolic dysfunction, cardiac dysfunction, cardiac valve disease, reduction of the cardiac output, exercise intolerance, conduction disturbances, or sudden death.

In one embodiment, the individual metabolic cardiac profile of a subject involves metabolite concentration, hormone concentration, enzyme activity, protein expression, hormones, protein profile of a heart tissue from a subject, and/or individual parameter, wherein individual parameter comprise cardiac parameter, individual history, medication, laboratory parameter.

In one embodiment, metabolite concentrations can be obtained from database, published literature, and/or determined in a sample from a subject, wherein said sample comprise body fluid, blood, plasma, serum, heart tissue, preferably blood and/or heart tissue.

In one embodiment, the protein expression can be obtained from database, published literature, and/or determined in a sample from a subject, wherein said sample comprise body fluid, blood, plasma, serum, heart tissue, preferably blood and/or heart tissue.

In one embodiment, the enzyme activity can be obtained from database, published literature, and/or a sample from a subject, wherein said sample comprise body fluid, blood, plasma, serum, heart tissue, preferably blood and/or heart tissue.

In one embodiment, the protein profile of a subject is usually determined in the heart tissue from the subject.

In one embodiment, the hormone concentration can be obtained from database, published literature, and/or a sample from a subject, wherein said sample comprise body fluid, blood, plasma, serum, heart tissue, preferably blood and/or heart tissue.

In one embodiment, the subject is a human subject. A particular advantage of the present invention is the possible application of the method for determining the individual cardiac metabolic profile in the heart tissue of a human subject. For that, heart tissue sample from a human subject is used ex vivo. The skilled person knows about the importance and the special value of heart tissue samples from a human being and that access to these samples is a special challenge. The person skilled in the art is familiar with methods for obtaining heart tissue samples from a human being and knows that these are usually obtained as part of a planned surgery. Preferred collection and preparation of the heart tissue samples is described in the Examples. In the prior art, therefore, heart tissue samples from animals, such as mice, are generally used. The skilled person is aware of the lack of consistency of biological data from animals compared to humans and that this often leads to errors in the choice of therapeutic options, development of drugs, assessment of disease progression and nutritional recommendations. The person skilled in the art knows from the literature that protein profiles from mice can match humans about only half. This provides the invention with even more essentiality, correctness and usefulness.

In one embodiment, individual parameters, as used herein, include patient age, smoking behavior (either the mere fact of being an (inhalant) smoker or the number of cigarettes per day), systolic and/or diastolic blood pressure, HDL cholesterol level (either concentration or particle number), blood glucose concentration, triglyceride concentrations, subject sex, and (blood pressure) medication.

As generally known by a skilled person, patients with cardiovascular related diseases frequently have decreased ATP production in cardiac muscle cells as well as other abnormalities in cardiac metabolism, including cell death. Under physiological conditions, more than 95% of adenosine triphosphate (ATP) is generated from oxidative phosphorylation in the heart, mainly by utilization of fatty acids (FAs), with the remaining 5% produced by glycolysis. The high rate of ATP production and turnover in the heart is required to maintain its continuous mechanical work. Disturbances in ATP-generating processes may therefore directly affect contractile function. Characterization of cardiac metabolism in heart disease, such as heart failure (HF), revealed several metabolic changes termed metabolic remodeling, ranging from altered substrate utilization to mitochondrial dysfunction, ultimately leading to ATP deficiency and impaired contractility.

In the prior art, no method is available to measure myocardial ATP production capacity in vivo.

In one embodiment, the heart tissue sample can be a left ventricle, a right ventricle, a septum, a left atrium, a right atrium heart tissue sample obtained during a myocardium examination or cardiac surgery, preferably a cardiac catheter examination.

In one embodiment, a sample from a subject have been obtained from a subject with cardiovascular disease or pathophysiological state of the heart. In one embodiment, a sample from a subject have been obtained from a non-diseased subject (control, normal). The sample may have been obtained from another person and given to the person (or machine) performing the procedure.

In one embodiment, the heart tissue sample can be left ventricular septum biopsies specimen from patients admitted in need for aortic or mitral valve replacement surgery or from healthy donor heart control subjects.

In one embodiment, said heart tissue sample is a left ventricular septum sample.

In one embodiment, said heart tissue sample is a right ventricular septum sample.

Many suitable sample types are evident to a skilled person. In one embodiment of the invention, the sample is a heart tissue sample. In one embodiment, the heart tissue sample can be selected from a group of a left ventricle, a right ventricle, a septum, a left atrium, a right atrium heart tissue sample obtained during a myocardium examination, a heart transplantation, an insertion of a pacemaker, an insertion of a defibrillator or a cardiac surgery, preferably a cardiac catheter examination. Methods for storing and lysing of heart tissue samples and protein extraction from heart tissue samples are well-known to a skilled worker. A preferred method storing and lysing of heart tissue samples and protein extraction from of heart tissue samples is provided in the Examples.

In one embodiment, the sample is a blood sample, such as whole blood, plasma, or serum (plasma from which clotting factors have been removed). For example, peripheral, arterial or venous plasma or serum may be used. In another embodiment, the sample is urine, sweat, or other body fluid in which proteins are sometimes removed from the bloodstream. In one embodiment, metabolites are determined in blood samples. In one embodiment, hormones are determined in blood samples.

A particular advantage of the invention is the provision of a method in which, in order to determine the individual cardiac metabolic profile of a subject, the data are obtained from the cardiac tissue samples used and the individual parameters are obtained from the same subject.

In one embodiment, the method comprises additionally quantitatively determining of metabolites in plasma, blood, or serum sample, preferably plasma sample, from said subject, wherein said metabolites can be selected from a group of glucose, lactate, pyruvate, glycerol, fatty acids, glutamate, glutamine, leucin, isoleucine, valine, acetate, B-hydroxybutyrate, catecholamines, or insulin.

In one embodiment, the quantitatively determined metabolite of a diseased subject (patient, affected) can be compared to the quantitatively determined metabolites of a non-diseased subject (control, normal).

In one embodiment, the metabolite can be determined in plasma. In one embodiment, the metabolite can be determined in blood. In one embodiment, the metabolite can be determined in serum. In one embodiment, the metabolite can be determined in the heart tissue sample. The metabolite concentration may vary with time. In one embodiment, the time variation of the metabolite concentration comprises a change of the input value in a time course that both shifts the output signal in time and changes other parameters and behavior. In one embodiment, the metabolite concentration

In one embodiment, metabolite concentrations from a subject can be absent for the determination of the individual cardiac metabolic profile. Mathematical modeling can also be performed with metabolite concentrations obtained from databases and/or from the published literature and is known for a variety of kinetic models and metabolic pathways. This represents a particular advantage of the invention, as accurate calculation and modeling is possible even in the absence of data from the subject, and thus the mathematical model always provides more accurate, reproducible and reliable calculations. In one embodiment, metabolite concentrations from the subject can be added to the reference data set. In one embodiment, metabolite concentration from databases and/or from literature can be added to the reference data set.

In one embodiment, the computation-based method comprises additionally quantitatively determining of an individual cardiac parameter comprising heart rate, blood pressure, pressure-volume loops, and/or heart power.

In one embodiment, the quantitatively determined cardiac parameter of a diseased subject (patient, affected) can be compared to the quantitatively determined cardiac parameter of a non-diseased subject (control, normal).

In one embodiment, individual cardiac parameters comprise ventricular end diastolic volume, ventricular end systolic volume, stroke volume, heart rate, cardiac output, preload, afterload, contractility, ejection fraction, blood pressure, pressure-volume loops, and/or heart power.

The individual cardiac parameter, provided herein, is particularly advantage for studying the activity and regulation of the heart subject to the individual cardiac metabolic profile.

In one embodiment, the protein quantity of the heart tissue sample from the subject is determined using a protein quantification method selected from the group of mass spectrometry, large scale mass spectrometry, immunoassay, Western blot, microfluidics/nanotechnology sensor, and aptamer capture assay, preferably large scale mass spectrometry, wherein said method comprises:
a) Solubilizing the heart tissue sample,
b) Extracting proteins from solubilized heart tissue sample of step a) according to the protein quantification method, wherein said proteins are preferably fragmented into peptides,
c) Transferring said extracted proteins and/or peptides from step b) to a device, preferably a mass spectrometer, of said protein quantification method identifying and quantifying the proteins and/or peptides in said sample, preferably the peptides, and
wherein said protein quantification method provides a protein profile of said sample from the subject.

In one embodiment, the quantitatively determined protein profile of a diseased subject (patient, affected) can be compared to the quantitatively determined protein profile of a non-diseased subject (control, normal).

Several protein quantification methods are known in the prior art. In one embodiment, mass spectrometry is preferably used for quantifying proteins of the heart tissue sample from the subject, more preferably large scale mass spectrometry analyses. Mass spectrometry based proteomics has become a method of choice to study proteins in a global manner. Mass spectrometry is not inherently quantitative but methods have been developed to address this limitation to a certain extent. In one embodiment, the large scale mass spectrometry analyses is used for determining absolute protein quantities. Absolute quantification is technically more challenging than relative quantification and could so far only be performed accurately for a single or a small number of proteins at a time. Typical applications of absolute quantification are the determination of cellular copy numbers of proteins (important for systems biology) or the concentration of biomarkers in body fluids (important for medical applications). In addition, any precise method of absolute quantification, when performed in more than one sample, also provides the relative amounts of protein between these samples. Several methods for absolute quantification have emerged in recent years, including HR/AM-SIM, iSRM, AQUA, QConCAT, PSAQ, absolute SILAC, and FlexiQuant.

In one embodiment, an absolute quantification method is used. In one embodiment, proteins of the heart tissue sample are also absolutely quantified using HR/AM-SIM with an Orbitrap instrument. The protein quantification method, as used here, allows the most challenging samples (low abundance, high complexity) to be analyzed to find more compounds in less time, perform more accurate quantifications, and elucidate structures more thoroughly. The method for protein extraction and quantification as used herein is described in the Examples.

A particular advantage of the invention is use of a proteomics-based abundance of metabolic enzymes in heart tissue sample to generate the individual cardiac metabolic profile.

In one embodiment, the protein profile, the individual cardiac parameters and/or the metabolites of the subject are introduced into the mathematical model.

Another advantage of the invention is the comprehensiveness of the data on which the mathematical model of the invention is based, wherein said data comprises left ventricular septum heart tissue samples from 75 human subjects. As described in the Examples, heart tissue samples from the left ventricular septum were collected during surgical aortic or mitral valve replacement from 41 patients with aortic valve stenosis and 17 patients with mitral valve insufficiency, and 17 control subjects.

In one embodiment, the individual metabolic cardiac profile can be calculated for a plurality of cardiac workloads, including rest, stress or cardiac pacing, wherein individual cardiac parameter including heart rate, blood pressure, heart power are determined at said cardiac workloads.

In one embodiment, the individual metabolic cardiac profile can be calculated for a plurality of cardiac workloads, wherein said cardiac workload determines the heart under a physiological condition, including sleep, rest, activity, stress or cardiac pacing. The individual metabolic cardiac profile is dependent on a cardiac workload. In one embodiment, the maximal workload is also used as the highest utilization of the heart.

In one embodiment, the individual metabolic cardiac profile at the cardiac workload of a diseased subject (patient, affected) can be compared to the individual metabolic cardiac profile at the same cardiac workload of a non-diseased subject (control, normal).

A further advantage of the invention is that the mathematical modelling can be performed for a plurality of cardiac workloads, particularly for any cardiac workload, wherein the mathematical model of the invention can be adapted to the workload of the heart. This increases the accuracy of the individual metabolic cardiac profile, in particular the accuracy of the prediction for cardiovascular disease or treatment selection.

In one embodiment, the mathematical model of the individual metabolic cardiac profile of the subject comprises
Inputting a cardiac kinetic model and providing metabolic parameters relating to the cardiac kinetic model, and/or
Providing individual cardiac parameters at cardiac workload,
Parametrizing said mathematical model to the heart tissue sample of said subject by calculating a maximal activity Vₘₐₓ of said subject, and
Computing a cardiac energy expenditure profile of said subject at cardiac workload,
wherein said individual metabolic cardiac profile of said subject is preferably compared to a non-diseased subject at cardiac workload.

In one embodiment, the mathematical model of the individual metabolic cardiac profile of the subject comprises
a) Loading a cardiac kinetic model and providing metabolic parameters relating to the cardiac kinetic model, wherein said model comprises reference protein expression levels and metabolic parameters (from other subjects), and
b) Inputting the protein expression levels quantified of said subject to the model, and
c) Providing optionally individual cardiac parameters at cardiac workload to the model, and
d) Parametrizing said mathematical model to the heart tissue sample of said subject by calculating a maximal activity Vₘₐₓ of said subject by applying the protein expression levels of step b) to said model, and
e) Computing a cardiac energy expenditure profile of said subject at cardiac workload,
   wherein said individual metabolic cardiac profile of said subject is preferably compared to a non-diseased subject at cardiac workload.

In some embodiments of the present invention, the mathematical model comprises one or more parameters relating to the cardiac tissue sample and/or to the subject; one or more kinetic models, preferably cardiac kinetic model, protein profile data comprising data points relating to the cardiac tissue and/or the subject such that an update uses the data; one or more algorithms using one or more of the parameters, one or more kinetic models, preferably cardiac kinetic model, and the data as input, such that the algorithms enable determination of the individual cardiac metabolic profile; and code to implement the algorithms.

Data comprise protein quantities, peptide quantities, protein labels, cardiac parameter labels, numeric cardiac parameter, clinical laboratory parameter labels, numeric clinical laboratory parameter, numeric metabolites, metabolite label, cardiac workload label, cardiac kinetic model.

In one embodiment, computing the maximal activity Vmax for model parametrization for the heart tissue sample of the subject comprises
a) Input of the protein profile of the subject according to claim 5, and
b) Loading at least one reference data set, wherein said reference data set comprises a reference data set containing the quantities of data entries, wherein each data entry of the quantity contains at least one correlated compatible protein label and/or metabolite label, and
c) Computing the maximal enzyme activity Vmax of the subject, wherein Vmax is calculated by the formula

$${V}_{ma x}^{s ubject} = {V}_{ma x}^{r e f} \frac{{E}^{s ubject}}{{E}^{r e f}}$$

by applying protein quantities according to claim 5 of the subject to to E^{subject} and by applying Vₘₐₓ^{ref} and protein quantities to E^{ref} of any of the reference data sets.

In some embodiments, the mathematical model is a metabolic model.

In one embodiment, reference data are primary data for all inputs, parameters, quantities, kinetic data, model variables (dependent or independent), even under different workloads. In some embodiments, reference data comprise published experimental data of mammalian hearts, comprise literature data, experimental data of mammalian tissue sample, preferably heart tissue sample, at physiological state and/or experimental data of mammalian tissue sample, preferably heart tissue sample, at pathological state. Reference data sets are usually stored in databases.

In some embodiments, the mathematical model was parametrized for individual heart tissue sample by proteomics-derived protein profiles of enzymes and transporter proteins by computing the maximal activity (Vₘₐₓ) of the enzyme. The maximal activities vₘₐₓ^{normal} of the reference data set comprising the average of heart tissue samples of control subjects were obtained by fitting of the model to experimental data.

In one embodiment, Vₘₐₓ values may vary due to variable protein profiles of subjects. In one embodiment, the maximum enzyme activity is proportional to the abundance of the protein.

The “Vₘₐₓ” refers to the maximal activity of an enzyme that is related to the protein concentration (E) by

$${v}_{m α x} = {k}_{cαt} E$$

wherein k_{cat} is the catalytic rate constant (“turnover number”) of the enzyme/transporter.

In one embodiment, time course of model variables (concentration of metabolites and ions) is governed by first-order differential equations. In one embodiment, the time-variations of small ions are modeled by kinetic equations of the Goldman-Hodgkin-Katz type. In one embodiment, the rate laws for enzymes and membrane transporters were either taken from the literature. In one embodiment, the rate laws for enzymes and membrane transporters were constructed based on published experimental data for the mammalian heart.

Kinetic equations and model parameters are usually set up for individual pathways. Numerical values for all other parameters of the enzymatic rate laws were taken from kinetic studies of the isolated enzymes reported in the literature.

In one embodiment, calculated metabolite profiles and fluxes are adjusted to experimental data from independent experiments with perfused hearts and in vivo measurements. In one embodiment, metabolite concentrations were constrained to experimentally determined ranges. In some embodiments, short-term regulation of key regulatory enzymes by the hormones insulin and catecholamines (epinephrine, nor-epinephrine) are included into the model by phenomenological mathematical functions relating the enzyme's phosphorylation state and the abundance of the GLUT4 transporter in the sarcolemma to the plasma concentrations of glucose (insulin) and the exercise level (catecholamines).

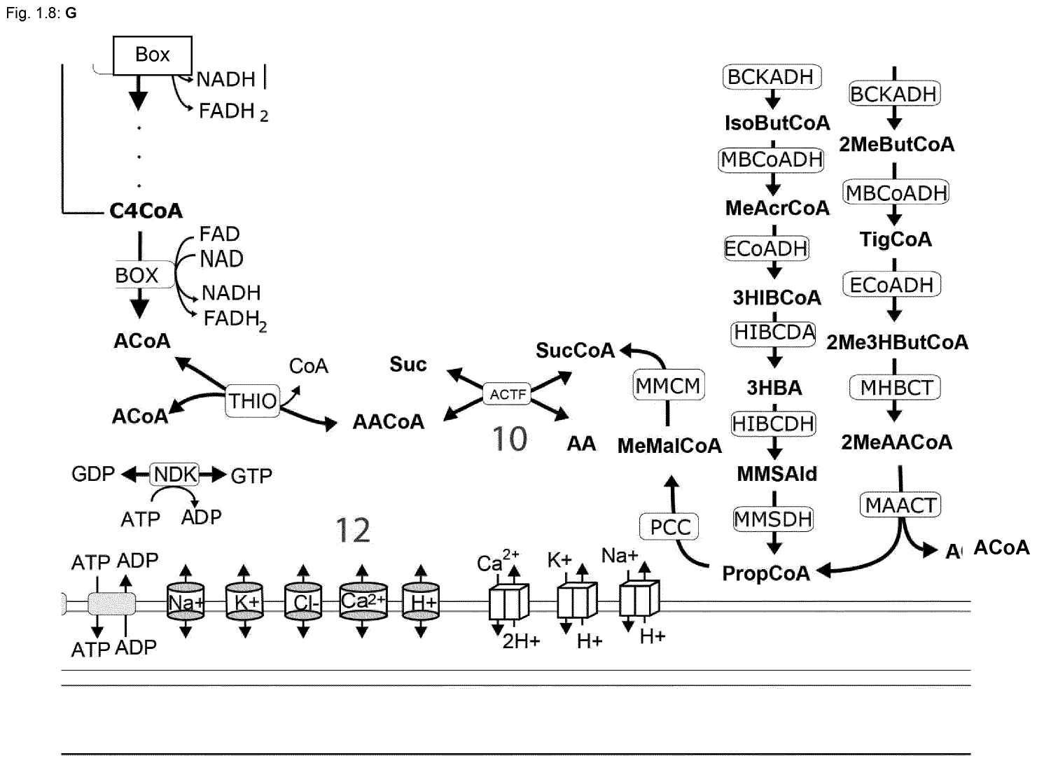

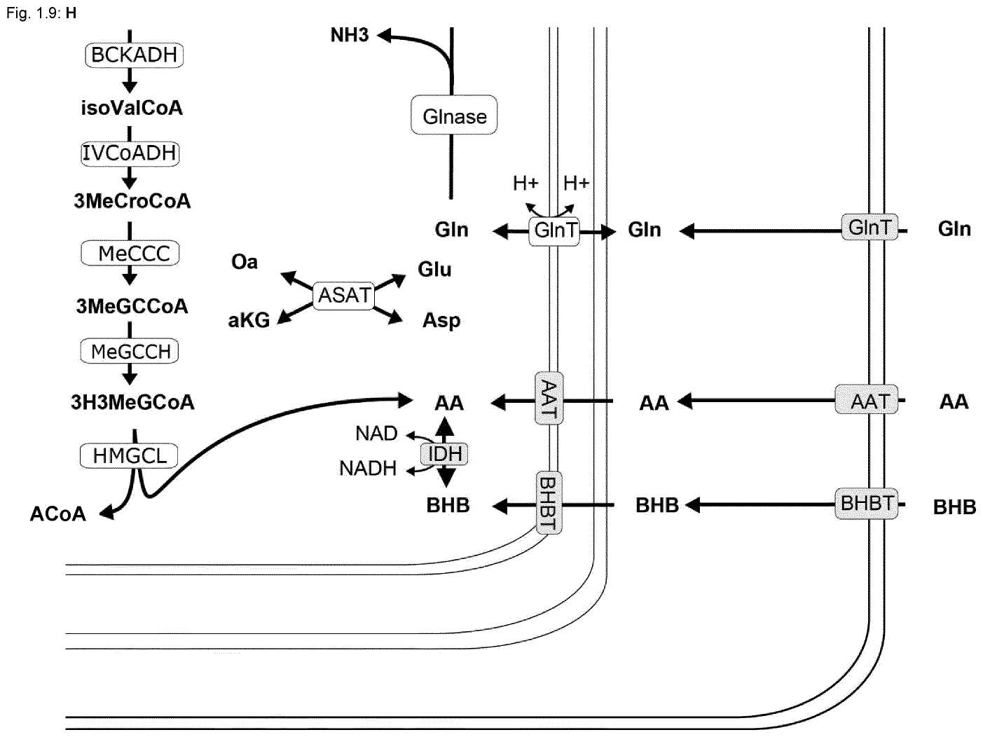

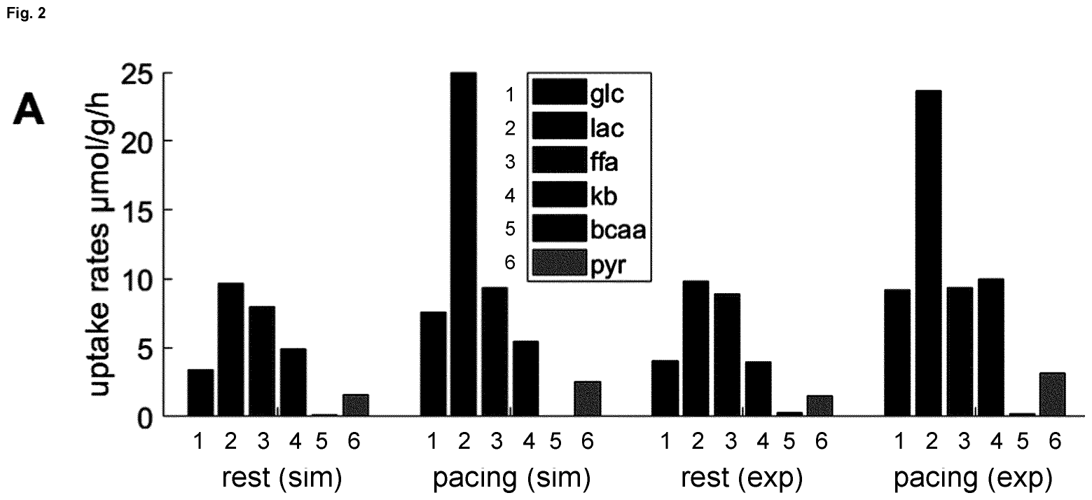

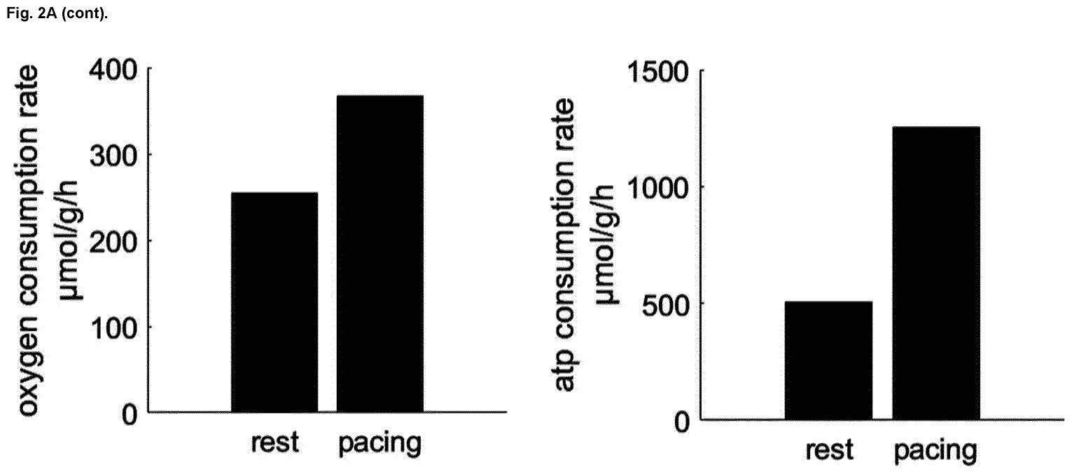

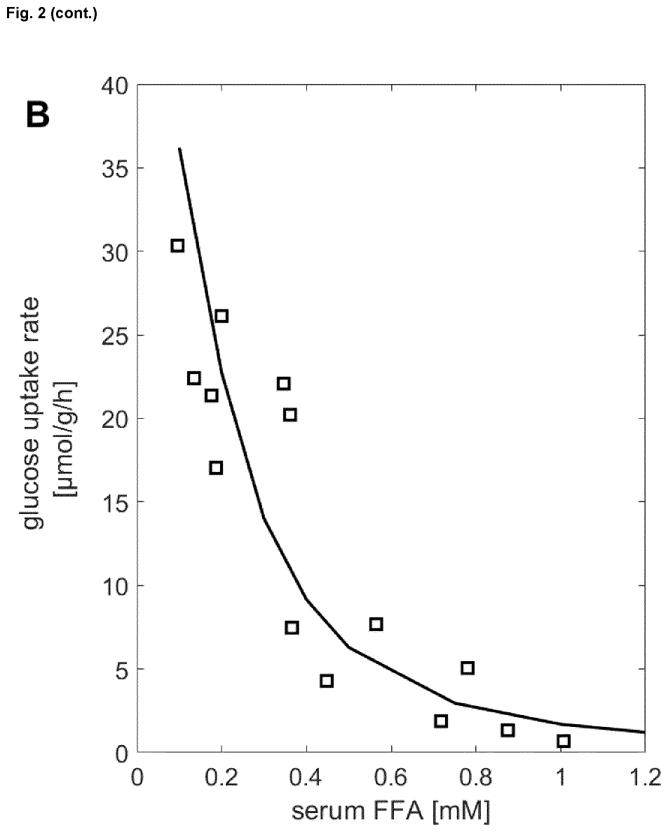

The mathematical model of the present invention shows a significant fit of model predictions to experimental data (FIG. 2). The examples demonstrate the ability of the heart to ensure cardiac functionality at varying cardiac workload and varying plasma concentrations of energy substrates.

In one embodiment, the individual cardiac metabolic profile comprises a substrate uptake rate, a myocardial ATP consumption, a myocardial ATP production reserve, a myocardial ATP production at said cardiac workload, and a myocardial ATP production at maximal workload, wherein the myocardial ATP production reserve is calculated as the difference between the myocardial ATP-production at said cardiac workload and the myocardial ATP production at maximal workload.

The present invention further relates to the use of the model for computation of the individual metabolic cardiac profile comprising computing a specific uptake rate of substrates, a specific ATP production rate at rest (MV_{ATP}(rest)), a specific ATP production rate at maximal ATP (MV_{ATP}(max)) workload, and myocardial ATP production reserve (MAPR).

Despite the high medical need, the state of the art currently does not provide means to determine the metabolic profile of the heart of a subject, e.g. the rate of ATP production MV_{ATP} in the heart and thus to assess the ability of the heart tissue to increase MV_{ATP} in response to an increase in ATP demand. Currently, no method is available to measure MV_{ATP} in vivo.

The myocardial ATP production reserve (MAPR) is calculated as the difference between the myocardial ATP-production at a cardiac workload and the myocardial ATP production at maximal workload. In some embodiments, the cardiac parameter can be determined for each cardiac workload.

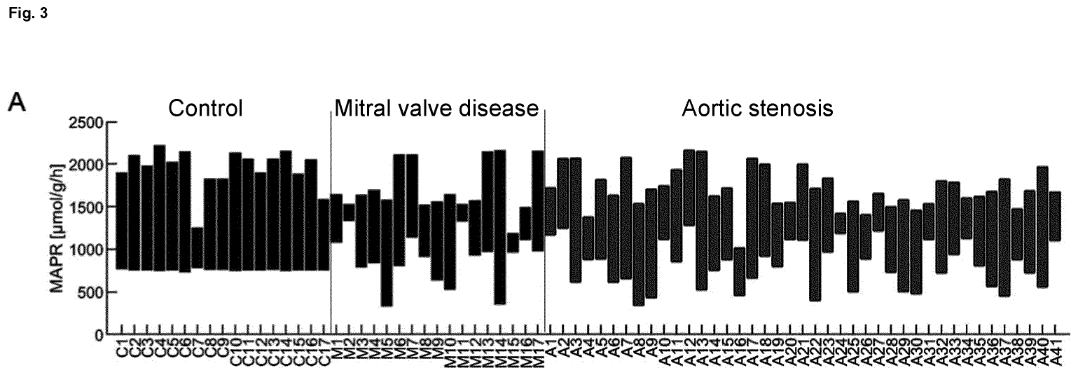

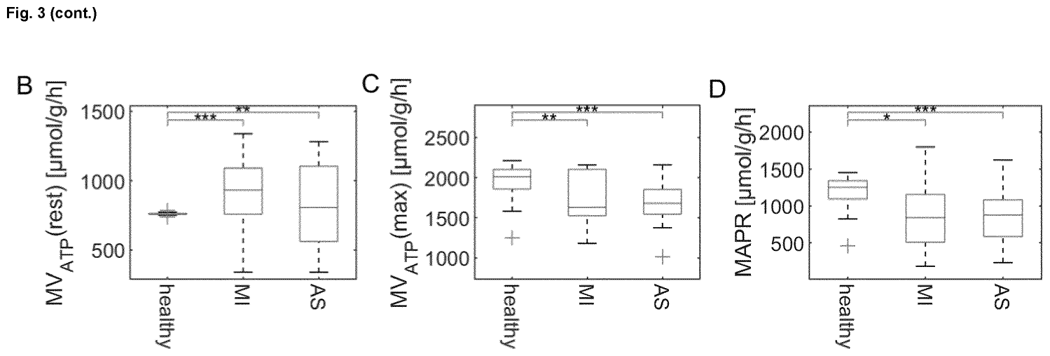

The specific energetic parameters MV_{ATP}(rest), MV_{ATP}(max) and MAPR after 60 min pacing are described in the Examples for each subject tested (FIG. 3).

As described in the Examples, the computed substrate uptake profile of the normal human heart is compared with the mean of experimental data taken from several in vivo studies (FIG. 2A). The glucose uptake can be correlated to the plasma FFA concentration (FIG. 2B).

As a further advantage, the mathematical model can be used to determine changes in the substrate preference and accompanying altered metabolic capacity of the heart at the physiological or at the pathological state, as shown in the example.

As described in the Examples, through the individual cardiac metabolic profile, a correlation can be achieved between (e.g., increased) ATP production capacity, (e.g., increased) mechanical work of the pressure/volume overloaded heart tissue (e.g., left ventricle), and cardiac output. This particular energetic state of the myocardium even in hearts, if compared to a non-diseased subject, provides clinical state and ventricular function of the heart tissue from the subject (patient).

In one embodiment, a plurality of said mathematical models can be used in said computations for the heart at physiological state, including normal post-absorptive, post prandial, and fasted, and for the heart at pathological state, including ischemic or diabetic.

In one embodiment, the computations can be performed for a normal post-absorptive state (overnight fast), as described in the Examples, characterized by the following metabolite and hormone: glucose, fatty acids, lactate, glutamine, valine, leucine, isosleucine, β-hydroxybutyrate, acetoacetate, and catecholamines at rest and at workload. The concentration of said metabolites and hormones may be obtained by the skilled person from a database, from the published literature, or from a suitable sample as described herein.

In one embodiment, the method can be used for calculating prognosis of a cardiovascular related disorder, an effect of a change in nutritional interventions, activity and/or therapeutic interventions on protein expression and on the time variation of a metabolic parameter in the heart tissue sample of the subject.

In one embodiment, therapeutic intervention, nutritional intervention, activity suitable for a subject that produces a result that in and of itself helps to prevent, to treat and/or to cure a disease. These include risk factor reduction (e.g., diet, exercise, stress reduction), pharmacologic therapy (drugs), acupuncture, invasive and interventional therapies as practiced by cardiologists and surgeons (e.g., bypass surgery, transcutaneous electric nerve stimulation (TENS), spinal cord stimulation (SCS)).

In one embodiment, the method is used to prevent, ascertain, prognose or treat a cardiovascular related disorder or to detect a perturbation of a normal biological state of the heart from the subject.

In one embodiment, a cardiovascular-related disorder can be selected from a group of, arrhythmias, vascular disease, myocardial infarction, heart failure, myocarditis, atherosclerosis, restenosis, coronary heart disease, coronary artery disease, atherosclerotic cardiovascular disease, arterial hypertension, cardiac fibrosis, stroke, sudden cardiac death syndrome, heart failure, ischemic heart disease, ischemic cardiomyopathy, myocardial infarction, coronary artery calcification.

In one embodiment, a symptom of a cardiovascular related discorder can be one of, but not limited to, long-term pressure, cardiac volume overload, cardiac dysfunction, myocardial infarction, myocardial hypertrophy congestive heart failure, survived cardiac arrest, arrhythmias, cardiovascular events, chest pain, palpitations (rapid rhythms or skips), breath disabilities, fatigue, and has an increased risk of death. In some embodiments, said patients suffer from valve diseases, e.g. aortic stenosis (AS) or mitral valve insufficiency (MI)

In one embodiment, a perturbation of a normal biological state of the heart from the subject can be one of, but not limited to, a reduced gene expression of key proteins involved in cardiac energy metabolism, increased gene expression of key proteins involved in cardiac energy metabolism, decreased levels of central metabolic enzymes, increased levels of central metabolic enzymes, reduced levels of cardiac energy-rich phosphates, elevated levels of cardiac energy-rich phosphates.

In one embodiment, a treatment is successful when the levels of protein markers, metabolites, hormones, and/or cardiac ATP capacity usually increase, provided that these levels were previously decreased compared to a reference. In one embodiment, a treatment is successful when the levels of protein markers, metabolites, hormones, and/or cardiac ATP capacity usually decrease, provided that these levels were previously increased compared to a reference.

In one embodiment, the method is preferably used for calculating prognosis of a mitral valve disease of said human subject, wherein the heart tissue sample used is preferably a ventricular septum sample of the heart of said subject.

In one embodiment, the method is preferably used for calculating prognosis of an aortic stenosis of said human subject, wherein the heart tissue sample used is preferably a ventricular septum sample of the heart of said subject.

In one embodiment, the method is preferably used for calculating occurrence of a mitral valve disease of said human subject, wherein the heart tissue sample used is preferably a ventricular septum sample of the heart of said subject.

In one embodiment, the method is used for calculating occurrence of an aortic stenosis disease of said human subject, wherein the heart tissue sample used is preferably a ventricular septum sample of the heart of said subject.

In one embodiment, the method is used for calculating the effects of a therapeutic intervention in a mitral valve disease of said human subject, wherein the heart tissue sample used is preferably a ventricular septum sample of the heart of said subject.

In one embodiment, method is used for calculating the effects of a therapeutic intervention in an aortic stenosis disease of said human subject, wherein the heart tissue sample used is preferably a ventricular septum sample of the heart of said subject.

In one embodiment, the method can be used for
(i) Selecting a nutritional or a therapeutic intervention, and
(ii) Evaluating or preventing a therapeutic intervention.

In one embodiment, the individual cardiac metabolic profile can be determined during the treatment for evaluating the effectiveness of the treatment.

In one embodiment, the individual cardiac metabolic profile can be determined before treatment for selecting a treatment.

In one embodiment, the individual cardiac metabolic profile can be determined after the treatment for evaluating effectiveness of the treatment, wherein said effectiveness of the treatment comprise an improved cardiac metabolism, improved cardiac output, activity tolerance, gene expression of cardiac genes at levels of physiological cardiac state, metabolite concentration at levels of physiological cardiac state, an increased myocardial ATP reserve, preferably an increased myocardial ATP production capacity as compared to myocardial ATP production capacity before treatment.

The invention further relates to a computer program adapted to execute a mathematical modelling algorithm that will be performed by a computing device/module to produce outputs of given data provided as inputs according to preceding claims, wherein said computer program, preferably MATLAB, is written in a programming language selected from a group comprising Fortran, C #, C/C++, High Level Shading Language, or Python.

The invention further relates to a computer program comprising instructions which, when the program is executed by a computer, cause the computer to carry out a mathematical modelling algorithm that will be performed by a computing device/module to produce outputs of given data provided as inputs described herein, wherein said computer program, preferably MATLAB, is written in a programming language selected from a group comprising Fortran, C #, C/C++, High Level Shading Language, or Python, and wherein the mathematical modelling algorithm provides cardiac energy expenditure profile for calculating prognosis of a cardiovascular related disorder, an effect of a change in nutritional interventions, activity and/or therapeutic interventions on protein expression and on the time variation of a metabolic parameter in the heart tissue sample of the subject.

The invention further relates to a computer program comprising instructions which, when the program is executed by a computer, cause the computer to carry out a mathematical modelling algorithm that will be performed by a computing device/module to produce outputs of given data provided as inputs described herein, wherein said computer program, preferably MATLAB, is written in a programming language selected from a group comprising Fortran, C #, C/C++, High Level Shading Language, or Python, and wherein the mathematical modelling algorithm provides cardiac energy expenditure profile and said profile is used for (i) selecting a nutritional or a therapeutic intervention, and/or (ii) evaluating or preventing a therapeutic intervention.

In some embodiments, a processor-readable medium there is provided comprising code representing instructions for causing a processor to use in one or more mathematical models one or more parameters related to determining the individual cardiac metabolic profile of a subject during a cardiac workload.

In one embodiment, input into the mathematical model data comprise the protein profile of a cardiac tissue sample, the cardiac parameters, and/or metabolites of the subject relating to determination of the individual cardiac metabolic profile, loading a reference data set.

In one embodiment, executing the algorithm for mathematical modelling comprise parameterizing and updating the models to the cardiac tissue sample of the subject so that the updating uses said data; so that the algorithms enable determination of the individual cardiac metabolic profile of a subject at a cardiac workload.

In one embodiment, output of the algorithm comprises the individual cardiac metabolic profile of a subject at a cardiac workload.

As described herein, determining an individual's metabolic cardiac profile is particularly useful for the clinical prognosis, evaluation or treatment of heart diseases.

Further examples of the computation-based method for determining an individual metabolic cardiac profile are described in detail below.

### DETAILED DESCRIPTION OF THE INVENTION

The present invention relates to a computation-based method for determining an individual metabolic cardiac profile in a subject.

All words and terms used herein shall have the same meaning commonly given to them by the person skilled in the art, unless the context indicates a different meaning. All terms used in the singular shall include the plural of that term and vice versa.

In accordance with the present disclosure, there is provided herein a computation-based method for determining an individual metabolic cardiac profile from a subject, and if compared to a non-diseased subject, used to prevent, ascertain, prognose or treat a cardiovascular related disorder or to detect a perturbation of a normal biological state of the heart, in particular heart failure, valve disease, e.g. aortic stenosis and mitral valve insufficiency.

In heart failure, gene expression of key proteins involved in cardiac energy metabolism as well as important metabolic enzymes (e.g., creatine kinase, CK) and cardiac energy-rich phosphates (ATP, CrP) are often downregulated. These findings suggest a mismatch between cardiac ATP demand and cardiac ATP production, and therefore in the individual metabolic cardiac profile. There is a high medical need to determine this mismatch to identify risks for heart diseases, such as valve disease, and/or heart failure. The individual cardiac metabolic profile comprises a substrate uptake rate, a myocardial ATP consumption, a myocardial ATP production reserve, a myocardial ATP production. It has proven very difficult in the art to determine the cardiac ATP production rate and the degree of reduction of cardiac ATP production in the heart tissue of a subject.

If such a method for determining the individual metabolic cardiac profile of a subject is possible, it may help to prevent, ascertain, prognose or treat a cardiovascular related disorder or to detect a perturbation of a normal biological state of the heart or possibly enables therapeutic inventions for reversing an associated disorder, such as a cardiovascular related disorder. Such a method is found herein for determining the individual metabolic cardiac profile of a subject according to the disclosure, which is further described below.

The individual metabolic cardiac profile, if compared to a non-diseased subject, providing information about cardiac metabolic changes in the heart from the subjects can be used for (i) selecting a nutritional or a therapeutic intervention, and (ii) evaluating or preventing a therapeutic intervention. Cardiovascular related disorders or a perturbation of a normal biological state of the heart is characterized by cardiac metabolic changes.

### Metabolites, Metabolic Cardiac Profile, Cardiac Parameter

In the determination of an individual metabolic profile for the assessment of the risk for the occurrence of cardiovascular diseases and/or changes in the physiological state of the heart, various clinical (“metabolites”, “cardiac parameter”) and individual parameters are usually determined.

For cardiovascular disease, “individual parameters”, as used herein, include patient age, smoking behavior (either the mere fact of being an (inhalant) smoker or the number of cigarettes per day), systolic and/or diastolic blood pressure, HDL cholesterol level (either concentration or particle number), blood glucose concentration, triglyceride concentrations, subject sex, and (blood pressure) medication.

A “metabolite” is defined as a substance being formed as intermediate or as degradation product of the subject's internal processes and can be selected from a group of glucose, lactate, pyruvate, glycerol, fatty acids, glutamate, glutamine, leucin, isoleucine, valine, acetate, B-hydroxybutyrate, catecholamines, creatine kinase, or insulin. In one embodiment, the metabolite can be determined in plasma. In one embodiment, the metabolite can be determined in blood. In one embodiment, the metabolite can be determined in serum. In one embodiment, the metabolite can be determined in the heart tissue sample. A metabolic parameter may vary with time. The “time variation of a metabolic parameter”, as used herein, comprises a change of the input value in a time course that both shifts the output signal in time and changes other parameters and behavior.

In one embodiment, the cardiac parameter, as used herein, comprise heart rate, blood pressure, pressure-volume loops, and/or heart power.

### Cardiovascular Disorder, Cardiac Workload, Cardiac Energy Expenditure

The terms “disorder” or “disease”, as used herein, can be used interchangeably.

As used herein, the terms “cardiovascular disorder”, “heart disease”, “cardiac disease” are interchangeable with, or at least related to, the terms “cardiopathy” or “cardiovascular related disorders”. Cardiovascular disorders are a large class of diseases that affect the heart and/or blood vessels (arteries and veins). Cardiovascular disorders include arrhythmias, vascular disease, myocardial infarction, heart failure, myocarditis, atherosclerosis, restenosis, coronary heart disease, coronary artery disease, atherosclerotic cardiovascular disease, arterial hypertension, cardiac fibrosis, stroke, sudden cardiac death syndrome, heart failure, ischemic heart disease, ischemic cardiomyopathy, myocardial infarction, coronary artery calcification. These diseases have similar causes, mechanisms, and treatments. Most cardiovascular disorders have common risk factors, including inflammation, fibrosis, diabetes, cholesterol, and vascular deposits. The terms “myocardial” and “cardiac” are used interchangeably.

The “myocardial ATP-production” refers to ATP levels produced by cardiac cells. Cellular ATP pools depend on the balance between ATP utilization and ATP production. The heart has an absolute requirement for aerobic ATP production to maintain adequate ATP concentrations because the anaerobic capacity of the heart is limited. Cellular ATP levels decrease when there is insufficient O2 for aerobic ATP production or when there is an increase in ATP utilization (increased ATP hydrolysis) that is not offset by a parallel increase in ATP synthesis.

The heart can use a variety of substrates for oxidative regeneration of ATP, depending on availability. In the postabsorptive state, several hours after a meal, the heart utilizes fatty acids (60-70%) and carbohydrates (˜30%). After a carbohydrate-rich meal, the heart may adapt to utilize almost exclusively carbohydrates (primarily glucose). Lactate can be used in place of glucose and becomes a very important substrate during exercise. The heart can also utilize amino acids and ketones instead of fatty acids. Ketone bodies (e.g. acetoacetate) are particularly important in diabetic acidosis.

The term “individual cardiac metabolic profile” comprises a substrate uptake rate, a myocardial ATP consumption, a myocardial ATP production reserve, a myocardial ATP production at cardiac workload, and a myocardial ATP production at maximal workload. “Cardiac workload”, as used herein, is termed as the utilization of the heart under a physiological condition, including rest, stress or cardiac pacing. In one embodiment, the “maximal workload” is also used as the highest utilization of the heart. The myocardial ATP production reserve is calculated as the difference between the myocardial ATP-production at a cardiac workload and the myocardial ATP production at maximal workload. In some embodiments, the cardiac parameter can be determined for each cardiac workload. The term “cardiac parameter”, as used herein, describes the quantitatively determined physical number useful for studying the activity and regulation of the heart, comprising ventricular end diastolic volume, ventricular end systolic volume, stroke volume, heart rate, cardiac output, preload, afterload, contractility, ejection fraction, blood pressure, pressure-volume loops, and/or heart power.

The term “pathological state” and “diseased” are used interchangeably and comprise ischemic or diabetic state of the heart. In one embodiment, the individual cardiac metabolic profile is determined for the heart at pathological state. In one embodiment, the individual cardiac metabolic profile is determined for the heart at physiological state. The terms “physiological state” and “normal biological state” are used interchangeably and comprise normal post-absorptive, post prandial, and fasted states of the heart. The normal biological state of the heart comprises maintenance of cardiac homeostasis.

The term “cardia metabolic derangement”, “metabolic changes in the heart”, “changes in cardiac metabolism” are used interchangeably and comprises changes in cardiac enzyme activity, cardiac gene expression, cardiac substrate uptake rate, cardiac hormone concentration (e.g. insulin, catecholamines), cardiac metabolite concentration, cardiac ATP consumption, cardiac oxygen consumption, cardiac NO, cardiac ion exchange, cardiac energy-rich phosphates, and/or cardiac ATP production capacity. “Changes in cardiac metabolism” are associated to a pathological state of the heart and may lead to congestive heart failure, compromised cardiac function, cardio-embolism, vascular and cardiac damage, diastolic dysfunction, cardiac dysfunction, cardiac valve disease, reduction of the cardiac output, exercise intolerance, conduction disturbances, and/or sudden death.

### Sample Preparation, Quantifying Metabolites, Peptides and Proteins

Quantitative proteomics requires the analysis of complex protein samples. In the case of cardiac metabolic profile determination, the ability to obtain appropriate samples for use in the mathematical model is important for the ease and accuracy of cardiac metabolic profiling.

A “provided” sample may have been obtained from another person and given to the person (or machine) performing the procedure. A “sample” (e.g., a test sample) from a subject means a sample that might be expected to have been obtained from a subject with cardiovascular disease or a non-diseased subject (“control,” “normal”). Many suitable sample types will be evident to a skilled worker. In one embodiment of the invention, the sample is a heart tissue sample. In one embodiment, the heart tissue sample can be selected from a group of a left ventricle, a right ventricle, a septum, a left atrium, a right atrium heart tissue sample obtained during a myocardium examination, a heart transplantation, an insertion of a pacemaker, an insertion of a defibrillator or a cardiac surgery, preferably a cardiac catheter examination. Methods for storing and lysing of heart tissue samples and protein extraction from heart tissue samples are well-known to a skilled worker. A preferred method storing and lysing of heart tissue samples and protein extraction from of heart tissue samples is provided in the Examples.

In one embodiment, the sample is a blood sample, such as whole blood, plasma, or serum (plasma from which clotting factors have been removed). For example, peripheral, arterial or venous plasma or serum may be used. In another embodiment, the sample is urine, sweat, or other body fluid in which proteins are sometimes removed from the bloodstream. In one embodiment, metabolites are determined in blood samples. In one embodiment, hormones are determined in blood samples.

Protein quantities are a number, e.g. an integral number, a decimal number, of proteins determined by an appropriate protein quantification method well-known to a skilled worker method or obtained from a public database or obtained from published literature. Peptide quantities are a number, e.g. an integral number, a decimal number, of proteins determined by an appropriate protein quantification method well-known to a skilled worker method or obtained from a public database or obtained from published literature. Examples for proteins and peptides with corresponding concentrations are provided herein. Metabolite concentrations are a number, e.g. an integral number, a decimal number, of proteins determined by an appropriate quantification method well-known to a skilled worker method or obtained from a public database or obtained from published literature. Examples for metabolites and corresponding concentrations are provided herein.

In one embodiment, the protein profile of the heart tissue sample is determined by a method, as provided by the Examples described herein, wherein the method comprises (i) solubilizing the heart tissue sample, (ii) extracting proteins from solubilized heart tissue sample of step (i) according to the protein quantification method, wherein said proteins are preferably fragmented into peptides, (iii) transferring said extracted proteins and/or peptides from step (ii) to a device, preferably a mass spectrometer, of said protein quantification method identifying and quantifying the proteins and/or peptides in said sample, preferably the peptides.

The properties and amino acid sequences of the proteins in the protein profiles of the subject are well-known and can be determined routinely, as well as downloaded from various known databases. See. e.g., the database, International Protein Index (IPI) at the world wide web site, ebi.ac.uk/IPI/xrefs.html, https://prosite.expasy.org. Information to some of the proteins discussed herein, is provided in the Examples. This information is accurate as of the date of filing of this application. Although much of the data presented in the Examples herein are directed to particular forms of proteins of interest (or peptides thereof), it will be evident to a skilled worker that a variety of forms of these proteins may be indicative of the presence of cardiovascular-related disorder in a subject. For example, the protein may be an intact, full-length protein. If a protein undergoes processing naturally (e.g., is converted from a pre-pro-hormone to a pro-hormone to a fully processed hormone; the N-terminal methionine is cleaved off; the signal sequence is removed, often accompanied by a post-translational modification, such as acetylation; etc.). Furthermore, in some instances, a protein of the invention may be broken down or degraded (e.g., proteins that are found in the urine). In such a case, an investigator can determine the level of one or more of the fragments or degradation products. A “peptide,” as used herein, refers to sequence of two or more amino acids, generally derived from a larger polypeptide or protein. The peptide is unique to the protein being identified, as detected by a method described herein. A “significant” difference in a value, as used herein, can refer to a difference which is reproducible or statistically significant, as determined using statistical methods that are appropriate and well-known in the art, generally with a probability value of less than five percent chance of the change being due to random variation. In general, a statistically significant value is at least two standard deviations from the value in a “normal” control subject or reference. Suitable statistical tests will be evident to a skilled worker. For example, a significant difference in the amount of a protein compared to a baseline value can be about 50% less, or 2-fold higher.

It is generally not practical in a clinical or research setting to use patient samples as sources for baseline controls. Therefore, one can use any of variety of references as described herein. Those skilled in the art are aware that a baseline or normal value need not be established for each method for determining proteins, peptides, hormones, or metabolites when it is performed, but that reference or normal values may be established by reference to some form of stored information about a previously determined reference value for a particular protein or panel of proteins, such as a reference value established by one of the methods described. Such a form of stored information may include, for example, a reference table, a listing or electronic file of population or individual data relating to “normal values” (control) or positive controls, a medical record for the patient in which data from previous evaluations are recorded, a receiver operator characteristic (ROC) curve, or any other source of data relating to reference values that is useful to the patient. In an embodiment in which the progress of a treatment is monitored, a reference value may be based on previous measurements of the same subject before the treatment was administered.

In one embodiment, the protein quantification method can be selected from a group of large-scale protein quantification, mass spectrometry, large scale mass spectrometry, immunoassay, Western blot, microfluidics/nanotechnology sensor, and aptamer capture assay, preferably large scale mass spectrometry such as inductively coupled plasma mass spectrometry, MALDI-MS/MS, LC-MS, LC-MS/MS, and ESI-MS/MS. In one embodiment, the protein quantification method is a large-scale protein quantification method that can be selected from a group of SILAC, ICAT, NeuCode SILAC, Label-free, Metal-coded tags (MeCAT), TMTduplex, TMTsixplex, TMT10plex and TMT11plex, and aminoxyTMT measured using a mass spectrometry technique.

As described herein, left ventricular septum biopsies specimens were taken from patients admitted to clinic in need for aortic or mitral valve replacement surgery or from healthy heart control subjects (controls). Protein extraction and quantitative proteomics was performed as described in the Examples. In one embodiment, the methods for protein extraction and quantitative proteomics described herein are preferably used in the present invention.

Among the many types of suitable immunoassays are immunohistochemical staining, ELISA, Western blot (immunoblot), immunoprecipitation, radioimmuno assay (RIA), fluorescence-activated cell sorting (FACS), etc. Assays used in a method of the invention can be based on colorimetric readouts, fluorescent readouts, mass spectrometry, visual inspection, etc. Assays can be carried out, e.g., with suspension beads, or with arrays, in which antibodies or cell or blood samples are attached to a surface such as a glass slide or a chip.

In one embodiment, mass spectrometry is used to determine the amount of a protein or a peptide. Mass spectrometry (MS) can also be used to determine the amount of a protein, using conventional methods. Some typical such methods are described in the Examples herein. Relative ratio between multiple samples can be determined using label free methods (as done in the present Examples), based on spectral count (and the number of unique peptides and the number of observations of each peptide). In the Examples herein, an Orbitrap Fusion (individual samples) and Q Exactive HF-X Orbitrap instrument (reference sample) was used (LC/MS/MS instrument to obtain the data. Alternatively, quantitive data can be obtained using multiple reaction monitoring (MRM), most often carried out using a triple quadripole mass spectrometer. In this case, peptides that are unique to a given protein are selected in the MS instrument and quantified. Absolute quantification can be obtained if a known labeled synthetic peptide is used. For detailed methods see, e.g., Qin Fu and JE Van Eyk, in Clinical Proteomics: from diagnostics to therapy (Van Eyk J E and Dunn M, eds), Wiley and Son Press; Current Protocols in Molecular Biology, Preparation of Proteins and Peptides for Mass Spectrometry Analysis in a Bottom-Up Proteomics Workflow, Gundry et al., chapter 10, 2009).

### MS Data Analysis

MS data are preferably analysed using MaxQuant sofware package. For searching MS² spectra against a decoy human UniProt database (HUMAN.2019-01, with isoform annotations) containing forward and reverse sequences, the internal Andromeda search engine is preferably used. The search included variable modifications of oxidation (M), N-terminal acetylation, deamidation (N and Q) and fixed modification of carbamidomethyl cysteine. Minimal peptide length was set to six amino acids and a maximum of three missed cleavages was allowed. The FDR (false discovery rate) was set to 1% for peptide and protein identifications. Unique and razor peptides were considered for quantification. Retention times were recalibrated based on the built-in nonlinear time-rescaling algorithm. MS² identifications were transferred between runs with the “Match between runs” option, in which the maximal retention time window was set to 0.7 min. The integrated LFQ quantitation algorithm was applied. Gene Symbols assigned by MaxQuant were substituted with gene symbols of the reported UniProt IDs from the FASTA file used.

In general, molecular biology methods referred to herein are well-known in the art and are described, e.g., in Sambrook el al., Molecular Cloning: A Laboratory Manual, current edition, Cold Spring Harbor Laboratory, Cold Spring Harbor, NY, and Ausubel et al., Current Protocols in Molecular Biology, John Wiley & sons, New York, NY.

The computation-based method of the invention can be adapted for many uses. For example, it can be used to follow the progression of cardiovascular related disorders. In one embodiment of the invention, the detection is carried out both before (or at approximately the same time as), and after, the administration of a treatment, and the method is used to monitor the effectiveness of the treatment. A subject can be monitored in this way to determine the effectiveness for that subject of a particular drug regimen, or a drug or other treatment modality can be evaluated in a pre-clinical or clinical trial.

In one embodiment, a treatment is successful when the levels of protein markers, metabolites, hormones, and/or cardiac ATP capacity usually increase, provided that these levels were previously decreased compared to a reference. In one embodiment, a treatment is successful when the levels of protein markers, metabolites, hormones, and/or cardiac ATP capacity usually decrease, provided that these levels were previously increased compared to a reference.

Data Analysis: Mathematical (Kinetic) Model and Applying that Mathematical Mode

Mathematical modelling and applying that mathematical mode to data, parameter, references is preferably carried out using a version of the published mathematical algorithm [19].

In one embodiment, a mathematical model of cardiac energy metabolism is used to quantify the metabolic changes caused by the abundance changes of metabolic enzymes. In one embodiment, the mathematical model of cardiac energy metabolism includes all pathways involved in the catabolism of the energy-providing substrates glucose, lactate, fatty acids, KBs, and BCAAs, as well as in the synthesis of endogenous energy stores (glycogen, triacylglycerol). Kinetic data, pathways, metabolite fluxes, ion fluxes, and protein abundances can be downloaded from public databases and are well-known to the skilled-person, e.g., KEGG ENZYME, NIST Chemical Kinetics Database, SABIO Biochemical Reaction Kinetics Database, BRENDA, DAVID.

In one embodiment, the mathematical model also takes into account the short-term regulation of metabolic enzymes and transporters, e.g. by the hormones insulin and catecholamines. In one embodiment, the mathematical model also incorporates electrophysiological processes at the inner mitochondrial membrane including the generation of the proton gradient by the respiratory chain, the synthesis of ATP by FoF1-ATPase, and the membrane transport of various ions.

### Kinetic and Enzyme Activity

Enzyme kinetics describes the study of rates of enzyme-catalyzed chemical reactions, measuring reaction rates and examining the effects of varying reaction conditions. Michaelis-Menten kinetics is one common model of enzyme kinetics.

In some embodiments, the time course of the concentration of metabolites and ions can be determined by first-order differential equations. The term “first order” usually means that the first derivative appears, but no derivatives of higher order. A first order differential equation may be an equation of the form dx/dt=f(x, t), where x denotes the vector of metabolites and hormones and t denotes the time.

In some embodiments, the time variation of small ions is modeled by Goldman-Hodgkin-Katz type kinetic equations, according to the publication of the inventors [Peterzan, M. A., et al 2020]. In one embodiment, the rate laws for enzymes and membrane transporters were either taken from the literature. In another embodiment, the rate laws for enzymes and membrane transporters were constructed based on published experimental data for the mammalian heart. The “Goldman” equation or “Goldman-Hodgkin-Katz” equation after David Eliot Goldman (1910-1998), Alan Lloyd Hodgkin and Bernard Katz can be used for calculating the membrane potential considering multiple permeating ions. The Goldman equation allows the calculation of a membrane potential for a membrane permeable to different ions, including sodium, potassium, calcium, and chloride ions. The Goldman equation is based on the principle of a steady state. The sum of all ionic currents must equal zero. Other assumptions of the Goldman equation are the independence of the ions from each other, and a linear decrease of the potential across the membrane thickness—because of the resulting constant field, this is often referred to as a “constant field equation”. It makes allowance for the fact that at rest membrane potentials the currents pass through individual channels. In the Goldman equation, the ion current is approximated as a function of ion concentration and a coefficient called permeability P. The permeability is derived from Fick's law of diffusion. Goldman, D. E. (1943): Potential, impedance, and rectification in membranes. In: Journal of General Physiology, 27:37-60]. In addition to the Goldman-Hodgkin-Katz equation, various calculations may be used, as well as Ohm's law, to further refine the model of transmembrane potential difference, such as the “Nernst” or “constant field” equation and Gibbs-Donnan equilibrium. See Selkurt, ed. (1984) Physiology 5th Edition, Chapter 1, Little, Brown, Boston, Mass. (ISBN 0-316-78038-3) and Hille at e.g., chapters 10-13.

The Goldman-Hodgkin-Katz voltage equation describes how the various ion gradients contribute to the resting membrane potential of a cell permeable to potassium, sodium, and chloride ions.

It shows that the membrane potential of a cell is not only determined by the quotients of the ion concentrations on both sides of the cell membrane, but primarily by the permeability (P) of the membrane for the respective ion. The greater the permeability (often also called conductivity) of the membrane for a particular ion, the greater the contribution of this ion to the membrane potential. Thus, in a sense, the membrane potential represents the weighted average of the equilibrium potentials for the various ions.

The permeability of the membrane to the particular ion species is determined solely by the number and activity of the corresponding ion channels conducting that ion. In the resting state, only very few open sodium, chloride or calcium channels are found in the cell membrane.

A change in the membrane potential of a cell occurs when the permeability of the membrane to an ion species changes. For example, activation of sodium channels shifts the membrane potential toward the equilibrium potential for sodium—the membrane potential becomes more positive. The membrane potential can also take on a new value when the intracellular or extracellular concentration of an ion changes. The membrane potential is affected by the extracellular concentration of potassium, sodium, chloride or calcium.

Ion channels are found in every cell type of an organism, with potassium channels being the largest and most diverse ion channel family. The activity is modulated by different physiological stimuli depending on the channel type, e.g., membrane potential changes (voltage-gated channels), G-proteins, calcium ions, nucleotides (ATP), etc. In most cells, the resting membrane potential is determined by potassium channels. Potassium channels decisively influence the frequency and time course of action potentials, as well as their transmission, especially in neurons and muscle cells, including cardiac muscle cells. They also regulate the electrical excitability of these cells and they play a crucial role in some secretory and metabolic processes, such as insulin secretion.

Drugs/active ingredients, food intake, and physical activity can affect the activity of potassium channels in different ways. For example, drugs with a channel blocker as the active ingredient usually block the channel pore directly from the intracellular or extracellular side of the membrane. In addition, however, the interaction of a channel blocker with an accessory subunit, for example, can cause the channel pore to close or not be opened by physiological stimuli. Disturbances in the opening and closing of ion channels in cardiac muscle cells can lead to disturbances in cardiac function and thus to cardiac diseases, such as cardiac arrhythmias and hypertension. Therefore, the provision of the mathematical model for the determination of the individual cardiac metabolic profile of the present invention, which includes the activation and inactivation of ion channels, has an enormous potential for the development of new and highly effective therapeutic concepts.

The term “reference data” as used here includes primary data for all inputs, parameters, quantities, kinetic data, model variables (dependent or independent), even under different workloads. In some embodiments, reference data comprise published experimental data of mammalian hearts, comprise literature data, experimental data of mammalian tissue sample, preferably heart tissue sample, at physiological state and/or experimental data of mammalian tissue sample, preferably heart tissue sample, at pathological state. Reference data sets are usually stored in databases.

### Model Parametrization

“Mathematical model parametrizing”, as used herein, also describes finding and fitting a set of model parameters that describe the system and its behavior, and can usually be achieved by cross-referencing model predictions with actual measurements on the system, wherein this cross-referencing can be a comparison with a reference and/or a control. In one embodiment, model parameters, including kinetic rate constants, substrate affinities, affinities for allosteric regulators, were taken from reported kinetic studies of the isolated enzyme from mammalian heart tissue.

In some embodiments, the mathematical model was parametrized for individual heart tissue sample by proteomics-derived protein profiles of enzymes and transporter proteins by computing the maximal activity (Vₘₐₓ) of the enzyme by the equation 1

$${V}_{ma x}^{subject} = {V}_{m ax}^{normal} \frac{{E}^{subject}}{〈 {E}^{control} 〉} \qquad ( 1 )$$

wherein [symbol] E^{control} [symbol] describes the average protein intensity of the enzyme derived from heart tissue samples of control subjects and E^{subject} describes the protein concentration of the enzyme in the individual subject, wherein said individual subject can be control or patient.

The “Vₘₐₓ” refers to the maximal activity of an enzyme that is related to the protein concentration (E) by

$${v}_{max} = {k}_{cat} E \qquad ( 2 )$$

wherein k_{cat} is the catalytic rate constant (“turnover number”) of the enzyme/transporter.

The maximal activities vₘₐₓ^{normal} of the reference data set comprising the average of heart tissue samples of control subjects were obtained by fitting of the model to experimental data. Equation (2) deduces that the maximum enzyme activity is proportional to the abundance of the protein.

In one embodiment, Vₘₐₓ values may vary due to variable protein profiles of subjects.

In one embodiment, the Vₘₐₓ values indicating the maximal activity of each enzyme are estimated by fitting the model to measurements of exchange fluxes and internal metabolites obtained in different experimental setups. In one embodiment, the validity of the model is tested by comparing the simulated exchange fluxes and metabolite concentrations with experimental data. Since the heart switches its substrate uptake rates depending on substrate availability, different simulations with variable substrate availability must be performed. Model simulation for selected substrate uptakes, e.g. glucose are given in the Examples.

### Energetic Capacities

The present invention further relates to the use of the model for computation of the individual metabolic cardiac profile comprising computing a specific uptake rate of substrates, a specific ATP production rate at rest, a specific ATP production rate at maximal ATP workload. The “substrate” also refers to a reactant in a chemical reaction processed by an enzyme. The term “individual metabolic cardiac profile”, as used herein, also refers to the energetic capacity of the heart from the subject. In the Examples, the computation of the energetic capacities of controls and patients with valve disease, aortic valve stenosis or mitral valve insufficiency, is provided.

The term “MV_{ATP}(rest)”, as used herein, refers to the specific ATP production rate at rest. The term “MV_{ATP}(max)”, as used herein, refers to the specific ATP production rate at maximal ATP workload. The term “MAPR” (equation 3), as used herein, also refers to as myocardial ATP production reserve and characterize the capacity of the heart tissue to increase the ATP production with increasing workload

$$MAPR = {MV}_{ATP} ( max ) - {MV}_{ATP} ( rest ) \qquad ( 3 )$$

The term “specific energy parameters”, as used herein, subsumes MV_{ATP}(rest), MV_{ATP}(max), and MAPR for quantifying the energetic capacity per mass unit of the heart tissue sample (given in μmol/g/h). The term “total energy parameters as used herein, subsumes tMV_{ATP}(rest), tMV_{ATP}(max), and tMAPR for quantifying the energetic capacity of the heart tissue sample (given in mmol/h).

$${tMV}_{ATP} ( rest ) = {MV}_{ATP} ( rest ) \times heart tissue sample mass / 1000 \qquad ( 4 )$$

$${tMV}_{ATP} ( max ) = {MV}_{ATP} ( max ) \times heart tissue sample mass / 1000 \qquad ( 5 )$$

$$tMAPR = MAPR \times heart tissue sample mass / 1000 \qquad ( 6 )$$

In one embodiment, the computations can be performed for a normal post-absorptive state (overnight fast), as described in the Examples, characterized by the following metabolite and hormone: glucose, fatty acids, lactate, glutamine, valine, leucine, isosleucine, β-hydroxybutyrate, acetoacetate, and catecholamines at rest and at workload. The concentration of said metabolites and hormones may be obtained by the skilled person from a database, from the published literature, or from a suitable sample as described herein.

As described in the Examples, subject's MV_{O2} was estimated by the 2-factor approximation as described in Nelson et al. 1974 [31], wherein said subject's oxygen consumption MV_{O2} was used as value for computing the cardiac ATP consumption of the stationary resting state, MV_{ATP}(rest).

$${MV}_{O 2} = γ \cdot HR \cdot BP \qquad ( 7 )$$

“HR” refers to the heart rate. “BP” refers to the peak systolic blood pressure. “y” refers to a proportionality factor. As described in the Examples, the resting MV_{O2} of normal hearts was found in the range of 0.8-1.2 ml/min/g [2-4]. Thus, with a mean MV_{O2}=0.1 ml/min/g, HR=70/min and normal BP=125 mm, we set γ to 1.143×10-5 ml/mmHg/g.

In one embodiment, the metabolic response of the myocardial sample to an additional workload (pacing) was assessed by calculating the temporal changes in metabolic state triggered by an increase in ATP consumption rate above resting levels.

In one embodiment, the ATP consumption rate is also modeled by a generic hyperbolic rate law:

$${v}_{ATP} = {k}_{load} \cdot \frac{ATP}{ATP + {K}_{m}} \qquad ( 8 )$$

(see Examples). In one embodiment, the continuous parameter k_{load} is also a natural number denoting the energetic demand, that can be stepwise increased until MV_{ATP} converged to the maximum, MV_{ATP}(max).

As an Example, the kinetic rate equation for the “Carrier mediated FATP” (“CD36”) can be calculated as

$${v}_{CD 36} = {V}_{max}^{CD 36} \cdot \frac{{ffa}_{ext} - c {16}_{cyt}}{{1}^{′} + \frac{{ffa}_{ext}}{{K}_{m}^{{ffa}_{ext}}} + \frac{c {16}_{cyt}}{{K}_{m}^{c {16}_{cyt}}}} \qquad ( 9 )$$

$${V}_{max}^{CD 36} = 4 , 20 E + 01 μmol / g / h$$

$${K}_{m}^{{ffa}_{ext}} = 0.000085 [ 61 ]$$

$${K}_{m}^{c {16}_{cyt}} = 0.004 [ 62 ] ,$$

wherein “ffa” refers to free fatty acid, “ext” refers to extracellular, “cyt” refers to “cytosolic, “c16” refers to long-chain fatty acid with a 16-carbon backbone, e.g. palmitic acid.

In one embodiment, a mechanical burden of the heart is evaluated, wherein the internal myocardial power, which describes the energy required for cardiac contraction for the individual hearts, is calculated as described in the methods used in Lee et al. [32].

In one embodiment, model simulations are performed using MATLAB.

### Computer Program

A further aspect of the invention relates to a computer program adapted to execute a mathematical modelling algorithm that will be performed by a computing device/module to produce outputs given data provided as inputs according to preceding claims, wherein said computer program, preferably MATLAB, is written in a programming language selected from a group comprising Fortran, C #, C/C++, High Level Shading Language, or Python.

In some embodiments, a “computer” or “computing device” may be used. Such a computer may be, for example, a mainframe computer, a desktop computer, a notebook or laptop computer, a portable device such as a data acquisition and storage device, or a processing device integrated into another device such as a scanner for tomography. In some cases, the computer may be a “dumb” terminal used to access data or processors over a network.

In some embodiments of the present invention, the mathematical model comprises processor-readable media comprising: one or more parameters relating to the cardiac tissue sample and/or the subject; one or more kinetic models, preferably cardiac kinetic model, protein profile data comprising data points relating to the cardiac tissue and/or the subject such that an update uses the data; one or more algorithms using one or more of the parameters, one or more kinetic models, preferably cardiac kinetic model, and the data as input, such that the algorithms enable determination of the individual cardiac metabolic profile; and code to implement the algorithms.

Also provided are one or more processors in communication with the processor-readable medium and configured to execute the code. In some embodiments of the present invention, a method is provided for applying a mathematical model that relates a state to time for a cardiac tissue sample of a subject. In some embodiments of the present invention, there is provided a method for applying a mathematical model that relates a condition for a cardiac tissue sample of a subject to cardiovascular disease or cardiovascular disorder.

In some embodiments of the present invention, the mathematical model uses the MATLAB program. In some embodiments of the present invention, there is provided a method for implementing a mathematical model that produces and updates a mathematical relationship between a protein profile of a cardiac tissue sample, parameters related to subject, metabolite concentration, and ion concentration, and a time course. In some embodiments of the present invention, there is provided a method for implementing a mathematical model that produces and updates a mathematical relationship between a protein profile of a cardiac tissue sample, parameters related to the subject, metabolite concentration, and ion concentration, and a cardiovascular disease or cardiovascular disorder. The procedure includes: 1) selecting a cardiac workload of a subject 2) selecting the protein profile of a cardiac tissue sample, cardiac parameters, and/or metabolites of the subject for determining the individual cardiac metabolic profile; 3) selecting at least one kinetic model, preferably a cardiac kinetic model; 4) selecting at least one parameter related to the kinetic model; 5) selecting a reference data set; 6) loading the reference data set containing the sets of data entries, wherein each data entry of the set contains at least one correlated compatible protein label and/or metabolite label; 7) inputting the protein profile of a cardiac tissue sample, the cardiac parameters, and/or metabolites of the subject; 8) calculating the subject's maximum enzyme activity Vmax, where Vmax is calculated according to formula X using the inputs from steps 6 and 7; 9) parameterizing the mathematical model to the subject's cardiac tissue sample by applying the subject's Vmax from step 8; 10) rerunning the model; 11) calculating a subject's cardiac energy expenditure profile that includes the data points from step 7; 12) comparing to a non-diseased subject at cardiac workload; 13) determining a subject's individual cardiac metabolic profile using steps 11 and 12; and 14) repeating steps 1,2, 7 through 12 as needed to update the model during determination of a subject's individual cardiac metabolic profile, for example at a different cardiac workload.

In some embodiments of the present invention, there is provided a processor-readable medium comprising code representing instructions for causing a processor to use in one or more mathematical models one or more parameters related to determining the individual cardiac metabolic profile of a subject during a cardiac workload; inputting into the models data comprising the protein profile of a cardiac tissue sample, the cardiac parameters, and/or metabolites of the subject relating to determination of the individual cardiac metabolic profile, loading a reference data set; parameterizing and updating the models to the cardiac tissue sample of the subject so that the updating uses said data; so that the algorithms enable determination of the individual cardiac metabolic profile of a subject at a cardiac workload.

The term “input” as used herein, describes a function to enter data for applying a mathematical model and returns a reference to the data in the form of a string. Data comprise protein quantities, peptide quantities, protein labels, cardiac parameter labels, numeric cardiac parameter, clinical laboratory parameter labels, numeric clinical laboratory parameter, numeric metabolites, metabolite label, cardiac workload label, cardiac kinetic model. The term “output”, as used herein, describes a value produced by an algorithm, preferably a human readable value, more preferably a value defining a metabolic cardiac profile.

The term “plurality”, as used herein, means the state of being numerous.

Through a mathematical model, problems and its instances (in the algorithmic sense) may be formulated, and analyzed for properties. An algorithm is usually a step-by-step process with well-defined steps, and takes an input instance of a problem instance (a mathematical model) and produces an output, wherein an algorithm can be implemented into a computer program. A computer program is generally a series of instructions, complying with the rules of a particular programming language, to perform or solve certain functions or tasks or problems with the help of a computer.

The term “execute” also means the process by which a computer or virtual machine executes the instructions of a computer program, written in a programming language, to see the output, including, wherein the programming language can be selected from a group of Java, Fortran, C, C++, Python, C #, JavaScript, VB .NET, R, PHP, High Level Shading Language, and MATLAB. In one embodiment, the mathematical modelling algorithm for calculating individual metabolic cardiac profile is programmed and executed in MATLAB or any distribute of MATLAB. In one embodiment, the mathematical modelling algorithm for calculating individual metabolic cardiac profile is programmed and executed in Python or any distribute of Python.

### Medical Indications and Therapy:

In one aspect of the present invention there is provided an individual metabolic cardiac profile according to the invention as herein described for use in the treatment of a medical condition associated with changes in cardiac metabolism, wherein the medical condition associated with changes in cardiac metabolism is preferably a cardiovascular related disorder, cardiac ATP production capacity associated cardiovascular related disorder or a cardiovascular pathology.

As used herein, the “patient” or “subject” refers to a human, preferably a patient receiving dialysis, but can also be any other mammal, such as a domestic animal (e.g. a dog, cat or the like), a farm animal (e.g. a cow, sheep, pig, horse or the like) or a laboratory animal (e.g. a monkey, rat, mouse, rabbit, guinea pig or the like). The term “patient” preferably refers to a “subject” suffering from or suspected of suffering from a cardiovascular disease and/or changes in cardiac metabolism.

In the present invention “treatment” or “therapy” generally means to obtain a desired pharmacological effect and/or physiological effect. The effect may be prophylactic in view of completely or partially preventing a disease and/or a symptom, for example by reducing the risk of a subject having a disease or symptom or may be therapeutic in view of partially or completely curing a disease and/or adverse effect of the disease. In the present invention, “ascertain” means to discover cardiovascular disease, cardiac ATP production capacity associated cardiovascular related disorder or a cardiovascular pathology, abnormal cardiac ATP production, and/or changes in cardiac metabolism. In the present invention, the individual metabolic cardiac profile for “prognosis” comprise to predict the course of a disease or to predict the effect of a treatment. The term “evaluating”, as used herein, usually means determining whether expected outcomes were met and comprise measuring effectiveness of a medical care, a treatment, a nutritional intervention, and a physical activity.

In the present invention, “therapy” includes arbitrary treatments of diseases or conditions in mammals, in particular, humans, for example, the following treatments (a) to (c): (a) Prevention of onset of a disease, condition or symptom in a patient; (b) Inhibition of a symptom of a condition, that is, prevention of progression of the symptom; (c) Amelioration of a symptom of a condition, that is, induction of regression of the disease or symptom.

The phrase “therapeutically effective” is intended to include, within the scope of sound medical judgment, excessive toxicity, irritation, allergic reactions, and/or other problems or complications, but commensurate with a reasonable benefit/risk ratio. As used herein to refer to the therapeutic invention, nutritional intervention, activity suitable for a subject that produces a result that in and of itself helps to prevent, to treat and/or to cure a disease. These include risk factor reduction (e.g., diet, exercise, stress reduction), pharmacologic therapy (drugs), acupuncture, invasive and interventional therapies as practiced by cardiologists and surgeons (e.g., bypass surgery, transcutaneous electric nerve stimulation (TENS), spinal cord stimulation (SCS)).

In particular, the treatment relates to prevent or ameliorate cardiovascular related disorders or cardiac metabolic changes either by activity, nutritional intervention, cardiac surgery, drug therapy, mechanic therapeutic intervention, electronic heart regulation (e.g. cardiac pacemaker) according to the individual cardiac metabolic profile of the present invention. The prophylactic therapy as described herein is intended to encompass prevention or reduction of risk of cardiovascular related disorders or cardiac metabolic changes. In one embodiment, the individual cardiac metabolic profile can be determined during the treatment for evaluating the effectiveness of the treatment. In one embodiment, the individual cardiac metabolic profile can be determined before treatment for selecting a treatment. In one embodiment, the individual cardiac metabolic profile can be determined after the treatment for evaluating effectiveness of the treatment, wherein said effectiveness of the treatment comprise an improved cardiac metabolism, improved cardiac output, activity tolerance, gene expression of cardiac genes at levels of physiological cardiac state, metabolite concentration at levels of physiological cardiac state, an increased myocardial ATP reserve, preferably an increased myocardial ATP production capacity as compared to myocardial ATP production capacity before treatment.

As used herein, a “patient with symptoms of a cardiovascular-related disorder” is a subject who presents with one or more of, without limitation, reduced gene expression of key proteins involved in cardiac energy metabolism, increased gene expression of key proteins involved in cardiac energy metabolism, decreased levels of central metabolic enzymes, increased levels of central metabolic enzymes, reduced levels of cardiac energy-rich phosphates, elevated levels of cardiac energy-rich phosphates, long-term pressure, cardiac volume overload, cardiac dysfunction, myocardial infarction, myocardial hypertrophy congestive heart failure, survived cardiac arrest, arrhythmias, cardiovascular events, chest pain, palpitations (rapid rhythms or skips), breath disabilities, fatigue, and has an increased risk of death. In some embodiments, said patients suffer from valve diseases, e.g. aortic stenosis (AS) or mitral valve insufficiency (MI).

In some embodiments, said patient has symptoms of reduced gene expression of proteins involved in cardiac energy metabolism or symptoms of increased gene expression of proteins involved in cardiac energy metabolism or symptoms of decreased levels of central metabolic enzymes or symptoms of reduced levels of cardiac energy-rich phosphates or symptoms of increased levels of central metabolic enzymes or symptoms of elevated levels of cardiac energy-rich phosphates or symptoms of long-term pressure or symptoms of cardiac volume overload or symptoms of cardiac dysfunction or symptoms of myocardial infarction or symptoms of myocardial hypertrophy or symptoms of congestive heart failure or symptoms of survived cardiac arrest or symptoms of arrhythmia symptoms of cardiovascular events or symptoms of chest pain or symptoms of palpitations (rapid rhythms or skips) or symptoms of breath disabilities or symptoms of fatigue or symptoms of an increased risk of death.

In some embodiments, said patient has no symptoms of reduced gene expression of proteins involved in cardiac energy metabolism or no symptoms of increased gene expression of proteins involved in cardiac energy metabolism or no symptoms of decreased levels of central metabolic enzymes or no symptoms of reduced levels of cardiac energy-rich phosphates or no symptoms of increased levels of central metabolic enzymes or no symptoms of elevated levels of cardiac energy-rich phosphates or no symptoms of long-term pressure or no symptoms of cardiac volume overload or no symptoms of cardiac dysfunction or no symptoms of myocardial infarction or no symptoms of myocardial hypertrophy or no symptoms of congestive heart failure or no symptoms of survived cardiac arrest or no symptoms of arrhythmias or no symptoms of cardiovascular events or no symptoms of chest pain or no symptoms of palpitations (rapid rhythms or skips) or no symptoms of breath disabilities or no symptoms of fatigue or no symptoms of an increased risk of death.

In some embodiments, said patient at risk of developing symptoms of reduced gene expression of key proteins involved in cardiac energy metabolism or symptoms of increased gene expression of key proteins involved in cardiac energy metabolism or symptoms of decreased levels of central metabolic enzymes or symptoms of increased levels of central metabolic enzymes or symptoms of reduced levels of cardiac energy-rich phosphates or symptoms of elevated levels of cardiac energy-rich phosphates or symptoms of long-term pressure or symptoms of cardiac volume overload or symptoms of cardiac dysfunction or symptoms of myocardial infarction or symptoms of myocardial hypertrophy or symptoms of congestive heart failure or symptoms of survived cardiac arrest or symptoms of arrhythmia symptoms of cardiovascular events or symptoms of chest pain or symptoms of palpitations (rapid rhythms or skips) or symptoms of breath disabilities or symptoms of fatigue or symptoms of an increased risk of death presents changes in cardiac metabolism at therapeutically relevant level.

### FIGURES

The invention is demonstrated by way of the example through the figures disclosed herein. The figures provided represent particular, non-limiting embodiments and are not intended to limit the scope of the invention.

### SHORT DESCRIPTION OF THE FIGURES

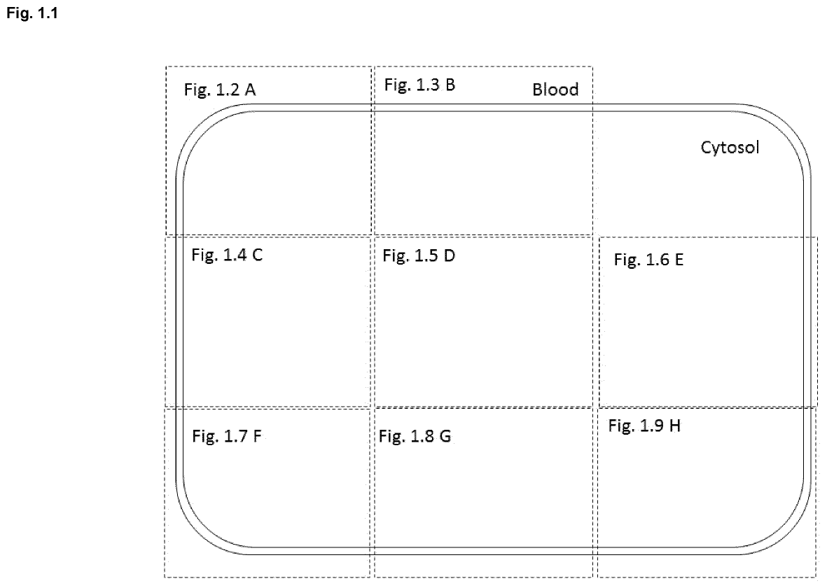

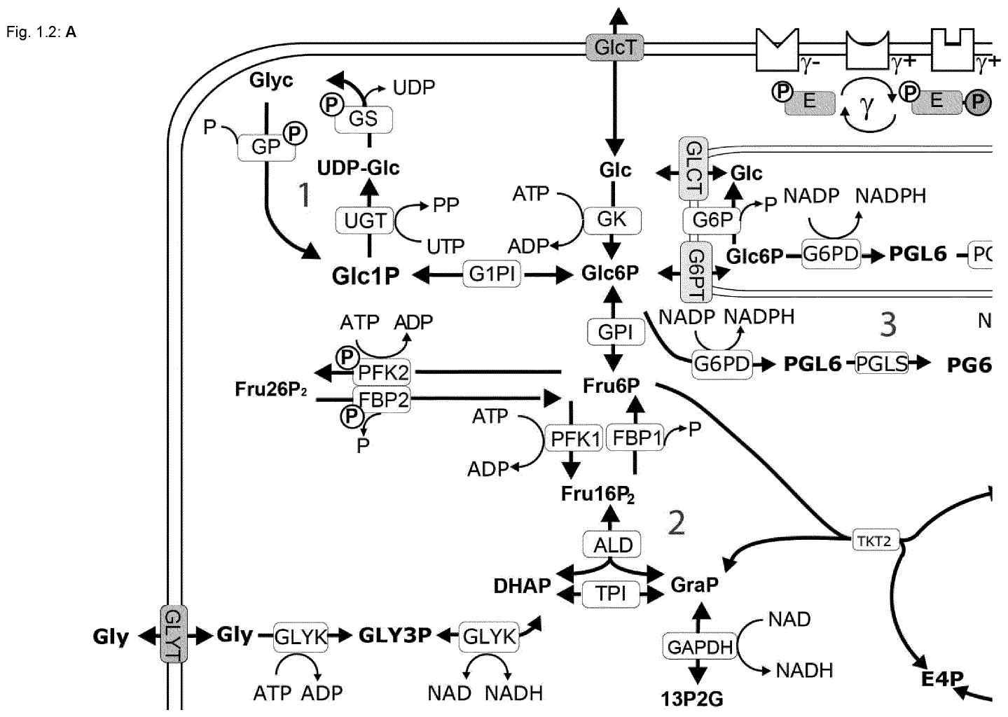

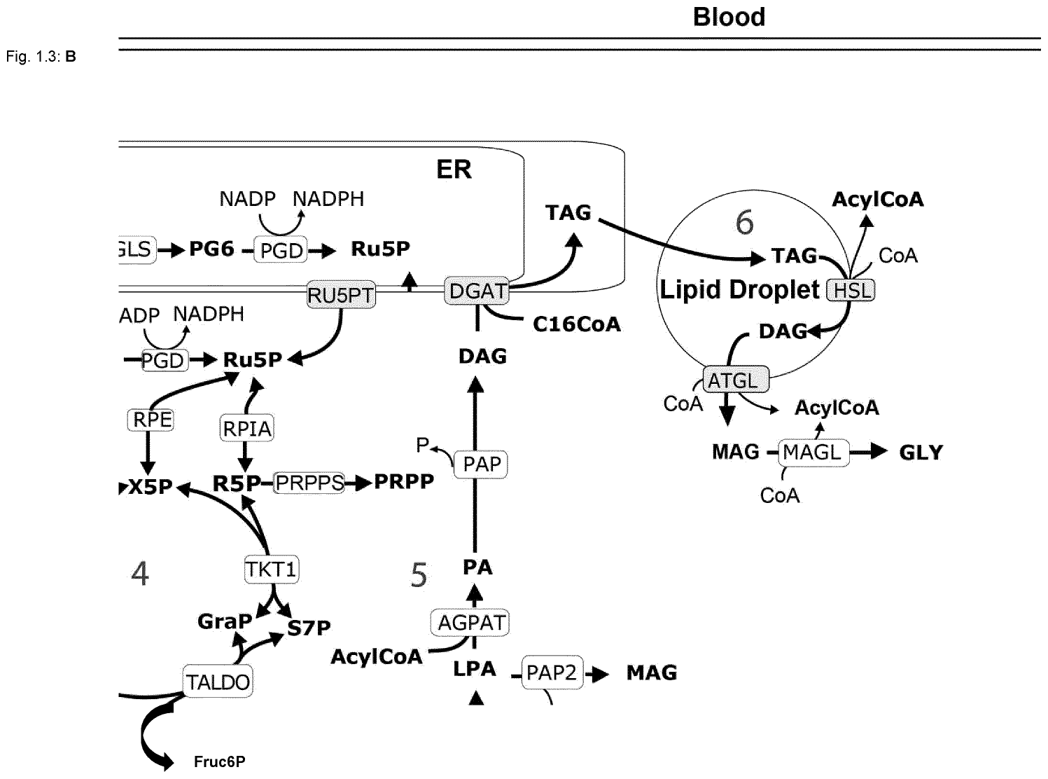

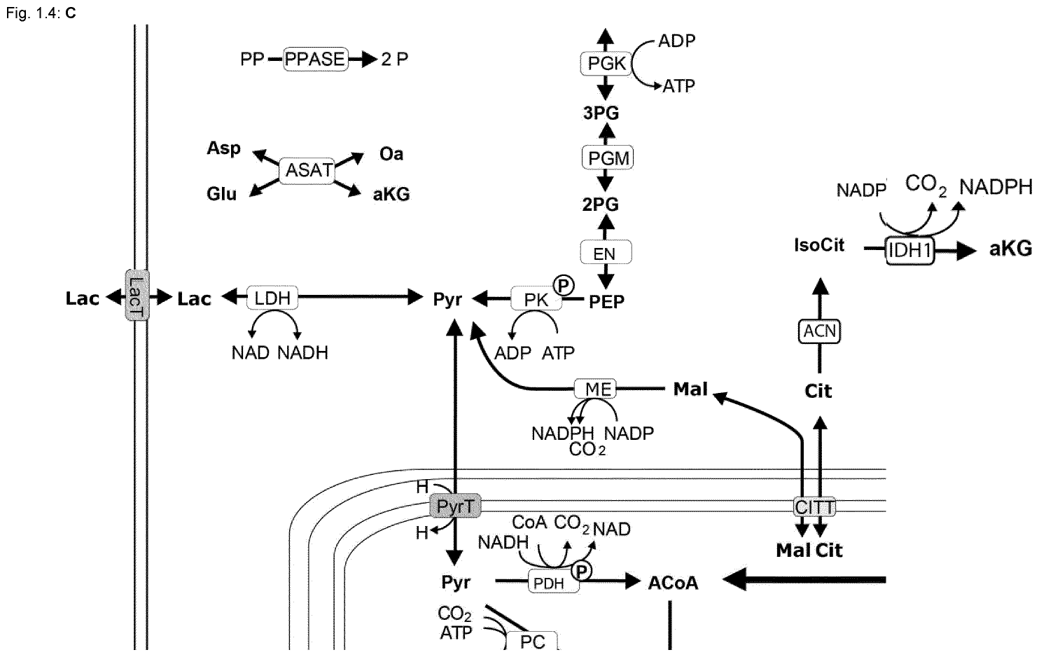

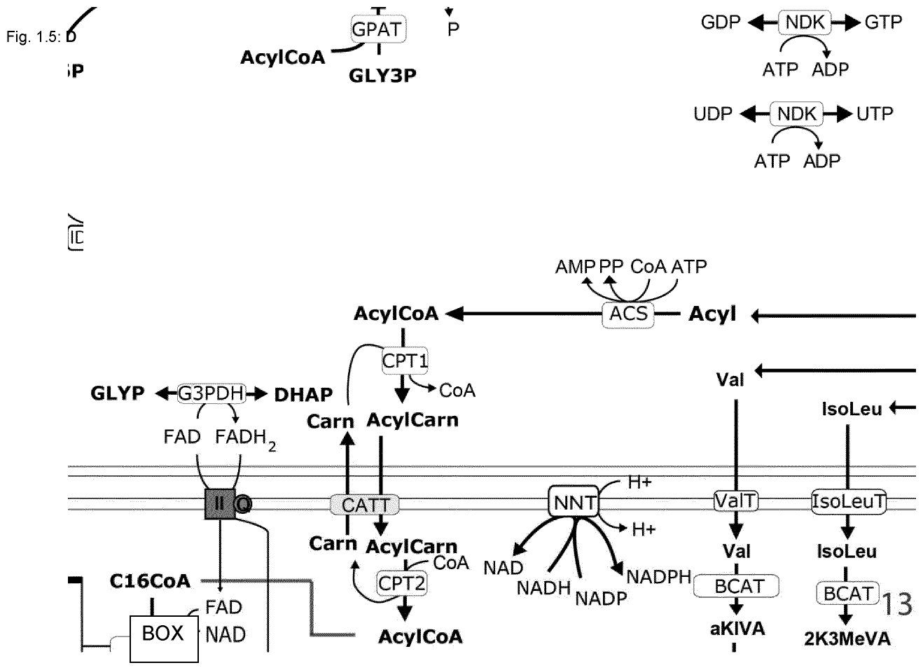

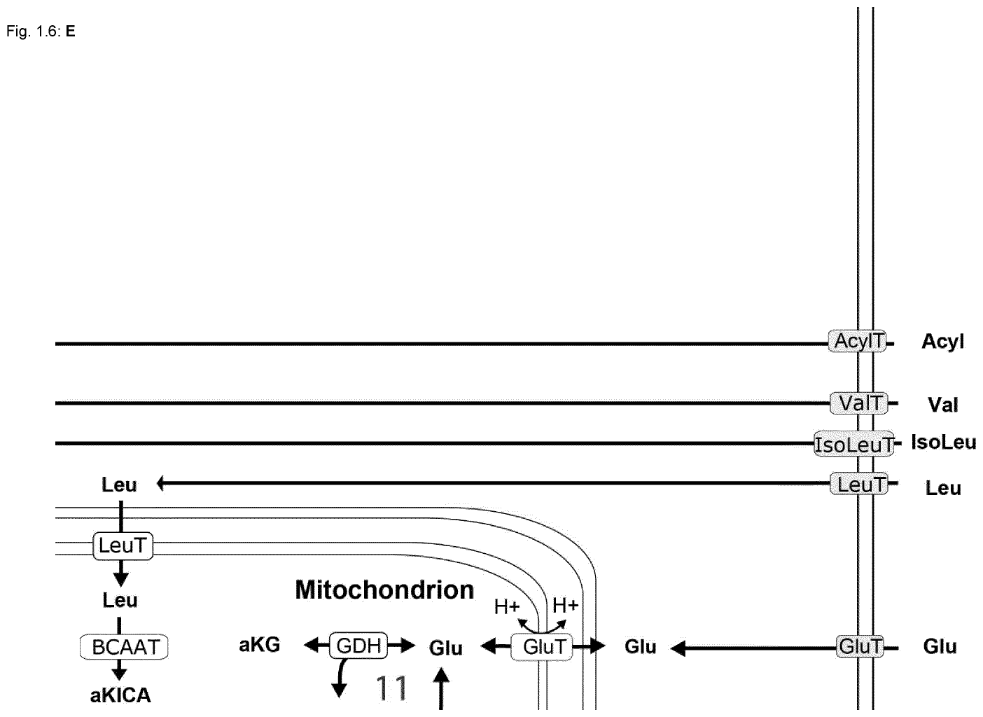

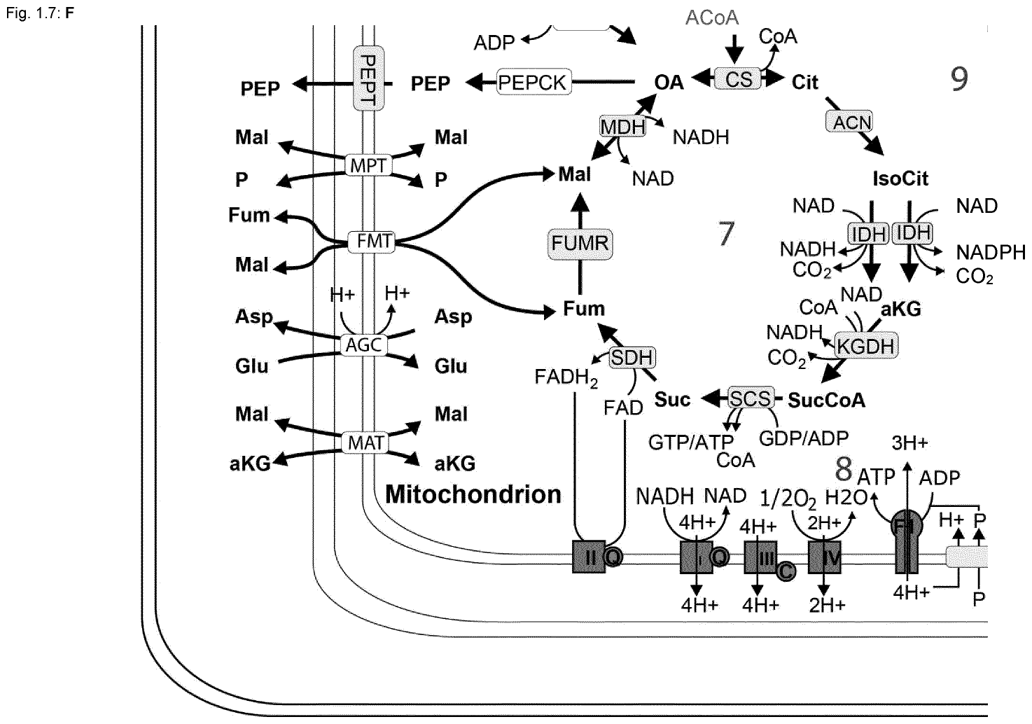

FIG. 1: Reaction scheme of the metabolic model

FIG. 2: Simulated and measured myocardial substrate uptake rates in vivo

FIG. 3: MV_{ATP}(rest) and MV_{ATP}(max) for controls and patients with mitral valve disease and aortic stenosis

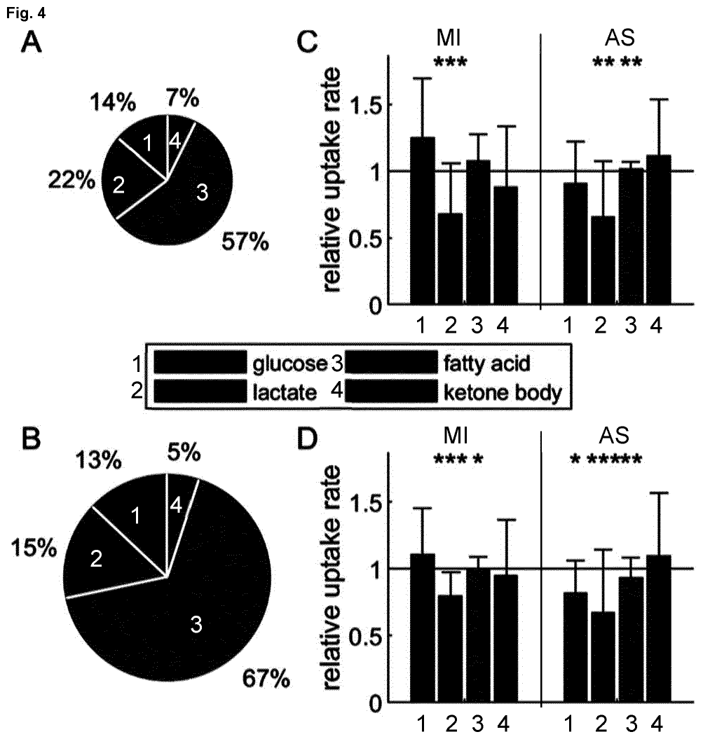

FIG. 4: Contribution of energy delivering substrates

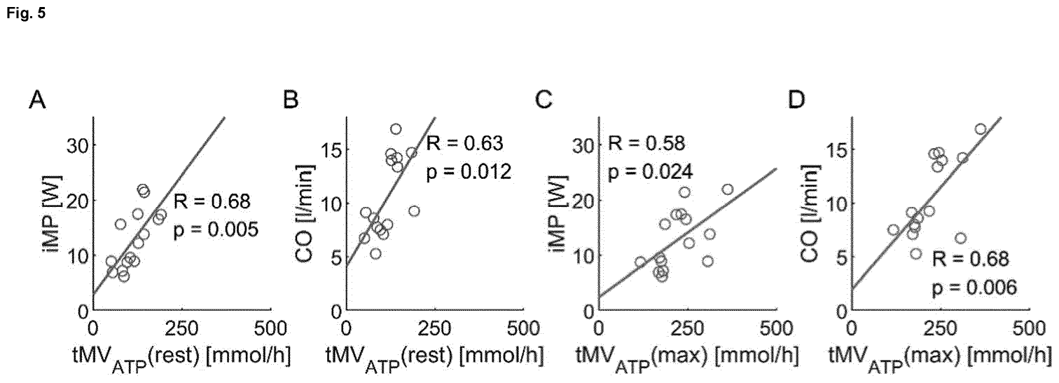

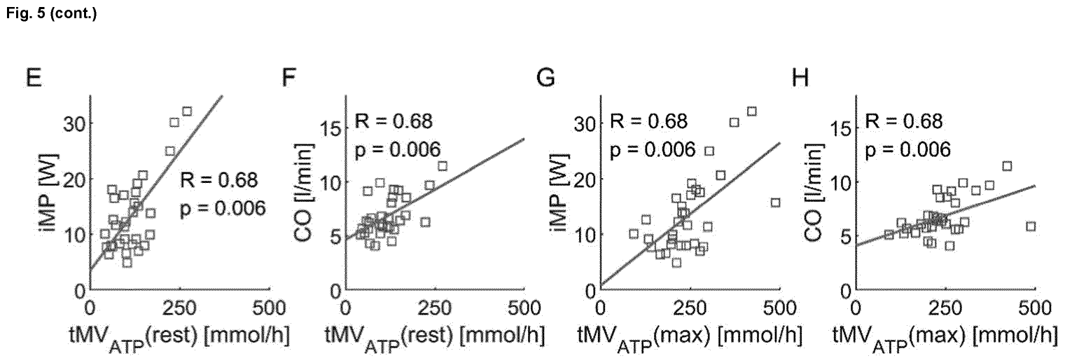

FIG. 5: Correlation between tMVATP(rest) as well as tMVATP(max) and internal myocardial power (iMP) as well as cardiac output (CO) for MI patients (A-D) and AS patients (E-H)

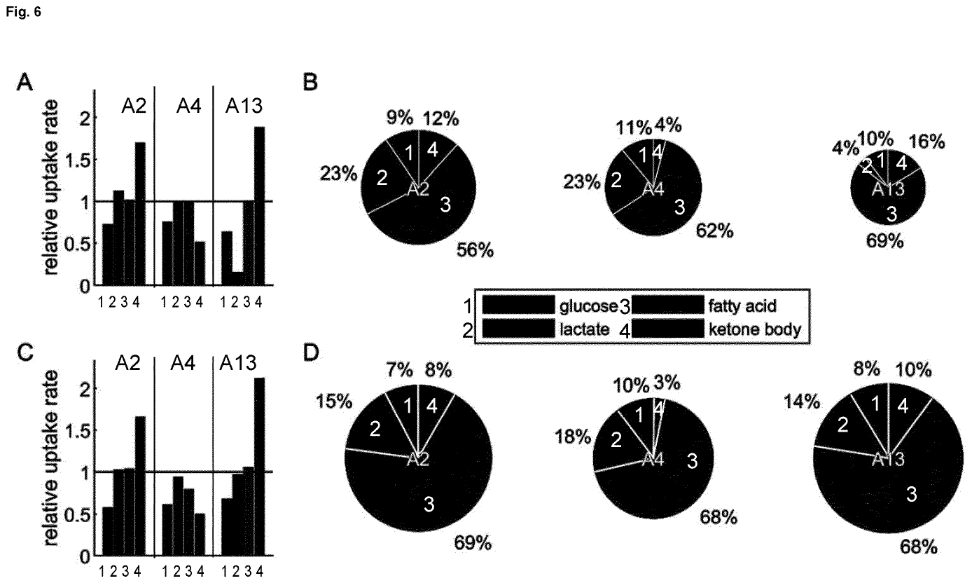

FIG. 6: Metabolic characterization of three patients with AS

### DETAILED DESCRIPTION OF THE FIGURES

FIG. 1: Reaction scheme of the metabolic model. FIG. 1.1 represents an overview of FIG. 1 for all parts shown in FIG. 1.2:A to FIG. 1.9:H. Arrows symbolize reactions and transport processes between compartments. 1) glycogen metabolism, (2) glycolysis, (3) oxidative pentose phosphate pathway in the endoplasmic reticulum and cytosol, (4) non-oxidative pentose phosphate pathway, (5) triglyceride synthesis, (6) synthesis and degradation of lipid droplets, (7) tricarbonic acid cycle, (8) respiratory chain and oxidative phosphorylation, (9) B-oxidation of fatty acids, (10) ketone body utilization, (11) glutamate metabolism, (12) mitochondrial electrophysiology (membrane transport of ions, (13) Utilization of branched-chain amino acids. Small cylinders and cubes symbolize ion channels and ion transporters. Double-arrows indicate reversible reactions, which according to the value of the thermodynamic equilibrium constant and cellular concentrations of their reactants may proceed in both directions. Reactions are labeled by the short names of the catalyzing enzyme or membrane transporter given in the small boxes attached to the reactions arrow. Metabolites are denoted by their short names. Full names and kinetic rate laws of reaction rates are outlined in Table 8. Full names of metabolites and a comparison of experimentally determined and calculated cellular metabolite concentrations are given in Table 9.

FIG. 2: Simulated and measured myocardial substrate uptake rates in vivo. (A) Substrate uptake rates at rest and at moderate pacing (50% maxV_{O2}). The experimental data represent the mean of various studies [40-46]. They were computed from reported extraction rates (=1−arterial concentration/concentration in coronary sinus) putting the coronary blood flow to 0.8 ml/min/g and heart weight to 300 g. (B) Dependence of the glucose uptake rate from the plasma concentration of FFAs. The solid line represents model values, squares symbolize in vivo data taken from Nuutila et al. [38].

FIG. 3: MV_{ATP}(rest) and MV_{ATP}(max) for controls and patients with mitral valve disease and aortic stenosis. MV_{ATP}(rest) and MV_{ATP}(max) for controls and patients with mitral valve disease and aortic stenosis (A) Bottom values of the bars refer to MV_{ATP}(rest), top values refer to MV_{ATP}(max). The bar length indicates the myocardial ATP production reserve, MAPR=MV_{ATP}(max)−MV_{ATP}(rest), of the subject. (B-D) Box plots showing mean values, upper and lower quartiles and total span of MV_{ATP}(rest), MV_{ATP}(max) and MAPR for controls and patients with MI and AS.

FIG. 4: Contribution of energy delivering substrates. The panels A and B show the relative contribution of the energy delivering substrates to total energy expenditure at MV_{ATP}(rest) and MV_{ATP}(max) for the control group for 60 min pacing. Area of the pie charts represent total energy expenditure. Changes of substrate uptake rates of MI and AS patients relative to controls are shown at rest (C) and during maximal pacing (D). Bar plots represent the relative change of substrate uptake rates of glucose (1), lactate (2), fatty acids (3) and ketone bodies (4) for patients with MI and AS during rest and at maximal ATP production rate after 60 min of pacing. Relative uptake rates are normalized to control values (i.e. all control values are equal to 1).

FIG. 5: Correlation between tMV_{ATP}(rest) as well as tMV_{ATP}(max) and internal myocardial power (iMP) as well as cardiac output (CO) for MI patients (A-D) and AS patients (E-H). Correlation between tMV_{ATP}(rest) as well as tMV_{ATP}(max) and internal myocardial power (iMP) as well as cardiac output (CO) for MI patients (A-D) and AS patients (E-H).

FIG. 6: Metabolic characterization of three patients with AS. Relative substrate utilization rates compared to healthy controls at rest (A) and at maximal load (C) as well as the relative contribution of the different substrates (glucose (1), lactate (2), fatty acids (3) and ketone bodies (4)) to overall ATP production rate at rest (B) and maximal load (D). Area of pie diagrams represent total ATP production rate.

### EXAMPLES

The invention is demonstrated through the examples disclosed herein. The examples provided represent particular embodiments and are not intended to limit the scope of the invention. The examples are to be considered as providing a non-limiting illustration and technical support for carrying out the invention.

The examples below present a physiology-based mathematical model of the myocardial energy metabolism. The model encompasses all pathways along which the possible energy-delivering substrates glucose, long-chain fatty acids, ketone bodies (KBs), acetate (AC) and branched-chain amino acids (BCAAs) are utilized. The method described herein allows to assess the capability of the left ventricular septum of patients and controls to increase MV_{ATP} in response to an increase of the ATP demand. Based on LV samples from controls and patients with MI and AS, it is shown that the ATP production capacity of the LV is reduced in patients and correlates positively with mechanical energy demand and cardiac output and is consistent with the clinical data.

### Methods

### Patient Characteristics

We investigated 75 human left ventricular myocardial biopsies. In patients, myocardial samples from the LV septum were collected during surgical aortic or mitral valve replacement from 41 patients with aortic valve stenosis (AS) and 17 patients with mitral valve insufficiency (MI). Patient characteristics are described in Table 1. For the controls (n=17), samples were taken from 44±15 year-old donors without cardiac diseases but whose hearts were not used for transplantation. All samples were frozen immediately in liquid nitrogen until further processing.

The study protocol was in agreement with the principles outlined in the Declaration of Helsinki and was approved by the Medical Ethics Review Committee. All patients gave written informed consent prior to inclusion.

TABLE 1
Patients Characteristics
Data are presented as total numbers and percentage in case of categorical and as mean and standard
deviation (SD) in case of numeric values. Parameter differences between the two patient groups were
evaluated by means of two-sided, two-sample Wilcoxon-rank test in case of numeric data and via
Chi-squared test with Yates' continuity correction in case of categorical data (p-values given in the
right column). ACE-inhibitor = angiotensin converting enzyme-inhibitor, AS = aortic stenosis,
BMI = body mass index, MI = mitral valve insufficiency

| Patient characteristic and | | SD/ | | SD/ | p- |
|---|---|---|---|---|---|
| preoperative function parameters | AS | % | MI | % | value |
| Age at Surgery in years | 68 | 9 | 60 | 14 | 0.032 |
| BMI | 28 | 4 | 27 | 3 | 0.343 |
| Gender female | 23 | 56% | 6 | 35% | 0.414 |
| NYHA (stage I, II, III, IV) | (5, 17, | n.a. | (2, 7, | n.a. | 0.593 |
| | 15, 1) | | 6, 2) | | |
| Blood pressure systolic in mm[Hg] | 140 | 19 | 131 | 16 | 0.123 |
| Blood pressure diastolic in mm[Hg] | 74 | 11 | 75 | 13 | 0.675 |
| EDVi in ml/m2 | 73 | 17 | 108 | 34 | <0.001 |
| ESVi in ml/m2 | 30 | 11 | 40 | 14 | 0.015 |
| EF in % | 60 | 7 | 62 | 9 | 0.048 |
| Cl in l/min/m2 | 3 | 1 | 5 | 2 | <0.001 |
| CO in l/min | 6 | 2 | 9 | 4 | <0.001 |
| Internal myocardial power | 13 | 7 | 13 | 5 | 1 |
| Myocardial mass (i) in g/m2 | 71 | 20 | 67 | 15 | 0.484 |
| Mean pressure gradient | 56 | 15 | 4 | 8 | <0.001 |
| aortic valve, mm[Hg] | | | | | |
| Mitral valve insufficiency | (41, | n.a. | (0, | n.a. | |
| (none/mild, moderate, severe) | 0, 0) | | 10, 7) | | |
| Aortic valve insufficiency | (36, | n.a. | (17, | n.a. | |
| (none/mild, moderate, severe) | 5, 0) | | 0, 0) | | |
| Serum Creatinine [mg/dl] | 0.91 | 0.15 | 1.0 | 0.20 | 0.065 |
| Hypertension | 27 | 66% | 11 | 65% | 0.826 |
| Dyslipidemia | 8 | 20% | 3 | 18% | 0.839 |
| Diabetes type 2 | 7 | 17% | 2 | 12% | 0.913 |
| Coronary Artery Disease | 1 | 2% | 2 | 12% | 0.419 |
| Atrial fibrillation paroxysmal | 2 | 5% | 2 | 12% | 0.709 |
| Atrial fibrillation permanent | 0 | 0% | 2 | 12% | 0.149 |
| Medication ACE inhibitor | 15 | 37% | 5 | 29% | 0.826 |
| Medication beta blocker | 20 | 49% | 10 | 59% | 0.683 |
| Medication diuretics | 12 | 29% | 5 | 29% | 0.760 |

### Quantitative Proteomics of Tissue Samples

Heart biopsies were taken from patients admitted in need for aortic or mitral valve replacement surgery or from healthy donor heart control subjects. Left ventricular septum biopsies were extracted at time of surgery, frozen directly in liquid nitrogen and kept at −80° C. For protein extraction, biopsies were lysed in 200 μl lysis buffer containing: 2% SDS, 50 mM ammonium bicarbonate buffer and EDTA-free Protease Inhibitor Cocktail (Complete, Roche). Samples were homogenized at room temperature using FastPrep-24™ 5G Homogenizer (MP Biomedicals) with 10 cycles of 20 s and 5 s pause between cycles. After heating the samples for 5 min at 95° C., 5 freeze-thaw cycles were applied. 25 U of Benzonase (Merck) was added to each sample and after an incubation for 30 min the lysates were clarified by centrifugation at 16,000 g for 40 min at 4° C. Protein concentration was measured (Bio-Rad DC Protein assay) and 100 μg of each sample was further processed using the SP3 clean-up and digestion protocol as previously described [20]. Briefly, each sample was reduced with dithiothreitol (10 mM final, Sigma) for 30 min, followed by alkylation with chloroacetamide (40 mM final, Sigma) for 45 min and quenching with dithiothreitol (20 mM final, Sigma). Beads (1 mg) and acetonitrile (70% final concentration) were added to each sample and after 20 min of incubation on an over-head rotor bead-bound protein were washed with 70% ethanol and 100% acetonitrile. 2 μg sequence-grade Trypsin (Promega) and 2 μg Lysyl Endopeptidase LysC (Wako) in 50 mM HEPES (pH 8) were added and after an overnight incubation at 37° C. peptides were collected, acidified with trifluoroacetic acid and cleaned up using StageTips protocol [21].

### Heart Reference Sample for Matching Library

A peptide mix for each experimental group (Control, AS and MI) was generated by collecting 10 μg peptides from each individual sample belonging to the corresponding group. Equal peptide amounts from each group mixture were combined, desalted using a C18 SepPak column (Waters, 100 mg) and dried down using a SpeedVac instrument. Peptides were reconstituted in 20 mM ammonium formate (pH 10) and 2% acetonitrile, loaded on a XBridge C18 4.6 mm×250 mm column (Waters, 3.5 μm bead size) and separated on an Agilent 1290 HPLC instrument by basic reversed-phase chromatography, using a 90 min gradient with a flow rate of 1 ml/min, starting with solvent A (2% acetonitrile, 5 mM ammonium formate, pH 10) followed by increasing concentration of solvent B (90% acetonitrile, 5 mM ammonium formate, pH 10). The 96 fractions were collected and concatenated by pooling equal interval fractions. The final 26 fractions were dried down and resuspended in 3% acetonitrile/0.1% formic acid for LC-MS/MS analyses.

### LC-MS/MS Analyses

Peptide samples were eluted from stage tips (80% acetonitrile, 0.1% formic acid), and after evaporating organic solvent peptides were resolved in sample buffer (3% acetonitrile/0.1% formic acid). Peptide separation was performed on a 20 cm reversed-phase column (75 μm inner diameter, packed with ReproSil-Pur C18-AQ; 1.9 μm, Dr. Maisch GmbH) using a 200 min gradient with a 250 nl/min flow rate of increasing Buffer B concentration (from 2% to 60%) on a High Performance Liquid Chromatography (HPLC) system (ThermoScientific). Peptides were measured on an Orbitrap Fusion (individual samples) and Q Exactive HF-X Orbitrap instrument (reference sample) (ThermoScientific). On the Orbitrap Fusion instrument, peptide precursor survey scans were performed at 120K resolution with a 2×10⁵ ion count target. MS² scans were performed by isolation at 1.6 m/z with the quadrupole, HCD fragmentation with normalized collision energy of 32, and rapid scan analysis in the ion trap. The MS² ion count target was set to 2×10³ and the max injection time was 300 ms. The instrument was operated in Top speed mode with 3 s cycle time, meaning the instrument would continuously perform MS² scans until the list of non-excluded precursors diminishes to zero or 3 s. On the Q Exactive HF-X Orbitrap instrument, full scans were performed at 60K resolution using 3×10⁶ ion count target and maximum injection time of 10 ms as settings. MS² scans were acquired in Top 20 mode at 15K resolution with 1×10⁵ ion count target, 1.6 m/z isolation window and maximum injection time of 22 ms as settings. Each sample was measured twice, and these two technical replicates were combined in subsequent data analyses.

Data were analyzed using MaxQuant sofware package (v1.6.2.6) [22]. The internal Andromeda search engine was used to search MS² spectra against a decoy human UniProt database (HUMAN.2019-01, with isoform annotations) containing forward and reverse sequences. The search included variable modifications of oxidation (M), N-terminal acetylation, deamidation (N and Q) and fixed modification of carbamidomethyl cysteine. Minimal peptide length was set to six amino acids and a maximum of three missed cleavages was allowed. The FDR (false discovery rate) was set to 1% for peptide and protein identifications. Unique and razor peptides were considered for quantification. Retention times were recalibrated based on the built-in nonlinear time-rescaling algorithm. MS² identifications were transferred between runs with the “Match between runs” option, in which the maximal retention time window was set to 0.7 min. The integrated LFQ quantitation algorithm was applied. Gene Symbols assigned by MaxQuant were substituted with gene symbols of the reported UniProt IDs from the FASTA file used.

### Description of the Mathematical Model (CARDIOKIN1)

For the quantification of the metabolic changes caused by the abundance changes of metabolic enzymes, we developed a mathematical model of the cardiac energy metabolism, which comprises all pathways involved in the catabolism of the energy-delivering substrates glucose, lactate, fatty acids, KBs and BCAAs as well as the synthesis of endogenous energy stores (glycogen, triacylglycerol) (see FIG. 1). The model also takes into account the short-term regulation of metabolic enzymes and transporters by the hormones insulin and catecholamines and key electrophysiological processes at the inner mitochondrial membrane including the generation of the proton gradient by the respiratory chain, the synthesis of ATP by the FoF1-ATPase and the membrane transport of various ions.

The time course of model variables (=concentration of metabolites and ions) is governed by first-order differential equations. Time-variations of small ions are modeled by kinetic equations of the Goldman-Hodgkin-Katz type as used in our previous work [13]. The rate laws for enzymes and membrane transporters were either taken from the literature or constructed based on published experimental data for the mammalian heart.

Model calibration for individual hearts We used the proteomics-derived protein profiles of enzymes and transporters for model calibration by computing the maximal activities (Vₘₐₓ) of the enzymes by the relation

$${V}_{max}^{subject} = {V}_{max}^{normal} \frac{{E}^{subject}}{〈 {E}^{control} 〉} \qquad ( 1 )$$

where [symbol] E^{control} [symbol] is the average protein intensity of the enzyme in the group of control hearts and E^{Subject} is the protein concentration of the enzyme in the individual (control or patient). The maximal activities vₘₐₓ^{normal} of the reference model for the average normal heart were obtained by fitting of the model to experimental data. Equation (1) follows from the fact that the maximal enzyme activity is proportional to the abundance of the protein.

Energetic Capacities of Controls and Patients with Valve Diseases

We used the model to compute the specific uptake rates of substrates and the specific ATP production rate at rest, MV_{ATP}(rest), and at maximal ATP workload, MV_{ATP}(max), for the LV of controls (N=17) and patients with MI (N=17) or AS (N=41). As third parameter to characterize the capacity of the LV to increase the ATP production with increasing workload, we used the span between MV_{ATP}(max) and MV_{ATP}(rest), which we will refer to as myocardial ATP production reserve, MAPR (MAPR=MV_{ATP}(max)−MV_{ATP}(rest)). In the following, we will distinguish between specific energy parameters MV_{ATP}(rest), MV_{ATP}(max) and MAPR quantifying the energetic capacity per mass unit of the LV (given in μmol/g/h) and total energy parameters tMV_{ATP}(rest), tMV_{ATP}(max) and tMAPR quantifying the energetic capacity of the LV (given in mmol/h), i.e. tMV_{ATP}(rest)=MV_{ATP}(rest)×LV mass/1000 etc.

The computations were performed for a normal post-absorptive state (overnight fast) characterized by the following metabolite and hormone concentrations: glucose 5.8 mM [23], fatty acids 0.5 mM [24], lactate 0.8 mM [23], glutamine 0.5 mM [25, 26], valine 0.2 mM [25, 26], leucine 0.15 mM [25, 26], isosleucine 0.06 mM [25, 26], ß-hydroxybutyrate 0.08 mM [27, 28], acetoacetate 0.04 mM. The concentration of catecholamines at rest was 0.75 nM [29, 30] and increased with growing workload (Example 2).

The myocardial ATP consumption of the stationary resting state, MV_{ATP}(rest), was chosen in a way that the computed oxygen consumption (MV_{O2}) was identical with the subject's MV_{O2}, which we estimated by the 2-factor approximation

$${MV}_{O 2} = γ \cdot HR \cdot BP \qquad ( 7 )$$

HR and BP are the heart rate and the peak systolic blood pressure and γ is a proportionality factor. Resting MV_{O2} of normal hearts was found in the range of 0.8-1.2 ml/min/g [2-4]. Thus, with a mean MV_{O2}=0.1 ml/min/g, HR=70/min and normal BP=125 mm, we set γ to 1.143×10-5 ml/mmHg/g.

The metabolic response of the ventricle to an additional workload (pacing) was evaluated by computing the temporal changes of the metabolic state elicited by an increase of the ATP consumption rate above the resting value. The ATP consumption rate was modeled by a generic hyperbolic rate law

$${v}_{ATP} = {k}_{load} \cdot \frac{ATP}{ATP + {K}_{m}} \qquad ( 8 )$$

The parameter k_{load} was stepwise increased until MV_{ATP} converged to the maximum, MV_{ATP}(max).

To evaluate the mechanical burden of the heart, we calculated the internal myocardial power, which describes the energy required for cardiac contraction for the individual hearts (see methods used in Lee et al. [32]).

All model simulations were performed using MATLAB, Release R2011b, The MathWorks, Inc., Natick, Massachusetts, United States.

### Example 1: A Novel Method to Assess the Myocardial ATP Producing Capacity

Currently, no method is available to measure MV_{ATP} in vivo. Invasive techniques, such as the determination of substrate extraction rates from coronary sinus, arterial concentration differences or the oxidation rates of 14C-labeled substrates from the rates of 14C_{O2}, have been applied in healthy subjects and patients with heart diseases [45, 47, 49]. However, such data cannot be directly converted into rates of ATP production. The same holds true for the measurement of the myocardial oxygen consumption rate MV_{O2} reflecting the overall myocardial oxidative metabolism. The MV_{O2} does not capture the glycolytic ATP contribution, which is low under normoxic conditions but may increase 5-fold during development of heart failure or even 20-fold during the transition from aerobic to anaerobic energy production [51]. Moreover, the ATP/_{O2} ratio may change considerably with increasing workload owing to increasing cardiac preference for carbohydrates. This makes it difficult to convert O2 consumption rates into ATP consumption rates. In addition, the maximal MV_{O2} can be low due to restrictions imposed to heart performance by the non-metabolic factors. To close this methodological gap, we applied here a novel approach to assess to energetic capacity of the LV of the human heart by combining kinetic modelling with protein abundance data of metabolic enzymes determined in cardiac tissue.

Except for the maximal enzyme activities (Vₘₐₓ values), which may vary owing to variable gene expression, the numerical values for all other parameters of the enzymatic rate laws were taken from reported kinetic studies of the isolated enzymes. Numerical values for the Vₘₐₓ values were estimated by the same procedure that was used for the calibration of our metabolic liver model [19]: Calculated metabolite profiles and fluxes were adjusted to experimental data from independent experiments with perfused hearts and in vivo measurements (see Table 2) while the metabolite concentrations were constrained to experimentally determined ranges. Short-term regulation of key regulatory enzymes by the hormones insulin and catecholamines (epinephrine, nor-epinephrine) was included into the model by phenomenological mathematical functions relating the enzyme's phosphorylation state and the abundance of the GLUT4 transporter in the sarcolemma to the plasma concentrations of glucose (insulin) and the exercise level (catecholamines) (see Example 2).

TABLE 2
Model simulations, which correctly
recapitulate metabolic measurements
obtained with perfused hearts and in human in vivo studies
FFAs = free fatty acids, KBs = ketone bodies,
BCAAs = branched-chain amino acids

| Measurements | Data source |
|---|---|
| Glucose utilization at varying | [33, 34] |
| exogenous glucose concentrations | |
| Lactate utilization and lactate/O₂ ratio at varying | [35] |
| exogenous lactate concentrations | |
| Utilization of FFAs at varying | [36, 37] |
| exogenous FFA concentrations | |
| Glucose utilization in response to varying exogenous | [38] |
| concentration of FFAs (glucose-FFA-competition) | |
| KBs utilization at varying exogenous β- | [39] |
| hydroxybutyrate concentrations | |
| Utilization rates of glucose, lactate, FFAs, KBs and | [40-46] |
| BCAAs under post-absorptive resting conditions | |
| Utilization rates of glucose, lactate, FFAs, KBs and | [40, 43, 44] |
| BCAAs at moderate pacing | |

Details of all validation simulations listed in Table 2 are given in below. As the heart switches its substrate uptake rates in dependence of substrate availability, we performed different simulations with variable substrate availability.

### Glucose Uptake:

First, we simulated the glucose uptake of cardiac muscle in dependence of glucose availability. To match experimental conditions, we assumed that glucose and oxygen are the only available substrates, assumed that there are no hormones present and that the ATP demand is constant with a moderate demand. All external conditions are given in the Table 3.

TABLE 3
External conditions for simulation of cardiac glucose uptake

| | | | |
|---|---|---|---|
| Glucose [mM] | 0.5-25 | Valine [mM] | 0 |
| Lactate [mM] | 0 | Leucine [mM] | 0 |
| Fatty acids [mM] | 0 | Isoleucine [mM] | 0 |
| B-hydroxybuterate [mM] | 0 | Insulin [nM] | 0-1500 |
| Acetoacetate [mM] | 0 | | TF (Example 2) |
| | | k-load | 5 |

### Lactate Uptake:

The next most abundant carbohydrate available to the heart is lactate. Therefore, we used the model to investigate the utilization of this important fuel, when the supply with alternative substrate is limited. We varied the external availability of lactate between 0 mM and 12 mM, keeping the glucose concentration at a low value of 2 mM and putting the fatty acid concentration to 0.1 mM (Table 4). Lactate to oxygen consumption rate (OCR) ratio increases up to 4 mM plasma lactate concentration when saturation is reached. This means that in the physiological range (<2 mM) lactate uptake is limited by substrate availability.

TABLE 4
External conditions for simulation of cardiac lactate uptake

| | | | |
|---|---|---|---|
| Glucose [mM] | 2 | Valine [mM] | 0 |
| Lactate [mM] | 0.2-12 | Leucine [mM] | 0 |
| Fatty acids [mM] | 0.1 | Isoleucine [mM] | 0 |
| B-hydroxybuterate [mM] | 0 | Insulin [nM] | 1 |
| Acetoacetate [mM] | 0 | | TF (Example 2) |
| | | k-load | 0 |

### Fatty Acid Uptake:

Next, we checked the ability of the model to recapitulate fatty acid uptake. We monitored the fatty acid uptake when systematically varying the plasma fatty acid concentrations between 0 and 2 mM while assuming a moderate ATP demand (Table 5). As the majority of fatty acids in the plasma are bound to albumin, but only free fatty acids are taken up by the heart, we used the transfer function depicted in Example 2 to calculate the free fatty acid concentration from the plasma fatty acid concentration.

TABLE 5
External conditions for simulation of cardiac fatty acid uptake

| | | | |
|---|---|---|---|
| Glucose [mM] | 7.63 | Valine [mM] | 0 |
| Lactate [mM] | 1 | Leucine [mM] | 0 |
| Fatty acids [mM] | 0-2 | Isoleucine [mM] | 0 |
| B-hydroxybuterate [mM] | 0 | Insulin [nM] | 757 |
| Acetoacetate [mM] | 0 | | TF (Example 2) |
| | | k-load | 5 |

### Suppression of Glucose Uptake by Plasma Fatty Acids:

After checking that the model correctly recapitulates the substrate utilization for glucose and fatty acids in the absence of the other substrate, the next step was to investigate the interplay of the different substrates. Therefore, we simulated the uptake of glucose in the presence of varying fatty acid concentrations in the plasma. With increasing fatty acid availability, the model correctly recapitulates the replacement of glucose with fatty acids. This strongly supports the view that fatty acids are the preferred substrate for the heart, and that glucose is used only when fatty acid availability is limited. (Table 6)

TABLE 6
External conditions for simulation of the uptake of glucose in
the presence of varying fatty acid concentrations in the plasma.

| | | | |
|---|---|---|---|
| Glucose [mM] | 5.8 | Valine [mM] | 0 |
| Lactate [mM] | 0.8 | Leucine [mM] | 0 |
| Fatty acids [mM] | 0-1.2 | Isoleucine [mM] | 0 |
| B-hydroxybuterate [mM] | 0 | Insulin [nM] | 257 |
| Acetoacetate [mM] | 0 | | TF (Example 2) |
| | | k-load | 2 |

### Ketone Body Utilization:

Ketone bodies represent an important substrate for the heart especially during fasting conditions when glucose and lactate are not available or need to be saved for the utilization by other organs (i.e. gluconeogenesis form lactate in the liver or glycolysis in the brain). Assuming moderate glucose levels and moderate load, we systematically varied the plasma ketone body concentration (B-hydroxybuterate) from 0 to 5.5 mM and monitored the ketone body uptake rates. (Table 7)

TABLE 7
External conditions for simulation of the ketone body uptake.

| | | | |
|---|---|---|---|
| Glucose [mM] | 4 | Valine [mM] | 0 |
| Lactate [mM] | 0.8 | Leucine [mM] | 0 |
| Fatty acids [mM] | 0-5 | Isoleucine [mM] | 0 |
| B-hydroxybuterate [mM] | 0 | Insulin [nM] | 36 |
| Acetoacetate [mM] | 0 | | TF (Example 2) |
| | | k-load | 3 |

### Substrate Utilization in the Human Heart:

Next, we tested whether the model correctly recapitulates substrate uptake of the human heart under physiological conditions when all substrates (glucose, lactate, fatty acids, ketone bodies and branched chain amino acids) are present at the same time. We simulated the substrate utilization rates of the human heart at rest and moderate pacing in an overnight fasted state and compared the simulated rates to experimental data for the human heart (Table 7). FIG. 2 shows that the model calculations recapitulate the substrate uptake profile of the normal human heart as reported in several in vivo studies [8-14] (FIG. 2A). At rest, lactate is utilized with the highest rate, followed by fatty acids and ketone bodies. Increased energy demand during pacing is predominantly fueled by increased uptake by carbohydrates (glucose, lactate, pyruvate), while fatty acid utilization remains almost constant. Branched chain amino acids do not contribute significantly to the energy expenditure of the heart (<1%).

TABLE 7
External conditions for simulation of the substrate
utilization rates of the human heart.

| | | | |
|---|---|---|---|
| Glucose [mM] | 5.8 | Valine [mM] | 0.2 |
| Lactate [mM] | 0.8 | Leucine [mM] | 0.15 |
| Fatty acids [mM] | 0.5 | Isoleucine [mM] | 0.06 |
| B-hydroxybuterate [mM] | 0.08 | Insulin [nM] | 100 |
| Acetoacetate [mM] | 0.04 | | TF (Example 2) |
| | | k-load | 0.5/3 |

FIG. 2 shows two model validations highlighting the good concordance of model predictions with experimental data. The examples demonstrate the ability of the heart to ensure cardiac functionality at varying cardiac workload and varying plasma concentrations of energy substrates. In FIG. 2A, the computed substrate uptake profile of the normal human heart is compared with the mean of experimental data taken from several in vivo studies [41-48]. At rest, lactate is utilized with the highest rate, followed by free fatty acids (FFAs) and KBs. Counted in moles ATP per moles substrate (glucose—38, lactate—18, palmitate—138, text book values), FFAs represent the dominating energy source. At moderate pacing, the uptake of the carbohydrates is more than doubled whereas the uptake of FFAs remains essentially unaltered. The energetic contribution of BCAAs was less than 1% at rest and pacing. FIG. 2B shows the relationship between glucose uptake and plasma FFA concentration. The uptake rate of glucose is suppressed with increasing levels of plasma FFAs by inhibition of glucose uptake ensuring the preferential utilization of fatty acids (FIG. 2B).

For patient-specific model calibration, we used protein intensity profiles (defined through LFQ intensities, see Methods) of 17 control hearts, 41 patients with AS and 17 patients with MI. Using two dimensional liquid chromatography prior to tandem mass spectrometry analysis we identified, in total, 9.133 distinct protein groups, from which a subset of 321 proteins was used for model calibration.

### Example 2: External Conditions and Transfer Functions

Glucose-Insulin: The plasma concentrations of the hormone insulin determine the phosphorylation state of the inter-convertible enzymes. Insulin is secreted by the pancreas into the portal vein and the secretion rate is mainly controlled by the glucose concentration of the blood. Therefore, we used the empirical glucose hormone transfer function (GHT), which describes the relationship between the plasma level of glucose and the plasma levels of insulin established in Bulik et al., 2016 [63]:

$$Ins = 1.55 nM * \frac{{( {Glc}_{ext} )}^{5.7}}{{( {Glc}_{ext} )}^{5.7} + {( 7.7 mM )}^{5.7}}$$

Enzyme phosphorylation state: The concentration of insulin determines the phosphorylation state of the interconvertible enzymes [63]. The phosphorylation state γ of interconvertible enzymes is given by:

$$γ = 1 - \frac{{Ins}^{0.65}}{{Ins}^{0.65} + {( 0.4 nM )}^{0.65}}$$

AMP-dependent phosphorylation: In addition to hormone dependent phosphorylation, phosphorylation in dependence of the energetic state of the cell is achieved by the AMP-dependent kinase. Therefore, we introduced AMP dependent phosphorylation by

$${γ}_{AMP} = \frac{AMP}{AMP + 0.04}$$

Glucose-fatty acids: The plasma concentration of fatty acids (FA) is largely determined by the rate of triglyceride lipolysis in the adipose tissue, which is mainly controlled by insulin and glucagon through the activity of the hormone sensitive lipases (HSL). Based on measured relations between the plasma levels of plasma and FA we constructed an empirical glucose-FA transfer function (GFT):

$${tfa}_{plasma} = 1.2 mM - 1.1 mM \frac{{Glc}_{ext}^{4}}{{Glc}_{ext}^{4} + {( 6.5 mM )}^{4}}$$

Conversion of total plasma fatty acids to free plasma fatty acids: Plasma fatty acids are largely bound to plasma albumin or lipoproteins, but only free fatty acids are taken up by the heart. We calculated the free fatty acid concentration (ffαₚₗₐₛₘₐ) from total plasma fatty acid concentration (tfαₚₗₐₛₘₐ) assuming a linear relationship between the two. In this way, we can recapitulate hyperbolic saturation kinetics in the cardiac fatty acid uptake rates when depicted against total plasma fatty acid concentration or against free fatty acid plasma concentration:

$${ffa}_{plasma} = 3.125 \cdot {10}^{- 4} \cdot {tfa}_{plasma}$$

Epinephrine: The plasma concentrations of the hormone epinephrine is an important determinant for the activity of glucose transport capacity in cardiomyocytes. As epinephrine increases cardiac pacing [64], we describe epinephrine levels in dependence of cardia pacing (load) by a transfer function.

### Example 3: Energetic Capacities of the LV of Controls and Patients with Valve Diseases

FIG. 3 depicts the specific energetic parameters MV_{ATP}(rest), MV_{ATP}(max) and MAPR for each subject after 60 min pacing. Compared with controls, the individual variations of these parameters were much larger for the two patient groups (see box plots in FIG. 3 B-D). For MI patients, the mean value of the parameter MV_{ATP}(rest) was significantly higher (890±292 versus 761±10 μmol/g/h), while MV_{ATP}(max) was significantly lower when compared to control values (1713±245 versus 1941±238 μmol/g/h) (two-sample Kolmogorov-Smirnov test). For AS patients, the mean value of the parameter MV_{ATP}(rest) was also significantly higher (800±270 versus 761±10 μmol/g/h) and MV_{ATP}(max) was also significantly lower (1513±257 versus 1941±238 μmol/g/h). For both groups of patients, the parameter MAPR was on the average significantly lower compared to the control (826±448 in MI and 904±340 in AS versus 1180±245 μmol/g/h). Hence, both groups of patients had on the average a reduced ATP production reserve, which was caused by increased MV_{ATP}(rest) and decreased MV_{ATP}(max).

MV_{ATP}(rest) was significantly increased in the MI and AS group and MV_{ATP}(max) was significantly decreased in both groups, resulting in a significant reduction of the specific ATP production reserve MAPR (see FIG. 3). The general decrease of MV_{ATP}(max) in both groups of patients can be accounted for by a decrease of the oxidative phosphorylation capacity as none of the investigated LVs showed excessive glycolytic activity. A decreased expression of the PGC-1α/PPARα transcription cascade has been identified as an important mechanism responsible for the downregulation of the oxidative phosphorylation in the failing myocardium [3].

### Example 4: Substrate Uptake of Patients at Rest and at Maximal Workload

Next, we investigated changes in the substrate preference of the LV accompanying altered metabolic capacity (see FIG. 4). In the resting state, the largest differences occurred for the uptake rates of glucose and lactate for patients with MI. Especially glucose uptake was increased by more than 20%. At rest, there was a significant decrease in lactate utilization in patients with AS. In general, variances in substrate utilization rates were large, again pointing to individually differing metabolic phenotypes. The reduction in lactate utilization was significant also at maximal load in MI and AS patients, while glucose rates were significantly reduced for patients with AS only. FIG. 4 also shows how the different substrates contribute to overall energy production. The contribution of fatty acids accounts for up to two third, while BCAAs always account for <1% of total energy expenditure and are therefore not shown.

Both groups of patients exhibited significant changes in the myocardial utilization of the main energy substrates. Generally, there was a trend towards higher uptake rates for glucose and decreased uptake rates for lactate in patients with MI, and a decrease in glucose as well as lactate utilization for AS patients. Ketone body utilization rates showed a high variability, but were in general increased in AS patients. This is in line with recent studies suggesting that increased reliance on ketone body metabolism offers a metabolic advantage in the failing heart and an ergogenic aid for exercise performance [55, 56].

### Example 5: Association of MV_{ATP} with Clinical Parameters Evaluating the Mechanical Work and the Systolic Performance of the LV

In valve disease, the LV is exposed to chronic pressure load (AS) and/or volume overload (MI). This results in a higher mechanical workload, which can be quantified by the surrogate internal myocardial power estimating the power of the LV required for cardiac contraction [32]. Our analysis provided evidence for a strong correlation between tMV_{ATP} at rest and at maximal pacing and the internal myocardial power (see FIG. 5). Importantly, a significant correlation of the energy parameters has also been found with the cardiac output (see FIG. 5). Taken together, these findings demonstrate a close association between increased ATP production capacity, increased mechanical work of the pressure/volume overloaded LV and cardiac output.

The central findings of our approach are that even in patients with valvular dysfunction but preserved systolic function and no sign of heart failure, the energy metabolism is already deteriorated (see FIG. 3) and closely associated with mechanical power and systolic performance (see FIG. 5). The first finding is in line with several studies (reviewed in [52]) which have established that a reduction in the ATP production capacity already occurs in early phases of heart failure development. The second finding identifies the capability of the cardiac metabolic network to generate ATP as the key link between systolic function and energy metabolism of the LV rather than the intracellular transport capacity of energy-rich phosphates by the CK shuttle, which was found to not be significantly different in AS patients with preserved and reduced systolic function [10].

### Example 6: Metabolic Profiling of Individual Patients

Despite the general trend of the energy parameters in the patients' LV outlined in the preceding section, substantial differences in the metabolic profiles of individual patients occur. As an example, FIG. 6 depicts the individual energetic profiles of three patients with AS with largely differing values of their cardiac energy parameters (see FIG. 3A). Patients A2 and A4 are characterized by impaired MAPR, while patient A13 has a MAPR comparable to healthy hearts (see FIG. 3). The impaired MAPR of patient A2 results from an increased MV_{ATP}(rest) with a normal MV_{ATP}(max), while the impaired MAPR of patient A4 results from an increased MV_{ATP}(rest) and a decreased MV_{ATP}(max). Patient A13 with a normal MAPR has normal MV_{ATP}(rest) and normal MV_{ATP}(max). The individual alterations in the energetics of the LV are also associated with marked differences in substrate utilization rates. For example, while A13 has normal MV_{ATP}(rest), its resting carbohydrate utilization rates (glucose and lactate) are strongly decreased and compensated by an increase KB utilization rate. This increased KB utilization is also maintained at MV_{ATP}(max) and is even more pronounced in A2. In contrast, patient A4 shows a decreased utilization rate for all substrates at MV_{ATP}(max).

Our analysis revealed in both groups of patients a large variability of the energy parameters (see FIGS. 3 and 6), likely reflecting larger differences in the patient-specific functional and structural response of the LV to pressure/volume overload. Whereas some patients present with signs of hypertrophy, myocardial thickening and ventricular dilation, others may show alterations in contraction time or only modest signs of remodeling [53, 54]. The large intra-individual variability of cardiac energetics in patients with valvular dysfunction necessitates an individual assessment of the metabolic status (see FIG. 6).

### REFERENCES

1. Evans, R. D. and K. Clarke, Myocardial substrate metabolism in heart disease. Front Biosci (Schol Ed), 2012. 4: p. 556-80.
2. Lopaschuk, G. D. and J. C. Russell, Myocardial function and energy substrate metabolism in the insulin-resistant JCR:LA corpulent rat. J Appl Physiol (1985), 1991. 71(4): p. 1302-8.
3. Neubauer, S., The failing heart—an engine out of fuel. N Engl J Med, 2007. 356(11): p. 1140-51.
4. Stanley, W. C., F. A. Recchia, and G. D. Lopaschuk, Myocardial substrate metabolism in the normal and failing heart. Physiol Rev, 2005. 85(3): p. 1093-129.
5. Taegtmeyer, H., et al., Assessing Cardiac Metabolism: A Scientific Statement From the American Heart Association. Circ Res, 2016. 118(10): p. 1659-701.
6. Ingwall, J. S. and R. G. Weiss, Is the failing heart energy starved? On using chemical energy to support cardiac function. Circ Res, 2004. 95(2): p. 135-45.
7. HERRMANN, G. and G. M. DECHERD JR, The chemical nature of heart failure. Annals of Internal Medicine, 1939. 12(8): p. 1233-1244.
8. Ning, X.-H., et al., Signaling and expression for mitochondrial membrane proteins during left ventricular remodeling and contractile failure after myocardial infarction. Journal of the American College of Cardiology, 2000. 36(1): p. 282-287.
9. Quigley, A. F., et al., Mitochondrial respiratory chain activity in idiopathic dilated cardiomyopathy. Journal of cardiac failure, 2000. 6(1): p. 47-55.
10. Peterzan, M. A., et al., Cardiac Energetics in Patients With Aortic Stenosis and Preserved Versus Reduced Ejection Fraction. Circulation, 2020. 141(24): p. 1971-1985.
11. Abdurrachim, D. and J. J. Prompers, Evaluation of cardiac energetics by non-invasive 31P magnetic resonance spectroscopy. Biochimica et Biophysica Acta (BBA)-Molecular Basis of Disease, 2018. 1864(5): p. 1939-1948.
12. Ingwall, J. S., Phosphorus nuclear magnetic resonance spectroscopy of cardiac and skeletal muscles. American Journal of Physiology-Heart and Circulatory Physiology, 1982. 242(5): p. H729-H744.
13. Peterzan, M. A., et al., Non-invasive investigation of myocardial energetics in cardiac disease using (31)P magnetic resonance spectroscopy. Cardiovasc Diagn Ther, 2020. 10(3): p. 625-635.
14. Karlstadt, A., et al., CardioNet: a human metabolic network suited for the study of cardiomyocyte metabolism. BMC Syst Biol, 2012. 6: p. 114.
15. Cortassa, S., et al., An integrated model of cardiac mitochondrial energy metabolism and calcium dynamics. Biophys J, 2003. 84(4): p. 2734-55.
16. Beard, D. A., Modeling of oxygen transport and cellular energetics explains observations on in vivo cardiac energy metabolism. PLOS Comput Biol, 2006. 2(9): p. e107.
17. Wu, F., et al., Computer modeling of mitochondrial tricarboxylic acid cycle, oxidative phosphorylation, metabolite transport, and electrophysiology. J Biol Chem, 2007. 282(34): p. 24525-37.
18. Wu, F., et al., Phosphate metabolite concentrations and ATP hydrolysis potential in normal and ischaemic hearts. J Physiol, 2008. 586(17): p. 4193-208.
19. Berndt, N., et al., HEPATOKIN1 is a biochemistry-based model of liver metabolism for applications in medicine and pharmacology. Nat Commun, 2018. 9(1): p. 2386.
20. Hughes, C. S., et al., Single-pot, solid-phase-enhanced sample preparation for proteomics experiments. Nat Protoc, 2019. 14(1): p. 68-85.
21. Rappsilber, J., Y. Ishihama, and M. Mann, Stop and go extraction tips for matrix-assisted laser desorption/ionization, nanoelectrospray, and LC/MS sample pretreatment in proteomics. Analytical Chemistry, 2003. 75(3): p. 663-670.
22. Tyanova, S., T. Temu, and J. Cox, The MaxQuant computational platform for mass spectrometry-based shotgun proteomics. Nat Protoc, 2016. 11(12): p. 2301-2319.
23. Moors, C., et al., Impaired insulin sensitivity is accompanied by disturbances in skeletal muscle fatty acid handling in subjects with impaired glucose metabolism. International journal of obesity, 2012. 36(5): p. 709-717.
24. Gordon, R. S. and A. Cherkes, Unesterified fatty acid in human blood plasma. The Journal of clinical investigation, 1956. 35(2): p. 206-212.
25. Imaizumi, A., et al., Genetic basis for plasma amino acid concentrations based on absolute quantification: a genome-wide association study in the Japanese population. European Journal of Human Genetics, 2019. 27(4): p. 621-630.
26. Nishioka, M., et al., The overnight effect of dietary energy balance on postprandial plasma free amino acid (PFAA) profiles in Japanese adult men. PLOS One, 2013. 8(5).
27. Ottosson, F., et al., Postprandial levels of branch chained and aromatic amino acids associate with fasting glycaemia. Journal of amino acids, 2016. 2016.
28. Ravikumar, B., et al., Real-time assessment of postprandial fat storage in liver and skeletal muscle in health and type 2 diabetes. American Journal of Physiology-Endocrinology and Metabolism, 2005. 288(4): p. E789-E797.
29. Bauer, B. A., et al., Exercise training produces changes in free and conjugated catecholamines. Medicine and science in sports and exercise, 1989. 21(5): p. 558-562.
30. Dimsdale, J. E. and J. Moss, Plasma catecholamines in stress and exercise. Jama, 1980. 243(4): p. 340-342.
31. Nelson, R. R., et al., Hemodynamic predictors of myocardial oxygen consumption during static and dynamic exercise. Circulation, 1974. 50(6): p. 1179-89.
32. Lee, C. B., et al., Surrogates for myocardial power and power efficiency in patients with aortic valve disease. Sci Rep, 2019. 9(1): p. 16407.
33. Henderson, M. J., H. E. Morgan, and C. R. Park, Regulation of glucose uptake in muscle. V. The effect of growth hormone on glucose transport in the isolated, perfused rat heart. J Biol Chem, 1961. 236: p. 2157-61.
34. Morgan, H. E., et al., Regulation of glucose uptake in muscle. I. The effects of insulin and anoxia on glucose transport and phosphorylation in the isolated, perfused heart of normal rats. J Biol Chem, 1961. 236: p. 253-61.
35. Drake, A. J., J. R. Haines, and M. I. Noble, Preferential uptake of lactate by the normal myocardium in dogs. Cardiovasc Res, 1980. 14(2): p. 65-72.
36. Vyska, K., et al., Fatty acid uptake in normal human myocardium. Circ Res, 1991. 69(3): p. 857-70.
37. Stremmel, W., Fatty acid uptake by isolated rat heart myocytes represents a carrier-mediated transport process. J Clin Invest, 1988. 81(3): p. 844-52.
38. Nuutila, P., et al., Effect of antilipolysis on heart and skeletal muscle glucose uptake in overnight fasted humans. Am J Physiol, 1994. 267(6 Pt 1): p. E941-6.
39. Sultan, A. M., Effects of diabetes and insulin on ketone bodies metabolism in heart. Mol Cell Biochem, 1992. 110(1): p. 17-23.
40. Camici, P., et al., Coronary hemodynamics and myocardial metabolism during and after pacing stress in normal humans. Am J Physiol, 1989. 257(3 Pt 1): p. E309-17.
41. Voros, G., et al., Increased Cardiac Uptake of Ketone Bodies and Free Fatty Acids in Human Heart Failure and Hypertrophic Left Ventricular Remodeling. Circ Heart Fail, 2018. 11(12): p. e004953.
42. Bing, R. J., et al., Metabolism of the human heart. II. Studies on fat, ketone and amino acid metabolism. Am J Med, 1954. 16(4): p. 504-15.
43. Mudge, G. H., Jr., et al., Alterations of myocardial amino acid metabolism in chronic ischemic heart disease. J Clin Invest, 1976. 58(5): p. 1185-92.
44. Gertz, E. W., et al., Myocardial substrate utilization during exercise in humans. Dual carbon—labeled carbohydrate isotope experiments. J Clin Invest, 1988. 82(6): p. 2017-25.
45. Bergman, B. C., et al., Myocardial glucose and lactate metabolism during rest and atrial pacing in humans. J Physiol, 2009. 587(Pt 9): p. 2087-99.
46. Mizuno, Y., et al., The diabetic heart utilizes ketone bodies as an energy source. Metabolism, 2017. 77: p. 65-72.
47. Bergman, B. C., et al., Myocardial FFA metabolism during rest and atrial pacing in humans. Am J Physiol Endocrinol Metab, 2009. 296(2): p. E358-66.
48. Camici, P., E. Ferrannini, and L. H. Opie, Myocardial metabolism in ischemic heart disease: basic principles and application to imaging by positron emission tomography. Prog Cardiovasc Dis, 1989. 32(3): p. 217-38.
49. Kaijser, L., M. Ericsson, and G. Walldius, Fatty acid turnover in the ischaemic compared to the non-ischaemic human heart. Mol Cell Biochem, 1989. 88(1-2): p. 181-4.
50. Fillmore, N., et al., Uncoupling of glycolysis from glucose oxidation accompanies the development of heart failure with preserved ejection fraction. Mol Med, 2018. 24(1): p. 3.
51. Kubler, W. and P. G. Spieckermann, Regulation of glycolysis in the ischemic and the anoxic myocardium. J Mol Cell Cardiol, 1970. 1(4): p. 351-77.
52. Sankaralingam, S. and G. D. Lopaschuk, Cardiac energy metabolic alterations in pressure overload-induced left and right heart failure (2013 Grover Conference Series). Pulm Circ, 2015. 5(1): p. 15-28.
53. Dweck, M. R., et al., Left ventricular remodeling and hypertrophy in patients with aortic stenosis: insights from cardiovascular magnetic resonance. J Cardiovasc Magn Reson, 2012. 14: p. 50.
54. Enache, R., et al., CME: long-term outcome in asymptomatic patients with severe aortic regurgitation, normal left ventricular ejection fraction, and severe left ventricular dilatation. Echocardiography, 2010. 27(8): p. 915-22.
55. Aubert, G., et al., The Failing Heart Relies on Ketone Bodies as a Fuel. Circulation, 2016. 133(8): p. 698-705.
56. Harvey, K. L., L. E. Holcomb, and S. C. Kolwicz, Jr., Ketogenic Diets and Exercise Performance. Nutrients, 2019. 11(10).
57. Deussen, A., et al., Heterogeneity of metabolic parameters in the left ventricular myocardium and its relation to local blood flow. Basic Res Cardiol, 2001. 96(6): p. 564-74.
58. Bach, D. S., et al., Heterogeneity of ventricular function and myocardial oxidative metabolism in nonischemic dilated cardiomyopathy. J Am Coll Cardiol, 1995. 25(6): p. 1258-62.
59. Berndt, N. and H. G. Holzhutter, Dynamic Metabolic Zonation of the Hepatic Glucose Metabolism Is Accomplished by Sinusoidal Plasma Gradients of Nutrients and Hormones. Front Physiol, 2018. 9: p. 1786.
60. Berndt, N., et al., A multiscale modelling approach to assess the impact of metabolic zonation and microperfusion on the hepatic carbohydrate metabolism. PLOS Comput Biol, 2018. 14(2): p. e1006005.
61. Stremmel, W., Fatty-Acid Uptake by Isolated Rat-Heart Myocytes Represents a Carrier-Mediated Transport Process. Journal of Clinical Investigation, 1988. 81(3): p. 844-852.]
62. Kampf, J. P. and A. M. Kleinfeld, Fatty acid transport in adipocytes monitored by imaging intracellular free fatty acid levels. Journal of Biological Chemistry, 2004. 279(34): p. 35775-35780.
63. Bulik, S., H. G. Holzhutter, and N. Berndt, The relative importance of kinetic mechanisms and variable enzyme abundances for the regulation of hepatic glucose metabolism— insights from mathematical modeling. BMC Biol, 2016. 14: p. 15.
64. Clutter, W. E., et al., Epinephrine plasma metabolic clearance rates and physiologic thresholds for metabolic and hemodynamic actions in man. J Clin Invest, 1980. 66(1): p. 94-101.
65. WO 2009/124403 A 1 (UNIV BRITISH COLUMBIA [CA]; MCMANUS BRUCE [CA] ET AL.) 15 Oct. 2009 (2009-10-15)
66. WO 2009/124404 A 1 (UNIV BRITISH COLUMBIA [CA]; MCMANUS BRUCE [CA] ET AL.) 15 Oct. 2009 (2009-10-15)
67. US 2017/258526 A 1 (LANG PHILIPP K [US]) 14 Sep. 2017 (2017-09-14)
68. WO 2016/011335 A1 (SCHENTAG JEROME [US]; FAYAD JOSEPH M [US]) 21 Jan. 2016 (2016-01-21)

TABLE 8
Information to Kinetic rate laws (Table 8 uses separate reference numbering)
Fatty acid uptake
Carrier mediated FATP
${v}_{CD 36} = {V}_{max}^{CD 36} \cdot \frac{( {ffa}_{ext} - c {16}_{cyt} )}{1 + \frac{{ffa}_{ext}}{{K}_{m}^{{ffa}_{ext}}} + \frac{c {16}_{cyt}}{{K}_{m}^{c {16}_{cyt}}}}$
Vₘₐₓ^{CD36} for numerical value see Table 10 Kₘ^{ffa}^{ₑₓₜ} = 0.000085 [1] Kₘ^{c16}^{_{cyt}} = 0.004 [2]
(Long-chain) acyl-coa synthetase
${v}_{ACS 1} = {V}_{max}^{ACS 1} \cdot \frac{c {16}_{cyt}}{c {16}_{cyt} + {K}_{m}^{c {16}_{cyt}}} \cdot \frac{{atp}_{cyt}}{{atp}_{cyt} + {K}_{m}^{{atp}_{cyt}}} \cdot \frac{{coa}_{cyt}}{{coa}_{cyt} + {K}_{m}^{{coa}_{cyt}}}$
Vₘₐₓ^{ACS1} for numerical value see Table 10
Kₘ^{c16}^{_{cyt}} = 0.033 [3] Kₘ^{atp}^{_{cyt}} = 0.320 [3] Kₘ^{coa}^{_{cyt}} = 0.0064 [3]
${v}_{FATP 1} = {V}_{max}^{FATP 1} \cdot \frac{c {16}_{cyt}}{c {16}_{cyt} + {K}_{m}^{c {16}_{cyt}}} \cdot \frac{{atp}_{cyt}}{{atp}_{cyt} + {K}_{m}^{{atp}_{cyt}}} \cdot \frac{{coa}_{cyt}}{{coa}_{cyt} + {K}_{m}^{{coa}_{cyt}}}$
${V}_{max}^{FATP 1} = \frac{{V}_{max}^{ACS 1}}{27} [ 3 ] {K}_{m}^{c {16}_{cyt}} = 0.021 [ 3 ] {K}_{m}^{{atp}_{cyt}} = 0.85 [ 3 ] {K}_{m}^{{coa}_{cyt}} = 0.0083 [ 3 ]$
${v}_{FATP 4} = {V}_{max}^{FATP 4} \cdot \frac{c {16}_{cyt}}{c {16}_{cyt} + {K}_{m}^{c {16}_{cyt}}} \cdot \frac{{atp}_{cyt}}{{atp}_{cyt} + {K}_{m}^{{atp}_{cyt}}} \cdot \frac{{coa}_{cyt}}{{coa}_{cyt} + {K}_{m}^{{coa}_{cyt}}}$
Vₘₐₓ^{FATP4} = 1.3 · Vₘₐₓ^{ASC1} [4] Kₘ^{c16}^{_{cyt}} = 0.013 [4] Kₘ^{atp}^{_{cyt}} = 1.4 [4]
Kₘ^{coa}^{_{cyt}} = 0.047 [4]
Beta-oxidation
Carnitine palmitoyltransferase I (muscle isoform)
${v}_{CPT 1} = {V}_{max}^{CPT 1} \cdot \frac{c 16 {coa}_{cyt} \cdot {car}_{cyt}}{( c 16 {coa}_{cyt} + {K}_{m}^{c 16 {coa}_{cyt}} ) \cdot ( {car}_{cyt} + {K}_{m}^{{car}_{cyt}} )}$
Vₘₐₓ^{CPT1} for numerical value see Table 10
${K}_{m}^{c 16 {coa}_{cyt}} = {K}_{0}^{c 16 {coa}_{cyt}} \cdot ( 1 + \frac{malcoa {2}_{imm}}{{K}_{i}^{malcoa {2}_{imm}}} )$
K₀^{c16coa}^{_{cyt}} = 0.073 [5] Kᵢ^{malcoa2}^{ᵢₘₘ} = 0.0001 [6] Kₘ^{car}^{_{cyt}} = 0.19 [7]
Carnitine acylcarnitine translocase
${v}_{CACT} = {V}_{max}^{CACT} \cdot ( \frac{{car}_{mito} \cdot c 16 {car}_{cyt} - 1 / {K}_{eq}^{CACT} \cdot {car}_{cyt} \cdot c 16 {car}_{mito}}{( 1 + \frac{{car}_{mito}}{{K}_{m}^{{car}_{mito}}} ) ( 1 + \frac{c 16 {car}_{cyt}}{{K}_{m}^{c 16 {coa}_{cyt}}} ) + ( 1 + \frac{{car}_{cyt}}{{K}_{m}^{{car}_{cyt}}} ) ( 1 + \frac{c 16 {car}_{mito}}{{K}_{m}^{c 16 {car}_{mito}}} ) - 1} )$
Vₘₐₓ^{CACT} for numerical value see Table 10
K_{eq}^{CACT} = 1.6 [8] Kₘ^{car}^{ₘᵢₜₒ} = 5.8 [9] Kₘ^{c16car}^{_{cyt}} = 0.001 [10] Kₘ^{car}^{_{cyt}} = 1.3 [11]
Kₘ^{c16car}^{ₘᵢₜₒ} = 0.0051 [12]
Carnitine palmitoyltransferase 2
${v}_{CPT 2} = {V}_{max}^{CPT 2} \cdot ( \frac{c 16 {car}_{mito} \cdot {coa}_{mito} - 1 / {K}_{eq}^{CPT 2} \cdot c 16 {coa}_{mito} \cdot {car}_{mito}}{( 1 + \frac{c 16 {car}_{mito}}{{K}_{m}^{c 16 {car}_{mito}}} ) ( 1 + \frac{{coa}_{mito}}{{K}_{m}^{{coa}_{mito}}} ) + ( 1 + \frac{c 16 {coa}_{mito}}{{K}_{m}^{c 16 {coa}_{mito}}} ) ( 1 + \frac{{car}_{mito}}{{K}_{m}^{{car}_{mito}}} ) - 1} )$
Vₘₐₓ^{CPT2} for numerical value see Table 10
K_{eq}^{CPT2} = 2 [13] Km^{c16car}^{ₘᵢₜₒ} = 0.12 [14] Kₘ^{coa}^{ₘᵢₜₒ} = 0.0055 [15] Kₘ^{c16coa}^{ₘᵢₜₒ} = 0.191 [16]
Kₘ^{car}^{ₘᵢₜₒ} = 0.46 [17]
Short chain acyl-coa dehydrogenase (c4) (identical to liver enzyme [18])
${v}_{c 4 coa - scdh} = {V}_{max}^{c 4 coa - dh} \cdot ( \frac{c 4 {coa}_{mito}}{c 4 {coa}_{mito} + {K}_{m}^{c 4 {coa}_{mito}}} ) \cdot ( \frac{{etffad}_{mito}}{{etffad}_{mito} + {K}_{m}^{{etffad}_{mito}}} )$
Vₘₐₓ^{c4coa-dh} for numerical value see Table 10
Kₘ^{c4coa}^{ₘᵢₜₒ} = 0.0107 [19] Kₘ^{etffad}^{ₘᵢₜₒ} = 0.0038 [19]
Short chain acyl-coa dehydrogenase (c5) (identical to liver enzyme [18])
${v}_{c 5 coa - scdh} = {V}_{max}^{c 5 coa - dh} \cdot ( \frac{c 5 {coa}_{mito}}{c 5 {coa}_{mito} + {K}_{m}^{c 5 {coa}_{mito}}} ) \cdot ( \frac{{etffad}_{mito}}{{etffad}_{mito} + {K}_{m}^{{etffad}_{mito}}} )$
Vₘₐₓ^{c5coa-dh} for numerical value see Table 10
Kₘ^{c5coa}^{ₘᵢₜₒ} = 0.01 Kₘ^{etffad}^{ₘᵢₜₒ} = 0.0038 [19]
Medium chain acyl-coa dehydrogenase (c6) (identical to liver enzyme [18])
${v}_{c 6 coa - mcdh} = {V}_{max}^{c 6 coa - dh} \cdot ( \frac{c 6 {coa}_{mito}}{c 6 {coa}_{mito} + {K}_{m}^{c 6 {coa}_{mito}}} ) \cdot ( \frac{{etffad}_{mito}}{{etffad}_{mito} + {K}_{m}^{{etffad}_{mito}}} )$
Vₘₐₓ^{c6coa-dh} for numerical value see Table 10
${K}_{m}^{c {6}_{{coa}_{mito}}} = 0.0094 [ 19 ] \qquad {K}_{m}^{{etffad}_{mito}} = 0.0 \; 045 [ 19 ]$
Medium chain acyl-coa dehydrogenase (c8) (identical to liver enzyme [18])
${v}_{c 8 coa - mcdh} = {V}_{max}^{c 8 coa - dh} \cdot ( \frac{c 8 {coa}_{mito}}{c 8 {coa}_{mito} + {K}_{m}^{c 8 {coa}_{mito}}} ) \cdot ( \frac{{etffad}_{mito}}{{etffad}_{mito} + {K}_{m}^{{etffad}_{mito}}} )$
Vₘₐₓ^{c8coa-dh} for numerical value see Table 10
Kₘ^{c8coa}^{ₘᵢₜₒ} = 0.004 [19] K^{metffad}^{ₘᵢₜₒ} = 0.0045 [19]
Medium chain acyl-coa dehydrogenase (c10) (identical to liver enzyme [18])
${v}_{c 10 coa - mcdh} = {V}_{max}^{c 10 coa - dh} \cdot ( \frac{c 10 {coa}_{mito}}{c 10 {coa}_{mito} + {K}_{m}^{c 10 {coa}_{mito}}} ) \cdot ( \frac{{etffad}_{mito}}{{etffad}_{mito} + {K}_{m}^{{etffad}_{mito}}} )$
Vₘₐₓ^{c10coa-dh} for numerical value see Table 10
Kₘ^{c10coa}ₘᵢₜₒ = 0.0054 [19] Kₘ^{etffad}^{ₘᵢₜₒ} = 0.0045 [19]
Medium chain acyl-coa dehydrogenase (c12) (identical to liver enzyme [18])
${v}_{c 12 coa - mcdh} = {V}_{max}^{c 12 coa - dh} \cdot ( \frac{c 12 {coa}_{mito}}{c 12 {coa}_{mito} + {K}_{m}^{c 12 {coa}_{mito}}} ) \cdot ( \frac{{etffad}_{mito}}{{etffad}_{mito} + {K}_{m}^{{etffad}_{mito}}} )$
Vₘₐₓ^{c12coa-dh} for numerical value see Table 10
Kₘ^{c12coa}^{ₘᵢₜₒ} = 0.0057 [19] Kₘ^{etffad}^{ₘᵢₜₒ} = 0.0045 [19]
Long chain acyl-coa dehydrogenase (c10) (identical to liver enzyme [18])
${v}_{c 10 coa - lcdh} = {V}_{max}^{c 10 coa - dh} \cdot ( \frac{c 10 {coa}_{mito}}{c 10 {coa}_{mito} + {K}_{m}^{c 10 {coa}_{mito}}} ) \cdot ( \frac{{etffad}_{mito}}{{etffad}_{mito} + {K}_{m}^{{etffad}_{mito}}} )$
${V}_{max}^{c 10 coa - dh} for numerical value see Table 10 {K}_{m}^{c 10 {coa}_{mito}} = {K}_{0}^{c 10 {coa}_{mito}} \cdot ( 1 + \frac{kc 16 {coa}_{mito}}{{K}_{i}^{kc 16 {coa}_{mito}}} )$
Kᵢ^{kc16coa}^{ₘᵢₜₒ} = 0.00047 [20] K₀^{c10coa}^{ₘᵢₜₒ} = 0.0243 [19] Kₘ^{etffad}^{ₘᵢₜₒ} = 0.0083 [19]
Long chain acyl-coa dehydrogenase (c12) (identical to liver enzyme [18])
${v}_{c 12 coa - lcdh} = {V}_{max}^{c 12 coa - dh} \cdot ( \frac{c 12 {coa}_{mito}}{c 12 {coa}_{mito} + {K}_{m}^{c 12 {coa}_{mito}}} ) \cdot ( \frac{{etffad}_{mito}}{{etffad}_{mito} + {K}_{m}^{{etffad}_{mito}}} )$
${V}_{max}^{c 12 coa - dh} for numerical value see Table 10 {K}_{m}^{c 12 {coa}_{mito}} = {K}_{0}^{c 12 {coa}_{mito}} \cdot ( 1 + \frac{kc 16 {coa}_{mito}}{{K}_{i}^{kc 16 {coa}_{mito}}} )$
kᵢ^{kc16coa}^{ₘᵢₜₒ} = 0.00047 [20] K₀^{c12coa}^{ₘᵢₜₒ} = 0.009 [19] Kₘ^{etffad}^{ₘᵢₜₒ} = 0.0083 [19]
Long chain acyl-coa dehydrogenase (c14) (identical to liver enzyme [18])
${v}_{c 14 coa - lcdh} = {V}_{max}^{c 14 coa - dh} \cdot ( \frac{c 14 {coa}_{mito}}{c 14 {coa}_{mito} + {K}_{m}^{c 14 {coa}_{mito}}} ) \cdot ( \frac{{etffad}_{mito}}{{etffad}_{mito} + {K}_{m}^{{etffad}_{mito}}} )$
Vₘₐₓ^{c14coa-dh} for numerical value see Table 10
${K}_{m}^{c 14 {coa}_{mito}} = {K}_{0}^{c 14 {coa}_{mito}} \cdot ( 1 + \frac{kc 16 {coa}_{mito}}{{K}_{i}^{kc 16 {coa}_{mito}}} )$
Kᵢ^{kc16coa}^{ₘᵢₜₒ} = 0.00047 [20] K₀^{c14coa}^{ₘᵢₜₒ} = 0.0074 [19] Kₘ^{etffad}^{ₘᵢₜₒ} = 0.0083 [19]
Long chain acyl-coa dehydrogenase (c16) (identical to liver enzyme [18])
${v}_{c 16 coa - lcdh} = {V}_{max}^{c 16 coa - dh} \cdot ( \frac{c 16 {coa}_{mito}}{c 16 {coa}_{mito} + {K}_{m}^{c 16 {coa}_{mito}}} ) \cdot ( \frac{{etffad}_{mito}}{{etffad}_{mito} + {K}_{m}^{{etffad}_{mito}}} )$
Vₘₐₓ^{c16coa-dh} for numerical value see Table 10
${K}_{m}^{c 16 {coa}_{mito}} = {K}_{0}^{c 16 {coa}_{mito}} \cdot ( 1 + \frac{kc 16 {coa}_{mito}}{{K}_{i}^{kc 16 {coa}_{mito}}} )$
Kᵢ^{kc16coa}^{ₘᵢₜₒ} = 0.00047 [20] K₀^{c16coa}^{ₘᵢₜₒ} = 0.0025 [19] Kₘ^{etffad}^{ₘᵢₜₒ} = 0.0083 [19]
Enoyl-coa hydratase (Crontonase) (ec4)
${v}_{ehyd - ec 4} = {V}_{max}^{ehyd - ec 4} \cdot ( \frac{ec 4 {coa}_{mito} - 1 / {K}_{eq}^{ehyd - ec 4} \cdot lc 4 {coa}_{mito}}{ec 4 {coa}_{mito} + {K}_{m}^{ec 4 {coa}_{mito}}} )$
Vₘₐₓ^{ehyd-ec4} for numerical value see Table 10
${K}_{eq}^{ehyd - ec 4} = 0.25 [ 21 ] {K}_{m}^{ec 4 {coa}_{mito}} = {K}_{0}^{ec 4 {coa}_{mito}} \cdot ( 1 + \frac{kc 4 {coa}_{mito}}{{K}_{i}^{kc 4 {coa}_{mito}}} )$
K₀^{ec4coa}^{ₘᵢₜₒ} = 0.013 [22] Kᵢ^{kc4coa}^{ₘᵢₜₒ} = 0.025 [23]
Enoyl-coa hydratase (Crontonase) (ec6)
${v}_{ehyd - ec 6} = {V}_{max}^{ehyd - ec 6} \cdot ( \frac{ec 6 {coa}_{mito} - 1 / {K}_{eq}^{ehyd - ec 6} \cdot lc 6 {coa}_{mito}}{ec 6 {coa}_{mito} + {K}_{m}^{ec 6 {coa}_{mito}}} )$
${V}_{max}^{ehyd - ec 6} = {V}_{max}^{ehyd - ec 4} \cdot \frac{1280}{1670} [ 22 ] \qquad {K}_{eq}^{ehyd - ec 6} = 2 [ 21 ]$
${K}_{m}^{ec 6 {coa}_{mito}} = {K}_{0}^{ec {6}_{{coa}_{mito}}} \cdot ( 1 + \frac{kc 4 {coa}_{mito}}{{K}_{i}^{kc 4 {coa}_{mito}}} )$
K₀^{ec6coa}^{ₘᵢₜₒ} = 0.029 [22] Kᵢ^{kc4coa}^{ₘᵢₜₐ} = 0.025 [23]
Enoyl-coa hydratase (Crontonase) (ec8)
${v}_{ehyd - ec 8} = {V}_{max}^{ehyd - ec 8} \cdot ( \frac{ec 8 {coa}_{mito} - 1 / {K}_{eq}^{ehyd - ec 8} \cdot lc 8 {coa}_{mito}}{ec 8 {coa}_{mito} + {K}_{m}^{ec 8 {coa}_{mito}}} )$
${V}_{max}^{ehyd - ec 8} = {V}_{max}^{ehyd - ec 4} \cdot \frac{910}{1670} [ 22 ] \qquad {K}_{eq}^{ehyd - ec 8} = 2 [ 21 ]$
${K}_{m}^{ec 8 {coa}_{mito}} = {K}_{0}^{ec 8 {coa}_{mito}} \cdot ( 1 + \frac{kc 4 {coa}_{mito}}{{K}_{i}^{kc 4 {coa}_{mito}}} )$
k₀^{ec8coa}^{ₘᵢₜₒ} = 0.029 [22] Kᵢ^{kc4coa}^{ₘᵢₜₒ} = 0.025 [23]
Enoyl-coa hydratase (Crontonase) (ec10)
${v}_{ehyd - ec 10} = {V}_{max}^{ehyd - ec 10} \cdot ( \frac{ec 10 {coa}_{mito} - 1 / {K}_{eq}^{ehyd - ec 10} \cdot lc 10 {coa}_{mito}}{ec 10 {coa}_{mito} + {K}_{m}^{ec 10 {coa}_{mito}}} )$
${V}_{max}^{ehyd - ec 10} = {V}_{max}^{ehyd - ec 4} \cdot \frac{540}{1670} [ 22 ] {K}_{eq}^{ehyd - ec 10} = 2 [ 21 ]$
${K}_{m}^{ec 10 {coa}_{mito}} = {K}_{0}^{ec 10 {coa}_{mito}} \cdot ( 1 + \frac{kc 4 {coa}_{mito}}{{K}_{i}^{kc 4 {coa}_{mito}}} ) {K}_{0}^{ec 10 {coa}_{mito}} = 0.029 [ 22 ] {K}_{i}^{kc 4 {coa}_{mito}} = 0.025 [ 23 ]$
Enoyl-coa hydratase (Crontonase) (ec12)
${v}_{ehyd - ec 12} = {V}_{max}^{ehyd - ec 12} \cdot ( \frac{ec 12 {coa}_{mito} - 1 / {K}_{eq}^{ehyd - ec 12} \cdot lc 12 {coa}_{mito}}{ec 12 {coa}_{mito} + {K}_{m}^{ec 12 {coa}_{mito}}} )$
${V}_{max}^{ehyd - ec 12} = {V}_{max}^{ehyd - ec 4} \cdot \frac{160}{1670} [ 22 ] {K}_{eq}^{ehyd - ec 12} = 2 [ 21 ]$
${K}_{m}^{ec 12 {coa}_{mito}} = {K}_{0}^{ec 12 {coa}_{mito}} \cdot ( 1 + \frac{kc 4 {coa}_{mito}}{{K}_{i}^{kc 4 {coa}_{mito}}} ) {K}_{0}^{ec 12 {coa}_{mito}} = 0.03 [ 22 ] {K}_{i}^{kc 4 {coa}_{mito}} = 0.025 [ 23 ]$
Enoyl-coa hydratase (Crontonase) (ec14)
${v}_{ehyd - ec 14} = {V}_{max}^{ehyd - ec 14} \cdot ( \frac{ec 14 {coa}_{mito} - 1 / {K}_{eq}^{ehyd - ec 14} \cdot lc 14 {coa}_{mito}}{ec 14 {coa}_{mito} + {K}_{m}^{ec 14 {coa}_{mito}}} )$
${V}_{max}^{ehyd - ec 14} = {V}_{max}^{ehyd - ec 16} \cdot \frac{5}{2.3} [ 24 ] {K}_{eq}^{ehyd - ec 14} = 2 [ 21 ]$
${K}_{m}^{ec 14 {coa}_{mito}} = {K}_{0}^{ec 14 {coa}_{mito}} \cdot ( 1 + \frac{kc 4 {coa}_{mito}}{{K}_{i}^{kc 4 {coa}_{mito}}} )$
K₀^{ec14coa}^{ₘᵢₜₒ} = 0.025 [23] Kᵢ^{kc4coa}^{ₘᵢₜₒ} = 0.025 [23]
Enoyl-coa hydratase (Crontonase) (ec16)
${v}_{ehyd - ec 16} = {V}_{max}^{ehyd - ec 16} \cdot ( \frac{ec 16 {coa}_{mito} 1 / {K}_{eq}^{ehyd - ec 16} \cdot lc 16 {coa}_{mito}}{ec 16 {coa}_{mito} + {K}_{m}^{ec 16 {coa}_{mito}}} )$
${V}_{max}^{ehyd - ec 16} = {V}_{max}^{ehyd - ec 4} \cdot \frac{40}{1670} [ 22 ] {K}_{eq}^{ehyd - ec 16} = 2 [ 21 ]$
${K}_{m}^{ec 16 {coa}_{mito}} = {K}_{0}^{ec 16 {coa}_{mito}} \cdot ( 1 + \frac{kc 4 {coa}_{mito}}{{K}_{i}^{kc 4 {coa}_{mito}}} )$
K₀^{ec16coa}^{ₘᵢₜₒ} = 0.030 [22] Kᵢ^{kc4coa}^{ₘᵢₜₒ} = 0.025 [23]
3-hydroxyacyl-coa dehydrogenase (Ic4) (identic with liver enzyme [25])
${v}_{3 hdh - lc 4} = {V}_{max}^{3 hdh - lc 4} \cdot ( \frac{lc 4 {coa}_{mito} \cdot {nad}_{mito} - 1 / {K}_{eq}^{3 hdh - lc 4} \cdot kc 4 {coa}_{mito} \cdot {nadh}_{mito}}{\begin{matrix}( 1 + \frac{lc 4 {coa}_{mito}}{{K}_{m}^{lc 4 {coa}_{mito}}} ) \cdot ( 1 + \frac{{nad}_{mito}}{{K}_{m}^{{nad}_{mito}}} ) + \\ ( 1 + \frac{kc 4 {coa}_{mito}}{{K}_{m}^{{kc 4 coa}_{mito}}} ) \cdot ( 1 + \frac{{nadh}_{mito}}{{K}_{m}^{{nadh}_{mito}}} ) - 1\end{matrix}} )$
Vₘₐₓ^{3hdh-ic4} for numerical value see Table 10
${K}_{eq}^{3 hdh - lc 4} = \frac{1}{0.012} [ 26 ] {K}_{m}^{lc 4 {coa}_{mito}} = 0.0072 [ 27 ]$
Kₘ^{nad}^{ₘᵢₜₒ} = 0.0154 [27] Kₘ^{kc4coa}^{ₘᵢₜₒ} = 0.0169 [28] = Kₘ^{nadh}^{ₘᵢₜₒ} = 0.0118 [27]
3-hydroxyacyl-coa dehydrogenase (Ic6) (identic with liver enzyme [25])
${v}_{3 hdh - lc 6} = {V}_{max}^{3 hdh - lc 6} \cdot ( \frac{lc 6 {coa}_{mito} \cdot {nad}_{mito} - 1 / {K}_{eq}^{3 hdh - lc 6} \cdot kc 6 {coa}_{mito} \cdot {nadh}_{mito}}{\begin{matrix}( 1 + \frac{lc 6 {coa}_{mito}}{{K}_{m}^{lc 6 {coa}_{mito}}} ) \cdot ( 1 + \frac{{nad}_{mito}}{{K}_{m}^{{nad}_{mito}}} ) + \\ ( 1 + \frac{kc 6 {coa}_{mito}}{{K}_{m}^{{kc 6 coa}_{mito}}} ) \cdot ( 1 + \frac{{nadh}_{mito}}{{K}_{m}^{{nadh}_{mito}}} ) - 1\end{matrix}} )$
Vₘₐₓ^{3hdh-lc6} for numerical value see Table 10
${K}_{eq}^{3 hdh - lc 6} = \frac{1}{8 \cdot {10}^{- 4}} [ 29 ] {K}_{m}^{lc 6 {coa}_{mito}} = 0.0286 [ 30 ] {K}_{m}^{{nad}_{mito}} = 0.015 [ 27 ]$
Kₘ^{kc6coa}^{ₘᵢₜₒ} 0.0057 [31] Kₘ^{nadh}^{ₘᵢₜₒ} = 0.011 [27]
3-hydroxyacyl-coa dehydrogenase (Ic8) (identic with liver enzyme [25])
${v}_{3 hdh - lc 8} = {V}_{max}^{3 hdh - lc 8} \cdot ( \frac{lc 8 {coa}_{mito} \cdot {nad}_{mito} - 1 / {K}_{eq}^{3 hdh - lc 8} \cdot kc 8 {coa}_{mito} \cdot {nadh}_{mito}}{\begin{matrix}( 1 + \frac{lc 8 {coa}_{mito}}{{K}_{m}^{lc 8 {coa}_{mito}}} ) \cdot ( 1 + \frac{{nad}_{mito}}{{K}_{m}^{{nad}_{mito}}} ) + \\ ( 1 + \frac{kc 8 {coa}_{mito}}{{K}_{m}^{{kc 8 coa}_{mito}}} ) \cdot ( 1 + \frac{{nadh}_{mito}}{{K}_{m}^{{nadh}_{mito}}} ) - 1\end{matrix}} )$
Vₘₐₓ^{3hdh-lc8} for numerical value see Table 10
${K}_{eq}^{3 hdh - lc 8} = \frac{1}{{10}^{- 3}} {K}_{m}^{lc 8 {coa}_{mito}} = 0.0163 [ 28 ] {K}_{m}^{{nad}_{mito}} = 0.015 [ 27 ]$
Kₘ^{kc8coa}^{ₘᵢₜₒ} = 0.0031 [31] Kₘ^{nadh}^{ₘᵢₜₒ} = 0.011 [27]
3-hydroxyacyl-coa dehydrogenase (Ic10) (identic with liver enzyme [25])
${v}_{3 hdh - lc 10} = {V}_{max}^{3 hdh - lc 10} \cdot ( \frac{lc 10 {coa}_{mito} \cdot {nad}_{mito} - 1 / {K}_{eq}^{3 hdh - lc 10} \cdot kc 10 {coa}_{mito} \cdot {nadh}_{mito}}{\begin{matrix}( 1 + \frac{lc 10 {coa}_{mito}}{{K}_{m}^{lc 10 {coa}_{mito}}} ) \cdot ( 1 + \frac{{nad}_{mito}}{{K}_{m}^{{nad}_{mito}}} ) + \\ ( 1 + \frac{kc 10 {coa}_{mito}}{{K}_{m}^{{kc 10 coa}_{mito}}} ) \cdot ( 1 + \frac{{nadh}_{mito}}{{K}_{m}^{{nadh}_{mito}}} ) - 1\end{matrix}} )$
Vₘₐₓ^{3hdh-lc10} for numerical value see Table 10
${K}_{eq}^{3 hdh - lc 10} = \frac{1}{{10}^{- 3}} {K}_{m}^{lc 10 {coa}_{mito}} = 0.0029 [ 27 ] {K}_{m}^{{nad}_{mito}} = 0.0104 [ 27 ]$
${K}_{m}^{kc {10}_{{coa}_{mito}}} = 0.0018 [ 31 ] \qquad {K}_{m}^{{nadh}_{mito}} = 0.0011 [ 27 ]$
3-hydroxyacyl-coa dehydrogenase (Ic12) (identic with liver enzyme [25])
${v}_{3 hdh - lc 12} = {V}_{max}^{3 hdh - lc 12} \cdot ( \frac{lc 12 {coa}_{mito} \cdot {nad}_{mito} - 1 / {K}_{eq}^{3 hdh - lc 12} \cdot kc 12 {coa}_{mito} \cdot {nadh}_{mito}}{\begin{matrix}( 1 + \frac{lc 12 {coa}_{mito}}{{K}_{m}^{lc 12 {coa}_{mito}}} ) \cdot ( 1 + \frac{{nad}_{mito}}{{K}_{m}^{{nad}_{mito}}} ) + \\ ( 1 + \frac{kc 12 {coa}_{mito}}{{K}_{m}^{{kc 12 coa}_{mito}}} ) \cdot ( 1 + \frac{{nadh}_{mito}}{{K}_{m}^{{nadh}_{mito}}} ) - 1\end{matrix}} )$
Vₘₐₓ^{3hdh-lc12} for numerical value see Table 10
${K}_{eq}^{3 hdh - lc 12} = \frac{1}{{10}^{- 3}} {K}_{m}^{lc 12 {coa}_{mito}} = 0.0018 [ 31 ] {K}_{m}^{{nad}_{mito}} = 0.015 [ 27 ]$
Kₘ^{kc12coa}^{ₘᵢₜₒ} = 0.0018 [31] Kₘ^{nadh}^{ₘᵢₜₒ} = 0.011[27]
3-hydroxyacyl-coa dehydrogenase (Ic14) (identic with liver enzyme [25])
${v}_{3 hdh - lc 14} = {V}_{max}^{3 hdh - lc 14} \cdot ( \frac{lc 14 {coa}_{mito} \cdot {nad}_{mito} - 1 / {K}_{eq}^{3 hdh - lc 14} \cdot kc 14 {coa}_{mito} \cdot {nadh}_{mito}}{\begin{matrix}( 1 + \frac{lc 14 {coa}_{mito}}{{K}_{m}^{lc 14 {coa}_{mito}}} ) \cdot ( 1 + \frac{{nad}_{mito}}{{K}_{m}^{{nad}_{mito}}} ) + \\ ( 1 + \frac{kc 14 {coa}_{mito}}{{K}_{m}^{{kc 14 coa}_{mito}}} ) \cdot ( 1 + \frac{{nadh}_{mito}}{{K}_{m}^{{nadh}_{mito}}} ) - 1\end{matrix}} )$
Vₘₐₓ^{3hdh-lc14} for numerical value see Table 10
${K}_{eq}^{3 hdh - lc 14} = \frac{1}{{10}^{- 3}} {K}_{m}^{lc 14 {coa}_{mito}} = 0.0015 [ 31 ] {K}_{m}^{{nad}_{mito}} = 0.015 [ 27 ]$
Kₘ^{kc14coa}^{ₘᵢₜₒ} = 0.0013[31] Kₘ^{nadh}^{ₘᵢₜₒ} = 0.011[27]
3-hydroxyacyl-coa dehydrogenase (Ic16) (identic with liver enzyme [25])
${v}_{3 hdh - lc 16} = {V}_{max}^{3 hdh - lc 16} \cdot ( \frac{lc 16 {coa}_{mito} \cdot {nad}_{mito} - 1 / {K}_{eq}^{3 hdh - lc 16} \cdot kc 16 {coa}_{mito} \cdot {nadh}_{mito}}{\begin{matrix}( 1 + \frac{lc 16 {coa}_{mito}}{{K}_{m}^{lc 16 {coa}_{mito}}} ) \cdot ( 1 + \frac{{nad}_{mito}}{{K}_{m}^{{nad}_{mito}}} ) + \\ ( 1 + \frac{kc 16 {coa}_{mito}}{{K}_{m}^{{kc 16 coa}_{mito}}} ) \cdot ( 1 + \frac{{nadh}_{mito}}{{K}_{m}^{{nadh}_{mito}}} ) - 1\end{matrix}} )$
Vₘₐₓ^{3hdh-lc16} for numerical value see Table 10
${K}_{eq}^{3 hdh - lc 16} = \frac{1}{{10}^{- 3}} {K}_{m}^{lc {16}_{{coa}_{mito}}} = 0.003 [ 27 ]$
${K}_{m}^{{nad}_{mito}} = 0.0145 [ 27 ] \qquad {K}_{m}^{kc {16}_{{coa}_{mito}}} = 0.0013 [ 31 ] \qquad {K}_{m}^{{nadh}_{mito}} = 0.0 \; 11 [ 27 ]$
3-ketoacyl-coa thiolase I (kc4)
${v}_{3 kt}^{kc 4 coa} = {V}_{max}^{3 kt - kc {4}_{coa}} \cdot ( \frac{{coa}_{mito} \cdot kc 4 {coa}_{mito} - 1 / {K}_{eq}^{3 kt} \cdot {acoa}_{mito}^{2}}{( 1 + \frac{{coa}_{mito}}{{K}_{m}^{{coa}_{mito}}} ) \cdot ( 1 + \frac{kc 4 {coa}_{mito}}{{K}_{m}^{{kc 4 coa}_{mito}}} ) + ( 1 + \frac{{acoa}_{mito}}{{K}_{m}^{{acoa}_{mito}}} ) - 1} )$
Vₘₐₓ^{3kt-kc4} for numerical value see Table 10 K_{eq}^{3kt} = 2500 [32] Kₘ^{coa}^{ₘᵢₜₒ} = 0.0087 [33]
${K}_{m}^{kc 4 {coa}_{mito}} = {K}_{0}^{kc 4 {coa}_{mito}} \cdot ( 1 + \frac{{acoa}_{mito}}{{K}_{i}^{{acoa}_{mito}}} ) {K}_{i}^{{acoa}_{mito}} = 0.125 [ 34 ] {K}_{0}^{kc 4 {coa}_{mito}} = 0.017 [ 33 ]$
${K}_{m}^{{acoa}_{mito}} = {K}_{0}^{{acoa}_{mito}} \cdot ( 1 + \frac{kc 4 {coa}_{mito}}{{K}_{i}^{kc 4 {coa}_{mito}}} ) {K}_{0}^{{acoa}_{mito}} = 0.3 [ 35 ] {K}_{i}^{kc 4 {coa}_{mito}} = 0.0022 [ 36 ]$
3-ketoacyl-coa thiolase II (kc4)
${v}_{3 ktII}^{kc 4 coa} = {V}_{max}^{3 ktII - kc {4}_{coa}} \cdot ( \frac{{coa}_{mito} \cdot kc 4 {coa}_{mito} - 1 / {K}_{eq}^{3 kt} \cdot {acoa}_{mito}^{2}}{( 1 + \frac{{coa}_{mito}}{{K}_{m}^{{coa}_{mito}}} ) \cdot ( 1 + \frac{kc 4 {coa}_{mito}}{{K}_{m}^{{kc 4 coa}_{mito}}} ) + ( 1 + \frac{{acoa}_{mito}}{{K}_{m}^{{acoa}_{mito}}} ) - 1} )$
Vₘₐₓ^{3ktll-kc4c} for numerical value see Table 10 K_{eq}^{3kt} = 2500 [32] Kₘ^{coa}^{ₘᵢₜₒ} = 0.0513 [33]
${K}_{m}^{kc 4 {coa}_{mito}} = {K}_{0}^{kc 4 {coa}_{mito}} \cdot ( 1 + \frac{{acoa}_{mito}}{{K}_{i}^{{acoa}_{mito}}} ) {K}_{i}^{{acoa}_{mito}} = 0.125 [ 34 ] {K}_{0}^{kc 4 {coa}_{mito}} = 0.0135 [ 33 ]$
${K}_{m}^{{acoa}_{mito}} = {K}_{0}^{{acoa}_{mito}} \cdot ( 1 + \frac{kc 4 {coa}_{mito}}{{K}_{i}^{kc 4 {coa}_{mito}}} ) {K}_{0}^{{acoa}_{mito}} = 0.3 [ 35 ] {K}_{i}^{kc 4 {coa}_{mito}} = 0.0022 [ 36 ]$
3-ketoacyl-coa thiolase I (kc6)
${v}_{3 kt}^{kc 6 coa} = {V}_{max}^{3 kt - c {6}_{coa}} \cdot ( \frac{kc 6 {coa}_{mito} \cdot {coa}_{mito} - 1 / {K}_{eq}^{3 kt} \cdot {acoa}_{mito} \cdot c 4 {coa}_{mito}}{( {coa}_{mito} + {K}_{m}^{{coa}_{mito}} ) \cdot ( kc 6 {coa}_{mito} + {K}_{m}^{kc 6 {coa}_{mito}} )} )$
Vₘₐₓ^{3kt-kc6} = 2.4 · Vₘₐₓ^{3kt-kc4} [33] K_{eq}^{3kt} = 2500 [32] Kₘ^{coa}^{ₘᵢₜₒ} 0.0087 [33] Kₘ^{kc6coa}^{ₘᵢₜₒ} =
0.0083[33]
3-ketoacyl-coa thiolase I (kc8)
${v}_{3 kt}^{kc 8 coa} = {V}_{max}^{3 kt - kc 8} \cdot ( \frac{kc 8 {coa}_{mito} \cdot {coa}_{mito} - 1 / {K}_{eq}^{3 kt} \cdot {acoa}_{mito} \cdot c 6 {coa}_{mito}}{( {coa}_{mito} + {K}_{m}^{{coa}_{mito}} ) \cdot ( kc 8 {coa}_{mito} + {K}_{m}^{kc 8 {coa}_{mito}} )} )$
Vₘₐₓ^{3kt-kc8} = 2.2 · Vₘₐₓ^{3kt-kc4} [33] K_{eq}^{3kt} = 2500 [32] Kₘ^{coa}^{ₘᵢₜₒ} = 0.0024[33]
Kₘ^{kc8coa}^{ₘᵢₜₒ} = 0.025 [37]
3-ketoacyl-coa thiolase I (kc10)
${v}_{3 kt}^{kc 10 coa} = {V}_{max}^{3 kt - kc 10} \cdot ( \frac{kc 10 {coa}_{mito} \cdot {coa}_{mito} - 1 / {K}_{eq}^{3 kt} \cdot {acoa}_{mito} \cdot c 8 {coa}_{mito}}{( {coa}_{mito} + {K}_{m}^{{coa}_{mito}} ) \cdot ( kc 10 {coa}_{mito} + {K}_{m}^{kc 10 {coa}_{mito}} )} )$
Vₘₐₓ^{3kt-kc10} = 2 3 · Vₘₐₓ^{3kt-kc4} [33] K_{eq}^{3kt} = 2500 [32] Kₘ^{coa}^{ₘᵢₜₒ} = 0.0087 [33]
Kₘ^{kc10coa}^{ₘᵢₜₒ} = 0.0018[33]
3-ketoacyl-coa thiolase I (kc12)
${v}_{3 k t}^{k c 12 c o a} = {V}_{ma x}^{3 k t - k c 12} \cdot ( \frac{k c 12 c o {a}_{mito} \cdot {coa}_{mito} - \frac{1}{{K}_{e q}^{3 k t}} \cdot {acoa}_{mito} \cdot c 10 {coa}_{mito}}{( c o {a}_{m i t o} + {K}_{m}^{c o {a}_{mito}} ) \cdot ( kc 12 {coa}_{m i t o} + {K}_{m}^{kc 12 {coa}_{mito}} )} )$
Vₘₐₓ^{3kt-kc12} = 2.1 · Vₘₐₓ^{3kt-kc4} [37] K_{eq}^{3kt} = 2500 [32] Kₘ^{coa}^{ₘᵢₜₒ} = 0.0087 [33]
Kₘ^{kc12coa}^{ₘᵢₜₒ} = 0.006 [37]
3-ketoacyl-coa thiolase I (kc14)
${v}_{3 k t}^{k c 14 c o a} = {V}_{ma x}^{3 k t - k c 14} \cdot ( \frac{k c 14 c o {a}_{mito} \cdot {coa}_{mito} - \frac{1}{{K}_{e q}^{3 k t}} \cdot {acoa}_{mito} \cdot c 12 {coa}_{mito}}{( c o {a}_{mito} + {K}_{m}^{c o {a}_{m i t o}} ) \cdot ( kc 14 {coa}_{mito} + {K}_{m}^{k c 14 c o {a}_{m i t o}} )} )$
Vₘₐₓ^{3kt-kc14} = 1.7 · Vₘₐₓ^{3kt-kc4} [37] K_{eq}^{3kt} = 2500 [32] Kₘ^{coa}^{ₘᵢₜₒ} = 0.0087 [33]
Kₘ^{kc14coa}^{ₘᵢₜₒ} = 0.0065 [37]
3-ketoacyl-coa thiolase I (kc16)
${v}_{3 k t}^{k c 16 c o a} = {V}_{ma x}^{3 k t - k c 16} \cdot ( \frac{k c 16 c o {a}_{m i t o} \cdot {coa}_{m i t o} - \frac{1}{{K}_{e q}^{3 k t}} \cdot {acoa}_{mito} \cdot c 14 {coa}_{mito}}{( c o {a}_{mito} + {K}_{m}^{c o {a}_{mito}} ) \cdot ( kc 16 {coa}_{mito} + {K}_{m}^{k c 16 c o {a}_{mito}} )} )$
Vₘₐₓ^{3kt-kc16} for numerical value see Table 10
K_{eq}^{3kt} = 2500 [32] Kₘ^{coa}^{ₘᵢₜₒ} = 0.0087 [33] Kₘ^{kc16coa}^{ₘᵢₜₒ} = 0.0011 [38]
ETF-FAD
V_{ETF-FAD} = Vₘₐₓ^{ETF-FAD} · (etffadh2ₘᵢₜₒ · etfqₘᵢₜₒ − 1/K_{eq}^{ETF-FAD} · etfqh2ₘᵢₜₒ · etffadₘᵢₜₒ)
Vₘₐₓ^{ETF-FAD} for numerical value see Table 10
${K}_{e q}^{ETF - FAD} = exp ( \frac{( - n \cdot {E}_{0}^{{etffad}_{mito} / etffadh {2}_{mito}} - n \cdot {E}_{0}^{etfqh {2}_{mito} / {etfq}_{mito}} ) \cdot F}{R \cdot T} )$
E₀^{etfqh2}^{ₘᵢₜₒ}^{/etfq}^{ₘᵢₜₒ} = −25 mV [39] E₀^{etffad}^{ₘᵢₜₒ}^{/etffadh2}^{ₘᵢₜₒ} = −23 mV [40] n = 2
ETF-QO
${v}_{ETF - QO} = {V}_{ma x}^{ETF - QO} \cdot ( etfqh {2}_{mito} \cdot {q}_{m m} - \frac{1}{{K}_{e q}^{ETF - Q O}} \cdot {etfq}_{mito} \cdot {qh}_{{2}_{m m}} )$
Vₘₐₓ^{ETF-QO} for numerical value see Table 10
${K}_{e q}^{ETF - QO} = exp ( \frac{( n \cdot {E}_{0}^{{q}_{m m} / q h {2}_{m m}} + n \cdot {E}_{0}^{etfqh {2}_{m i t o} / {etfq}_{mito}} ) \cdot F}{R \cdot T} )$
E₀^{q}^{ₘₘ}^{/qh2}^{ₘₘ} = 87 mV [41] E₀^{etfqh2}^{ₘᵢₜₒ}^{/etfq}^{ₘᵢₜₒ} = −25 mV [39] n = 2
Citric acid cycle
Pyruvate dehydrogenase complex
v_{pdhc} = Y_{pdhc} · V_{pdhc-np}
${v}_{pdhc - np} = {V}_{ma x}^{pdhc - np} \cdot ( \frac{{pyr}_{mito}}{{pyr}_{mito} + {K}_{m - pyr}^{pdhc - np}} ) \cdot ( \frac{{nad}_{mito}}{{nad}_{mito} + {K}_{m - nad}^{pdhc - np}} ) \cdot ( \frac{{coa}_{mito}}{{coa}_{mito} + {K}_{m - coa}^{pdhc - np}} ) \cdot ( 1 + {k}_{ca} \frac{{ca}_{mito}}{{ca}_{mito} + {K}_{a}^{ca}} )$
Vₘₐₓ^{pdhc-up} for numerical value see Table 10
${K}_{m - p y r}^{pdhc - np} = 0.0 \; 77 [ 42 ] {K}_{m - nad}^{pdhc - np} = {K}_{0}^{nad} \cdot ( 1 + \frac{{nadh}_{mito}}{{K}_{i}^{{nadh}_{mito}}} ) {K}_{0}^{nad} = 0.07 [ 42 ]$
${K}_{i}^{{nadh}_{mito}} = 0.3 [ 43 ] {K}_{m - coa}^{pdhc - np} = {K}_{0}^{coa} \cdot ( 1 + \frac{{acoa}_{mito}}{{K}_{i}^{{acoa}_{mito}}} ) {K}_{0}^{coa} = 0.0122 [ 42 ]$
Kᵢ^{acoa}^{ₘᵢₜₒ} = 0.029 [44] k_{ca} = 1.7 [45] Kₐ^{ca} = 0.001 [46] γ_{pdhc} = γ_{pdhc}^{acoa} · γ_{pdhc}^{nadh}
${γ}_{pdhc}^{acoa} = 1 - {f}_{1} \cdot \frac{\frac{{acoa}_{mito}}{{coa}_{mito}}}{\frac{{acoa}_{mito}}{{coa}_{mito}} + {K}_{i}^{( \frac{{acoa}_{mito}}{{coa}_{mito}} )}} [ 47 ] {K}_{i}^{( \frac{{acoa}_{mito}}{{coa}_{mito}} )} = 0.4 [ 47 ] {f}_{1} = 0.71 [ 47 ]$
${γ}_{pdhc}^{nadh} = 1 - {f}_{2} \cdot \frac{\frac{{nadh}_{mito}}{{nad}_{mito}}}{\frac{{nadh}_{mito}}{{nad}_{mito}} + {K}_{i}^{( \frac{{nadh}_{mito}}{{nad}_{mito}} )}} {f}_{2} = 0.75 [ 47 ] {K}_{i}^{( \frac{{nadh}_{mito}}{{nad}_{mito}} )} = 0.5 [ 47 ]$
Citrate synthase
${v}_{c s} = {V}_{ma x}^{c s} \cdot ( \frac{o a {a}_{mito}}{o a {a}_{mito} + {K}_{m}^{o a {a}_{mito}}} ) \cdot ( \frac{a c o {a}_{mito}}{a c o {a}_{mito} + {K}_{m}^{{acoa}_{mito}}} )$
${V}_{ma x}^{c s} = \frac{{V}_{0}^{c s}}{( 1 + \frac{c 16 {coa}_{mito}}{{K}_{i}^{c 16 {coa}_{mito}}} ) \cdot ( 1 + \frac{{atp}_{mito}}{{K}_{i}^{{atp}_{mito}}} )}$
V₀^{cs} for numerical value see Table 10 Kᵢ^{c16coa}^{ₘᵢₜₒ} = 0.0042 [48] Kᵢ^{atp}^{ₘᵢₜₒ} = 0.7 [49]
${K}_{m}^{o a {a}_{mito}} = {K}_{0}^{o a {a}_{m i t o}} \cdot ( 1 + \frac{c i {t}_{mito}}{{K}_{i}^{{cit}_{mito}}} ) {K}_{i}^{{cit}_{mito}} = 1.6 [ 50 ] {K}_{0}^{o a {a}_{mito}} = 0.0036 [ 49 ]$
${K}_{m}^{a c o {a}_{mito}} = {K}_{0}^{a c o {a}_{mito}} \cdot ( 1 + \frac{{succoa}_{mito}}{{K}_{i}^{{succoa}_{mito}}} ) {K}_{i}^{c o {a}_{mito}} = 0.0 \; 67 [ 51 ]$
Kᵢ^{atp}^{ₘᵢₜₒ} = 0.95 [51] Kᵢ^{succoa}^{ₘᵢₜₒ} = 0.13 [51] K₀^{acoa}^{ₘᵢₜₒ} = 0.006 [52]
Aconitase
${v}_{a c} = {V}_{ma x}^{a c} \cdot ( \frac{{cit}_{mito} - \frac{1}{{K}_{e q}^{a c}} \cdot {isocit}_{mito}}{1 + \frac{c i {t}_{m i t o}}{{K}_{m}^{c i {t}_{mito}}} + \frac{i s o c i {t}_{mito}}{{K}_{m}^{{isocit}_{mito}}}} )$
Vₘₐₓ^{ac} for numerical value see Table 10
K_{eq}^{ac} = 0.1 [53] Kₘ^{cit}^{ₘᵢₜₒ} = 0.62 [54] Kₘ^{isocit}^{ₘᵢₜₒ} = 0.2 [54]
NAD-dependent isocitrate dehydrogenase
${v}_{{idh}_{nad}} = {V}_{ma x}^{{idh}_{nad}} \cdot ( \frac{{isocit}_{mito}^{n}}{{isocit}_{mito}^{n} + {( {K}_{m}^{{isocit}_{mito}} )}^{n}} ) \cdot ( \frac{{nad}_{mito}}{{nad}_{mito} + {K}_{m}^{{nad}_{mito}}} )$
Vₘₐₓ^{idh}^{_{nad}} for numerical value see Table 10
${K}_{m}^{{isocit}_{mito}} = {K}_{0}^{{isocit}_{mito}} \cdot ( 1 - {n}_{{adp}_{mito}} \frac{{adp}_{mito}}{{adp}_{mito} + {K}_{a}^{{adp}_{mito}}} ) \cdot ( 1 - {n}_{{cit}_{mito}} \frac{{cit}_{mito}}{{cit}_{mito} + {K}_{a}^{{cit}_{mito}}} )$
K₀^{isocit}^{ₘᵢₜₒ} = 0.21 [55] n = 3 [55] n_{adp}_{ₘᵢₜₒ} = 0.67 [56] Kₐ^{adp}^{ₘᵢₜₒ} = 0.1 [56]
n_{cit}_{ₘᵢₜₒ} = 0.85 [57] Kₐ^{cit}^{ₘᵢₜₒ} = 0.033 [57]
${K}_{m}^{{nad}_{mito}} = {K}_{0}^{{nad}_{mito}} \cdot ( 1 + \frac{{nadh}_{mito}}{{K}_{i}^{{nadh}_{mito}}} ) {K}_{0}^{{nad}_{mito}} = 0.06 [ 56 ] {K}_{i}^{{nadh}_{mito}} = 0.0 \; 043 [ 58 ]$
NADP-dependen isocitrate dehydrogenase
${v}_{{idh}_{nadph}} = {V}_{ma x}^{{idh}_{nadp}} \cdot ( \frac{{isocit}_{mito}}{{isocit}_{mito} + {K}_{m}^{{isocit}_{mito}}} ) \cdot ( \frac{{nadp}_{mito}}{{nadp}_{mito} + {K}_{m}^{{nad}_{mito}}} )$
Vₘₐₓ^{idh}^{_{nadp}} for numerical value see Table 10
${K}_{m}^{{isocit}_{mito}} = {K}_{0}^{{isocit}_{mito}} \cdot ( 1 + \frac{{cit}_{mito}}{{K}_{i}^{{cit}_{mito}}} ) \cdot ( 1 + \frac{{akg}_{mito}}{{K}_{i}^{{akg}_{mito}}} )$
${K}_{0}^{{isocit}_{mito}} = 0.045 [ 59 ] {K}_{m}^{{nadp}_{mito}} = {K}_{0}^{{nadp}_{mito}} \cdot ( 1 + \frac{{nadph}_{mito}}{{K}_{i}^{{nadph}_{mito}}} ) {K}_{0}^{{nadp}_{mito}} = 0.0 \; 46 [ 59 ]$
Kᵢ^{nadph}^{ₘᵢₜₒ} = 0.125 [58] Kᵢ^{cit}^{ₘᵢₜₒ} = 0.159 [59] Kᵢ^{akg}^{ₘᵢₜₒ} = 0.08 [59]
α-ketogluterate dehydrogenase
${v}_{kgdhc} = {V}_{m x}^{kgdhc} \cdot ( \frac{{akg}_{mito}}{{akg}_{mito} + {K}_{m}^{{akg}_{mito}} \cdot ( 1 + \frac{{nadh}_{mito}}{{K}_{i 2}^{{nadh}_{mito}}} )} ) \cdot \\ ( \frac{{nad}_{mito}}{{nad}_{mito} + {K}_{m}^{nad} \cdot ( 1 + \frac{{nadh}_{mito}}{{K}_{i}^{{nadh}_{mito}}} )} ) \cdot ( \frac{{coa}_{mito}}{{coa}_{mito} + {K}_{m}^{{coa}_{mito}} \cdot ( 1 + \frac{{succoa}_{mito}}{{K}_{i}^{{succoa}_{mito}}} )} )$
Vₘₓ^{kgdhc} for numerical value see Table 10
Kᵢ₂^{nadh}^{ₘᵢₜₒ} = 0.0127 [60] Kₘ^{akg}^{ₘᵢₜₒ} = 0.6 [61] Kₘ^{nad}^{ₘᵢₜₒ} = 0.021[60] Kᵢ^{nadh}^{ₘᵢₜₒ} = 0.0045 [60]
Kₘ^{coa}^{ₘᵢₜₒ} = 0.0027 [60] Kᵢ^{succoa}^{ₘᵢₜₒ} = 0.0069[60]
Succinyl-Coa synthetase [62]
${v}_{s c s - atp} = {V}_{ma x}^{scs - atp} \cdot ( \frac{\begin{matrix}{succoa}_{mito} \cdot {adp}_{mito} \cdot {p}_{mito} - \\ \frac{1}{{K}_{eq}^{suc - atp}} \cdot {suc}_{mito} \cdot {coa}_{mito} \cdot {atp}_{mito}\end{matrix}}{\begin{matrix}( 1 + \frac{{succoa}_{mito}}{{K}_{m}^{{succoa}_{mito}}} ) \cdot ( 1 + \frac{{adp}_{mito}}{{K}_{m}^{{adp}_{mito}}} ) \cdot ( 1 + \frac{{p}_{mito}}{{K}_{m}^{{p}_{mito}}} ) + \\ ( 1 + \frac{s u {c}_{mito}}{{K}_{m}^{{suc}_{mito}}} ) \cdot ( 1 + \frac{{coa}_{mito}}{{K}_{m}^{{coa}_{mito}}} ) \cdot ( 1 + \frac{{atp}_{mito}}{{K}_{m}^{{atp}_{mito}}} ) - 1\end{matrix}} )$
${V}_{ma x}^{scs - atp} = {V}_{0}^{scs - atp} \cdot ( \frac{{p}_{mito}^{n}}{{p}_{mito}^{n} + {K}_{a}^{{p}^{n}}} )$
V₀^{scs-atp} for numerical value see Table 10
Kₐ^{p}^{ⁿ} = 2.3 [63] n = 2.4 [63] K_{eq}^{scs-atp} = 1/0.27 [64] Kₘ^{succoa}^{ₘᵢₜₒ} = 0.041 [65]
Kₘ^{adp}^{ₘᵢₜₒ} = 0.25 [65] Kₘ^{p}^{ₘᵢₜₒ} = 0.72 [65] Kₘ^{suc}^{ₘᵢₜₒ} = 5.1[65]
Kₘ^{coa}^{ₘᵢₜₒ} = 0.032 [65] Kₘ^{atp}^{ₘᵢₜₒ} = 0.055 [65]
${v}_{scs - gtp} = {V}_{ma x}^{scs - gtp} \cdot ( \frac{\begin{matrix}{succoa}_{mito} \cdot {gdp}_{mito} \cdot {p}_{mito} - \\ \frac{1}{{K}_{e q}^{scs - atp}} \cdot {suc}_{mito} \cdot {coa}_{mito} \cdot {gtp}_{mito}\end{matrix}}{\begin{matrix}( 1 + \frac{{succoa}_{mito}}{{K}_{m}^{{succoa}_{mito}}} ) \cdot ( 1 + \frac{{gdp}_{mito}}{{K}_{m}^{{gdp}_{mito}}} ) \cdot ( 1 + \frac{{p}_{mito}}{{K}_{m}^{{p}_{mito}}} ) + \\ ( 1 + \frac{{suc}_{mito}}{{K}_{m}^{{suc}_{mito}}} ) \cdot ( 1 + \frac{{coa}_{mito}}{{K}_{m}^{{coa}_{mito}}} ) \cdot ( 1 + \frac{{gtp}_{mito}}{{K}_{m}^{{gtp}_{mito}}} ) - 1\end{matrix}} )$
${V}_{ma x}^{scs - gtp} = {V}_{0}^{scs - gtp} \cdot ( \frac{{p}_{mito}^{n}}{{p}_{mito}^{n} + {K}_{a}^{{p}^{n}}} )$
V₀^{scs-gtp} = 0.11 · V₀^{scs-atp} [65] Kₐ^{p}^{ₘᵢₜₒ} = 2.3 [63] n = 2.4 [63]
K_{eq}^{scs-gtp} = 1/0.27 [66] Kₘ^{succoa}^{ₘᵢₜₒ} = 0.086 [65] Kₘ^{gdp}^{ₘᵢₜₒ} = 0.007 [65]
Kₘ^{p}^{ₘᵢₜₒ} = 2.26[65] Kₘ^{suc}^{ₘᵢₜₒ} = 0.49 [65] Kₘ^{coa}^{ₘᵢₜₒ} = 0.036 [65] Kₘ^{gtp}^{ₘᵢₜₒ} = 0.036 [65]
Succinate dehydrogenase
${v}_{succdh} = {V}_{ma x}^{s u c c a h} \cdot ( \frac{{suc}_{mito} \cdot {q}_{mm} - \frac{1}{{K}_{e q}^{succdh}} \cdot {fum}_{mito} \cdot {qh}_{{2}_{m m}}}{( {suc}_{mito} + {K}_{m}^{{suc}_{mito}} ) \cdot ( {q}_{m m} + {K}_{m}^{{q}_{m m}} )} )$
Vₘₐₓ^{succdh} for numerical value see Table 10
${K}_{eq}^{succdh} = exp ( \frac{{E}_{0}^{{fum}_{mito} / {suc}_{mito}} - {E}_{0}^{{q}_{m m} / qh {2}_{mm}}}{R \cdot T} \cdot F ) {E}_{0}^{{fum}_{mito} / {suc}_{mito}} - {E}_{0}^{{q}_{m m} / q h {2}_{m m}} = 25 mV$
${K}_{0}^{{suc}_{mito}} = 1.3 [ 67 ] {K}_{m}^{{suc}_{mito}} = \\ {K}_{0}^{{suc}_{mito}} \cdot ( 1 + \frac{{mal}_{mito}}{{K}_{i}^{{mal}_{mito}}} ) {K}_{i}^{{mal}_{mito}} = 2.2 [ 68 ] {K}_{m}^{{q}_{m m}} = 0.0 \; 005 [ 69 ]$
Fumarase
${v}_{fum} = {V}_{ma x}^{fum} \cdot ( \frac{{fum}_{mito} - \frac{1}{{K}_{eq}^{fum}} \cdot {mal}_{mito}}{1 + \frac{{fum}_{mito}}{{K}_{m}^{{fum}_{mito}}} + \frac{{mal}_{mito}}{{K}_{m}^{{mal}_{mito}}}} )$
Vₘₐₓ^{fum} for numerical value see Table 10
K_{eq}^{fum} = 4.2 [70] Kₘ^{fum}^{ₘᵢₜₒ} = 0.333 [71] Kₘ^{mal}^{ₘᵢₜₒ} = 0.59 [71]
Malate dehydrogenase (mitochondrial)
${v}_{{mdh}_{mito}} = {V}_{ma x}^{{mdh}_{mito}} \cdot ( \frac{{mal}_{mito} \cdot {nad}_{mito} - \frac{1}{{K}_{eq}^{{mdh}_{mito}}} \cdot {oaa}_{mito} \cdot {nadh}_{mito}}{\begin{matrix}( 1 + \frac{{mal}_{mito}}{{K}_{m}^{{mal}_{mito}}} ) \cdot ( 1 + \frac{{nad}_{mito}}{{K}_{m}^{{nad}_{mito}}} ) + \\ ( 1 + \frac{{oaa}_{mito}}{{K}_{m}^{{oaa}_{mito}}} ) \cdot ( 1 + \frac{{nadh}_{mito}}{{K}_{m}^{{nadh}_{mito}}} ) - 1\end{matrix}} )$
vₘₐₓ^{mdh}^{ₘᵢₜₒ} for numerical value see Table 10
${K}_{eq}^{{mdh}_{mito}} = 1 \cdot {10}^{- 5} \cdot ( \frac{{h}_{cyt}}{{h}_{mito}} ) [ 72 ]$
Kₘ^{mal}^{ₘᵢₜₒ} = 0.4 [73] Kₘ^{nad}^{ₘᵢₜₒ} = 0.06 [74] Kₘ^{oaa}^{ₘᵢₜₒ} = 0.017 [74] Kₘ^{nadh}^{ₘᵢₜₒ} = 0.044 [74]
Transdehydrogenase [75]
${v}_{tdh} = {V}_{ma x}^{tdh} \cdot ( {nadh}_{mito} \cdot {nadp}_{mito} - \frac{1}{{K}_{e q}^{tdh}} \cdot {nad}_{mito} \cdot {nadph}_{mito} )$
Vₘₐₓ^{tdh}^{ₘᵢₜₒ} for numerical value see Table 10
${K}_{eq}^{{tdh}_{mito}} = {K}_{0}^{tdh} \cdot exp ( - \frac{{v}_{m m} \cdot F}{R \cdot T} ) \cdot ( \frac{{h}_{cyt}}{{h}_{mito}} ) {K}_{0}^{tdh} = 1.5 [ 76 ]$
Mitochondrial electrophysiology and ATP synthesis
Chloride
${I}_{c {l}_{e d}} = {P}_{c l} \cdot {A}_{m} \cdot U \cdot F \cdot ( \frac{{cl}_{cy𝔱} - {cl}_{mito} \cdot exp ( - U )}{1 - exp ( - U )} ) U = \frac{{v}_{m m} \cdot F}{R \cdot T} {P}_{c l} = 5 \cdot {10}^{- 10} m / s$
Sodium
${I}_{n a}^{pump} = {V}_{ma x}^{N a - pump} \cdot ( \frac{{na}_{cyt} \cdot {h}_{mito} - {na}_{mito} \cdot {h}_{cyt}}{1 + \frac{{na}_{cyt}}{{K}_{m}^{na}} + \frac{{na}_{mito}}{{K}_{m}^{na}}} )$
Vₘₐₓ^{Na-pump} for numerical value see Table 10
${K}_{m}^{n a} = 32.4 [ 77 ] {I}_{n {a}_{e d}} = {P}_{n a} \cdot {A}_{m} \cdot U \cdot F \cdot ( \frac{n {a}_{cyt} - n {a}_{mito} \cdot exp ( U )}{exp ( U ) - 1} )$
$U = \frac{{v}_{m m} \cdot F}{R \cdot T} {P}_{n a} = 1 \cdot {10}^{- 10} m / s {I}_{n a} = {I}_{n a}^{pump} + {I}_{n {a}_{e d}}$
Potassium
I_{K}^{pump} = Vₘₐₓ^{K-pump} · (k_{cyt} · hₘᵢₜₒ − kₘᵢₜₒ · h_{cyt})
Vₘₐₓ^{K-pump} for numerical value see Table 10
${I}_{{k}_{ed}} = {P}_{k} \cdot {A}_{m} \cdot U \cdot F \cdot ( \frac{{k}_{cyt} - {k}_{mito} \cdot exp ( U )}{exp ( U ) - 1} )$
$U = \frac{{v}_{m m} \cdot F}{R \cdot T} {P}_{K} = 5 \cdot {10}^{- 10} m / s {I}_{k} = {I}_{k}^{pump} + {I}_{{k}_{e d}}$
F0F1 synthetase
${v}_{F 0 F 1} = {V}_{ma x}^{F 0 F 1} \cdot ( \frac{{adp}_{mito} \cdot {p}_{mito} - \frac{1}{{K}_{eq}^{F 0 F 1}} \cdot {atp}_{mito}}{( {K}_{m}^{{adp}_{mito}} + {adp}_{mito} ) \cdot ( {K}_{m}^{{p}_{mito}} + {p}_{mito} )} )$
${V}_{ma x}^{F 0 F 1} = {V}_{F 0 F 1} \cdot ( 0.114 + 0.886 \frac{{( \vert {V}_{m m} \vert )}^{n}}{{( \vert {V}_{m m} \vert )}^{n} + {( {K}_{m}^{{V}_{m m}} )}^{n}} ) [ 78 ]$
V_{F0F1} for numerical value see Table 10
n = 10 [78] Kₘ^{V}^{ₘₘ} = 140 mV [78]
${K}_{e q}^{F 0 F 1} = exp ( ( \frac{- {E}_{0}^{ATP}}{R \cdot T} ) - {n}_{H} \cdot ( \frac{{V}_{m m} \cdot F}{R \cdot T} ) ) \cdot {( \frac{{H}_{cyt}}{{H}_{mito}} )}^{{n}_{H}} {mM}^{- 1}$
n_{H} = 3 E₀^{ATP} = 30500 J/mol Kₘ^{adp}^{ₘᵢₜₒ} = 0.025 [79] Kₘ^{p}^{ₘᵢₜₒ} = 6.1 [79]
ATP-ADP nucleotide exchanger [80]
${v}_{nex} = {V}_{ma x}^{nex} \cdot ( \frac{1 - \frac{{atp}_{cyt} \cdot {adp}_{mito}}{{adp}_{cyt} \cdot {atp}_{mito}} \cdot exp ( \frac{{v}_{m m} \cdot F}{R \cdot T} )}{1 + \frac{{atp}_{cyt}}{{adp}_{cyt}} \cdot exp ( f \cdot \frac{{v}_{m m} \cdot F}{R \cdot T} ) \cdot ( 1 + \frac{{adp}_{mito}}{{atp}_{mito}} )} )$
Vₘₐₓ^{nex} for numerical value see Table 10 f = 0.2
Phosphate exchanger
${v}_{P - ex} = {V}_{ma x}^{P - ex} \cdot ( \frac{{p}_{cyt} \cdot {h}_{cyt} - {p}_{mito} \cdot {h}_{mito}}{( {p}_{cyt} + {K}_{m}^{{p}_{cy𝔱}} )} )$
Vₘₐₓ^{P-ex} for numerical value see Table 10 Kₘ^{p}^{_{cyt}} = 1.89 [81]
Complex I
${v}_{c x l} = {V}_{ma x}^{c x l} \cdot ( \frac{{nadh}_{mito} \cdot {q}_{m m} - \frac{1}{{K}_{e q}^{c x l}} \cdot {nad}_{m i t o} \cdot {qh}_{{2}_{m m}}}{( {nadh}_{mito} + {K}_{m}^{{nadh}_{mito}} ) \cdot ( {q}_{m m} + {K}_{m}^{{q}_{m m}} )} )$
Vₘₐₓ^{cxI} for numerical value see Table 10
${K}_{e q}^{cxl} = exp ( \frac{( n \cdot {E}_{0}^{nadh / nad} + n \cdot {E}_{0}^{Q / Q {H}_{2}} + {n}_{H} \cdot {V}_{m m} ) \cdot F}{R \cdot T} ) \cdot {( \frac{{h}_{mito}}{{h}_{cyt}} )}^{{n}_{H}}$
n = 2 E₀^{nadh/nad} = 320 mV [82] E₀^{Q/QH}^{₂} = 87 mV [41]
N_{H} = 4 Kₘ^{nadh}^{ₘᵢₜₒ} = 0.0017 [83] Kₘ^{q}^{ₘₘ} = 0.013 [83]
Complex II
see succinate dehydrogenase
Complex III
${v}_{cxIII} = {V}_{ma x}^{cxIII} \cdot ( \frac{qh {2}_{m m} \cdot {cyc}_{o {x}_{m m}} - \frac{1}{{K}_{eq}^{cxIII}} \cdot {q}_{m m} \cdot {cytc}_{{red}_{m m}}}{( q h {2}_{m m} + {K}_{m}^{q h {2}_{m m}} ) \cdot {( {cytc}_{o {x}_{m m}} + {K}_{m}^{{cytc}_{o x}} )}^{2}} )$
Vₘₐₓ^{cxIII} for numerical value see Table 10
${K}_{eq}^{cxIII} = exp ( \frac{( - n \cdot {E}_{0}^{Q / Q {H}_{2}} + n \cdot {E}_{0}^{{cytC}_{o x} / c y t {C}_{red}} + n \cdot {v}_{m m} ) \cdot F}{R \cdot T} ) \cdot {( \frac{{h}_{mito}}{{h}_{∅}} )}^{{n}_{{h}_{mito}}} \cdot {( \frac{{h}_{∅}}{{h}_{cyt}} )}^{{n}_{{h}_{cyt}}}$
n = 2 E₀^{cytC}^{ₒₓ}^{/cytC}^{_{red}} = 255 mV [84]
E₀^{Q/QH}^{₂} = 87 mV [41] nₕ_{ₘᵢₜₒ} = 2 nₕ_{_{cyt}} = 4 h_{ø} = 10⁻⁴ mM [symbol] pH 7
Kₘ^{qh}₂_{ₘₘ} = 0.013 [85] Kₘ^{cytc}^{ₒₓ} = 0.014 [85]
Complex IV
${v}_{cxIV} = {V}_{ma x}^{cxIV} \cdot ( \frac{{cytc}_{red}}{{cytc}_{red} + {K}_{m}^{{cytc}_{red}}} ) \cdot ( \frac{{O}_{2}}{{O}_{2} + {K}_{m}^{{O}_{2}}} )$
${V}_{ma x}^{cxIV} = {V}_{0}^{cxIV} \cdot exp ( - \frac{dGp \cdot F}{R \cdot T} ) dGp = - {v}_{m m} + \frac{R \cdot T}{F} \cdot log ( \frac{{h}_{cyt}}{{h}_{mito}} )$
V₀^{cxIV} for numerical value see Table 10
Kₘ^{cytc}^{_{red}} = 0.007 [86] Kₘ⁰^{₂} = 2 mmHg
Adenylate kinase
${v}_{{ak}_{cyc}} = {V}_{ma x}^{{ak}_{cyt}} \cdot ( \frac{{atp}_{cyt} \cdot {amp}_{cyt} - \frac{1}{{K}_{eq}^{ak}} \cdot {adp}_{cyt} \cdot {adp}_{cyt}}{( 1 + \frac{{atp}_{cyt}}{{K}_{m}^{{atp}_{cyt}}} ) \cdot ( 1 + \frac{{amp}_{cyt}}{{K}_{m}^{{amp}_{cyt}}} ) + {( 1 + \frac{{adp}_{cyt}}{{K}_{m}^{{adp}_{cyt}}} )}^{2} - 1} )$
Vₘₐₓ^{ak}^{_{cyt}} for numerical value see Table 10
K_{eq}^{ak} = 1 [87] Kₘ^{atp}^{_{cyt}} = 0.039 [88] Kₘ^{adp}^{_{cyt}} = 0.112 [88] Kₘ^{amp}^{_{cyt}} = 0.026 [88]
Pyrophosphatase
${v}_{ppase} = {V}_{ma x}^{ppase} \cdot ( \frac{{pp}_{cyt}}{{pp}_{cyt} + {K}_{m}^{{pp}_{cyt}}} )$
Vₘₐₓ^{ppase} for numerical value see Table 10 Kₘ^{pp}^{_{cyt}} = 0.016 [89]
ATP usage
${v}_{atp - usage} = {V}_{ma x}^{atp - usage} \cdot ( \frac{{atp}_{cyt}}{{atp}_{cyt} + {K}_{m}^{{atp}_{cyt}}} ) \cdot ( 1 + {k}_{load} )$
Vₘₐₓ^{atp-usage} for numerical value see Table 10 Kₘ^{atp}^{_{cyt}} = 2
O₂ diffusion
${v}_{{O}_{{2}_{diff}}} = {V}_{max}^{{O}_{2} - diff} \cdot ( o {2}_{ext} - o {2}_{cyt} )$
Vₘₐₓ^{O}^{₂}^{-diff} for numerical value see Table 10
Proton fluxes
I_{H}^{pump} = 4 · v_{cxl} + 2 · V_{cxIII} + 4 · v_{cxIV}
${I}_{{H}_{e d}} = {P}_{H} \cdot {A}_{m} \cdot U \cdot F \cdot ( \frac{{H}_{cyt} - {H}_{mito} \cdot exp ( U )}{exp ( U ) - 1} )$
P_{H} = 3 · 10⁻⁴ m/s
Mitochondrial membrane potential
${v}_{{V}_{m m}} = \frac{1 {0}^{- 1}}{{c}_{m} \cdot {A}_{m}} \cdot ( - {I}_{{C}_{e d}} + {I}_{{K}_{e d}} + {I}_{{H}_{e d}} + {I}_{{Na}_{ed}} + {I}_{H}^{pump} + {v}_{n e x} + 3 \cdot {v}_{syn} + F \cdot 10 \cdot {v}_{pepT} \cdot {Vol}_{cyt} )$
Glycolysis
Glut1 glucose transporter (Glut1)
${v}_{gluT 1} = {V}_{ma x}^{gluT 1} \cdot \frac{{glc}_{ext} - {glc}_{cyt}}{1 + \frac{{glc}_{ext}}{{n}_{m}^{{glc}_{ext}}} + \frac{{glc}_{cyt}}{{K}_{m}^{{glc}_{cyt}}}} {V}_{ma x}^{gluT 1} = {V}_{0}^{gluT 1} \cdot ( 1 - \frac{c 1 {6}_{ext}^{n}}{c 1 {6}_{ext}^{n} + {K}_{i}^{c {16}_{ext}}} )$
V₀^{gluT1} for numerical value see Table 10
n = 2 [90] Kᵢ^{c16}^{ₑₓₜ} = 0.2 [90] Kₘ^{glc}^{_{cyt}} = 5 [91] Kₘ^{glc}^{ₑₓₜ} = 5 [91, 92]
Glut4 glucose transporter (Glut4)
${v}_{gluT 4} = {V}_{ma x}^{gluT 4} \cdot \frac{{glc}_{ext} - {glc}_{cyt}}{1 + \frac{{glc}_{ext}}{{K}_{m}^{{glc}_{ext}}} + \frac{{glc}_{cyt}}{{K}_{m}^{{glc}_{cyt}}}}$
${V}_{ma x}^{gluT 4} = {V}_{0}^{gluT 4} \cdot ( 1 - \frac{c 1 {6}_{ext}^{n}}{c 1 {6}_{ext}^{n} + {K}_{i}^{c {16}_{ext}}} ) \cdot \\ ( 1 - γ \cdot ( 1 - \frac{{epi}_{ext}}{{epi}_{ext} + {K}_{a}^{{epi}_{ext}}} ) \cdot ( 1 - \frac{{amp}_{cyt}}{{amp}_{cyt} + {K}_{a}^{{amp}_{cyt}}} ) ) [ 93 ]$
V₀^{gluT4} [94] for numerical value see Table 10n = 2 [90] Kᵢ^{c16}^{ₑₓₜ} = 0.2 [90]
Kₐ^{epi}^{ₑₓₜ} = 200 pM Kₐ^{amp}^{_{cyt}} = 0.2 Kₘ^{glc}^{_{cyt}} = 5 [91] Kₘ^{glc}^{ₑₓₜ} = 5 [91]
Hexokinase A
${v}_{hkA} = {V}_{ma x}^{hkA} \cdot ( \frac{{glc}_{cyt}}{{glc}_{cyt} + {K}_{m}^{{glc}_{cyt}}} ) \cdot ( \frac{{atp}_{cyt}}{{atp}_{cyt} + {K}_{m}^{{atp}_{cyt}}} )$
${V}_{ma x}^{hkA} = \frac{{V}_{0}^{hkA}}{1 + \frac{glc 6 {p}_{cyt}}{{K}_{i}^{hk}}} {V}_{0}^{hkA}$
for numerical value see Table 10
Kᵢ^{glc}^{_{cyt}} = 0.05 [95] Kₘ^{glc}^{_{cyt}} = 0.25 [96]
${K}_{m}^{{atp}_{cyt}} = {K}_{0}^{{atp}_{cyt}} \cdot ( 1 + \frac{{glc6p}_{cyt}}{{K}_{i}^{glc 6 {p}_{cyt}}} ) \cdot ( 1 + \frac{{adp}_{cyt}}{{K}_{i}^{{adp}_{cyt}}} )$
K₀^{atp}^{_{cyt}} = 0.75 [96] Kᵢ^{adp}^{_{cyt}} = 0.22 [96] Kᵢg^{lc6p}^{_{cyt}} = 0.021 [96]
Hexokinase 1
${v}_{hkB} = {V}_{ma x}^{hkB} \cdot ( \frac{{glc}_{cyt}}{{glc}_{cyt} + {K}_{m}^{{glc}_{cyt}}} ) \cdot ( \frac{{atp}_{cyt}}{{atp}_{cyt} + {K}_{m}^{{atp}_{cyt}}} )$
${V}_{ma x}^{hkB} = \frac{{V}_{0}^{hkB}}{1 + \frac{glc 6 {p}_{cyt}}{{K}_{i}^{hk}}} {V}_{0}^{hkB} = 10 \cdot {V}_{0}^{hkA} [ 97 ] {K}_{i}^{hk} = 0.05 [ 95 ] {K}_{m}^{{glc}_{cyt}} = 0.02 [ 95 ]$
${K}_{m}^{{atp}_{cyt}} = {K}_{0}^{{atp}_{cyt}} \cdot ( 1 + \frac{glc 6 {p}_{cyt}}{{K}_{i}^{glc 6 {p}_{cyt}}} ) \cdot ( 1 + \frac{{adp}_{cyc}}{{K}_{i}^{{adp}_{cyt}}} )$
K₀^{atp}^{_{cyt}} = 0.44 [95] Kᵢ^{adp}^{_{cyt}} = 0.62 [96] Kᵢ^{glc6p}^{_{cyt}} = 0.02 [95]
D-Glucose-6-phosphate isomerase (Gpi)
${v}_{gpi} = {V}_{ma x}^{gpi} \cdot \frac{glc 6 {p}_{cyt} - \frac{fru 6 {p}_{cyt}}{{K}_{eq}^{gpi}}}{1 + \frac{glc 6 {p}_{cyt}}{{K}_{m}^{{glp}_{cyt}}} + \frac{fru 6 {p}_{cyt}}{{K}_{m}^{fru 6 {p}_{cyt}}}}$
Vₘₐₓ^{gpi} for numerical value see Table 10
K_{eq}^{Gpi} = 0.3 [98] Kₘ^{glc6p}^{_{cyt}} = 0.55 [99] Kₘ^{fru6p}^{_{cyt}} = 0.12 [99]
Phosphofructokinase 2 (Pfk2)
v_{pfk2} = Vₘₐₓ^{pfk2}^{ₙₐₜᵢᵥₑ} · (1 − γ^{Pfk2}) · v_{pfk2}^{native} + Vₘₐₓ^{pfk2}^{ₚ} (γ^{pfk2} · v_{pfk2}^{p})
γ^{pfk2} = γ · (1 − γ^{amp}) Vₘₐₓ^{pfk2}^{ₙₐₜᵢᵥₑ} for numerical value see Table 10
Vₘₐₓ^{pfk2}^{ₚ} = 2.3 · Vₘₐₓ^{pfk2}^{ₙₐₜᵢᵥₑ} [100]
${v}_{pfk 2}^{native} = \frac{fru 6 {p}_{cyt}}{fru 6 {p}_{cyt} + {K}_{m}^{fru 6 {p}_{cyt}}} \cdot \frac{{atp}_{cyt}}{{atp}_{cyt} + {K}_{m}^{{atp}_{cyt}}} \cdot ( 1 - \frac{{cit}_{cyt}}{{cit}_{cyt} + {K}_{i}^{{cit}_{cyt}}} )$
Kₘ^{fru6p}^{_{cyt}} = 0.121 [100] Kₘ^{atp}^{_{cyt}} = 0.63 [100] Kᵢ^{cit}^{_{cyt}} = 0.029 [100]
${v}_{pfk 2}^{p} = \frac{fru 6 {p}_{cyt}}{fru 6 {p}_{cyt} + {K}_{m}^{fru 6 {p}_{cyt}}} \cdot \frac{{atp}_{cyt}}{{atp}_{cyt} + {K}_{m}^{{atp}_{cyt}}} \cdot ( 1 - \frac{{cit}_{cyt}}{{cit}_{cyt} + {K}_{i}^{{cit}_{cyt}}} )$
Kₘ^{fru6p}^{_{cyt}} = 0.061 [100] Kₘ^{atp}^{_{cyt}} = 0.63 [100] Kᵢ^{cit}^{_{cyt}} = 0.061 [100]
Fructose-2,6-bisphosphatase (FBP2)
v_{fbp2} = Vₘₐₓ^{fbp2}^{ₚₕₒₛₚₕₒ} · γ^{fbp2} · v_{fbp2}^{p} γ^{fbp2} = γ · (1 − γ^{amp})
${V}_{ma x}^{fbp {2}_{phospho}} = {V}_{ma x}^{fbp {2}_{native}} [ 101 ] {v}_{fbp 2}^{p} = \frac{fru 26 {bp}_{cyt}}{fru 26 {bp}_{cyt} + {K}_{m}^{fru 26 {bp}_{cyt}}}$
Kₘ^{fru26p}^{_{cyt}} = 0.026 [102]
Phosphofructokinase 1 (Pfk1):
v_{pfk1} = Vₘₐₓ^{pfk1} · ((1 − γ^{Pfk1}) · v_{pfk1}^{native} + γ^{pfk1} · v_{pfk1}^{p})
γ^{pfk1} = γ · (1 − γ^{amp})
Vₘₐₓ^{pfk1} for numerical value see Table 10
${v}_{pfk 1}^{native} = \\ \frac{fru {26}_{cyr}^{n}}{fru {26}_{cyt}^{n} + {( {K}_{a}^{fru {26}_{cyt}} )}^{n}} \cdot \frac{{atp}_{cyt}}{{atp}_{cyt} + {K}_{m}^{{atp}_{cyt}}} \cdot ( 1 - {f}_{atp} \frac{{atp}_{cyt}^{{n}_{atp}}}{{atp}_{cyt}^{{n}_{atp}} + {( {K}_{i}^{{atp}_{cyt}} )}^{{n}_{atp}}} ) \cdot \\ ( 1 - {f}_{cit} \frac{{cit}_{cyt}^{{n}_{cit}}}{{cit}_{cyt}^{{n}_{cit}} + {( {K}_{i}^{{cit}_{cyt}} )}^{{n}_{cit}}} ) \cdot \frac{{( fru 6 {p}_{cyt} )}^{{n}_{fru 6 {p}_{cyt}}}}{{( fru 6 {p}_{cyt} )}^{{n}_{fru 6 {p}_{cyt}}} + {( {K}_{m}^{fru 6 {p}_{cyt}} )}^{{n}_{fru 6 {p}_{cyt}}}}$
n = 2 [103]
${K}_{a}^{fru {26}_{cyt}} = {K}_{0}^{fru {26}_{cyt}} \cdot ( \frac{{atp}_{cyt}^{{n}_{fru 26}}}{{atp}_{cyt}^{{n}_{fru 26}} + {( {K}_{{i}_{fru 26}}^{{atp}_{cyt}} \cdot ( 1 + \frac{{cit}_{cyt}}{{cit}_{cyt} + {K}_{{i}_{atp}}^{{cit}_{cyt}}} ) )}^{{n}_{fru 26}}} )$
${K}_{0}^{fru {26}_{cyt}} = 0.0 \; 10 [ 104 ] {n}_{fru 26} = {n}_{0} - \frac{{cit}_{cyt}}{{cit}_{cyt} + {K}_{cit}^{{n}_{fru 26}}} {n}_{0} = 5 [ 104 ]$
K_{cit}ⁿ^{_{fru26}} = 0.05 [104] Kᵢ_{_{fru26}}^{atp}^{_{cyt}} = 3.4 [104] Kᵢ_{ₐₜₚ}^{cit}^{_{cyt}} = 0.065 [104] Kₘ^{atp}^{_{cyt}} = 0.2 [105]
fₐₜₚ = 0.95 [103] nₐₜₚ = 6 [103] Kᵢ^{atp}^{_{cyt}} = 1.3 [103] n_{cit} = 4 [103]
f_{cit} = 0.485 [103] Kᵢ^{cit}^{_{cyt}} = 0.192 [103]
${K}_{m}^{fru 6 {p}_{cyt}} = {K}_{0} \cdot ( 1 - \frac{fru {26}_{cyt}}{fru {26}_{cyt} + {K}_{{a}_{fru 6 p}}^{fru {26}_{cyt}}} ) \cdot ( 1 - \frac{{p}_{cyt}}{{p}_{cyt} + {K}_{{a}_{fru 6 p}}^{{p}_{cyt}}} ) \cdot \\ ( 1 + {f}_{atp} \frac{{atp}_{cyt}^{{n}_{atp}}}{{atp}_{cyt}^{{n}_{atp}} + {( {K}_{{i}_{atp}}^{fru 26} )}^{{n}_{atp}}} ) \cdot ( 1 + {k}_{cit} \frac{{cit}_{cyt}^{{n}_{cit}}}{{cit}_{cyt}^{{n}_{cit}} + {( {K}_{i}^{{cit}_{cyt}} )}^{{n}_{cit}}} )$
K₀ = 7 [104] Kₐ_{_{fru6p}}^{fru26}^{_{cyt}} = 0.00015 [104] K_{afru6p}^{p}^{_{cyt}} = 0.15 [106] nₐₜₚ = 2 [106]
Kᵢ_{ₐₜₚ}^{fru26} = 0.2 [106] fₐₜₚ = 2 [106] k_{cit} = 8 [106] n_{cit} = 4 [106] Kᵢ^{cit}^{_{cyt}} = 0.13 [106]
${n}_{fru 6 {p}_{cyt}} = {n}_{0} \cdot ( 1 - f \frac{fru {26}_{cyt}}{fru {26}_{cyt} + {K}_{{n}_{fru 6 p}}^{fru 26 cyt}} ) \cdot \\ ( 1 + ( {f}_{atp} \frac{{atp}_{cyt}}{{atp}_{cyt} + {K}_{{n}_{fru 6 p}}^{fatp}} ) \cdot ( 1 - \frac{fru 6 {p}_{cyt}}{fru 6 {p}_{cyt} + {K}_{{n}_{fru 6 p}}^{fru 6}} ) \cdot ( 1 - \frac{{p}_{cyt}}{{p}_{cyt} + {K}_{{n}_{fru 6 p}}^{p}} ) )$
n₀ = 1 [104] f = 0.66 [104] fₐₜₚ = 2.75 [104] Kₙ_{_{fru6p}}^{fru26}^{_{cyt}} = 0.0001 [104]
Kₙ_{_{fru6p}}^{atp} = 0.1 [104] Kₙ_{_{fru6p}}^{fru6} = 0.4 [106] Kₙ_{_{fru6p}}^{p} = 0.5 [106]
${v}_{p f k 1}^{p} = \\ \frac{f r u 2 {6}_{c y t}^{n}}{f r u 2 {6}_{c y t}^{n} + {( {K}_{a}^{fru {26}_{c y 𝔱}} )}^{n}} \cdot \frac{a t {p}_{c y t}}{a t {p}_{c y t} + {K}_{m}^{a t {p}_{cy t}}} \cdot ( 1 - {f}_{a t p} \frac{a t {p}_{c y t}^{{n}_{atp}}}{a t {p}_{c y t}^{{n}_{atp}} + {( {K}_{i}^{a t {p}_{c y t}} )}^{{n}_{atp}}} ) \cdot ( 1 - {f}_{c i t} \frac{c i {t}_{c y t}^{{n}_{c i t}}}{c i {t}_{c y t}^{{n}_{c i t}} + {( {K}_{i}^{c i {t}_{c y t}} )}^{{n}_{c i 𝔱}}} ) \cdot \frac{{( f r u 6 {p}_{c y t} )}^{{n}_{fru 6 {p}_{c y t}}}}{{( f r u 6 {p}_{c y t} )}^{{n}_{fru 6 {p}_{c y t}}} + {( {K}_{m}^{fru 6 {p}_{c y 𝔱}} )}^{{n}_{fru 6 {p}_{c y t}}}}$
n = 2 [103]
${K}_{a}^{fru {26}_{c y 𝔱}} = {K}_{0}^{fru {26}_{c y 𝔱}} \cdot ( \frac{a t {p}_{c γ t}^{{n}_{f r u 26}}}{a t {p}_{c y t}^{{n}_{fru 26}} + {( {K}_{{i}_{fru 26}}^{a t {p}_{c y t}} \cdot ( 1 + \frac{c i {t}_{c y t}}{c i {t}_{c y t} + {K}_{{i}_{atp}}^{c i {t}_{c y t}}} ) )}^{{n}_{fru 26}}} )$
${K}_{0}^{f r u 2 {6}_{c y 𝔱}} = 0.01 [ 104 ] [ 103 ] {n}_{frτι 26} = {n}_{0} - \frac{c i {t}_{c y t}}{c i {t}_{c y t} + {K}_{c i t}^{{n}_{f r u 26}}} {n}_{0} = 5 [ 104 ]$
K_{cit}ⁿ^{_{fru26}} = 0.05 [104] Kᵢ_{_{fru26}}^{atp}^{_{cyt}} = 3.4 [104] Kᵢ_{ₐₜₚ}^{cit}^{_{cyt}} = 0.065 [104]
Kₘ^{atp}^{_{cyt}} = 0.2 [105] fₐₜₚ = 0.95 [103] nₐₜₚ = 5 [103] Kᵢ^{atp}^{_{cyt}} = 0.9 [103]
n_{cit} = 2 [103] f_{cit} = 0.55 [103] Kᵢ^{cit}^{_{cyt}} = 0.18 [103]
${K}_{m}^{f r u 6 {p}_{c y t}} = {K}_{0} \cdot ( 1 - \frac{f r u 2 {6}_{c y t}}{f r u 2 {6}_{c y t} + {K}_{{a}_{f r u 6 p}}^{f r u 2 {6}_{c y t}}} ) \cdot ( 1 - \frac{{p}_{c y t}}{{p}_{c y t} + {K}_{{a}_{fru 6 p}}^{{p}_{c y t}}} ) \cdot \\ ( 1 + {f}_{a t p} \frac{a t {p}_{c y t}^{{n}_{a t p}}}{a t {p}_{c y t}^{{n}_{atp}} + {( {K}_{{i}_{atp}}^{f r u 26} )}^{{n}_{atp}}} ) \cdot ( 1 + {k}_{c i t} \frac{c i {t}_{c y t}^{{n}_{c i t}}}{c i {t}_{c y t}^{{n}_{c i t}} + {( {K}_{i}^{c i {t}_{c y t}} )}^{{n}_{c i t}}} )$
K₀ = 5 [104] [103] Kₐ_{_{fru6p}}^{fru26}^{_{cyt}} = 0.00015 [104] Kₐ_{_{fru6p}}^{p}^{_{cyt}} = 0.15 [106]
nₐₜₚ = 2 [106] Kᵢ_{ₐₜₚ}^{fru26} = 0.2 [106] fₐₜₚ = 2 [106] k_{cit} = 8 [106]
n_{cit} = 4 [106] Kᵢ^{cit}^{_{cyt}} = 0.13 [106]
${n}_{fru 6 {p}_{c y t}} = {n}_{0} \cdot ( 1 - f \frac{f r u 2 {6}_{c y t}}{f r u 2 {6}_{c y t} + {K}_{{n}_{fru 6 p}}^{f r u 2 {6}_{c y t}}} ) \cdot \\ ( 1 + ( {f}_{a t p} \frac{a t {p}_{c y t}}{a t {p}_{c y t} + {K}_{{n}_{fru 6 p}}^{f a t p}} ) \cdot ( 1 - \frac{f r u 6 {p}_{c y t}}{f r u 6 {p}_{c y t} + {K}_{{n}_{f r u 6 p}}^{f r u 6}} ) \cdot ( 1 - \frac{{p}_{c y t}}{{p}_{c y t} + {K}_{{n}_{fru 6 p}}^{p}} ) )$
n₀ = 1 [104] f = 0.66 [104] fₐₜₚ = 2.75 [104] K_{nfru6p}^{fru26}^{_{cyt}} = 0.0001 [104]
Kₙ_{_{fru6p}}^{atp} = 0.1 [104] Kₙ_{_{fru6p}}^{fru6} = 0.4 [106] Kₙ_{_{fru6p}}^{p} = 0.5 [106]
Fructose-1,6-bisphosphatase (Fbp1)
v_{fbp1} = Vₘₐₓ^{vfbp1} · ((1 − γ^{fbp1}) · v_{fbp1}^{native} + γ^{fbp1} · v_{fbp1}^{p})
γ^{fbp1} = γ · (1 − γ^{amp})
Vₘₐₓ^{fbp1} for numerical value see Table 10
${v}_{f b ρ 1}^{n a t i v e} = \frac{f r u 16 b {p}_{c y t}}{f r u 16 b {p}_{c y t} + {K}_{m}^{f r u 16 b {ρ}_{cyt}}} / ( 1 + \frac{f r u 26 b {p}_{c y t}^{n}}{{( {K}_{i}^{fru 26 {bp}_{c y t}} )}^{n}} )$
Kₘ^{fru16bp}^{_{cyt}} = 0.0029 [107] Kᵢ^{fru26p}^{_{cyt}} = 0.00113 [107] n = 1.26 [107]
${v}_{f b ρ 1}^{p} = \frac{f r u 16 b {p}_{c y t}}{f r u 16 b {p}_{c y t} + {K}_{m}^{f r u 16 b {p}_{c y t}}} / ( 1 + \frac{f r u 26 b {p}_{c y t}^{n}}{{( {K}_{i}^{f r u 26 b {p}_{c y t}} )}^{n}} )$
Kₘ^{fru16bp}^{_{cyt}} = 0.0019 [107] Kᵢ^{fru26bp}^{_{cyt}} = 0.00113 [107] n = 1.26 [107]
Aldolase (Ald)
${v}_{ald} = {v}_{ma x}^{ald} \cdot \frac{f r u 16 b {p}_{c y t} - \frac{1}{{K}_{e q}^{a l d}} g r a {p}_{c y t} \cdot {dhap}_{c y t}}{( 1 + \frac{f r u 16 b {p}_{c y t}}{{K}_{m}^{f r u 16 b {p}_{cy t}}} ) + ( 1 + \frac{g r a {p}_{c y t}}{{K}_{m}^{g r a {p}_{c y t}}} ) \cdot ( 1 + \frac{d h a {p}_{c y t}}{{K}_{m}^{{dhap}_{c y t}}} ) - 1}$
vₘₐₓ^{ald} for numerical value see Table 10
K_{eq}^{ald} = 0.099 [108] Kₘ^{fru16bp}^{_{cyt}} = 0.004 [109] Kₘ^{grap}_{cyt} = 0.48 [110] Kₘ^{dhap}^{_{cyt}} = 0.38 [110]
Triosephosphate isomerase (Tpi)
${v}_{t ρ i} = {ν}_{m ax}^{t ρ i} \cdot \frac{d h a {p}_{c y t} - \frac{{grap}_{cyt}}{{K}_{eq}^{tpi}}}{1 + \frac{d h a {p}_{c y t}}{{K}_{m}^{d ℏ a {p}_{c y 𝔱}}} + \frac{gra {p}_{c y t}}{{K}_{m}^{g r a {p}_{c y t}}}}$
vₘₐₓ^{tpi} for numerical value see Table 10
K_{eq}^{tpi} = 0.04545 [108] Kₘ^{dhap}^{_{cyt}} = 0.58 [111] Kₘ^{grap}^{_{cyt}} = 0.4 [11]
D-Glyceraldehyde-3-phosphate:NAD⁺ oxidoreductase (Gapdh)
${v}_{gap dh} = {v}_{m ax}^{Gap dh} * \frac{{nad}_{cyt} \cdot {grap}_{cyt} \cdot {p}_{cyt} - \frac{1}{{K}_{eq}^{gapdh}} \cdot bpg {13}_{cyt} \cdot {nadh}_{cyt}}{( 1 + \frac{{nad}_{cyt}}{{K}_{m}^{{nad}_{cyt}}} ) \cdot ( 1 + \frac{{grap}_{cyt}}{{K}_{m}^{{grap}_{cyt}}} ) \cdot ( 1 + \frac{{p}_{cyt}}{{K}_{m}^{{p}_{cyt}}} ) + ( 1 + \frac{{nadh}_{cyt}}{{K}_{m}^{{nadh}_{cyt}}} ) \cdot ( 1 + \frac{bpg {13}_{cyt}}{{K}_{m}^{bpg {13}_{cyt}}} ) - 1}$
vₘₐₓ^{Gapdh} for numerical value see Table 10 K_{eq}^{gapdh} = 10⁻⁴ mM⁻¹ [112] Kₘ^{nad}^{_{cyt}} = 0.09 [113]
Kₘ^{grap}^{_{cyt}} = 0.044 [114] Kₘ^{p}^{_{cyt}} = 3.8 [115] Kₘ^{nadh}^{_{cyt}} = 0.006 [115] Kₘ^{bpg13}^{_{cyt}} = 0.01 [113]
Phosphoglycerate kinase (Pgk)
${v}_{pgk} = {v}_{m ax}^{pgk} \cdot \frac{{adp}_{cyt} \cdot bpg {13}_{cyt} - \frac{1}{{K}_{eq}^{pgk}} \cdot {atp}_{cyt} \cdot pg {3}_{cyt}}{( 1 + \frac{{adp}_{cyt}}{{K}_{m}^{{adp}_{cyt}}} ) \cdot ( 1 + \frac{bgp {13}_{cyt}}{{K}_{m}^{bpg {13}_{cyt}}} ) + ( 1 + \frac{{atp}_{cyt}}{{K}_{m}^{{atp}_{cyt}}} ) \cdot ( 1 + \frac{pg {3}_{cyt}}{{K}_{m}^{pg {3}_{cyt}}} ) - 1}$
vₘₐₓ^{pgk} for numerical value see Table 10 K_{eq}^{pgk} = 1830 [116] Kₘ^{adp}^{_{cyt}} = 0.35 [117] Kₘ^{bpg13}^{_{cyt}}
0.0022 [117] Kₘ^{atp}^{_{cyt}} = 0.151 [118] Kₘ^{pg3}^{_{cyt}} = 1.397 [118]
2-Phospho-D-glycerate 2,3 phosphomutase (Pgm)
${v}_{p g m} = {v}_{m ax}^{pgm} \cdot \frac{p g {3}_{c y t} - \frac{1}{{K}_{e q}^{p g m}} p g {2}_{c y t}}{p g {3}_{c y t} + {K}_{m}^{pg {3}_{c γ t}} \cdot ( 1 + \frac{p g {2}_{cy t}}{{K}_{m}^{pg 2}} )}$
vₘₐₓ^{pgm} for numerical value see Table 10
K_{eq}^{pgm} = 0.1725 [119] Kₘ^{pg3}^{_{cyt}} = 0.52 [120] Kₘ^{pg2} = 0.24 [120]
2-Phospho-D-glycerate hydrolase (Eno)
${v}_{e n o} = {ν}_{m ax}^{e n o} \cdot \frac{p g {2}_{c y t} - \frac{1}{{K}_{e q}^{en o}} \cdot {pep}_{c y t}}{1 + \frac{p g {2}_{c y t}}{{K}_{m}^{p g {2}_{c y t}}} + \frac{p e {p}_{c y t}}{{K}_{m}^{p e {p}_{cyt}}}}$
vₘₐₓ^{eno} for numerical value see Table 10
K_{eq}^{eno} = 1.7 [121] Kₘ^{pg2}^{_{cyt}} = 0.12 [122] Kₘ^{pep}^{_{cyt}} = 0.37 [122]
Pyruvate kinase (Pk)
${v}_{p k} = {v}_{ma x}^{P k} \frac{p e {p}_{c y t}}{p e {p}_{c y t} + {K}_{m}^{p e {p}_{cyt}} \cdot } \cdot \frac{a d {p}_{c y t}}{a d {p}_{c y t} + {K}_{m}^{a d {p}_{c y t}}}$
vₘₐₓ^{pk} for numerical value see Table 10 Kₘ^{pep}^{_{cyt}} = 0.08 [123] Kₘ^{adp}^{_{cyt}} = 0.3 [123]
Pyruvate carboxylase
${v}_{p c} = {ν}_{m ax}^{P c} \cdot \frac{a t {p}_{m i t o} \cdot {pyr}_{m i t o}}{( a t {p}_{m i t o} + {k}_{m}^{a t {p}_{m i t o}} ) \cdot ( {pyr}_{m i t o} + {k}_{m}^{p y {r}_{mito}} )}$
vₘₐₓ^{Pc} for numerical value see Table 10 Kₘ^{atp}^{ₘᵢₜₒ} = 0.14 [124] Kₘ^{pyr}^{ₘᵢₜₒ} = 0.33 [124]
Lactate dehydrogenase (Ldh):
${v}_{l d h} = {ν}_{m ax}^{l d ℏ} * \frac{p y {r}_{c y t} \cdot {nadh}_{c y t} - \frac{1}{{K}_{e q}^{l d h}} \cdot {lac}_{c y t} \cdot {nad}_{c y t}}{( 1 + \frac{n a d {h}_{c y t}}{{K}_{m}^{{nadh}_{cyt}}} ) \cdot ( 1 + \frac{p y {r}_{c y t}}{{K}_{m}^{p y {r}_{c y t}}} ) + ( 1 + \frac{l a {c}_{c y t}}{{K}_{m}^{l a {c}_{c y t}}} ) \cdot ( 1 + \frac{n a {d}_{c y t}}{{K}_{m}^{n a {d}_{cyt}}} ) - 1}$
vₘₐₓ^{ldh} for numerical value see Table 10 K_{eq}^{ldh} = 9000 [125] Kₘ^{nadh}^{_{cyt}} = 0.0121 [126] Kₘ^{pyr}^{_{cyt}} =
0.1 [127] Kₘ^{lac}^{_{cyt}} = 4.4 [126] Kₘ^{nad}^{_{cyt}} = 0.1 [126]
Lactate transport (LacT):
${v}_{lacT} = {v}_{m ax}^{lacT} \cdot \frac{l a {c}_{e x t} - {lac}_{cyt}}{1 + \frac{l a {c}_{c y t}}{{K}_{m}^{l a {c}_{c y t}}} + \frac{{lac}_{e x t}}{{K}_{m}^{{lac}_{e x t}}}}$
vₘₐₓ^{lacT} for numerical value see Table 10 Kₘ^{lac}^{_{cyt}} = 2.5 [128] Kₘ^{lac}^{ₑₓₜ} = 2.5 [128]
Pyruvate transport (PyrT):
${v}_{lacT} = {v}_{m ax}^{p y r T} \cdot \frac{p y {r}_{e x t} - {pyr}_{cyt}}{1 + \frac{p y {r}_{c y t}}{{K}_{m}^{p y {r}_{c y 𝔱}}} + \frac{{pyr}_{e x t}}{{K}_{m}^{p y {r}_{e x t}}}}$
vₘₐₓ^{pyrT} for numerical value see Table 10 Kₘ^{pyr}^{ₑₓₜ} = 0.1 [128] Kₘ^{pyr}^{_{cyt}} = 0.1 [128]
Mitochondrial pyruvate transport:
${v}_{{pyrT}_{mito}} = {v}_{m ax}^{p y r {T}_{mito}} \cdot \frac{p y {r}_{c y t} \cdot {h}_{c y t} - p y {r}_{m i t o} \cdot {h}_{m i t o}}{1 + \frac{p y {r}_{c y t}}{{K}_{m}^{p y {r}_{c y t}}} + \frac{p y {r}_{m i t o}}{{K}_{m}^{p y {r}_{m i t o}}}}$
vₘₐₓ^{PyrT}^{ₘᵢₜₒ} for numerical value see Table 10 Kₘ^{pyr}^{_{cyt}} = 0.15 [129] Kₘ^{pyr}^{ₘᵢₜₒ} = 0.15 [129]
Mitochondrial malate-phosphate transport
${v}_{m a l p T} = {v}_{m ax}^{m a l p T} \cdot ( \frac{{mal}_{m i t o} \cdot {p}_{cyt} - {mal}_{cyt} \cdot {p}_{mito}}{( 1 + \frac{{mal}_{m i t o}}{{K}_{m}^{{mal}_{mito}}} ) \cdot ( 1 + \frac{{p}_{c y t}}{{K}_{m}^{{p}_{c y t}}} ) + ( 1 + \frac{m a {l}_{c y t}}{{K}_{m}^{{mal}_{c y t}}} ) \cdot ( 1 + \frac{{p}_{m i t o}}{{K}_{m}^{{p}_{mito}}} ) - 1} )$
vₘₐₓ^{malpT} for numerical value see Table 10
Kₘ^{p}^{_{cyt}} = 1.41 [130] Kₘ^{mal}^{ₘᵢₜₒ} = 0.4 [131] Kₘ^{p}^{ₘᵢₜₒ} = 1.41 [130] Kₘ^{mal}^{_{cyt}} = 0.4 [131]
Malate-pyruvate antiport (MalPyrT)
${v}_{m a l - p y r T} = {v}_{ma x}^{mal - pyrT} * ( \frac{{mal}_{m i t o} \cdot {pyr}_{c y t} - m a {l}_{c y t} \cdot {pγr}_{mito}}{( 1 + \frac{{mal}_{m i t o}}{{K}_{m}^{m a {l}_{mito}}} ) \cdot ( 1 + \frac{p y {r}_{c y t}}{{K}_{m}^{{pyr}_{c y t}}} ) + ( 1 + \frac{m a {l}_{c y t}}{{K}_{m}^{m a {l}_{c y t}}} ) \cdot ( 1 + \frac{p y {r}_{m i t o}}{{K}_{m}^{p y {r}_{m i t o}}} ) - 1} )$
vₘₐₓ^{MalPyrT} for numerical value see Table 10
Kₘ^{pyr}^{_{cyt}} = 0.84 [132] Kₘ^{mal}^{_{cyt}} = 0.7 [133] Kₘ^{pyr}^{ₘᵢₜₒ} = 0.84 [132] Kₘ^{mal}^{_{cyt}} = 0.7 [133]
Cytosolic malate dehydrogenase (Mdh)
${v}_{m d ℏ} = {ν}_{m ax}^{M d h} \cdot \frac{{mal}_{cyt} \cdot {nad}_{cyt} - \frac{1}{{K}_{eq}^{{mdh}_{cyt}}} \cdot {oaa}_{cyt} \cdot {nadh}_{cyt}}{( 1 + \frac{m a {l}_{c y t}}{{K}_{m}^{m a {l}_{c y 𝔱}}} ) \cdot ( 1 + \frac{n a {d}_{c y t}}{{K}_{m}^{n a {d}_{c y t}}} ) + ( 1 + \frac{o a {a}_{cyt}}{{K}_{m}^{{oaa}_{cyt}}} ) \cdot ( 1 + \frac{n a d {h}_{cyt}}{{K}_{m}^{n a d {h}_{c y t}}} ) - 1}$
vₘₐₓ^{Mdh} for numerical value see Table 10 K_{eq}^{mdh}^{_{cyt}} = 10⁻⁵ [72] Kₘ^{mal}^{_{cyt}} = 0.47 [134]
Kₘ^{nad}^{_{cyt}} = 0.099 [134] Kₘ^{oaa}^{_{cyt}} = 0.042 [134] Kₘ^{nadh}^{_{cyt}} = 0.027 [134]
NADP-dependent malic enzyme (cytosol)
${v}_{m e} = {V}_{m ax}^{m e} \cdot ( \frac{m a {l}_{c y t} \cdot {nadp}_{cyt} - 1 / {K}_{eq}^{me} \cdot {pyr}_{cyt} \cdot {nadph}_{cyt} \cdot hco {3}_{cyt}}{( 1 + \frac{m a {l}_{c y t}}{{K}_{m}^{m a {l}_{c y t}}} ) \cdot ( 1 + \frac{n a d {p}_{c y t}}{{K}_{m}^{n a d {p}_{c y t}}} ) + ( 1 + \frac{p y {r}_{c y t}}{{K}_{m}^{p y {r}_{c y t}}} ) \cdot ( 1 + \frac{n a d p {h}_{c y t}}{{K}_{m}^{{nadph}_{c y t}}} ) \cdot ( 1 + \frac{h c o {3}_{c y t}}{{K}_{m}^{h c o {3}_{c y t}}} )} )$
Vₘₐₓ^{me} for numerical value see Table 10
K_{eq}^{me} = 34.4 [135] Kₘ^{mal}^{_{cyt}} = 0.12 [136] Kₘ^{nadp}^{_{cyt}} = 0.0092 [136] Kₘ^{pyr}^{_{cyt}} = 8 [137]
Kₘ^{nadph}^{_{cyt}} = 0.0053 [136] Kₘ^{hco3}^{_{cyt}} = 13 [137]
Nucleoside diphosphokinase (cytosolic)
${v}_{n d {k}_{cyt}} = {V}_{ma x}^{{ndk}_{c y t}} \cdot ( \frac{{atp}_{cyt} \cdot {gdp}_{cyt} - 1 / {K}_{e q}^{n d k} \cdot {adp}_{c y t} \cdot {gtp}_{c y t}}{( 1 + \frac{a t {p}_{c y t}}{{K}_{m}^{{atp}_{c y t}}} ) \cdot ( 1 + \frac{g d {p}_{c y t}}{{K}_{m}^{{gdp}_{c y t}}} ) + ( 1 + \frac{a d {p}_{c y t}}{{K}_{m}^{a d {p}_{c y t}}} ) \cdot ( 1 + \frac{g t {p}_{c y t}}{{K}_{m}^{g t {p}_{c y t}}} ) - 1} )$
Vₘₐₓ^{ndk}^{_{cyt}} for numerical value see Table 10 K_{eq}^{ndk} = 1 [66]
Kₘ^{atp}^{_{cyt}} = 1.8 [138] Kₘ^{gdp}^{_{cyt}} = 0.049 [138] Kₘ^{adp}^{_{cyt}} = 0.066 [138] Kₘ^{gtp}^{_{cyt}} = 0.15 [138]
Nucleoside diphosphokinase (mito)
${v}_{n d {k}_{m i t o}} = {V}_{ma x}^{{ndk}_{mito}} \cdot ( \frac{{atp}_{mito} \cdot {gdp}_{mito} - 1 / {K}_{e q}^{ndk} \cdot {adp}_{m i t o} \cdot {gtp}_{mito}}{( 1 + \frac{{atp}_{m i t o}}{{K}_{m}^{a t {p}_{m i t o}}} ) \cdot ( 1 + \frac{g d {p}_{m i t o}}{{K}_{m}^{g t {p}_{m i t o}}} ) + ( 1 + \frac{a d {p}_{m i t o}}{{K}_{m}^{{adp}_{m i t o}}} ) \cdot ( 1 + \frac{g t {p}_{m i t o}}{{K}_{m}^{{gtp}_{mito}}} ) - 1} )$
vₘₐₓ^{ndk}^{ₘᵢₜₒ} for numerical value see Table 10 K_{eq}^{ndk} = 1 [66]
Kₘ^{atp}^{ₘᵢₜₒ} = 1.66 [139] Kₘ^{gdp}^{ₘᵢₜₒ} = 0.036 [139] Kₘ^{adp}^{ₘᵢₜₒ} = 0.073 [139] Kₘ^{gtp}^{ₘᵢₜₒ} = 0.15 [138]
Glycogen metabolism
Alpha-D-Glucose 1-phosphate 1,6-phosphomutase:
${v}_{gpm} = {v}_{m ax}^{gpm} \cdot \frac{g l c 1 {p}_{c y t} - \frac{1}{{K}_{e q}^{g p m}} \cdot glc 6 {p}_{c y t}}{1 + \frac{g l c 1 {p}_{c y t}}{{K}_{m}^{glc 1 {p}_{cyt}}} + \frac{g l c 6 {p}_{c y t}}{{K}_{m}^{glc 6 {p}_{c y t}}}}$
vₘₐₓ^{gpm} for numerical value see Table 10
K_{eq}^{gpm} = 16.2 [140] Kₘg^{lc1p}^{_{cyt}} = 0.045 [141] Km^{glc6p}^{_{cyt}} = 0.67 [141]
UTP:Glucose-1-phosphate uridylyltransferase (UPGase):
${v}_{u p g a s e} = {v}_{m ax}^{upgase} \cdot \frac{{utp}_{cyt} \cdot glc 1 {p}_{cyt} - \frac{1}{{K}_{eq}^{upgase}} \cdot {udpglc}_{cyt} \cdot {pp}_{cyt}}{( 1 + \frac{u t {p}_{c y t}}{{K}_{m}^{u t {p}_{cyt}}} ) \cdot ( 1 + \frac{g l c 1 {p}_{c y t}}{{K}_{m}^{glc 1 {p}_{c y t}}} ) + ( 1 + \frac{u d p g l {c}_{c y t}}{{K}_{m}^{{udphlc}_{cyt}}} ) \cdot ( 1 + \frac{p {p}_{c y t}}{{K}_{m}^{{pp}_{c y t}}} ) - 1}$
vₘₐₓ^{UPGase} for numerical value see Table 10 K_{eq}^{upgase} = 0.3122 [142]
Kₘ^{utp}^{_{cyt}} = 0.2 [142] Kₘ^{glc1p}^{_{cyt}} = 0.055 [142] Kₘ^{udpglc}^{_{cyt}} = 0.06 [142] Kₘ^{pp}^{_{cyt}} = 0.084 [142]
Glycogen synthase (GS):
v_{gs} = Vₘₐₓ^{gs} · ((1 − γ^{gs}) · v_{gs}^{native} + γ^{gs} · v_{gs}^{p})
${γ}^{gs} = 1 - γ \cdot ( 1 - \frac{{epi}_{e x t}}{e p {i}_{e x t} + {K}_{i}^{epi}} ) {K}_{i}^{e p i} = 200 pM {V}_{m ax}^{gs} = {V}_{0}^{gs} \frac{( store - glyglc )}{( store - glyglc ) + 10 mM}$
V₀^{gs} for numerical value see Table 10 Geben Sie hier eine Formel ein. store = 5 mM
${v}_{g s}^{n a t i v e} = ( \frac{u d p g l {c}_{c y t}}{u d p g l {c}_{c y t} + {K}_{m - n a t i v e}^{{udpglc}_{cyt}}} ) \cdot ( \frac{glc 6 {p}_{c y t}}{g l c 6 {p}_{c y t} + {K}_{a}^{g l c 6 {p}_{c y t}}} )$
${K}_{m - n a t i ν e}^{{udpglc}_{c y t}} = {K}_{0 - n a t i ν e}^{u d p g l {c}_{c y t}} \cdot ( 1 - \frac{g l c 6 {p}_{c y t}}{glc 6 {p}_{c y t} + {K}_{a 2}^{glc 6 {p}_{c y t}}} ) + {K}_{b - n a t i v e}^{{udpglc}_{cyt}}$
K₀₋ₙₐₜᵢᵥₑ^{udpglc}^{_{cyt}} = 0.9 [143] Kₐ^{glc6p}^{_{cyt}} = 0.004 [143] Kₐ₂^{glc6p}^{_{cyt}} = 0.004 [143] K_{b-native}^{udpglc}^{_{cyt}} =
0.2 [143]
${v}_{gs}^{p} = ( \frac{u d p g l {c}_{c y t}}{u d p g l {c}_{c y t} + {K}_{m - p}^{{udpglc}_{cyt}}} ) \cdot ( \frac{g l c 6 {p}_{c y t}}{g l c 6 {p}_{c y t} + {K}_{a}^{g l c 6 {p}_{c y t}}} )$
Kₘ₋ₚ^{udpglc}^{_{cyt}} = K₀₋ₚ^{udpglc}^{_{cyt}} K₀₋ₚ^{udpglc}^{_{cyt}} = 0.9 [143] Kₐ^{glc6p}_{cyt} =2 [143]
Glycogen phosphorylase (GP):
v_{gp} = ((1 − γ^{gp}) · v_{gp}^{native} + γ^{gp} · v_{gp}^{p})
Y^{gp} = y^{gs}
${v}_{gp}^{n a t i v e} = {V}_{m ax}^{gp - native} \cdot \frac{glyglc \cdot {p}_{cyt} - \frac{1}{{K}_{eq}^{gp}} \cdot glc 1 {p}_{cyt}}{( 1 + \frac{glyglc}{{K}_{m - n a t i ν e}^{glycogen}} ) \cdot ( 1 + \frac{{p}_{c y t}}{{K}_{m - n a t i ν e}^{{p}_{c y t}}} ) + ( 1 + \frac{g l c 1 {p}_{c y t}}{{K}_{m - n a t i v e}^{glc 1 {p}_{c y t}}} ) - 1}$
${V}_{m ax - n a t i v e}^{gp} = \frac{{V}_{0}^{g p}}{{K}_{m - n a t i ν e}^{{p}_{c y t}} \cdot {K}_{m - native}^{glyplc}} \cdot ( \frac{a m {p}_{c y t}}{a m {p}_{c y t} + {K}_{a - n a t i v e}^{{amp}_{cyt}}} ) \cdot ( \frac{g l y g l c}{g l y g l c + 0.1 \cdot store} )$
V₀^{gp} for numerical value see Table 10 store = 5 mM
Kₐ₋ₙₐₜᵢᵥₑ^{amp} = 0.0022 [144] K_{eq}^{gp} = 0.21(mM)⁻¹ [145] Kₘ₋ₙₐₜᵢᵥₑ^{glyglc} = 2.5 [146] Kₘ₋ₙₐₜᵢᵥₑ^{p}^{_{cyt}} =
500 [146]
${K}_{m - n a t i ν e}^{g l c 1 {p}_{c y t}} = {K}_{0}^{g l c 1 p} \cdot ( 1 - \frac{a m {p}_{c y t}}{a m {p}_{c y t} + {K}_{a - glc 1 p}^{a m {p}_{c y t}}} ) {K}_{0}^{glc 1 p} = 250 [ 146 ] {K}_{a - glc1p}^{a m {p}_{c y t}} = 0.5 [ 146 ]$
${v}_{gp}^{p} = {V}_{ma x - p}^{gp} \cdot \frac{glyglc \cdot {p}_{c y t} - \frac{1}{{K}_{e q}^{g p}} \cdot glc 1 {p}_{c y t}}{( 1 + \frac{glyglc}{{K}_{m - p}^{glycogen}} ) \cdot ( 1 + \frac{{p}_{c y t}}{{K}_{m - p}^{{p}_{c y t}}} ) + ( 1 + \frac{g l c 1 {p}_{c y t}}{{K}_{m - p}^{glc 1 {p}_{cyt}}} ) - 1}$
${V}_{ma x - p}^{gp} = \frac{{V}_{0}^{g p}}{{K}_{m - p}^{p} \cdot {K}_{m - p}^{glyglc}} \cdot ( {k}_{1} + \frac{a m {p}_{c y t}}{a m {p}_{c y t} + {K}_{a - p}^{a m {p}_{c y t}}} ) \cdot ( \frac{g l y g l c}{g l y g l c + 0.1 \cdot store} )$
k₁ = 0.5 [144] Kₐ₋ₙₐₜᵢᵥₑ^{amp} = 0.22 [144]
Kₘ₋ₚ^{glyglc} = 0.27 [144] Kₘ₋ₚ^{p}^{_{cyt}} = 3.8 [144] Kₘ₋ₚ^{glc1p}^{_{cyt}} = 0.7 [146]
Nucleoside diphosphokinase (cytosolic) (udp)
${v}_{{ndk}_{cyt}} = {V}_{m ax}^{{ndk}_{cyt}} \cdot ( \frac{a t {p}_{c y t} \cdot {udp}_{cyt} - 1 / {K}_{eq}^{ndk} \cdot {adp}_{cyt} \cdot {utp}_{cyt}}{( 1 + \frac{a t {p}_{c y t}}{{K}_{m}^{a t {p}_{c y t}}} ) \cdot ( 1 + \frac{u d {p}_{c y t}}{{K}_{m}^{{udp}_{c y t}}} ) + ( 1 + \frac{a d {p}_{c y t}}{{K}_{m}^{{adp}_{c y t}}} ) \cdot ( 1 + \frac{u t {p}_{c y t}}{{K}_{m}^{u t {p}_{c y t}}} ) - 1} )$
Vₘₐₓ^{ndk}^{_{cyt}} for numerical value see Table 10 K_{eq}^{ndk} = 1 [66]
Kₘ^{atp}^{_{cyt}} = 0.5 [147] Kmudpcyt = 0.05 [147] Kₘ^{adp}^{_{cyt}} = 0.07 [147] Kₘ^{utp}^{_{cyt}} = 0.15 [147]
Malate-Aspartate shuttle
Aspartate-amino transferase (mitochondrial)
${v}_{{asat}_{mito}} = {V}_{m ax}^{a s a t} \cdot ( \frac{a s {p}_{m i t o} \cdot {akg}_{m i t o} - 1 / {K}_{eq}^{asat} \cdot {oaa}_{m i t o} \cdot {glu}_{m i t o}}{( 1 + \frac{a s {p}_{m i t o}}{{K}_{m}^{a s {p}_{m i t o}}} ) \cdot ( 1 + \frac{a k {g}_{m i t o}}{{K}_{m}^{a k {g}_{m i t o}}} ) + ( 1 + \frac{o a {a}_{m i t o}}{{K}_{m}^{o a {a}_{mito}}} ) \cdot ( 1 + \frac{g l {u}_{m i t o}}{{K}_{m}^{{glu}_{m i t o}}} ) - 1} )$
Vₘₐₓ^{asat}for numerical value see Table 10 K_{eq}^{asat} = 0.147 [148]
Kₘ^{asp}^{ₘᵢₜₒ} = 0.35 [149] Kₘ^{akg}^{ₘᵢₜₒ} = 1.1 [149] Kₘ^{oaa}^{ₘᵢₜₒ} = 1.84 [150] Kₘ^{glu}^{ₘᵢₜₒ} = 0.48 [150]
Aspartate-amino transferase (cytosolic)
${v}_{a s a t} = {V}_{ma x}^{a s a t} \cdot ( \frac{a s {p}_{c y t} \cdot {akg}_{cyt} - 1 / {K}_{eq}^{asat} \cdot {oaa}_{cyt} \cdot {glu}_{cyt}}{( 1 + \frac{a s {p}_{c y t}}{{K}_{m}^{a s {p}_{c y t}}} ) \cdot ( 1 + \frac{a k {g}_{c y t}}{{K}_{m}^{{akg}_{cyt}}} ) + ( 1 + \frac{o a {a}_{c y t}}{{K}_{m}^{o a {a}_{c y t}}} ) \cdot ( 1 + \frac{g l {u}_{cyt}}{{K}_{m}^{g l {u}_{c y t}}} ) - 1} )$
Vₘₐₓ^{asat} for numerical value see Table 10 K_{eq}^{asat} = 0.147 [148]
Kₘ^{asp}^{_{cyt}} = 3.9 [149] Kₘ^{akg}^{_{cyt}} = 0.57 [149] Kₘ^{oaa}^{_{cyt}} = 2.05 [150] Kₘ^{glu}^{_{cyt}} = 0.38 [150]
Aspartate-glutamate carrier
${v}_{agc} = {V}_{ma x}^{agc} \cdot ( \frac{a s {p}_{m i t o} \cdot {glu}_{cyt} - 1 / {K}_{eq}^{agc} \cdot {asp}_{cyt} \cdot {glu}_{mito}}{( 1 + \frac{a s {p}_{m i t o}}{{K}_{m}^{{asp}_{mi𝔱o}}} ) \cdot ( 1 + \frac{g l {u}_{c y t}}{{K}_{m}^{{glu}_{c y t}}} ) + ( 1 + \frac{a s {p}_{c y t}}{{K}_{m}^{{asp}_{c y t}}} ) \cdot ( 1 + \frac{g l {u}_{m i t o}}{{K}_{m}^{g l {u}_{mito}}} ) - 1} )$
Vₘₐₓ^{asat}for numerical value see Table 10
${K}_{e q}^{a s a t} = exp ( \frac{- {V}_{m m} \cdot F}{R \cdot T} ) \cdot ( \frac{{H}_{cyt}}{{H}_{mito}} ) {K}_{m}^{{asp}_{mito}} = {K}_{0}^{{asp}_{mito}} \cdot ( 1 + \frac{{glu}_{mito}}{{K}_{i}^{{glu}_{mito}}} )$
${K}_{0}^{{asp}_{mito}} = 0.05 [ 151 ] {K}_{i}^{{glu}_{mito}} = 0.5 [ 152 ] {K}_{m}^{{asp}_{cyt}} = {K}_{0}^{{asp}_{cyt}} \cdot ( 1 + \frac{{glu}_{cyt}}{{K}_{i}^{{glu}_{cyt}}} )$
K₀^{asp}^{_{cyt}} = 0.043 [152] Kᵢ^{glu}^{_{cyt}} = 0.5 [152] Kₘ^{glu}^{ₘᵢₜₒ} = 3 [153] Kₘ^{glu}^{_{cyt}} = 3.2 [153]
Malate - α-ketogluterate carrier
${v}_{m a c} = {V}_{m ax}^{m a c} \cdot ( \frac{m a {l}_{c y t} \cdot {akg}_{mito} - 1 / {K}_{eq}^{mac} \cdot {mal}_{mito} \cdot {akg}_{cyt}}{( 1 + \frac{m a {l}_{c y t}}{{K}_{m}^{m a {l}_{c y t}}} ) \cdot ( 1 + \frac{a k {g}_{m i t o}}{{K}_{m}^{{akg}_{mito}}} ) + ( 1 + \frac{m a {l}_{m i t o}}{{K}_{m}^{{mal}_{mito}}} ) \cdot ( 1 + \frac{a k {g}_{c y t}}{{K}_{m}^{a k {g}_{c y t}}} ) - 1} )$
Vₘₐₓ^{mac} for numerical value see Table 10 K_{eq}^{mac} = 1
Kₘ^{mal}^{_{cyt}} = 0.7 [154] Kₘ^{akg}^{ₘᵢₜₒ} = 0.17 [154] Kₘ^{mal}^{ₘᵢₜₒ} = 1.4 [154] Kₘ^{akg}^{_{cyt}} = 0.3 [154]
Glycerol-3-phosphate dehydrogenase (cytosolic)
${v}_{g3pdh} = {V}_{m ax}^{g 3 {pdh}_{cyt}} \cdot ( \frac{{dhap}_{cyt} \cdot {nad}_{cyt} - 1 / {K}_{eq}^{g 3 pdh} \cdot g 3 {p}_{cyt} \cdot {nad}_{cyt}}{( 1 + \frac{d h a {p}_{c y t}}{{K}_{m}^{{dhap}_{c y t}}} ) \cdot ( 1 + \frac{n a d {h}_{c y t}}{{K}_{m}^{{nadh}_{cyt}}} ) + ( 1 + \frac{g 3 {p}_{c y t}}{{K}_{m}^{g 3 {p}_{c y 𝔱}}} ) \cdot ( 1 + \frac{n a {d}_{c y t}}{{K}_{m}^{n a {d}_{c y t}}} ) - 1} )$
Vₘₐₓ^{g3pdh}^{_{cyt}} for numerical value see Table 10
${K}_{e q}^{g 3 {pdh}_{cyt}} = \frac{1}{3 \cdot {10}^{- 4}} [ 155 ]$
Kₘ^{dhap}^{_{cyt}} = 0.2 [156] Kₘ^{nadh}^{_{cyt}} = 0.1 [156] Kₘ^{g3p}^{_{cyt}} = 0.17 [156] K^{mnad}^{_{cyt}} = 0.063 [156]
Glycerol-3-phosphate dehydrogenase (mitochondrial)
${v}_{g 3 {pdh}_{mito}} = {y}_{m ax}^{{g3pdh}_{mito}} \cdot ( \frac{d h a {p}_{c y t} \cdot qh {2}_{m m} - 1 / {K}_{eq}^{g 3 pdh} \cdot g 3 {p}_{cyt} \cdot {q}_{m m}}{( 1 + \frac{d h a {p}_{c y t}}{{K}_{m}^{d h a {p}_{c y t}}} ) + ( 1 + \frac{g 3 {p}_{c y t}}{{K}_{m}^{g 3 {p}_{c y t}}} ) - 1} )$
Vₘₐₓ^{g3pdh}^{ₘᵢₜₒ} for numerical value see Table 10
${K}_{eq}^{g 3 {pdh}_{mito}} = {K}_{eq}^{g 3 p {dh}_{cyt}} \cdot exp ( \frac{( n \cdot {E}_{0}^{nad / nadh} + n \cdot {E}_{0}^{{QH}_{2} / Q} ) \cdot F}{R \cdot T} )$
Kₘ^{dhap}^{_{cyt}} = 0.23 [157] Kₘ^{g3p}^{_{cyt}} = 1.8 [158] E₀^{nad/nadh} = −320 mV [82]
E₀^{QH2/Q} = −87 mV [41] n = 2
Pentose phosphate shunt
Glucose-6-phosphate dehydrogenase
${v}_{g 6 pdh} = {V}_{m ax}^{g 6 pdh} \cdot ( ( \frac{glc 6 {p}_{cyt}}{glc 6 {p}_{cyt} + {K}_{m}^{glc {6}_{cyt}} \cdot ( 1 + \frac{c 16 {coa}_{cyt}}{{K}_{i}^{c 16 {coa}_{cyt}}} )} ) \cdot \\ ( \frac{n a d {p}_{c y t}}{n a d {p}_{c y t} + {K}_{m}^{{nadp}_{cyt}} \cdot ( 1 + \frac{n a d p {h}_{c y t}}{{K}_{i}^{{nadph}_{c y t}}} )} ) )$
Vₘₐₓ^{g6pdh} for numerical value see Table 10
K_{mglc6p}^{_{cyt}} = 0.013 [159] K_{ic16coa}^{_{cyt}} = 0.029 [160] Kₘ^{nadp}^{_{cyt}} = 0.013 [159] Kᵢ^{nadph}^{_{cyt}} = 0.01 [161]
6-Phosphogluconolactase
${v}_{pgls} = {V}_{ma x}^{ν gls} \cdot ( \frac{pgl {6}_{cyt}}{pgl {6}_{c y t} + {K}_{m}^{p g l {6}_{c y t}}} )$
Vₘₐₓ^{pgls}for numerical value see Table 10 Kₘ^{pg16}^{_{cyt}} = 0.7 [162]
6-Phosphogluconate dehydrogenase
${v}_{p g d h} = {V}_{ma x}^{pgdh} \cdot ( \frac{{nadp}_{cyt} \cdot pg {6}_{cyt} - 1 / {K}_{eq}^{pgdh} \cdot ru 5 {p}_{cyt} \cdot {nadph}_{cyt}}{( 1 + \frac{n a d {p}_{c y t}}{{K}_{m}^{n a d {p}_{c y 𝔱}} \cdot ( 1 + \frac{n a d p {h}_{c y t}}{{K}_{i}^{n a d p {h}_{c y t}}} )} ) \cdot ( 1 + \frac{p g {6}_{c y t}}{{K}_{m}^{pg {6}_{cyt}}} ) + \\ ( 1 + \frac{r u 5 {p}_{c y t}}{{K}_{m}^{r u 5 {p}_{c y t}}} ) \cdot ( 1 + \frac{c o {2}_{c y t}}{{K}_{m}^{c o {2}_{c y 𝔱}}} ) \cdot ( 1 + \frac{n a d p {h}_{c y t}}{{K}_{m}^{{nadph}_{c y t}}} ) - 1} )$
Vₘₐₓ^{pgdh} for numerical value see Table 10
K_{eq}^{pgdh} = 74 [163] Kₘ^{nadp}^{_{cyt}} = 0.028 [159] Kᵢ^{nadph} = 0.02 [164]
Kₘ^{pg6}^{_{cyt}} = 0.071 [164] Kₘ^{co2}^{_{cyt}} = 5 [165] Kₘ^{nadph}^{_{cyt}} = 0.001 [165] Kₘ^{ru5p}^{_{cyt}} = 0.123 [165]
Ribulose-phosphate-3-epimerase
${ν}_{r p e} = {V}_{m ax}^{r p e} \cdot ( \frac{ru 5 {p}_{cyt} - 1 / {K}_{eq}^{rpe} \cdot x 5 {p}_{cyt}}{1 + \frac{r u 5 {p}_{c y t}}{{K}_{m}^{r u 5 {p}_{c γ t}}} + \frac{x 5 {p}_{c y t}}{{K}_{m}^{x 5 {p}_{c y t}}}} )$
Vₘₐₓ^{rpe} for numerical value see Table 10 K_{eq}^{rpe} = 1.5 [166] Kₘ^{ru5p}^{_{cyt}} = 0.2 [167] Kₘ^{x5p}^{_{cyt}} = 0.5
Ribose-phosphate-isomerase
${v}_{r p i} = {V}_{m ax}^{r p i} \cdot ( \frac{r 5 {p}_{cyt} - 1 / {K}_{eq}^{rpi} \cdot ru 5 {p}_{cyt}}{1 + \frac{r 5 {p}_{c y t}}{{K}_{m}^{r 5 {p}_{c y t}}} + \frac{r u 5 {p}_{c y t}}{{K}_{m}^{r u 5 {p}_{c y t}}}} )$
Vₘₐₓ^{rpi} for numerical value see Table 10
K_{eq}^{rpi} = 0.32 [168] Kₘ^{r5p}^{_{cyt}} = 9.1 [169] Kₘ^{ru5p}^{_{cyt}} = 0.78 [169]
Transladolase
${v}_{taldo} = {V}_{ma x}^{taldo} \cdot ( \frac{s 7 {p}_{c y t} \cdot {grap}_{cyt} - 1 / {K}_{eq}^{taldo} \cdot e 4 {p}_{cyt} \cdot fru 6 {p}_{cyt}}{( 1 + \frac{s 7 {p}_{c y t}}{{K}_{m}^{s 7 {p}_{c y 𝔱}}} ) \cdot ( 1 + \frac{g r a {p}_{c y t}}{{K}_{m}^{g r a {p}_{c y t}}} ) + ( 1 + \frac{e 4 {p}_{c y t}}{{K}_{m}^{e 4 {ρ}_{cyt}}} ) \cdot ( 1 + \frac{f r u 6 {p}_{c y t}}{{K}_{m}^{f r u 6 {p}_{c y t}}} ) - 1} )$
Vₘₐₓ^{tado} for numerical value see Table 10 K_{eq}^{taldo} = 0.95 [170]
Kₘ^{s7p}^{_{cyt}} = 0.17 [170] Kₘ^{grap}^{_{cyt}} = 0.038 [171] Kₘ^{e4p}^{_{cyt}} = 0.13 [172] Kₘ^{fru6p}^{_{cyt}} = 0.3 [172]
Transketolase 1
${v}_{t k e t o 1} = {V}_{m ax}^{tketo 1} \cdot ( \frac{s 7 {p}_{c y t} \cdot {grap}_{c y t} - 1 / {K}_{e q}^{t k e t o 1} \cdot r 5 {p}_{c y t} \cdot x 5 {p}_{c y t}}{( 1 + \frac{s 7 {p}_{c y t}}{{K}_{m}^{s 7 {p}_{c y t}}} ) \cdot ( 1 + \frac{g r a {p}_{c y t}}{{K}_{m}^{{grap}_{cyt}}} ) + ( 1 + \frac{r 5 {p}_{c y t}}{{K}_{m}^{r 5 {p}_{cyt}}} ) \cdot ( 1 + \frac{x 5 {p}_{c y t}}{{K}_{m}^{x 5 {p}_{c y t}}} ) \cdot - 1} )$
Vₘₐₓ^{tketo1} for numerical value see Table 10 K_{eq}^{tketo1} = 0.845 [173]
Kₘ^{s7p}^{_{cyt}} = 0.285 [171] Kₘ^{grap}^{_{cyt}} = 0.38 [171] Kₘ^{r5p}^{_{cyt}} = 0.066 [174] Kₘ^{x5p}^{_{cyt}} = 0.15 [175]
Transketolase 2
${v}_{t k e t o 2} = {V}_{m ax}^{t k e t o 1} \cdot ( \frac{f r u 6 {p}_{c y t} \cdot {grap}_{cyt} - 1 / {K}_{e q}^{t k e t o 2} \cdot e 4 {p}_{c y t} \cdot {x5p}_{c y t}}{( 1 + \frac{f r u 6 {p}_{c y t}}{{K}_{m}^{f r u 6 {p}_{c y t}}} ) \cdot ( 1 + \frac{g r a {p}_{c y t}}{{K}_{m}^{{grap}_{c y t}}} ) + ( 1 + \frac{e 4 {p}_{c y t}}{{K}_{m}^{e 4 {p}_{c y t}}} ) \cdot ( 1 + \frac{x 5 {p}_{c y t}}{{K}_{m}^{x 5 {p}_{c y t}}} ) \cdot - 1} )$
Vₘₐₓ^{tketo2} for numerical value see Table 10 K_{eq}^{tketo2} = 0.084 [173]
Kₘ^{fru6p}^{_{cyt}} = 0.34 [176] Kₘ^{grap}^{_{cyt}} = 0.38 [171] Kₘ^{e4p}^{_{cyt}} = 0.044 [177] Kₘ^{x5p}^{_{cyt}} = 0.16 [177]
Fatty acid synthesis
Citrate-malate exchanger
${ν}_{c i t - m a l} = \frac{{V}_{ma x}^{c i t - m a l}}{{K}_{m}^{c i {t}_{m i t o}} \cdot {K}_{m}^{m a {l}_{cyt}}} \cdot ( \frac{c i {t}_{m i t o} \cdot {mal}_{cyt} - 1 / {K}_{eq}^{cit - mal} \cdot {cit}_{cyt} \cdot {mal}_{mito}}{( 1 + \frac{c i {t}_{m i t o}}{{K}_{m}^{c i {t}_{m i t o}}} ) \cdot ( 1 + \frac{m a {l}_{c y t}}{{K}_{m}^{{mal}_{cyt}}} ) + ( 1 + \frac{c i {t}_{c y t}}{{K}_{m}^{c i {t}_{c y t}}} ) \cdot ( 1 + \frac{m a {l}_{m i t o}}{{K}_{m}^{{mal}_{mito}}} ) \cdot - 1} )$
${V}_{ma x}^{cit - mal} = {V}_{0}^{c i t - mal} \cdot ( 1 - \frac{{( c 16 c o {a}_{c y t} )}^{n}}{{( c 16 c o {a}_{c y t} )}^{n} + {( {K}_{i}^{c 16 c o {a}_{c y t}} )}^{n}} )$
V₀^{cit-mal} for numerical value see Table 10 Kᵢ^{c16coa}^{_{cyt}} = 0.033 [178]
n = 3 [178] K_{eq}^{cit-mal} = 1
${K}_{m}^{c i {t}_{m i t o}} = {K}_{0}^{c i {t}_{mito}} \cdot ( 1 + \frac{s u {c}_{m i t o}}{{K}_{i}^{s u {c}_{mito}}} ) \cdot ( 1 + \frac{i s o c i {t}_{m i t o}}{{K}_{i}^{i s o c i {t}_{mito}}} ) \cdot ( 1 + \frac{p e {p}_{m i t o}}{{K}_{i}^{p e {p}_{mito}}} )$
K₀^{cit}^{ₘᵢₜₒ} = 0.14 [179] Kᵢ^{suc}^{ₘᵢₜₒ} = 2.5 [179] Kᵢ^{isocit}^{ₘᵢₜₒ} = 0.08 [179] Kᵢ^{pep}^{ₘᵢₜₒ} = 0.18 [179]
${K}_{m}^{m a {l}_{c y t}} = 0.76 [ 179 ] {K}_{m}^{c i {t}_{c y t}} = {K}_{0}^{c i {t}_{c y t}} \cdot ( 1 + \frac{p e {p}_{c y t}}{{K}_{i}^{p e {p}_{c y t}}} )$
K₀^{cit}^{_{cyt}} = 0.039 [180] Kᵢ^{pep}^{_{cyt}} = 0.18 [179] Kₘ^{mal}^{ₘᵢₜₒ} = 0.76 [179]
ATP dependent citrate lyase
v_{cit-lys} = Vₘₐₓ^{cit-lys} · ((1 − γ^{cit-lys}) · v^{cit-lysnative} + γ^{cit-lys} · v_{cit-lys}^{phospho})
γ^{cit-lys} = γ
Vₘₐₓ^{cit-lys} for numerical value see Table 10
${v}_{c i t - l y s}^{n a t i v e} = \frac{c i {t}_{c y t}^{n}}{c i {t}_{c y t}^{n} + {( {K}_{m}^{c i {t}_{c y t}} )}^{n}} \cdot \frac{c o {a}_{c y t}}{c o {a}_{c y t} + {K}_{m}^{c o {a}_{c y t}}} \cdot \frac{a t {p}_{c y t}}{a t {p}_{c y t} + {K}_{m}^{{atp}_{cyt}}}$
Kₘ^{cit}^{_{cyt}} = 0.154 [181] n = 0.65 [181] Kₘ^{coa}^{_{cyt}} = 0.0026 [181] Kₘ^{atp}^{_{cyt}} = 0.041 [181]
${v}_{c i t - l y s}^{phospho} = \frac{c i {t}_{c y t}^{n}}{c i {t}_{c y t}^{n} + {( {K}_{m}^{c i {t}_{c y t}} )}^{n}} \cdot \frac{c o {a}_{c y t}}{c o {a}_{c y t} + {K}_{m}^{c o {a}_{c y t}}} \cdot \frac{a t {p}_{c y t}}{a t {p}_{c y t} + {K}_{m}^{a t {p}_{cyt}}}$
Kₘ^{cit}^{_{cyt}} = 0.103 [181] n = 0.91 [181] Kₘ^{coa}^{_{cyt}} = 0.002 [181] Kₘ^{atp}^{_{cyt}} = 0.041 [181]
Acetyl-CoA carboxylase 1
v_{acc1} = γ · v_{acc1-p} + (1 − γ) · v_{acc1-up}
${ν}_{a c c 1 - p} = {V}_{m ax}^{a c c 1 - p} \cdot ( \frac{a t {p}_{c y t}}{a t {p}_{c y t} + {K}_{m}^{{atp}_{cyt}}} ) \cdot ( \frac{a c o {a}_{c y t}}{a c o {a}_{c y t} + {K}_{m}^{a c o {a}_{c y t}}} ) \cdot ( \frac{h c o {3}_{c y t}}{h c o {3}_{c y t} + {K}_{m}^{h c o {3}_{c y t}}} )$
${V}_{m ax}^{a c c 1 - p} = {V}_{a c c 1 - p} \cdot ( \frac{c i {t}_{c y t}}{c i {t}_{c y t} + {K}_{a}^{c i {t}_{c y t}}} ) \cdot ( 1 - \frac{{malcoa}_{c y t}}{m a l c o {a}_{c y t} + {K}_{i}^{{malcoa}_{c y t}}} ) \cdot ( 1 - \frac{c 16 c o {a}_{c y t}}{c 16 c o {a}_{c y t} + {K}_{i}^{c 16 c o {a}_{c y t}}} )$
V_{acc1-p} for numerical value see Table 10 Kᵢ^{c16coa}^{_{cyt}} = 0.0022 [182] Kₐ^{cit}^{_{cyt}} = 2.3 [183]
Kᵢ^{malcoa}^{_{cyt}} = 0.0106 [182] Kₘ^{atp}^{_{cyt}} = 0.057 [182] Kₘ^{acoa}^{_{cyt}} 0.18 [183 Kₘ^{hco3}^{_{cyt}} = 2.25 [1.82]
${v}_{a c c 1 - u p} = {V}_{ma x}^{acc 1 - up} \cdot ( \frac{a t {p}_{c y t}}{a t {p}_{c y t} + {K}_{m}^{a t {p}_{c y t}}} ) \cdot ( \frac{a c o {a}_{c y t}}{a c o {a}_{c y t} + {K}_{m}^{{acoa}_{c y t}}} ) \cdot ( \frac{h c o {3}_{c y t}}{h c o {3}_{c y t} + {K}_{m}^{h c o {3}_{c y t}}} )$
${V}_{m ax}^{a cc1 - up} = {V}_{a c c 1 - u p} \cdot ( 1 + {n}_{u p} \frac{c i {t}_{c y t}}{c i {t}_{c y t} + {K}_{a}^{c i {t}_{c y t}}} ) \cdot ( 1 - \frac{m a l c o {a}_{c y t}}{{malcoa}_{c y t} + {K}_{i}^{m a l c o {a}_{c y t}}} )$
V_{acc1-up} = 2.5 · V_{acc1-p} [184] nᵤₚ = 1.4 [184] Kₐ^{cit}^{_{cyt}} = 0.8 [184] Kₘ^{atp}^{_{cyt}} = 0.057 [182]
Kₘ^{acoa}^{_{cyt}} = 0.18 [183] Kᵢ^{malcoa}^{_{cyt}} = 0.0106 [182] Kₘ^{hco3}_{_{cyt}} = 2.25 [182]
Malonyl-CoA decarboxylase
${v}_{m c d c} = {V}_{ma x}^{mcdc} \cdot ( \frac{m a l c o {a}_{c y t}}{m a l c o {a}_{c y t} + {K}_{m}^{m a l c o {a}_{cyt}}} ) {K}_{m}^{{malcoa}_{c y t}} = 0.04 [ 185 ]$
Acetyl-CoA hydrolase
${v}_{a c o a h} = {V}_{ma x}^{a c o a h} \cdot ( \frac{a c o {a}_{c y t}}{a c o {a}_{c y t} + {K}_{m}^{a c o {a}_{c y t}}} ) {K}_{m}^{a c o {a}_{c y t}} = 0.153 [ 186 ]$
TAG synthesis
Glycerol-uptake
${v}_{glycT} = {V}_{m a x}^{glycT} \cdot ( \frac{{glyc}_{ext} - {glyc}_{cyt}}{1 + \frac{{glyc}_{ext}}{{K}_{m}^{{glyc}_{ext}}} + \frac{{glyc}_{cyt}}{{K}_{m}^{{glyc}_{cyt}}}} )$
Vₘₐₓ^{glycT} for numerical value see Table 10 Kₘ^{glyc}^{ₑₓₜ} = 0.012 [187] Kₘ^{glyc}^{_{cyt}} = 0.012 [187]
Glycerol kinase
${v}_{glycK} = {V}_{m a x}^{glycK} \cdot ( \frac{{glyc}_{cyt}}{{glyc}_{cyt} + {K}_{m}^{{glyc}_{cyt}} \cdot ( 1 + \frac{g 3 {p}_{cyt}}{{K}_{i}^{g 3 {p}_{cyt}}} )} ) \cdot ( \frac{{atp}_{cyt}}{{atp}_{cyt} + {K}_{m}^{{atp}_{cyt}}} )$
Vₘₐₓ^{glycK} or numerical value see Table 10
Kₘ^{glyc}^{_{cyt}} = 0.003 [188] Kᵢ^{g3p}^{_{cyt}} = 0.58 [188] Kₘ^{ATP}^{_{cyt}} = 0.058 [188]
Glycerophosphate acyltransferase
${v}_{gpat} = {V}_{m a x}^{gpat} \cdot ( \frac{g 3 {p}_{cyt}}{g 3 {p}_{cyt} + {K}_{m}^{g 3 {p}_{cyt}}} ) \cdot ( \frac{c 16 {coa}_{cyt}}{c 16 {coa}_{cyt} + {K}_{m}^{c 16 {coa}_{cyt}}} )$
Vₘₐₓ^{gpat} for numerical value see Table 10
Kₘ^{g3p}^{_{cyt}} = 0.67 [189] Kₘ^{c16coa}^{_{cyt}} = 0.02 [189]
Acetyl glycerol-3-phosphate acyltransferase
${v}_{agpat} = {V}_{m a x}^{agpat} \cdot ( \frac{{lpa}_{er}}{{lpa}_{er} + {K}_{m}^{{lpa}_{er}}} ) \cdot ( \frac{c 16 {coa}_{cyt}}{c 16 {coa}_{cyt} + {K}_{m}^{c 16 {coa}_{cyt}}} )$
Vₘₐₓ^{agpat} for numerical value see Table 10 Kₘ^{lpa}^{ₑᵣ} = 0.0065 [190] Kₘ^{c16coa}^{_{cyt}} = 0.004 [190]
Phosphatidic acid phosphatase
${v}_{pap} = {V}_{m a x}^{pap} \cdot ( \frac{{pa}_{er}^{n}}{{{pa}_{er}^{n} + {K}_{m}^{{pa}_{er}} )}^{n}} )$
Vₘₐₓ^{pap} for numerical value see Table 10 Kₘ^{pa}^{ₑᵣ} = 0.35 [191] n = 2.2 [191]
Diacylglycerol acyltransferase
${v}_{dgat} = {V}_{m a x}^{dgat} \cdot ( \frac{d a {g}_{er}}{d a {g}_{er} + {K}_{m}^{d a {g}_{er}}} ) \cdot ( \frac{c 16 {coa}_{cyt}}{c 16 {coa}_{cyt} + {K}_{m}^{c 16 {coa}_{cyt}}} )$
Vₘₐₓ^{dgat} for numerical value see Table 10 Kₘ^{dag}^{_{cyt}} = 0.03 [192] Kₘ^{c16coa}^{_{cyt}} = 0.1 [193]
ATGL [194]
${v}_{ATGL}^{tag} = {V}_{m a x - tag}^{ATGL} \cdot {Sur}_{ld} \cdot γ \cdot ( \frac{{tag}_{ld}}{{tag}_{ld} + {K}_{m}^{{tag}_{ld}}} )$
V_{max-tag}^{ATGL} for numerical value see Table 10
${K}_{m}^{{tag}_{ld}} = 10 {Sur}_{ld} = {( {tag}_{ld} + \frac{2}{3} \cdot {dag}_{ld} + \frac{1}{3} \cdot {mag}_{ld} + \frac{2}{3} {ce}_{ld} )}^{\frac{2}{3}}$
Hormone sensitive lipase (HSL) (dag) [195]
${v}_{HSL}^{tag} = {V}_{m a x - tag}^{HSL} \cdot {Sur}_{ld} \cdot γ \cdot ( \frac{d a {g}_{ld}}{d a {g}_{ld} + {K}_{m}^{d a {g}_{ld}}} )$
V_{max-tag}^{HSL} for numerical value see Table 10
${K}_{m}^{d a {g}_{ld}} = 10 {Sur}_{ld} = {( {tag}_{ld} + \frac{2}{3} \cdot d a {g}_{ld} + \frac{1}{3} \cdot {mag}_{ld} + \frac{2}{3} {ce}_{ld} )}^{\frac{2}{3}}$
Monoaclyglycerol lipase
${v}_{magl} = {V}_{m a x}^{magl} \cdot {Sur}_{ld} \cdot ( \frac{{mag}_{ld}}{{mag}_{ld} + {K}_{m}^{{mag}_{ld}}} )$
Vₘₐₓ^{magl} for numerical value see Table 10
${K}_{m}^{{mag}_{ld}} = 0.51 [ 196 ] {Sur}_{ld} = {( {tag}_{ld} + \frac{2}{3} \cdot d a {g}_{ld} + \frac{1}{3} \cdot {mag}_{ld} + \frac{2}{3} {ce}_{ld} )}^{\frac{2}{3}}$
Cholesterol ester esterase
${v}_{cee} = {V}_{m a x}^{cee} \cdot {Sur}_{ld} \cdot γ \cdot ( \frac{{ce}_{ld}}{{ce}_{ld} + {K}_{m}^{{ce}_{ld}}} )$
Vₘₐₓ^{cee} for numerical value see Table 10
${K}_{m}^{{ce}_{ld}} = 5 {Sur}_{ld} = {( {tag}_{ld} + \frac{2}{3} \cdot d a {g}_{ld} + \frac{1}{3} \cdot {mag}_{ld} + \frac{2}{3} {ce}_{ld} )}^{\frac{2}{3}}$
Ketone body utilization
B-Hydroxy butyrate dehydrogenase
${v}_{β hdh} = {V}_{m a x}^{β hdh} \cdot ( \frac{{acac}_{mito} \cdot {nadh}_{mito} - \frac{1}{{K}_{eq}^{β hdh}} \cdot {bhbut}_{mito} \cdot {nad}_{mito}}{( 1 + \text{} \frac{{acac}_{mito}}{{{K}_{m}}_{mito}^{acac}} ) \cdot \text{} ( 1 + \text{} \frac{{nadh}_{mito}}{{K}_{m}^{{nadh}_{mito}}} ) + \text{} ( 1 + \text{} \frac{{bhbut}_{mito}}{{K}_{m}^{{bhbut}_{mito}}} ) \cdot \text{} ( 1 + \text{} \frac{{nad}_{mito}}{{K}_{m}^{{nad}_{mito}}} ) - \text{} 1} )$
Vₘₐₓ^{βhdh} for numerical value see Table 10
${K}_{eq}^{β hdh} = \text{} 20.3 \cdot \frac{{h}_{mito}}{{h}_{cyt}} [ 125 ] {K}_{m}^{{acac}_{mito}} = \text{} 0.204 [ 197 ] {K}_{m}^{{nadh}_{mito}} = {K}_{0}^{{nadh}_{mito}} \cdot ( 1 + \frac{{nad}_{mito}}{{K}_{i}^{{nad}_{mito}}} )$
K₀^{nadh}^{ₘᵢₜₒ} = 0.017 [197] Kⁱ_{nad}^{ₘᵢₜₒ} 0.121 [197] Kₘ^{hbut}^{ₘᵢₜₒ} = 1.604 [197]
${K}_{m}^{{nad}_{mito}} = \text{} {K}_{0}^{{nad}_{mito}} \cdot ( 1 + \frac{{nadh}_{mito}}{{K}_{i}^{{nadh}_{mito}}} ) {K}_{0}^{{nad}_{mito}} = \text{} 0.067 [ 197 ] {K}_{i}^{{nadh}_{mito}} = 0.066 [ 197 ]$
Succinyl-CoA-oxaloacid CoA transferase
${v}_{scot} = {V}_{m a x}^{scot} \cdot ( \frac{{acac}_{mito} \cdot {succoa}_{mito} - \frac{1}{{K}_{eq}^{scot}} \cdot kc 4 {coa}_{mito} \cdot {succ}_{mito}}{( 1 + \frac{{acac}_{mito}}{{{K}_{m}}_{mito}^{acac}} ) \cdot ( 1 + \frac{{succoa}_{mito}}{{K}_{m}^{{succoa}_{mito}}} ) + \\ ( 1 + \frac{kc 4 {coa}_{mito}}{{K}_{m}^{kc 4 {coa}_{mito}}} ) \cdot ( 1 + \frac{{succ}_{mito}}{{K}_{m}^{{succ}_{mito}}} ) - 1} )$
Vₘₐₓ^{scot} for numerical value see Table 10
${{K}_{m}}_{mito}^{acac} = 0.44 [ 198 ] {K}_{m}^{{succoa}_{mito}} = {K}_{0}^{{succoa}_{mito}} \cdot ( 1 + \frac{{succ}_{mito}}{{K}_{i}^{{succ}_{mito}}} )$
${K}_{0}^{{succoa}_{mito}} = \text{} 0.28 [ 198 ] {K}_{i}^{{succ}_{mito}} = \text{} 0.72 [ 198 ] {K}_{m}^{kc 4 {coa}_{mito}} = {K}_{0}^{kc 4 {coa}_{mito}} \cdot ( 1 + \frac{{acac}_{mito}}{{K}_{i}^{{acac}_{mito}}} )$
K₀^{kc4coa}^{ₘᵢₜₒ} = 0.44 [198] Kᵢ^{acac}^{ₘᵢₜₒ} = 3.7 [198] Kₘ^{succ}^{ₘᵢₜₒ} = 34 [198]
Acetoacetate transport (mitochondrial)
${v}_{acacT} = {V}_{m a x}^{acacT} \cdot ( \frac{{acac}_{mito}}{{acac}_{mito} + {K}_{m}^{{acac}_{mito}}} )$
Vₘₐₓ^{acacT} for numerical value see Table 10 Kₘ^{acac}^{ₘᵢₜₒ} = 0.56 [199]
B-Hydroxy butyrate transport (mitochondrial)
${v}_{β hbT} = {V}_{m a x}^{β hbT} \cdot ( \frac{{bhbut}_{mito}}{{bhbut}_{mito} + {K}_{m}^{{bhbut}_{mito}}} )$
Vₘₐₓ^{βhbt} for numerical value see Table 10 Kₘ^{bhbut}^{ₘᵢₜₒ} = 0.8 [200]
Acetoacetate export (MCT1/MCT2)
${v}_{acac - ex} = {V}_{m a x}^{acac - ex} \cdot ( \frac{{acac}_{ext} - {acac}_{cyt}}{1 + \frac{{acac}_{cyt}}{{K}_{m}^{{acac}_{cyt}}} + \frac{{acac}_{ext}}{{K}_{m}^{{acac}_{ext}}}} )$
Vₘₐₓ^{acac-ex} for numerical value see Table 10 Kₘ^{acac}^{_{cyt}} = 1.2 [200] Kₘ^{acac}^{ₑₓₜ} = 1.2 [200]
B-Hydroxy butyrate export (MCT1/MCT2)
${v}_{β hb - ex} = {V}_{m a x}^{β hb - ex} \cdot ( \frac{{bhbut}_{ext} - {bhbut}_{cyt}}{1 + \frac{{bhbut}_{cyt}}{{K}_{m}^{{bhbut}_{cyt}}} + \frac{{bhbut}_{ext}}{{K}_{m}^{{bhbut}_{ext}}}} )$
Vₘₐₓ^{βhb-ex} for numerical value see Table 10 Kₘ^{bhbut}^{_{cyt}} = 0.8 [200] Kₘ^{bhbut}^{ₑₓₜ} 0.8 [200]
Branched chain amino acid metabolism
Valine transport
${v}_{valT} = {V}_{m a x}^{valT} \cdot ( \frac{{val}_{ext} - {val}_{cyt}}{1 + \frac{{val}_{ext}}{{K}_{m}^{{val}_{ext}}} + \frac{{val}_{cyt}}{{K}_{m}^{{val}_{cyt}}}} )$
Vₘₐₓ^{valT} for numerical value see Table 10 Kₘ^{val}^{ₑₓₜ} = 0.124 [201] Kₘ^{val}^{_{cyt}} = 0.124 [201]
Leucine transport
${v}_{leuT} = {V}_{m a x}^{leuT} \cdot ( \frac{{leu}_{ext} - {leu}_{cyt}}{1 + \frac{{leu}_{ext}}{{K}_{m}^{{leu}_{ext}}} + \frac{{leu}_{cyt}}{{K}_{m}^{{leu}_{cyt}}}} )$
Vₘₐₓ^{leuT} for numerical value see Table 10 Kₘ^{leu}^{ₑₓₜ} = 0.119 [201] Kₘ^{leu}^{_{cyt}} = 0.119 [201]
Isoleucine transport
${v}_{isoleuT} = {V}_{m a x}^{isoleuT} \cdot ( \frac{{isoleu}_{ext} - {isoleu}_{cyt}}{1 + \frac{{isoleu}_{ext}}{{K}_{m}^{{isoleu}_{ext}}} + \frac{{isoleu}_{cyt}}{{K}_{m}^{{isoleu}_{cyt}}}} )$
Vₘₐₓ^{isoleuT} for numerical value see Table 10 Kₘ^{isoleu}^{ₑₓₜ} = 0.0967 [201] Kₘ^{isoleu}^{_{cyt}} = 0.0967 [201]
Branched chain amino acid aminotransferase valine
${v}_{BCAAT - val} = {V}_{m a x}^{BCAAT - val} \cdot ( \frac{{val}_{cyt} \cdot {akg}_{cyt} - \frac{1}{{K}_{eq}^{BCAAT - val}} \cdot {aKIVA}_{cyt} \cdot {glu}_{cyt}}{( 1 + \frac{{val}_{cyt}}{{K}_{m}^{{val}_{cyt}}} ) \cdot ( 1 + \frac{{akg}_{cyt}}{{K}_{m}^{{akg}_{cyt}}} ) + ( 1 + \frac{{aKIVA}_{cyt}}{{K}_{m}^{{aKIVA}_{cyt}}} ) \cdot ( 1 + \frac{{glu}_{cyt}}{{K}_{m}^{{glu}_{cyt}}} ) - 1} )$
Vₘₐₓ^{BCAAT-val} for numerical value see Table 10 K_{eq}^{BCAAT-val} = 1 Kₘ^{val}^{_{cyt}} = 0.62 [202]
${K}_{m}^{{akg}_{cyt}} = 0.63 [ 203 ] {K}_{m}^{{aKIVA}_{cyt}} = {K}_{0}^{{aKIVA}_{cyt}} \cdot ( 1 + \frac{{aKICA}_{cyt}}{{K}_{i}^{{aKICA}_{cyt}}} ) \cdot ( 1 + \frac{{KMeVA}_{cyt}}{{K}_{i}^{{KMeVA}_{cyt}}} )$
K₀^{aKIVA}^{_{cyt}} = 0.11 [204] Kᵢ^{aKICA}^{_{cyt}} = 2.1 [204] Kᵢ^{KMeVA}^{_{cyt}} = 1.57 [204] Kₘ^{glu}^{_{cyt}} = 3.6 [203]
Branched chain amino acid aminotransferase leucine
${v}_{BCAAT - leu} = {V}_{m a x}^{BCAAT - leu} \cdot ( \frac{{leu}_{cyt} \cdot {akg}_{cyt} - \frac{1}{{K}_{eq}^{BCAAT - leu}} \cdot {aKICA}_{cyt} \cdot {glu}_{cyt}}{( 1 + \frac{{leu}_{cyt}}{{K}_{m}^{{leu}_{cyt}}} ) \cdot ( 1 + \frac{{akg}_{cyt}}{{K}_{m}^{{akg}_{cyt}}} ) + ( 1 + \frac{{aKICA}_{cyt}}{{K}_{m}^{{aKICA}_{cyt}}} ) \cdot ( 1 + \frac{{glu}_{cyt}}{{K}_{m}^{{glu}_{cyt}}} ) - 1} )$
Vₘₐₓ^{BCAAT-leu} for numerical value see Table 10
K_{eq}^{BCAAT-leu} = 1.75 [205] Kₘ^{leu}^{_{cyt}} = 3.8 [203] Kₘ^{akg}^{_{cyt}} = 0.63 [203]
${K}_{m}^{{aKIVA}_{cyt}} = {K}_{0}^{{aKIVA}_{cyt}} \cdot ( 1 + \frac{{aKICA}_{cyt}}{{K}_{i}^{{aKICA}_{cyt}}} ) \cdot ( 1 + \frac{{KMeVA}_{cyt}}{{K}_{i}^{{KMeVA}_{cyt}}} )$
K₀^{aKICA}^{_{cyt}} = 0.14 [204] Kᵢ^{aKICA}^{_{cyt}} = 4.19 [204] Kᵢ^{KMeVA}^{_{cyt}} = 1.57 [204] Kₘ^{glu}^{_{cyt}} = 6.65 [203]
Branched chain amino acid aminotransferase isoleucine
${v}_{BCAAT - isoleu} = {V}_{m a x}^{BCAAT - isoleu} \cdot ( \frac{{isoleu}_{cyt} \cdot {akg}_{cyt} - \frac{1}{{K}_{eq}^{BCAAT - leu}} \cdot {KMeVA}_{cyt} \cdot {glu}_{cyt}}{( 1 + \frac{{isoleu}_{cyt}}{{K}_{m}^{{isoleu}_{cyt}}} ) \cdot ( 1 + \frac{{akg}_{cyt}}{{K}_{m}^{{akg}_{cyt}}} ) + ( 1 + \frac{{KMeVA}_{cyt}}{{K}_{m}^{{KMeVA}_{cyt}}} ) \cdot ( 1 + \frac{{glu}_{cyt}}{{K}_{m}^{{glu}_{cyt}}} ) - 1} )$
Vₘₐₓ^{BCAAT-isoleu} for numerical value see Table 10 K_{eq}^{BCAAT-isoleu} = Kₘ^{isoleu}^{_{cyt}} = 3.8 [203]
${K}_{m}^{{akg}_{cyt}} = \text{} 0.63 [ 203 ] {K}_{m}^{{KMeVA}_{cyt}} = \text{} {K}_{0}^{{KMeVA}_{cyt}} \cdot ( 1 + \frac{{aKICA}_{cyt}}{{K}_{i}^{{aKICA}_{cyt}}} ) \cdot ( 1 + \frac{{aKIVA}_{cyt}}{{K}_{i}^{{aKICV}_{cyt}}} )$
K₀^{KMeVA}^{_{cyt}} = 0.07 [204] Kᵢ^{aKIVA}^{_{cyt}} 4.19 [204] Kᵢ^{aKICA}^{_{cyt}} 2.1 [204] Kₘ^{glu}^{_{cyt}} = 2.45 [203]
A-ketoisovalerate transport (mitochondrial)
${v}_{{aKIVAT}_{mito}} = {V}_{m a x}^{{aKIVAT}_{mito}} \cdot ( \frac{{aKIVA}_{cyt} - {aKIVA}_{mito}}{{aKIVA}_{cyt} + {K}_{m}^{{aKIVA}_{cyt}}} )$
Vₘₐₓ^{aKIVAT}^{ₘᵢₜₒ} for numerical value see Table 10 Kₘ^{aKIVA}^{_{cyt}} = 0.025 [206]
A-ketoisocaproate transport (mitochondrial)
${v}_{{aKICAT}_{mito}} = {V}_{m a x}^{{aKICAT}_{mito}} \cdot ( \frac{{aKICA}_{cyt} - {aKICA}_{mito}}{{aKICA}_{cyt} + {K}_{m}^{{aKICA}_{cyt}}} )$
Vₘₐₓ^{aKICAT} mito for numerical value see Table 10 Kₘ^{aKICA}^{_{cyt}} = 0.01 [206]
A-ketomethylvalerate transport (mitochondrial)
${v}_{{KMeVAT}_{mito}} = {V}_{m a x}^{{KMeVAT}_{mito}} \cdot ( \frac{{KMeVA}_{cyt} - {KMeVA}_{mito}}{{KMeVA}_{cyt} + {K}_{m}^{{KMeVA}_{cyt}}} )$
Vₘₐₓ^{KMeVAT}^{ₘᵢₜₒ} for numerical value see Table 10 Kₘ^{KMeVA}^{_{cyt}} = 0.01
Branched chain keto-amino acid dehydrogenase (aKIVA)
${v}_{bckadh - aKIVA} = {V}_{m a x}^{bckadh - aKIVA} \cdot ( \frac{{aKIVA}_{mito}}{{aKIVA}_{mito} + {K}_{m}^{{aKIVA}_{mito}}} ) \cdot ( \frac{{nad}_{mito}}{{nad}_{mito} + {K}_{m}^{{nad}_{mito}}} ) \cdot ( \frac{{coa}_{mito}}{{coa}_{mito} + {K}_{m}^{{coa}_{mito}}} )$
Vₘₐₓ^{bckadh-aKIVA} for numerical value see Table 10
Kₘ^{aKIVA}^{ₘᵢₜₒ} = 0.05 [207] Kₘ^{nad}^{ₘᵢₜₒ} = 0.04 [208] Kₘ^{coa}^{ₘᵢₜₒ} = 0.01 [208]
Branched chain keto-amino acid dehydrogenase (aKIVA)
${v}_{bckadh - aKICA} = {V}_{m a x}^{bckadh - aKICA} \cdot ( \frac{{aKICA}_{mito}}{{aKICA}_{mito} + {K}_{m}^{{aKICA}_{mito}}} ) \cdot ( \frac{{nad}_{mito}}{{nad}_{mito} + {K}_{m}^{{nad}_{mito}}} ) \cdot ( \frac{{coa}_{mito}}{{coa}_{mito} + {K}_{m}^{{coa}_{mito}}} )$
Vₘₐₓ^{bckadh-aKICA} for numerical value see Table 10
Kₘ^{aKICA}^{ₘᵢₜₒ} = 0.038 [209] Kₘ^{nad}^{ₘᵢₜₒ} = 0.04 [208] Kₘ^{coa}^{ₘᵢₜₒ} = 0.01 [208]
Branched chain keto-amino acid dehydrogenase (aKIVA)
${v}_{bckadh - KMeVA} = {V}_{m a x}^{bckadh - KMeVA} \cdot ( \frac{{KMeVA}_{mito}}{{aKICA}_{mito} + {K}_{m}^{{aKICA}_{mito}}} ) \cdot ( \frac{{nad}_{mito}}{{nad}_{mito} + {K}_{m}^{{nad}_{mito}}} ) \cdot ( \frac{{coa}_{mito}}{{coa}_{mito} + {K}_{m}^{{coa}_{mito}}} )$
Vₘₐₓ^{bckadh-KMeVA} for numerical value see Table 10
Kₘ^{KMeVA}mito = 0.035 [210] Kₘ^{nad}^{ₘᵢₜₒ} = 0.04 [208] Kₘ^{coa}^{ₘᵢₜₒ} = 0.01 [208]
2-methylacyl-CoA dehydrogenase (isobutyryl-CoA)
${v}_{MBCoADH - IsoButCoA} = {V}_{m a x}^{MBCoADH} \cdot ( \frac{{IsoButCoA}_{mito}}{{IsoButCoA}_{mito} + {K}_{m}^{{IsoButCoA}_{mito}}} ) \cdot ( \frac{{etffad}_{mito}}{{etffad}_{mito} + {K}_{m}^{{etffad}_{mito}}} )$
Vₘₐₓ^{MBCoADH} for numerical value see Table 10
Kₘ^{IsoButCoA}^{ₘᵢₜₒ} = 0.013 [211] K^{etffad}^{ₘᵢₜₒ} = 0.0083 [19]
2-methylacyl-CoA dehydrogenase (methylbutyryl-CoA)
${v}_{MBCoADH - MeButCoA} = {V}_{m a x}^{MBCoADH} \cdot ( \frac{{MeButCoA}_{mito}}{{MeButCoA}_{mito} + {K}_{m}^{{MeButCoA}_{mito}}} ) \cdot ( \frac{{etffad}_{mito}}{{etffad}_{mito} + {K}_{m}^{{etffad}_{mito}}} )$
Vₘₐₓ^{MBCoADH} for numerical value see Table 10
Kₘ^{MeButCoA}^{ₘᵢₜₒ} = 0.027 [211] Kₘ^{etffad}^{ₘᵢₜₒ} = 0.0083 [19]
Enoyl-CoA hydratase (methyl acrylyl CoA)
${v}_{ECoAH - MeAcrCoA} = {V}_{m a x}^{ECoAH - MeACrCoA} \cdot ( \frac{{MeAcrCoA}_{mito}}{{MeAcrCoA}_{mito} + {K}_{m}^{{MeAcrCoA}_{mito}}} )$
Vₘₐₓ^{ECoAH-MeACrCoA} for numerical value see Table 10 Kₘ^{MeAcrCoA}^{ₘᵢₜₒ} = 0.001 [211]
Enoyl-CoA hydratase (Tiglyl CoA)
${v}_{ECoAH - TigCoA} = {V}_{m a x}^{ECoAH - TigCoA} \cdot ( \frac{{TigCoA}_{mito}}{{TigCoA}_{mito} + {K}_{m}^{{TigCoA}_{mito}}} )$
Vₘₐₓ^{ECoAH-TigCoA} for numerical value see Table 10 Kₘ^{TigCoA}^{ₘᵢₜₒ} = 0.0047 [211]
3-hydroxyisobutyryl-CoA hydrolase
${v}_{HibCoAhyd} = {V}_{m a x}^{HibCoAhyd} \cdot ( \frac{{HibCoA}_{mito}}{{HibCoA}_{mito} + {K}_{m}^{{HibCoA}_{mito}}} )$
Vₘₐₓ^{HibCoAhyd} for numerical value see Table 10 Kₘ^{HibCoA}^{ₘᵢₜₒ} = 0.006 [212]
3-hydroxyisobutyrate dehydrogenase
${v}_{HibDh} = {V}_{m a x}^{HibDh} \cdot ( \frac{{Hib}_{mito}}{{Hib}_{mito} + {K}_{m}^{{Hib}_{mito}}} ) \cdot ( \frac{{nad}_{mito}}{{nad}_{mito} + {K}_{m}^{{nad}_{mito}}} )$
Vₘₐₓ^{HibDh} for numerical value see Table 10
${K}_{m}^{{Hib}_{mito}} = 0.061 [ 213 ] {K}_{m}^{{nad}_{mito}} = {K}_{0}^{{nad}_{mito}} \cdot ( 1 + \frac{{nadh}_{mito}}{{K}_{i}^{{nadh}_{mito}}} )$
K₀^{nadh}^{ₘᵢₜₒ} = 0.023 [213] Kᵢ^{nadh}^{ₘᵢₜₒ} = 0.0057 [213]
Methylmalonate-semialdehyde dehydrogenase
${v}_{mmsdh} = {V}_{m a x}^{mmsd} \cdot ( \frac{{mmsald}_{mito}}{{mmsald}_{mito} + {K}_{m}^{{mmsald}_{mito}}} ) \cdot ( \frac{{nad}_{mito}}{{nad}_{mito} + {K}_{m}^{{nad}_{mito}}} ) \cdot ( \frac{{coa}_{mito}}{{coa}_{mito} + {K}_{m}^{{coa}_{mito}}} )$
Vₘₐₓ^{mmsd} for numerical value see Table 10
Kₘ^{mmsald}^{ₘᵢₜₒ} = 0.0053 [214] Kₘ^{nad}^{ₘᵢₜₒ} = 0.15 [214] Kₘ^{coa}^{ₘᵢₜₒ} = 0.03 [214]
3-hydroxy-2-methylbutyryl-CoA dehydrogenase
${v}_{HMBCDH} = {V}_{m a x}^{HMBCDH} \cdot ( \frac{{MeHButCoA}_{mito}}{{MeHButCoA}_{mito} + {K}_{m}^{{MeHButCoA}_{mito}}} ) \cdot ( \frac{{nad}_{mito}}{{nad}_{mito} + {K}_{m}^{{nad}_{mito}}} )$
Vₘₐₓ^{HMBCDH} for numerical value see Table 10
Kₘ^{MeHButCoA}^{ₘᵢₜₒ} = 0.005 [215] Kₘ^{nad}^{ₘᵢₜₒ} = 0.01 [215]
acetyl-CoA C-acyltransferase
${v}_{MAACT} = {V}_{m a x}^{MAACT} \cdot ( \frac{{MeAACoA}_{mito}}{{MeAACoA}_{mito} + {K}_{m}^{{MeAACoA}_{mito}}} ) \cdot ( \frac{{coa}_{mito}}{{coa}_{mito} + {K}_{m}^{{coa}_{mito}}} )$
Vₘₐₓ^{MAACT} for numerical value see Table 10
Kₘ^{MeAACoA}^{ₘᵢₜₒ} = 0.008 [216] Kₘ^{coa}^{ₘᵢₜₒ} = 0.02 [216]
isovaleryl-CoA dehydrogenase
${v}_{IVCoADh} = {V}_{m a x}^{IVCoADh} \cdot ( \frac{{IsoValCoA}_{mito}}{{IsoValCoA}_{mito} + {K}_{m}^{{IsoValCoA}_{mito}}} ) \cdot ( \frac{{etffad}_{mito}}{{etffad}_{mito} + {K}_{m}^{{etffad}_{mito}}} )$
Vₘₐₓ^{IVCoADh} for numerical value see Table 10
Kₘ^{IsoValCoA}^{ₘᵢₜₒ} = 0.014 [217] Kₘ^{etffad}^{ₘᵢₜₒ} = 0.0083 [19]
Methylcrotonoyl-CoA carboxylase
${v}_{MECCC} = {V}_{m a x}^{MECCC} \cdot ( \frac{{MeCroCoA}_{mito}}{{MeCroCoA}_{mito} + {K}_{m}^{{MeCroCoA}_{mito}}} ) \cdot ( \frac{{atp}_{mito}}{{atp}_{mito} + {K}_{m}^{{atp}_{mito}}} )$
Vₘₐₓ^{MECCC} for numerical value see Table 10
Kₘ^{MeCroCoA}^{ₘᵢₜₒ} = 0.0747 [218] Kₘ^{atp}^{ₘᵢₜₒ} = 0.045 [218]
Methylglutaconyl-CoA hydratase
${v}_{MEGCCH} = {V}_{m a x}^{MEGCCH} \cdot ( \frac{{MeGCCoA}_{mito}}{{MeGCCoA}_{mito} + {K}_{m}^{{MeGCCoA}_{mito}}} )$
Vₘₐₓ^{MEGCCH} for numerical value see Table 10 Kₘ^{MeGCCoA}^{ₘᵢₜₒ} = 0.0083 [219]
Hydroxymethylglutaryl-CoA lyase
${v}_{HMGCL} = {V}_{m a x}^{HMGCL} \cdot ( \frac{{HMeGCoA}_{mito}}{{HMeGCoA}_{mito} + {K}_{m}^{{HMeGCoA}_{mito}}} )$
Vₘₐₓ^{HMGCL} for numerical value see Table 10 Kₘ^{HMeGCoA}^{ₘᵢₜₒ} = 0.0448 [220]
Propionyl-CoA carboxylase
${v}_{PCC} = {V}_{m a x}^{PCC} \cdot ( \frac{{ProCoA}_{mito}}{{ProCoA}_{mito} + {K}_{m}^{{ProCoA}_{mito}}} ) \cdot ( \frac{{atp}_{mito}}{{atp}_{mito} + {K}_{m}^{{atp}_{mito}}} )$
Vₘₐₓ^{PCC} for numerical value see Table 10 Kₘ^{ProCoA}^{ₘᵢₜₒ} = 0.2 [221] Kₘ^{atp}^{ₘᵢₜₒ} = 0.08 [222]
Methylmalonyl-CoA mutase
${v}_{MMCM} = {V}_{m a x}^{MMCM} \cdot ( \frac{{MeMalCoA}_{mito}}{{MeMalCoA}_{mito} + {K}_{m}^{{MeMalCoA}_{mito}}} )$
Vₘₐₓ^{MMCM} for numerical value see Table 10 Kₘ^{MeMalCoA}^{ₘᵢₜₒ} = 0.133 [223]
Glutamate transport
${v}_{{gluT}_{mito}} = {V}_{m a x}^{{gluT}_{mito}} \cdot ( \frac{{glu}_{cyt} \cdot {h}_{cyt} - {glu}_{mito} \cdot {h}_{mito}}{1 + \frac{{glu}_{cyt}}{{K}_{m}^{{glu}_{cyt}}} + \frac{{glu}_{mito}}{{K}_{m}^{{glu}_{mito}}}} )$
Vₘₐₓ^{gluT}^{ₘᵢₜₒ} for numerical value see Table 10 Kₘ^{glu}^{_{cyt}} = 5 [224] Kₘ^{glu}^{ₘᵢₜₒ} = 0.25 [225]
Glutamate dehydrogenase (nad-dependent)
${v}_{gdh} = {V}_{m a x}^{gdh} \cdot ( \text{} \frac{{glu}_{mito} \cdot {nad}_{mito} - \frac{1}{{K}_{eq}^{gdh}} \cdot {akg}_{mito} \cdot {nadh}_{mito} \cdot nh {3}_{mito}}{\begin{matrix}( 1 + \frac{{glu}_{mito}}{{K}_{m}^{{glu}_{mito}}} ) \cdot ( 1 + \frac{{nad}_{mito}}{{K}_{m}^{{nad}_{mito}}} ) + \\ ( 1 + \frac{{akg}_{mito}}{{K}_{m}^{{akg}_{mito}}} ) ( 1 + \frac{{nadh}_{mito}}{{K}_{m}^{{nadh}_{mito}}} ) \cdot ( 1 + \frac{nh {3}_{mito}}{{K}_{m}^{nh {3}_{mito}}} )\end{matrix}} \text{} )$
${V}_{m a x}^{gdh} = {V}_{0}^{gdh} \cdot ( 1 - \frac{c 16 {coa}_{mito}}{c 16 {coa}_{mito} + {K}_{i}^{c 16 {coa}_{mito}}} ) \cdot ( {A}_{0} + ( 1 - {A}_{0} ) ( 1 - \frac{{mal}_{mito}}{{mal}_{mito} + {K}_{i}^{{mal}_{mito}}} ) ) \cdot$
V₀^{gdh} for numerical value see Table 10 Kᵢ^{c16coa}^{ₘᵢₜₒ} = 0.0001 [226] A₀ = 0.7 [226]
Kᵢ^{mal}^{ₘᵢₜₒ} = 2 [226] K_{eq}^{gdh} = 0.00387 mM [125]
${K}_{m}^{{glu}_{mito}} = {K}_{0}^{{glu}_{mito}} \cdot ( 1 + \frac{{akg}_{mito}}{{K}_{i}^{{akg}_{mito}}} ) \cdot ( 1 + \frac{nh {3}_{mito}}{{K}_{i}^{nh {3}_{mito}}} )$
K₀^{glu}^{ₘᵢₜₒ} = 4.61 [227] Kᵢ^{akg}^{ₘᵢₜₒ} = 1.49 [228] Kᵢ^{nh3}^{ₘᵢₜₒ} = 3.1 [228]
${K}_{m}^{{nad}_{mito}} = {K}_{0}^{{nad}_{mito}} \cdot ( 1 + \frac{{nadh}_{mito}}{{K}_{i}^{{nadh}_{mito}}} ) {K}_{0}^{{nad}_{mito}} = 0.364 [ 227 ]$
Kᵢ^{nadh}^{ₘᵢₜₒ} = 0.0086 [228] Kₘ^{akg}^{ₘᵢₜₒ} = 0.18 [229] Kₘ^{nadh}^{ₘᵢₜₒ} = 0.03 [229] Kₘ^{nh3}^{ₘᵢₜₒ} = 20 [229]
Stoichiometric matrix:
$\frac{d}{dt} {acac}_{cyt} = + \frac{{Vol}_{mito}}{{Vol}_{cyt}} \cdot {v}_{acacT} + {v}_{acac - ex} \frac{d}{dt} {acac}_{ext} = 0$
$\frac{d}{dt} {acac}_{mito} = - {v}_{scot} - {v}_{β hdh} - {v}_{acacT} + {v}_{HMGCL} \frac{d}{dt} {acetate}_{cyt} = + {v}_{acoah} + {v}_{aceT}$
$\frac{d}{dt} {acoa}_{cyt} = + {v}_{cit - lys} - {v}_{acc 1} + {v}_{mcdc} + {v}_{acoah} + {v}_{HMGCL} + {v}_{MAACT}$
$\frac{d}{dt} {acoa}_{mito} = + 2 \cdot {v}_{3 kt}^{kc 4 coa} + 2 \cdot {v}_{3 ktII}^{kc 4 coa} + {v}_{3 kt}^{kc 6 coa} + \\ {v}_{3 kt}^{kc 8 coa} + {v}_{3 kt}^{kc 10 coa} + {v}_{3 kt}^{kc 12 coa} + {v}_{3 kt}^{kc 14 coa} + {v}_{3 kt}^{kc 16 coa} + {v}_{pdhc} - {v}_{cs}$
$\frac{d}{dt} {adp}_{cyt} = - \frac{{v}_{nex}}{10 \cdot F \cdot {Vol}_{cyt}} + {v}_{{ndk}_{cyt}} + 2 \cdot {v}_{{ak}_{cyt}} + {v}_{atp - usage} + {v}_{hka} + \\ {v}_{hkb} + {v}_{{ndk}_{cyt}}^{udp} + {v}_{pfk 2} - {v}_{pfk1} - {v}_{pgk} - {v}_{p k} + {v}_{cit - lys} + {v}_{acc 1} + {v}_{glycK}$
$\frac{d}{dt} {adp}_{mito} = - {v}_{scs - atp} - \frac{{v}_{F 0 F 1}}{10 \cdot F \cdot {Vol}_{mito}} + \frac{{v}_{nex}}{10 \cdot F \cdot {Vol}_{mito}} + {v}_{{ndk}_{mito}} + {v}_{pc} + {v}_{PCC} + {v}_{MECCC}$
$\frac{d}{dt} {akg}_{cyt} = - {v}_{asat} + {v}_{mac} - {v}_{BCAAT - val} - {v}_{BCAAT - leu} - {v}_{BCAAT - isoleu}$
$\frac{d}{dt} {akg}_{mito} = + {v}_{{idh}_{nad}} + {v}_{{idh}_{nadp}} - {v}_{kgdhc} - {v}_{{asat}_{mito}} - \frac{{Vol}_{cyt}}{{Vol}_{mito}} \cdot {v}_{mac} + {v}_{gdh}$
$\frac{d}{dt} {aKICA}_{cyt} = + {v}_{BCAAT - val} - {v}_{aKICAT}$
$\frac{d}{d t} {aKICA}_{m i t o} = + {v}_{aKICAT} \cdot \frac{V o {l}_{m i t o}}{V o {l}_{c y t}} - {v}_{b c k a d h - aKICA}$
$\frac{d}{dt} {aKIVA}_{cyt} = + {v}_{BCAAT - leu} - {v}_{aKIVAT}$
$\frac{d}{dt} {aKIVA}_{m i t o} = + {v}_{aKIVAT} \cdot \frac{V o {l}_{mito}}{V o {l}_{c y 𝔱}} - {v}_{bckadh - aKIVA}$
$\frac{d}{dt} {amp}_{cyt} = + {v}_{ACS 1} + {v}_{FAB 1} + {v}_{FAB 4} - {v}_{{ak}_{cyt}}$
$\frac{d}{d t} a s {p}_{cyt} = - {v}_{a s a t} + {v}_{agc}$
$\frac{d}{dt} {asp}_{mito} = - {v}_{{asat}_{mito}} - \frac{{Vol}_{cyt}}{{Vol}_{er}} \cdot {v}_{agc}$
$\frac{d}{dt} {atp}_{cyt} = \\ - {v}_{ACS 1} - {v}_{FAB 1} - {v}_{FAB 4} + \frac{{v}_{nex}}{10 \cdot F \cdot {Vol}_{cyt}} - {v}_{{ndk}_{cyt}} - {v}_{atp - usage} - {v}_{hka} - {v}_{hkb} - \\ {v}_{pfk 2} - {v}_{pfk 1} + {v}_{pgk} + {v}_{p k} - {v}_{{ndk}_{cyt}}^{udp} - {v}_{cit - lys} - {v}_{acc 1} - {v}_{glycK} - {v}_{{ak}_{cyt}}$
$\frac{d}{d t} a t {p}_{m i t o} = + {v}_{s c s - a t p} + \frac{{v}_{F 0 F 1}}{10 \cdot F \cdot {Vol}_{m i t o}} - \frac{{ν}_{n e x}}{10 \cdot F \cdot {Vol}_{m i t o}} - {v}_{{ndk}_{mito}} - {v}_{p c} - {v}_{P C C} - {v}_{M E C C C}$
$\frac{d}{d t} b h b u {t}_{c y t} = + \frac{V o {l}_{m i t o}}{{Vol}_{c y t}} \cdot {v}_{β h b T} + {v}_{β h b - e x}$
$\frac{d}{d t} b h b u {t}_{e x t} = 0$
$\frac{d}{d t} b h b u {t}_{m i t o} = + {v}_{β h a h} - {v}_{β h b T}$
$\frac{d}{d t} b p g 1 {3}_{cyt} = + {v}_{gapdh} - {v}_{p g k}$
$\frac{d}{d t} c 10 c o {a}_{mito} = - {v}_{C 10 c o a - mcdh} - {v}_{c 10 coa - lcdh} + {v}_{3 k t}^{k c 12 c o a}$
$\frac{d}{d t} c 12 c o {a}_{m i t o} = - {v}_{c 12 coa - mcdh} - {v}_{c 12 coa - lcdh} + {v}_{3 k t}^{k c 14 c o a}$
$\frac{d}{d t} c 14 c o {a}_{m i t o} = - {v}_{c 14 c o a - l c d h} + {v}_{3 k t}^{k c 16 c o a}$
$\frac{d}{d t} c 16 c a {r}_{c y t} = + {v}_{C P T 1} - {v}_{C A C T}$
$\frac{d}{d t} c 16 c a {r}_{m i t o} = + \frac{V o {l}_{c y t}}{V o {l}_{mito}} \cdot {v}_{CACT} - {v}_{C P T 2}$
$\frac{d}{d t} c 16 c o {a}_{c y 𝔱} = + {v}_{A C S 1} + {v}_{FATP 1} + {v}_{FATP 4} - {v}_{CPT 1} - {v}_{gpat} - {v}_{a g p a t} - {v}_{d g a t}$
$\frac{d}{d t} c 16 c o {a}_{m i t o} = + {v}_{C P T 2} - {v}_{c 16 coa - lcdh}$
$\frac{d}{d t} c 1 {6}_{e x t} = 0$
$\frac{d}{d t} c 1 {6}_{c y t} = + {v}_{C D 36} - {v}_{A C S 1} - {v}_{F A T P 1} - {v}_{F A T P 4} + \frac{V o {l}_{l d}}{V o {l}_{c y t}} \cdot {v}_{H S L}^{dag} + \frac{V o {l}_{ld}}{V o {l}_{c y t}} \cdot {v}_{A T G L}^{t a g} + \frac{{Vol}_{ld}}{{Vol}_{cyt}} \cdot {v}_{magl}$
$\frac{d}{d t} c 4 c o {a}_{mito} = - {v}_{c 4 c o a - scdh} + {v}_{3 k t}^{k c 6 c o a}$
$\frac{d}{d t} c 6 c o {a}_{m i t o} = - {v}_{c 6 c o a - m c d h} + {v}_{3 k t}^{k c 8 c o a}$
$\frac{d}{d t} c 8 c o {a}_{m i t o} = - {v}_{c 8 coa - mcdh} + {v}_{3 k t}^{k c 10 c o a}$
$\frac{d}{d t} c a {r}_{c γ t} = - {v}_{C P T 1} + {v}_{CACT}$
$\frac{d}{d t} c a {r}_{m i t o} = - \frac{V o {l}_{c y t}}{{Vol}_{mito}} \cdot {v}_{C A C T} + {v}_{C P T 2}$
$\frac{d}{d t} c i {t}_{c y t} = + \frac{V o {l}_{mito}}{{Vol}_{cyt}} \cdot {v}_{cit - mal} - {v}_{cit - lys}$
$\frac{d}{d t} c i {t}_{m i t o} = + {v}_{c s} - {v}_{a c} - {v}_{cit - mal}$
$\frac{d}{d t} {cl}_{c y t} = 0$
$\frac{d}{d t} c {l}_{m i t o} = + \frac{{I}_{{cI}_{ed}}}{10 \cdot F \cdot {Vol}_{mito}}$
$\frac{a}{d t} c o {a}_{c y t} = - {v}_{F A B P 1} - {v}_{F A B P 4} - {v}_{A C S 1} + {v}_{C P T 1} - {v}_{cit - lys} + {v}_{acoah} + {v}_{gpat} + {v}_{agpat} + {v}_{dgat}$
$\frac{d}{dt} {coa}_{mito} = - {v}_{CPT 2} - {v}_{3 kt}^{kc 4 coa} - {v}_{3 ktII}^{kc 4 coa} - {v}_{3 kt}^{kc 6 coa} - {v}_{3 kt}^{kc 8 coa} - {v}_{3 kt}^{kc 10 coa} - \\ {v}_{3 kt}^{kc 12 coa} - {v}_{3 kt}^{kc 14 coa} - {v}_{3 kt}^{kc 16 coa} - {v}_{pdhc} + {v}_{cs} - {v}_{kgdhc} + {v}_{scs - atp} + {v}_{scs - gtp} - \\ {v}_{bckadh - aKICA} - {v}_{bckadh - aKIVA} - {v}_{bckadh - KNeVA} - {v}_{HibDh} - {v}_{mmsdg} - {v}_{MMACT}$
$\frac{d}{dt} c o {2}_{c y t} = 0$
$\frac{d}{d t} c o {2}_{m i t o} = 0$
$\frac{d}{d t} c y t {c}_{o {x}_{m m}} = - \frac{2 \cdot {v}_{cxIII}}{10 \cdot F \cdot {Vol}_{membrane}} + \frac{{ν}_{cxIV}}{10 \cdot F \cdot {Vol}_{m e m b r a n e}}$
$\frac{d}{d t} c y t {c}_{{red}_{m m}} = + \frac{2 \cdot {v}_{cxIII}}{10 \cdot F \cdot {Vol}_{m e m b r a n e}} - \frac{{v}_{cxIV}}{10 \cdot F \cdot {Vol}_{m e m b r a n e}}$
$\frac{d}{d t} {dag}_{e r} = + \frac{V o {l}_{c y t}}{{Vol}_{e r}} \cdot {v}_{p a p} - \frac{V o {l}_{c y t}}{{Vol}_{e r}} \cdot {v}_{d g a t}$
$\frac{d}{d t} d a {g}_{ld} = + {v}_{A T G L}^{t a g} - {v}_{HSL}^{dag}$
$\frac{d}{d t} d h a {p}_{c y t} = + {v}_{ald} - {v}_{t p i} - {v}_{g 3 pdh} - {v}_{g 3 pd {h}_{m i t o}}$
$\frac{d}{d t} e c 10 c o {a}_{m i t o} = + {v}_{c 10 c o a - m c d h} + {v}_{c 10 coa - lcdh} - {v}_{ehyd - ec 10}$
$\frac{d}{d t} ec 12 {coa}_{m i t o} = + {v}_{c 12 coa - m c d h} + {v}_{c 12 coa - lcdh} - {v}_{ehyd - ec 12}$
$\frac{d}{d t} e c 14 c o {a}_{m i t o} = + {v}_{c 14 coa - lcdh} - {v}_{e hyd - ec 14}$
$\frac{d}{d t} e c 16 c o {a}_{m i t o} = + {v}_{c 16 coa - lcdh} - {v}_{e h y d - e c 16}$
$\frac{d}{d t} e c 4 c o {a}_{m i t o} = + {v}_{c 4 coa - scdh} - {v}_{ehyd - ec 4}$
$\frac{d}{d t} e c 6 c o {a}_{mito} = + {v}_{c 6 coa} - {v}_{ehyd - ec 6}$
$\frac{d}{dt} e c 8 c o {a}_{m i t o} = + {v}_{c 8 coa - mcdh} - {v}_{ehyd - ec 8}$
$\frac{d}{d t} e 4 {p}_{c y t} = + {v}_{taldo} + {v}_{t k e t o 2}$
$\frac{d}{dt} {etffad}_{mito} = - {v}_{c 4 coa - scdh} - {v}_{c 6 coa - mcdh} - {v}_{c 8 coa - mcdh} - {v}_{c 10 coa - mcdh} - \\ {v}_{c 12 coa - mcdh} - {v}_{c 10 coa - lcdh} - {v}_{c 12 coa - lcdh} - {v}_{c 14 coa - lcdh} - {v}_{c 16 coa - lcdh} + \\ {v}_{ETF - FAD} - {v}_{MBCoADH - IsoButCoA} - {v}_{M B C o A D H - MeButCoA} - {v}_{IVCoADh}$
$\frac{d}{dt} etffadh {2}_{mito} = + {v}_{c 4 coa - scdh} + {v}_{c 6 coa - mcdh} + {v}_{c 8 coa - mcdh} + {v}_{c 10 coa - mcdh} + \\ {v}_{c 12 coa - mcdh} + {v}_{c 10 coa - lcdh} + {v}_{c 12 coa - lcdh} + {v}_{c 14 coa - lcdh} + {v}_{c 16 coa - lcdh} - \\ {v}_{ETF - FAD} + {v}_{MBCoADH - IsoButCoA} + {v}_{M B C o A D H - MeButCoA} + {v}_{IVCoADh}$
$\frac{d}{d t} e t f {q}_{m i t o} = - {v}_{ETF - FAD} + {v}_{E T F - QO}$
$\frac{d}{d t} e t f q h {2}_{m i t o} = + {v}_{E T F - F F D} - {v}_{E T F - Q O}$
$\frac{d}{d t} fru 16 {bp}_{c y t} = + {v}_{p f k 1} - {v}_{f b p 1} - {v}_{ald}$
$\frac{d}{d t} f r u 26 b {p}_{c y t} = + {v}_{pfk 2} - {v}_{f b p 2}$
$\frac{d}{d t} f r u 6 {p}_{c y t} = + {v}_{gpi} - {v}_{p f k 2} + {v}_{f b p 2} - {v}_{p f k 1} + {v}_{f b p 1} + {v}_{t a l d o} - {v}_{t k e t o 2}$
$\frac{d}{d t} f u {m}_{m i t o} = + {v}_{s u c c d h} - {v}_{f u m}$
$\frac{d}{d t} g 3 {p}_{c y t} = + {v}_{g 3 pdh} + {v}_{g 3 pd {h}_{m i t o}} + {v}_{glycK} - {v}_{gpat}$
$\frac{d}{d t} g d {p}_{c y t} = - {v}_{{ndk}_{c y 𝔱}} + {v}_{pepck}$
$\frac{d}{d t} g d {p}_{mito} = - {v}_{scs - gtp} - {v}_{{ndk}_{mito}}$
$\frac{d}{d t} g l {c}_{c y t} = + {v}_{gluT 1} + {v}_{gluT 4} - {v}_{h k A} - {v}_{h k B}$
$\frac{d}{d t} {glc}_{e x t} = 0$
$\frac{d}{d t} g l c 1 {p}_{c y t} = - {v}_{gpm} - {v}_{u p g a s e} + {v}_{gp}$
$\frac{d}{d t} g l c 6 {p}_{c y t} = + {v}_{h k A} + {v}_{hkB} - {v}_{g p i} + {v}_{g p m} - {v}_{g 6 pdh}$
$\frac{d}{dt} {glu}_{cyt} = + {v}_{asat} - {v}_{agc} - {v}_{{gluT}_{mito}} + {v}_{BCAAT - val} + {v}_{BCAAT - leu} + {v}_{BCAAT - isoleu}$
$\frac{d}{d t} g l {u}_{e x t} = 0$
$\frac{d}{d t} g l {u}_{m i t o} = + {v}_{a s a {t}_{mito}} + \frac{V o {l}_{c y t}}{V o {l}_{m i t o}} \cdot {v}_{a g c} - {v}_{g d h} + \frac{V o {l}_{c y t}}{V o {l}_{m i t o}} \cdot {v}_{{gluT}_{mito}}$
$\frac{d}{dt} {glyc}_{cyt} = + {v}_{glycT} - {v}_{glycK} + \frac{{Vol}_{ld}}{{Vol}_{cyt}} \cdot {v}_{magl}$
$\frac{d}{d t} {glyc}_{e x t} = 0$
$\frac{d}{dt} glyglc = + {v}_{gs} - {v}_{gp}$
$\frac{d}{d t} g r {a}_{e x t} = 0$
$\frac{d}{d t} g r a {p}_{c y t} = + {v}_{ald} + {v}_{t p i} - {v}_{gapdh} - {v}_{taldo} - {v}_{tketo 1} - {v}_{t k e t o 2}$
$\frac{d}{dt} {gtp}_{cyt} = + {v}_{{ndk}_{cyt}} - {v}_{pepck}$
$\frac{d}{d t} g t {p}_{mito} = + {v}_{scs - gtp} + {v}_{n d {k}_{m i t o}}$
$\frac{d}{d t} {h}_{c y 𝔱} = 0$
$\frac{d}{d t} {h}_{m i t o} = + \frac{- {I}_{H}^{pump} + {I}_{{H}_{ed}} + {v}_{P - ex} - {I}_{k}^{p u m p} - {I}_{n a}^{p u m ρ} + 3 \cdot {v}_{F 0 F 1}}{10 \cdot F \cdot {Vol}_{mito}}$
$\frac{d}{d t} h c o {3}_{c y t} = 0 \frac{d}{dt} h c o {3}_{m i t o} = 0 \frac{d}{dt} h i {b}_{m i t o} = + {v}_{H i b C o A h y d} - {v}_{H i b D h}$
$\frac{d}{d t} H i b C o {A}_{m i t o} = + {v}_{ECoAH - MeAcrCoA} - {v}_{H i b C o A h y d} \\ \frac{1}{d t} H M e G C o {A}_{m i t o} = + {v}_{M E G C C H} - {v}_{H M G C L}$
$\frac{d}{dt} {IsoButCoA}_{m i t o} = + {v}_{bckadhh - aKIVA} - {v}_{M B C oADH - IsoButCoA}$
$\frac{d}{d t} i s o l e {u}_{c y t} = + {v}_{i s o l e u T} - {v}_{B CAAT - isoleu}$
$\frac{d}{dt} {isoleu}_{ext} = 0$
$\frac{d}{dt} {isocit}_{mito} = + {v}_{ac} - {v}_{{idh}_{nad}} - {v}_{{idh}_{nadp}}$
$\frac{d}{dt} {IsoValCoA}_{mito} = + {v}_{bckadh - aKICA} - {v}_{IVCoADh}$
$\frac{d}{dt} kc 10 {coa}_{mito} = + {v}_{3 hdh - lc 10} - {v}_{3 kt}^{kc 10 coa}$
$\frac{d}{dt} kc 12 {coa}_{mito} = + {v}_{3 hdh - lc 12} - {v}_{3 kt}^{kc 12 coa}$
$\frac{d}{dt} kc 14 {coa}_{mito} = + {v}_{3 hdh - lc 14} - {v}_{3 kt}^{kc 14 coa}$
$\frac{d}{dt} kc 16 {coa}_{mito} = + {v}_{3 hdh - lc 16} - {v}_{3 kt}^{kc 16 coa}$
$\frac{d}{dt} kc 4 {coa}_{mito} = + {v}_{3 hdh - lc 4} - {v}_{3 kt}^{kc 4 coa} - {v}_{3 ktII}^{kc 4 coa} + {v}_{scot}$
$\frac{d}{dt} kc 6 {coa}_{mito} = + {v}_{3 hdh - lc 6} - {v}_{3 kt}^{kc 6 coa}$
$\frac{d}{dt} kc 8 {coa}_{mito} = + {v}_{3 hdh - lc 8} - {v}_{3 kt}^{kc 8 coa}$
$\frac{d}{dt} {k}_{cyt} = 0$
$\frac{d}{dt} {k}_{mito} = + \frac{{I}_{K}^{pump} + {I}_{{k}_{ed}}}{10 \cdot F \cdot {Vol}_{mito}}$
$\frac{d}{dt} {KMeVA}_{cyt} = + {v}_{BCAAT - isoleu} - {v}_{KMeVAT}$
$\frac{d}{dt} {KMeVA}_{mito} = + {v}_{KMeVAT} \cdot \frac{{Vol}_{mito}}{{Vol}_{cyt}} - {v}_{bckadh - KMeVA}$
$\frac{d}{dt} lc 10 {coa}_{mito} = + {v}_{ehyd - ec 10} - {v}_{3 hdh - lc 10}$
$\frac{d}{dt} lc 12 {coa}_{mito} = + {v}_{ehyd - ec 12} - {v}_{3 hdh - lc 12}$
$\frac{d}{dt} lc 14 {coa}_{mito} = + {v}_{ehyd - ec 14} - {v}_{3 hdh - lc 14}$
$\frac{d}{dt} lc 16 {coa}_{mito} = + {v}_{ehyd - ec 16} - {v}_{3 hdh - lc 16}$
$\frac{d}{dt} lc 4 {coa}_{mito} = + {v}_{ehyd - ec 4} - {v}_{3 hdh - lc 4}$
$\frac{d}{dt} lc 6 {coa}_{mito} = + {v}_{ehyd - ec 6} - {v}_{3 hdh - lc 6}$
$\frac{d}{dt} lc 8 {coa}_{mito} = + {v}_{ehyd - ec 8} - {v}_{3 hdh - lc 8}$
$\frac{d}{dt} {lac}_{cyt} = + {v}_{ldh} + {v}_{lacT}$
$\frac{d}{dt} {lac}_{ext} = 0 \frac{d}{dt} {leu}_{cyt} = + {v}_{leuT} - {v}_{BCAAT - leu}$
$\frac{d}{dt} {leu}_{ext} = 0$
$\frac{d}{dt} {lpa}_{er} = + \frac{{Vol}_{cyt}}{{Vol}_{er}} \cdot {v}_{gpat} - \frac{{Vol}_{cyt}}{{Vol}_{er}} \cdot {v}_{agpat}$
$\frac{d}{dt} {mag}_{ld} = - {v}_{magl} + {v}_{HSL}^{dag}$
$\frac{d}{dt} {mal}_{cyt} = + {v}_{malpT} + {v}_{mal - pyrT} - {v}_{mdh} - {v}_{mac} - \frac{{Vol}_{mito}}{{Vol}_{cyt}} \cdot {v}_{cit - mal} - {v}_{me}$
$\frac{d}{dt} {mal}_{mito} = + {v}_{fum} - {v}_{{mdh}_{mito}} - \frac{{Vol}_{cyt}}{{Vol}_{mito}} \cdot {v}_{malpT} - \frac{{Vol}_{cyt}}{{Vol}_{mito}} \cdot {v}_{mal - pyrT} + \frac{{Vol}_{cyt}}{{Vol}_{mito}} \cdot {v}_{mac} + {v}_{cit - mal}$
$\frac{d}{dt} {malcoa}_{cyt} = + {v}_{acc 1} - {v}_{mcdc} \frac{d}{dt} {MeAACoA}_{mito} = + {v}_{HMBCDH} - {v}_{MAACT}$
$\frac{d}{dt} {MeAcrCoA}_{mito} = + {v}_{MBCoADH - IsoButCoA} - {v}_{ECoAH - MeAcrCoA}$
$\frac{d}{dt} {MeButCoA}_{mito} = + {v}_{bckadh - KMeVA} - {v}_{MBCoADH - MeButCoA}$
$\frac{d}{dt} {MeCroCoA}_{mito} = + {v}_{IVCoADh} - {v}_{MECCC}$
$\frac{d}{dt} {MeGCCoA}_{mito} = + {v}_{MECCC} - {v}_{MEGCCH}$
$\frac{d}{dt} {MeHButCoA}_{mito} = + {v}_{ECoAH - TigCoA} - {v}_{HMBCDH}$
$\frac{d}{dt} {MeMalCoA}_{mito} = + {v}_{PCC} - {v}_{MMCM}$
$\frac{d}{dt} {mmsald}_{mito} = + {v}_{HibDh} - {v}_{mmsdh}$
$\frac{d}{dt} {na}_{cyt} = 0$
$\frac{d}{dt} {na}_{mito} = + \frac{{I}_{na}^{pump} + {I}_{{na}_{ed}}}{10 \cdot F \cdot {Vol}_{mito}}$
$\frac{d}{dt} {nad}_{cyt} = - {v}_{gapdh} + {v}_{ldh} - {v}_{mdh} + {v}_{g 3 pdh}$
$\frac{d}{dt} {nad}_{mito} = - {v}_{3 hdh - lc 4} - {v}_{3 hdh - lc 6} - {v}_{3 hdh - lc 8} - \\ {v}_{3 hdh - lc 10} - {v}_{3 hdh - lc 12} - {v}_{3 hdh - lc 14} - {v}_{3 hdh - lc 16} - {v}_{pdhc} - {v}_{{idh}_{nad}} - \\ {v}_{kgdhc} - {v}_{{mdh}_{mito}} + {v}_{tdh} + \frac{{v}_{cxI}}{10 \cdot F \cdot {Vol}_{mito}} + {v}_{β hdh} - {v}_{bckadh - aKICA} - \\ {v}_{bckadh - aKIVA} - {v}_{bckadh - KMeVa} - {v}_{HibDh} - {v}_{mmsdh} - {v}_{HMBCDH} - {v}_{gdh}$
$\frac{d}{dt} {nadh}_{cyt} = + {v}_{gapdh} - {v}_{ldh} + {v}_{mdh}$
$\frac{d}{dt} {nad}_{mito} = + {v}_{3 hdh - lc 4} + {v}_{3 hdh - lc 6} + {v}_{3 hdh - lc 8} + \\ {v}_{3 hdh - lc 10} + {v}_{3 hdh - lc 12} + {v}_{3 hdh - lc 14} + {v}_{3 hdh - lc 16} + {v}_{pdhc} + {v}_{{idh}_{nad}} + \\ {v}_{kgdhc} + {v}_{{mdh}_{mito}} - {v}_{tdh} - \frac{{v}_{cxI}}{10 \cdot F \cdot {Vol}_{mito}} - {v}_{β hdh} + {v}_{bckadh - aKICA} + \\ {v}_{bckadh - aKIVA} + {v}_{bckadh - KMeVa} + {v}_{HibDh} - {v}_{mmsdh} + {v}_{HMBCDH} + {v}_{gdh}$
$\frac{d}{dt} {nadp}_{cyt} = - {v}_{g 6 pdh} - {v}_{pgdh} - {v}_{me} + {v}_{nadph - use}$
$\frac{d}{dt} {nadp}_{mito} = - {v}_{tdh} - {v}_{{idh}_{nadp}}$
$\frac{d}{dt} {nadph}_{cyt} = + {v}_{g 6 pdh} + {v}_{pgdh} + {v}_{me} - {v}_{nadph - use}$
$\frac{d}{dt} {nadph}_{mito} = + {v}_{tdh} + {v}_{{idh}_{nadp}}$
$\frac{d}{dt} o {2}_{cyt} = - \frac{1}{4} \cdot \frac{{v}_{cxIV}}{10 \cdot F \cdot {Vol}_{cyt}} + {v}_{{O}_{{2}_{diff}}} \frac{d}{dt} o {2}_{ext} = 0$
$\frac{d}{dt} {oaa}_{cyt} = {v}_{mdh} + {v}_{asat} + {v}_{cit - lys}$
$\frac{d}{dt} {oaa}_{mito} = + {v}_{{mdh}_{mito}} + {v}_{pc} + {v}_{{asat}_{mito}} - {v}_{cs}$
$\frac{d}{dt} {p}_{cyt} = - \frac{{v}_{P - ex}}{10 \cdot F \cdot {Vol}_{cyt}} + 2 \cdot {v}_{ppase} + {v}_{atp - usage} + \\ {v}_{fbp 2} + {v}_{fbp 1} - {v}_{gapdh} - {v}_{malpT} - {v}_{gp} + {v}_{cit - lys} + {v}_{acc 1} + {v}_{pap}$
$\frac{d}{dt} {p}_{mito} = - {v}_{scs - atp} - {v}_{scs - gtp} - \frac{{v}_{F 0 F 1}}{10 \cdot F \cdot {Vol}_{mito}} + \\ \frac{{v}_{P - ex}}{10 \cdot F \cdot {Vol}_{mito}} + {v}_{pc} + \frac{{Vol}_{cell}}{{Vol}_{mito}} \cdot {v}_{malpT} + {v}_{PCC} + {v}_{MECCC}$
$\frac{d}{dt} {pa}_{er} = + \frac{{Vol}_{cyt}}{{Vol}_{er}} \cdot {v}_{agpat} - \frac{{Vol}_{cyt}}{{Vol}_{er}} \cdot {v}_{pap} \frac{d}{dt} {pep}_{cyt} = + {v}_{eno} - {v}_{pk} \frac{d}{dt} pg {2}_{cyt} = + {v}_{pgm} - {v}_{eno}$
$\frac{d}{dt} pg {3}_{cyt} = + {v}_{pgk} - {v}_{pgm} \frac{d}{dt} pg {6}_{cyt} = + {v}_{pgls} - {v}_{pgdh} \frac{d}{dt} pgl {6}_{cyt} = + {v}_{g 6 pdh} - {v}_{pgls}$
$\frac{d}{dt} {pp}_{cyt} = + {v}_{ACS 1} + {v}_{FAB 1} + {v}_{FAB 4} - {v}_{ppase} + {v}_{upgase}$
$\frac{d}{dt} {propcoa}_{mito} = + {v}_{mmsdh} + {v}_{MAACT} - {v}_{PCC}$
$\frac{d}{dt} {pyr}_{cyt} = + {v}_{pk} - {v}_{ldh} - {v}_{mal - pyrT} + {v}_{pyrT} - {v}_{{pyrT}_{mito}} + {v}_{me}$
$\frac{d}{dt} {pyr}_{ext} = 0$
$\frac{d}{dt} {pyr}_{mito} = - {v}_{pdhc} - {v}_{pc} + \frac{{Vol}_{cell}}{{Vol}_{mito}} \cdot {v}_{pyrT} + \frac{{Vol}_{cell}}{{Vol}_{mito}} \cdot {v}_{mal - pyrT}$
$\frac{d}{dt} {q}_{mm} = - \frac{{Vol}_{mito}}{{Vol}_{membrane}} \cdot {v}_{ETF - QO} - \frac{{Vol}_{mito}}{{Vol}_{membrane}} \cdot {v}_{succdh} - \\ \frac{{v}_{cxI}}{10 \cdot F \cdot {Vol}_{membrane}} + \frac{{v}_{cxIII}}{10 \cdot F \cdot {Vol}_{membrane}} + \frac{{Vol}_{cyt}}{{Vol}_{membrane}} {v}_{g 3 {pdh}_{mito}}$
$\frac{d}{dt} qh {2}_{mm} = + \frac{{Vol}_{mito}}{{Vol}_{membrane}} \cdot {v}_{ETF - QO} + \frac{{Vol}_{mito}}{{Vol}_{membrane}} \cdot {v}_{succdh} + \\ \frac{{v}_{cxI}}{10 \cdot F \cdot {Vol}_{membrane}} - \frac{{v}_{cxIII}}{10 \cdot F \cdot {Vol}_{membrane}} - \frac{{Vol}_{cyt}}{{Vol}_{membrane}} {v}_{g 3 {pdh}_{mito}}$
$\frac{d}{dt} r 5 {p}_{cyt} = - {v}_{rpi} + {v}_{tketo 1}$
$\frac{d}{dt} ru 5 {p}_{cyt} = + {v}_{pgdh} - {v}_{rpe} + {v}_{rpi}$
$\frac{d}{dt} s 7 {p}_{cyt} = - {v}_{taldo} - {v}_{tketo 1}$
$\frac{d}{dt} {suc}_{mito} = + {v}_{scs - atp} + {v}_{scs - gtp} - {v}_{succdh} + {v}_{scot}$
$\frac{d}{dt} {succoa}_{mito} = + {v}_{kgdhc} - {v}_{scs - atp} - {v}_{scs - gtp} - {v}_{scot} + {v}_{MMCM}$
$\frac{d}{dt} {tag}_{er} = + \frac{{Vol}_{cyt}}{{Vol}_{er}} \cdot {v}_{dgat} - {v}_{LD - syn - tag}$
$\frac{d}{dt} {tag}_{ld} = + \frac{{Vol}_{er}}{{Vol}_{ld}} \cdot {v}_{LD - syn - tag} - {v}_{ATGL}^{tag}$
$\frac{d}{dt} {TigCoA}_{mito} = + {v}_{MBCoADH - MeButCoA} - {v}_{ECoAH - TigCoA}$
$\frac{d}{dt} {udp}_{cyt} = - {v}_{{ndk}_{cyt}}^{udp} + {v}_{gs}$
$\frac{d}{dt} {udpglc}_{cyt} = + {v}_{upgase} - {v}_{gs}$
$\frac{d}{dt} {utp}_{cyt} = + {v}_{{ndk}_{cyt}}^{udp} - {v}_{upgase}$
$\frac{d}{dt} {val}_{cyt} = + {v}_{valT} - {v}_{BCAAT - val}$
$\frac{d}{dt} {val}_{ext} = 0$
$\frac{d}{dt} {v}_{mm} = \frac{{10}^{- 1}}{{c}_{m} \cdot {A}_{m}} \cdot ( - {I}_{{C}_{ed}} + {I}_{{K}_{ed}} + {I}_{{H}_{ed}} + {I}_{{Na}_{ed}} + {I}_{H}^{pump} + {v}_{ex} + 3 \cdot {v}_{syn} )$
$\frac{d}{dt} x 5 {p}_{cyt} = + {v}_{rpe} + {v}_{tketo 1} + {v}_{tketo 2}$

### REFERENCES

1. Stremmel, W., Fatty-Acid Uptake by Isolated Rat-Heart Myocytes Represents a Carrier-Mediated Transport Process. Journal of Clinical Investigation, 1988. 81(3): p. 844-852.
2. Kampf, J. P. and A. M. Kleinfeld, Fatty acid transport in adipocytes monitored by imaging intracellular free fatty acid levels. Journal of Biological Chemistry, 2004. 279(34): p. 35775-35780.
3. Hall, A. M., A. J. Smith, and D. A. Bernlohr, Characterization of the acyl-CoA synthetase activity of purified murine fatty acid transport protein 1. Journal of Biological Chemistry, 2003. 278(44): p. 43008-43013.
4. Hall, A. M., et al., Enzymatic properties of purified murine fatty acid transport protein 4 and analysis of acyl-CoA synthetase activities in tissues from FATP4 null mice. Journal of Biological Chemistry, 2005. 280(12): p. 11948-11954.
5. Liu, H. Y., et al., Cysteine-scanning mutagenesis of muscle carnitine palmitoyltransferase I reveals a single cysteine residue (Cys-305) important for catalysis. Journal of Biological Chemistry, 2005. 280(6): p. 4524-4531.
6. Saggerson, E. D. and C. A. Carpenter, Carnitine Palmitoyltransferase and Carnitine Octanoyltransferase Activities in Liver, Kidney Cortex, Adipocyte, Lactating Mammary-Gland, Skeletal-Muscle and Heart—Relative Activities, Latency and Effect of Malonyl Coa. Febs Letters, 1981. 129(2): p. 229-232.
7. Mcmillin, J. B., et al., Kinetic-Properties of Carnitine Palmitoyltransferase-I in Cultured Neonatal Rat Cardiac Myocytes. Archives of Biochemistry and Biophysics, 1994. 312(2): p. 375-384.
8. Pieklik, J. R. and R. W. Guynn, Equilibrium constants of the reactions of choline acetyltransferase, carnitine acetyltransferase, and acetylcholinesterase under physiological conditions. J Biol Chem, 1975. 250(12): p. 4445-50.
9. Indiveri, C., A. Tonazzi, and F. Palmieri, The reconstituted carnitine carrier from rat liver mitochondria: evidence for a transport mechanism different from that of the other mitochondrial translocators. Biochim Biophys Acta, 1994. 1189(1): p. 65-73.
10. Murthy, M. S. and S. V. Pande, Mechanism of carnitine acylcarnitine translocase-catalyzed import of acylcarnitines into mitochondria. J Biol Chem, 1984. 259(14): p. 9082-9.
11. Pande, S. V., Mitochondrial Carnitine Acylcarnitine Translocase System. Proceedings of the National Academy of Sciences of the United States of America, 1975. 72(3): p. 883-887.
12. Indiveri, C., et al., Kinetic characterization of the reconstituted carnitine carrier from rat liver mitochondria. Biochim Biophys Acta, 1991. 1065(2): p. 231-8.
13. Norum, K. R., Palmityl-Coa—Carnitine Palmityltransferase—Purification from Calf-Liver Mitochondria+Some Properties of Enzyme. Biochimica Et Biophysica Acta, 1964. 89(1): p. 95-&.
14. McGarry, J. D. and N. F. Brown, The mitochondrial carnitine palmitoyltransferase system. From concept to molecular analysis. Eur J Biochem, 1997. 244(1): p. 1-14.
15. Kopec, B. and I. B. Fritz, Properties of a Purified Carnitine Palmitoyltransferase, and Evidence for Existence of Other Carnitine Acyltransferases. Canadian Journal of Biochemistry, 1971. 49(8): p. 941-&.
16. Brown, N. F., et al., Catalytically important domains of rat carnitine palmitoyltransferase II as determined by site-directed mutagenesis and chemical modification. Evidence for a critical histidine residue. J Biol Chem, 1994. 269(29): p. 19157-62.
17. Traufeller, K., F. N. Gellerich, and S. Zierz, Different sensitivities of CPT I and CPT II for inhibition by L-aminocarnitine in human skeletal muscle. Biochimica Et Biophysica Acta-Bioenergetics, 2004. 1608(2-3): p. 149-154.
18. Davidson, B. and H. Schulz, Separation, properties, and regulation of acyl coenzyme A dehydrogenases from bovine heat and liver. Arch Biochem Biophys, 1982. 213(1): p. 155-62.
19. Ikeda, Y., K. Okamura-Ikeda, and K. Tanaka, Purification and characterization of short-chain, medium-chain, and long-chain acyl-CoA dehydrogenases from rat liver mitochondria. Isolation of the holo- and apoenzymes and conversion of the apoenzyme to the holoenzyme. J Biol Chem, 1985. 260(2): p. 1311-25.
20. Osumi, T., T. Hashimoto, and N. Ui, Purification and properties of acyl-CoA oxidase from rat liver. J Biochem, 1980. 87(6): p. 1735-46.
21. Stern, J. R. and A. Del Campillo, Enzymes of fatty acid metabolism. II. Properties of crystalline crotonase. J Biol Chem, 1956. 218(2): p. 985-1002.
22. Fong, J. C. and H. Schulz, Purification and properties of pig heart crotonase and the presence of short chain and long chain enoyl coenzyme A hydratases in pig and guinea pig tissues. J Biol Chem, 1977. 252(2): p. 542-7.
23. Furuta, S., et al., Properties of mitochondria and peroxisomal enoyl-CoA hydratases from rat liver. J Biochem, 1980. 88(4): p. 1059-70.
24. Waterson, R. M. and R. L. Hill, Enoyl coenzyme A hydratase (crotonase). Catalytic properties of crotonase and its possible regulatory role in fatty acid oxidation. J Biol Chem, 1972. 247(16): p. 5258-65.
25. He, X. Y., et al., Identity of heart and liver L-3-hydroxyacyl coenzyme A dehydrogenase. Biochim Biophys Acta, 1999. 1437(2): p. 119-23.
26. Lynen, F. and O. Wieland, Beta-Ketoreductase. Methods in Enzymology, 1955. 1: p. 566-573.
27. He, X. Y., S. Y. Yang, and H. Schulz, Assay of L-3-hydroxyacyl-coenzyme A dehydrogenase with substrates of different chain lengths. Anal Biochem, 1989. 180(1): p. 105-9.
28. Osumi, T. and T. Hashimoto, Purification and properties of mitochondrial and peroxisomal 3-hydroxyacyl-CoA dehydrogenase from rat liver. Arch Biochem Biophys, 1980. 203(1): p. 372-83.
29. Wakil, S. J., et al., Studies on the Fatty Acid Oxidizing System of Animal Tissues 0.6. Beta-Hydroxyacyl Coenzyme a Dehydrogenase. Journal of Biological Chemistry, 1954. 207(2): p. 631-638.
30. !!! INVALID CITATION !!!.
31. Kobayashi, A., L. L. Jiang, and T. Hashimoto, Two mitochondrial 3-hydroxyacyl-CoA dehydrogenases in bovine liver. J Biochem, 1996. 119(4): p. 775-82.
32. Goldman, D. S., Studies on the Fatty Acid Oxidizing System of Animal Tissues 0.7. The Beta-Ketoacyl Coenzyme a Cleavage Enzyme. Journal of Biological Chemistry, 1954. 208(1): p. 345-357.
33. Staack, H., J. F. Binstock, and H. Schulz, Purification and properties of a pig heart thiolase with broad chain length specificity and comparison of thiolases from pig heart and Escherichia coli. J Biol Chem, 1978. 253(6): p. 1827-31.
34. Olowe, Y. and H. Schulz, Regulation of thiolases from pig heart. Control of fatty acid oxidation in heart. Eur J Biochem, 1980. 109(2): p. 425-9.
35. Gilbert, H. F., et al., The relation of acyl transfer to the overall reaction of thiolase I from porcine heart. J Biol Chem, 1981. 256(14): p. 7371-7.
36. Yamashita, H., et al., Acetate generation in rat liver mitochondria; acetyl-CoA hydrolase activity is demonstrated by 3-ketoacyl-CoA thiolase. Biochim Biophys Acta, 2006. 1761(1): p. 17-23.
37. Wrensford, L. V., C. Coppola, and V. E. Anderson, An acyl-coenzyme A chain length dependent assay for 3-oxoacyl-coenzyme A thiolases employing acetyldithio-coenzyme A. Anal Biochem, 1991. 192(1): p. 49-54.
38. Miyazawa, S., et al., Properties of peroxisomal 3-ketoacyl-coA thiolase from rat liver. J Biochem, 1981. 90(2): p. 511-9.
39. Usselman, R. J., et al., Impact of mutations on the midpoint potential of the [4Fe-4S](+1,+2) cluster and on catalytic activity in electron transfer flavoprotein-ubiquinone oxidoreductase (ETF-QO). Biochemistry, 2008. 47(1): p. 92-100.
40. Husain, M., M. T. Stankovich, and B. G. Fox, Measurement of the oxidation-reduction potentials for one-electron and two-electron reduction of electron-transfer flavoprotein from pig liver. Biochem J, 1984. 219(3): p. 1043-7.
41. Brown, G. C. and M. D. Brand, Proton-Electron Stoichiometry of Mitochondrial Complex-I Estimated from the Equilibrium Thermodynamic Force Ratio. Biochemical Journal, 1988. 252(2): p. 473-479.
42. Strumilo, S., J. Czerniecki, and P. Dobrzyn, Regulatory effect of thiamin pyrophosphate on pig heart pyruvate dehydrogenase complex. Biochem Biophys Res Commun, 1999. 256(2): p. 341-5.
43. Roche, T. E. and R. L. Cate, Purification of porcine liver pyruvate dehydrogenase complex and characterization of its catalytic and regulatory properties. Arch Biochem Biophys, 1977. 183(2): p. 664-77.
44. Wieland, O., B. Von Jagow-Westermann, and B. Stukowski, Kinetic and regulatory properties of heart muscle pyruvate dehydrogenase. Hoppe Seylers Z Physiol Chem, 1969. 350(3): p. 329-34.
45. Huang, H. M., et al., The role of cytosolic free calcium in the regulation of pyruvate dehydrogenase in synaptosomes. Neurochem Res, 1994. 19(1): p. 89-95.
46. Denton, R. M. and J. G. McCormack, The role of calcium in the regulation of mitochondrial metabolism. Biochem Soc Trans, 1980. 8(3): p. 266-8.
47. Pettit, F. H., J. W. Pelley, and L. J. Reed, Regulation of pyruvate dehydrogenase kinase and phosphatase by acetyl-CoA/CoA and NADH/NAD ratios. Biochem Biophys Res Commun, 1975. 65(2): p. 575-82.
48. Wieland, O. and L. Weiss, Inhibition of Citrate-Synthase by Palmityl-Coenzyme A. Biochem Biophys Res Commun, 1963. 13: p. 26-31.
49. Moriyama, T. and P. A. Srere, Purification of rat heart and rat liver citrate synthases. Physical, kinetic, and immunological studies. J Biol Chem, 1971. 246(10): p. 3217-23.
50. Reidenbe.Mm, et al., Species Differences in Pathway of Conjugation of Phenylacetic Acid. Federation Proceedings, 1971. 30(2): p. A392-&.
51. Smith, C. M. and Williams.Jr, Inhibition of Citrate Synthase by Succinyl-Coa and Other Metabolites. Febs Letters, 1971. 18(1): p. 35-&
52. Johansson, C. J., A. Mahlen, and G. Petterson, Kinetic studies on citrate synthase from pig heart. Biochim Biophys Acta, 1973. 309(2): p. 466-72.
53. Blair, J. M., Magnesium and the aconitase equilibrium: determination of apparent stability constants of mangnesium substrate complexes from equilibrium data. Eur J Biochem, 1969. 8(2): p. 287-91.
54. Villafranca, J. J. and A. S. Mildvan, The mechanism of aconitase action. II. Magnetic resonance studies of the complexes of enzyme, manganese(II), iron(II), and substrates. J Biol Chem, 1971. 246(18): p. 5791-8.
55. Nichols, B. J., M. Rigoulet, and R. M. Denton, Comparison of the effects of Ca2+, adenine nucleotides and pH on the kinetic properties of mitochondrial NAD(+)-isocitrate dehydrogenase and oxoglutarate dehydrogenase from the yeast Saccharomyces cerevisiae and rat heart. Biochem J, 1994. 303 (Pt 2): p. 461-5.
56. Plaut, G. W. and T. Aogaichi, Purification and properties of diphosphopyridine nuleotide-linked isocitrate dehydrogenase of mammalian liver. J Biol Chem, 1968. 243(21): p. 5572-83.
57. Gabriel, J. L. and G. W. Plaut, Citrate activation of NAD-specific isocitrate dehydrogenase from bovine heart. J Biol Chem, 1984. 259(3): p. 1622-8.
58. Gabriel, J. L. and G. W. Plaut, Inhibition of bovine heart NAD-specific isocitrate dehydrogenase by reduced pyridine nucleotides: modulation of inhibition by ADP, NAD+, Ca2+, citrate, and isocitrate. Biochemistry, 1984. 23(12): p. 2773-8.
59. Popova, T., et al., Regulation of mitochondrial NADP-isocitrate dehydrogenase in rat heart during ischemia. Mol Cell Biochem, 2007. 294(1-2): p. 97-105.
60. Smith, C. M., J. Bryla, and J. R. Williamson, Regulation of mitochondrial alpha-ketoglutarate metabolism by product inhibition at alpha-ketoglutarate dehydrogenase. J Biol Chem, 1974. 249(5): p. 1497-505.
61. Strumilo, S., et al., Kinetic and spectral investigation of allosteric interaction of coenzymes with 2-oxo acid dehydrogenase complexes. Journal of Molecular Structure, 2002. 614(1-3): p. 221-226.
62. Lambeth, D. O., et al., Expression of two succinyl-CoA synthetases with different nucleotide specificities in mammalian tissues. Journal of Biological Chemistry, 2004. 279(35): p. 36621-36624.
63. Phillips, D., et al., Succinyl-CoA Synthetase Is a Phosphate Target for the Activation of Mitochondrial Metabolism. Biochemistry, 2009. 48(30): p. 7140-7149.
64. Kaufman, S. and S. G. A. Alivisatos, Purification and Properties of the Phosphorylating Enzyme from Spinach. Journal of Biological Chemistry, 1955. 216(1): p. 141-152.
65. Johnson, J. D., W. W. Muhonen, and D. O. Lambeth, Characterization of the ATP- and GTP-specific succinyl-CoA synthetases in pigeon. The enzymes incorporate the same alpha-subunit. J Biol Chem, 1998. 273(42): p. 27573-9.
66. Lynn, R. and R. W. Guynn, Equilibrium-Constants under Physiological Conditions for Reactions of Succinyl Coenzyme-a Synthetase and Hydrolysis of Succinyl Coenzyme-a to Coenzyme-a and Succinate. Journal of Biological Chemistry, 1978. 253(8): p. 2546-2553.
67. King, T. E., Preparation of succinate dehydrogenase and reconstitution of succinate oxidase, in Methods in Enzymology. 1967, Academic Press. p. 322-331.
68. Vinogradov, A. D., et al., Regulation of Succinated Dehydrogenase and Tautomerization of Oxaloacetate. Advances in Enzyme Regulation, 1989. 28: p. 271-280.
69. Tushurashvili, P. R., et al., Studies on the Succinate Dehydrogenating System—Isolation and Properties of the Mitochondrial Succinate-Ubiquinone Reductase. Biochimica Et Biophysica Acta, 1985. 809(2): p. 145-159.
70. Bock, R. M. and R. A. Alberty, Studies of the Enzyme Fumarase. 1. Kinetics and Equilibrium. Journal of the American Chemical Society, 1953. 75(8): p. 1921-1925.
71. Kobayashi, K., T. Yamanishi, and S. Tuboi, Physicochemical, Catalytic, and Immunochemical Properties of Fumarases Crystallized Separately from Mitochondrial and Cytosolic Fractions of Rat-Liver. Journal of Biochemistry, 1981. 89(6): p. 1923-1931.
72. Raval, D. N. and R. G. Wolfe, Malic Dehydrogenase 0.4. Ph Dependence of Kinetic Parameters. Biochemistry, 1962. 1(6): p. 1118-&.
73. Thorne, C. J. R., Properties of Mitochondrial Malate Dehydrogenases. Biochimica Et Biophysica Acta, 1962. 59(3): p. 624-&.
74. Gelpi, J. L., et al., Kinetic-Studies of the Regulation of Mitochondrial Malate-Dehydrogenase by Citrate. Biochemical Journal, 1992. 283: p. 289-297.
75. Moyle, J. and P. Mitchell, Proton Translocating Nicotinamide-Adenine Dinucleotide (Phosphate) Transhydrogenase of Rat-Liver Mitochondria. Biochemical Journal, 1973. 132(3): p. 571-585.
76. Kaplan, N. O., S. P. Colowick, and E. F. Neufeld, Pyridine Nucleotide Transhydrogenase 0.3. Animal Tissue Transhydrogenases. Journal of Biological Chemistry, 1953. 205(1): p. 1-15.
77. Brierley, G. P., et al., Kinetic properties of the Na+/H+ antiport of heart mitochondria. Biochemistry, 1989. 28(10): p. 4347-54.
78. Seren, S., et al., Current-voltage relationships for proton flow through the FO sector of the ATP-synthase, carbonylcyanide-p-trifluoromethoxyphenylhydrazone or leak pathways in submitochondrial particles. Eur J Biochem, 1985. 152(2): p. 373-9.
79. Gao, Y. Q., W. Yang, and M. Karplus, A structure-based model for the synthesis and hydrolysis of ATP by F-1-ATPase. Cell, 2005. 123(2): p. 195-205.
80. Bohnensack, R., U. Kuster, and G. Letko, Rate-Controlling Steps of Oxidative-Phosphorylation in Rat-Liver Mitochondria—a Synoptic Approach of Model and Experiment. Biochimica Et Biophysica Acta, 1982. 680(3): p. 271-280.
81. Coty, W. A. and P. L. Pedersen, Phosphate transport in rat liver mitochondria. Kinetics and energy requirements. J Biol Chem, 1974. 249(8): p. 2593-8.
82. Burton, K. and T. H. Wilson, The Free-Energy Changes for the Reduction of Diphosphopyridine Nucleotide and the Dehydrogenation of L-Malate and L-Glycerol 1-Phosphate. Biochemical Journal, 1953. 54(1): p. 86-94.
83. Nakashima, Y., et al., Steady-state kinetics of NADH:coenzyme Q oxidoreductase isolated from bovine heart mitochondria. J Bioenerg Biomembr, 2002. 34(1): p. 11-9.
84. Hinkle, P. and P. Mitchell, Effect of membrane potential on equilibrium poise between cytochrome a and cytochrome c in rat liver mitochondria. J Bioenerg, 1970. 1(1): p. 45-60.
85. Fato, R., et al., Steady-state kinetics of ubiquinol-cytochrome c reductase in bovine heart submitochondrial particles: diffusional effects. Biochem J, 1993. 290 (Pt 1): p. 225-36.
86. Napiwotzki, J. and B. Kadenbach, Extramitochondrial ATP/ADP-ratios regulate cytochrome c oxidase activity via binding to the cytosolic domain of subunit IV. Biological Chemistry, 1998. 379(3): p. 335-339.
87. Derr, R. F. and L. Zieve, Adenylate Energy Charge—Relation to Guanylate Energy Charge and Adenylate Kinase Equilibrium Constant. Biochemical and Biophysical Research Communications, 1972. 49(6): p. 1385-&.
88. Font, B. and D. C. Gautheron, General and kinetic properties of pig heart mitochondrial adenylate kinase. Biochim Biophys Acta, 1980. 611(2): p. 299-308.
89. Unguryte, A., I. N. Smirnova, and A. A. Baykov, Kinetic models for the action of cytosolic and mitochondrial inorganic pyrophosphatases of rat liver. Arch Biochem Biophys, 1989. 273(2): p. 292-300.
90. Asrih, M., et al., Differential regulation of stimulated glucose transport by free fatty acids and PPARalpha or -delta agonists in cardiac myocytes. Am J Physiol Endocrinol Metab, 2012. 302(7): p. E872-84.
91. Zhao, F. Q. and A. F. Keating, Functional properties and genomics of glucose transporters. Curr Genomics, 2007. 8(2): p. 113-28.
92. Gould, G. W., et al., Expression of human glucose transporters in Xenopus oocytes: kinetic characterization and substrate specificities of the erythrocyte, liver, and brain isoforms. Biochemistry, 1991. 30(21): p. 5139-45.
93. Fischer, Y., et al., Insulin-induced recruitment of glucose transporter 4 (GLUT4) and GLUT1 in isolated rat cardiac myocytes. Evidence of the existence of different intracellular GLUT4 vesicle populations. J Biol Chem, 1997. 272(11): p. 7085-92.
94. Kraegen, E. W., et al., Glucose transporters and in vivo glucose uptake in skeletal and cardiac muscle: fasting, insulin stimulation and immunoisolation studies of GLUT1 and GLUT4. Biochem J, 1993. 295 (Pt 1): p. 287-93.
95. Easterby, J. S. and M. J. Obrien, Purification and Properties of Pig-Heart Hexokinase. European Journal of Biochemistry, 1973. 38(2): p. 201-211.
96. England, P. J. and P. J. Randle, Effectors of Rat-Heart Hexokinases and Control of Rates of Glucose Phosphorylation in Perfused Rat Heart. Biochemical Journal, 1967. 105(3): p. 907-&.
97. Vowles, D. T. and J. S. Easterby, Comparison of Type-I Hexokinases from Pig Heart and Kinetic Evaluation of the Effects of Inhibitors. Biochimica Et Biophysica Acta, 1979. 566(2): p. 283-295.
98. Tewari, Y. B., D. K. Steckler, and R. N. Goldberg, Thermodynamics of isomerization reactions involving sugar phosphates. J Biol Chem, 1988. 263(8): p. 3664-9.
99. Zalitis, J. and I. T. Oliver, Inhibition of Glucose Phosphate Isomerase by Metabolic Intermediates of Fructose. Biochemical Journal, 1967. 102(3): p. 753-&
100. Bertrand, L., et al., Heart 6-phosphofructo-2-kinase activation by insulin results from Ser-466 and Ser-483 phosphorylation and requires 3-phosphoinositide-dependent kinase-1, but not protein kinase B. Journal of Biological Chemistry, 1999. 274(43): p. 30927-30933.
101. Abe, Y., et al., Expression of Bovine Heart Fructose 6-Phosphate,2-Kinase-Fructose 2,6-Bisphosphatase and Determination of the Role of the Carboxyl-Terminus by Mutagenesis. Biochemistry, 1995. 34(8): p. 2553-2559.
102. Deprez, J., et al., Phosphorylation and activation of heart 6-phosphofructo-2-kinase by protein kinase B and other protein kinases of the insulin signaling cascades. Journal of Biological Chemistry, 1997. 272(28): p. 17269-17275.
103. Narabayashi, H., J. W. R. Lawson, and K. Uyeda, Regulation of Phosphofructokinase in Perfused Rat-Heart—Requirement for Fructose 2,6-Bisphosphate and a Covalent Modification. Journal of Biological Chemistry, 1985. 260(17): p. 9750-9758.
104. Bristow, J., D. M. Bier, and L. G. Lange, Regulation of Adult and Fetal Heart Phosphofructokinase (Pfk)—Permissive Effects of Fructose 2,6-Bisphosphate. Circulation, 1986. 74(4): p. 327-327.
105. Uyeda, K., E. Furuya, and L. J. Luby, The Effect of Natural and Synthetic D-Fructose 2,6-Bisphosphate on the Regulatory Kinetic-Properties of Liver and Muscle Phosphofructokinases. Journal of Biological Chemistry, 1981. 256(16): p. 8394-8399.
106. Pogson, C. I. and P. J. Randle, Control of Rat-Heart Phosphofructokinase by Citrate and Other Regulators. Biochemical Journal, 1966. 100(3): p. 683-&.
107. Meek, D. W. and H. G. Nimmo, Effects of phosphorylation on the kinetic properties of rat liver fructose-1,6-bisphosphatase. Biochem J, 1984. 222(1): p. 125-30.
108. Veech, R. L., et al., Disequilibrium in the triose phosphate isomerase system in rat liver. Biochem J, 1969. 115(4): p. 837-42.
109. Ikehara, Y., H. Endo, and Y. Okada, The identity of the aldolases isolated from rat muscle and primary hepatoma. Arch Biochem Biophys, 1970. 136(2): p. 491-7.
110. Malay, A. D., S. L. Procious, and D. R. Tolan, The temperature dependence of activity and structure for the most prevalent mutant aldolase B associated with hereditary fructose intolerance. Archives of Biochemistry and Biophysics, 2002. 408(2): p. 295-304.
111. Lee, E. W., et al., Purification and Properties of Liver Triose Phosphate Isomerase. Biochimica Et Biophysica Acta, 1971. 242(1): p. 261-&.
112. Cori, C. F., S. F. Velick, and G. T. Cori, The combination of diphosphopyridine nucleotide with glyceraldehyde phosphate dehydrogenase. Biochim Biophys Acta, 1950. 4(1-3): p. 160-9.
113. Ryzlak, M. T. and R. Pietruszko, Heterogeneity of Glyceraldehyde-3-Phosphate Dehydrogenase from Human-Brain. Biochimica Et Biophysica Acta, 1988. 954(3): p. 309-324.
114. Mochizuki, S. and J. R. Neely, Control of Glyceraldehyde-3-Phosphate Dehydrogenase in Cardiac-Muscle. Journal of Molecular and Cellular Cardiology, 1979. 11(3): p. 221-236.
115. Smith, C. M. and S. F. Velick, The glyceraldehyde 3-phosphate dehydrogenases of liver and muscle. Cooperative interactions and conditions for functional reversibility. J Biol Chem, 1972. 247(1): p. 273-84.
116. Cornell, N. W., M. Leadbetter, and R. L. Veech, Effects of free magnesium concentration and ionic strength on equilibrium constants for the glyceraldehyde phosphate dehydrogenase and phosphoglycerate kinase reactions. J Biol Chem, 1979. 254(14): p. 6522-7.
117. Krietsch, W. K. and T. Bucher, 3-phosphoglycerate kinase from rabbit sceletal muscle and yeast. Eur J Biochem, 1970. 17(3): p. 568-80.
118. Fritz, P. J. and E. L. White, 3-Phosphoglycerate kinase from rat tissues. Further characterization and developmental studies. Biochemistry, 1974. 13(3): p. 444-9.
119. Rodwell, V. W., J. C. Towne, and S. Grisolia, The kinetic properties of yeast and muscle phosphoglyceric acid mutase. J Biol Chem, 1957. 228(2): p. 875-90.
120. Fundele, R. and W. K. Krietsch, Purification and properties of the phosphoglycerate mutase isozymes from the mouse. Comp Biochem Physiol B, 1985. 81(4): p. 965-8.
121. Schuster, R. and H. G. Holzhutter, Use of mathematical models for predicting the metabolic effect of large-scale enzyme activity alterations. Application to enzyme deficiencies of red blood cells. Eur J Biochem, 1995. 229(2): p. 403-18.
122. Rider, C. C. and C. B. Taylor, Enolase isoenzymes in rat tissues. Electrophoretic, chromatographic, immunological and kinetic properties. Biochim Biophys Acta, 1974. 365(1): p. 285-300.
123. Imamura, K. and T. Tanaka, Pyruvate-Kinase Isozymes from Rat. Methods in Enzymology, 1982. 90: p. 150-165.
124. Wimhurst, J. M. and K. L. Manchester, Some aspects of the kinetics of rat liver pyruvate carboxylase. Biochem J, 1970. 120(1): p. 79-93.
125. Williamson, D. H., P. Lund, and H. A. Krebs, The redox state of free nicotinamide-adenine dinucleotide in the cytoplasm and mitochondria of rat liver. Biochem J, 1967. 103(2): p. 514-27.
126. Nisselbaum, J. and O. Bodansky, Purification and Properties of Human Heart Lactic Dehydrogenase. Journal of Biological Chemistry, 1961. 236(2): p. 323-&.
127. Singh, S. N. and M. S. Kanungo, Alterations in Lactate Dehydrogenase of Brain Heart Skeletal Muscle and Liver of Rats of Various Ages. Journal of Biological Chemistry, 1968. 243(17): p. 4526-&.
128. Halestrap, A. P., et al., Lactate transport in heart in relation to myocardial ischemia. Am J Cardiol, 1997. 80(3A): p. 17A-25A.
129. Halestrap, A. P., The mitochondrial pyruvate carrier. Kinetics and specificity for substrates and inhibitors. Biochem J, 1975. 148(1): p. 85-96.
130. Indiveri, C., et al., Kinetics of the Reconstituted Dicarboxylate Carrier from Rat-Liver Mitochondria. Biochimica Et Biophysica Acta, 1989. 977(2): p. 187-193.
131. Saint-Macary, M. and B. Foucher, Comparative partial purification of the active dicarboxylate transport system of rat liver, kidney and heart mitochondria. Biochem Biophys Res Commun, 1985. 133(2): p. 498-504.
132. Titheradge, M. A. and H. G. Coore, Mitochondrial Pyruvate Carrier, Its Exchange Properties and Its Regulation by Glucagon. Febs Letters, 1976. 63(1): p. 45-50.
133. Palmieri, F., et al., Kinetic Study of Tricarboxylate Carrier in Rat-Liver Mitochondria. European Journal of Biochemistry, 1972. 26(4): p. 587-&.
134. Englard, S. and H. H. Breiger, Beef-heartmalic dehydrogenases. II. Preparation and properties of crystalline supernatant malic dehydrogenase. Biochim Biophys Acta, 1962. 56: p. 571-83.
135. Veech, R. L., L. V. Eggleston, and H. A. Krebs, The redox state of free nicotinamide-adenine dinucleotide phosphate in the cytoplasm of rat liver. Biochem J, 1969. 115(4): p. 609-19.
136. Zelewski, M. and J. Swierczynski, Malic enzyme in human liver. Intracellular distribution, purification and properties of cytosolic isozyme. Eur J Biochem, 1991. 201(2): p. 339-45.
137. Frenkel, R., Bovine heart malic enzyme. I. Isolation and partial purification of a cytoplasmic and a mitochondrial enzyme. J Biol Chem, 1971. 246(9): p. 3069-74.
138. Fukuchi, T., et al., Recombinant rat nucleoside diphosphate kinase isoforms (alpha and beta): purification, properties and application to immunological detection of native isoforms in rat tissues. Biochim Biophys Acta, 1994. 1205(1): p. 113-22.
139. Kimura, N. and N. Shimada, Membrane-Associated Nucleoside Diphosphate Kinase from Rat-Liver -Purification, Characterization, and Comparison with Cytosolic Enzyme. Journal of Biological Chemistry, 1988. 263(10): p. 4647-4653.
140. Colowick, S. P. and E. W. Sutherland, Polysaccharide synthesis from glucose by means purified enzymes. Journal of Biological Chemistry, 1942. 144(2): p. 423-437.
141. Kashiwaya, Y., et al., Control of Glucose-Utilization in Working Perfused Rat-Heart. Journal of Biological Chemistry, 1994. 269(41): p. 25502-25514.
142. Turnquis.RI, T. A. Gillett, and R. G. Hansen, Uridine-Diphosphate Glucose Pyrophosphorylase-Crystallization and Properties of Enzyme from Rabbit Liver and Species Comparisons. Journal of Biological Chemistry, 1974. 249(23): p. 7695-7700.
143. Mitchell, J. W. and J. A. Thomas, Phosphorylation of bovine heart glycogen synthase by two protein kinases. Kinetic properties of phosphorylated forms of the enzyme. J Biol Chem, 1981. 256(12): p. 6160-9.
144. Berndt, N. and P. Rosen, Isolation and partial characterization of two forms of rat heart glycogen phosphorylase. Arch Biochem Biophys, 1984. 228(1): p. 143-54.
145. Maddaiah, V. T. and N. B. Madsen, Kinetics of purified liver phosphorylase. J Biol Chem, 1966. 241(17): p. 3873-81.
146. Tan, A. W. and F. Q. Nuttall, Characteristics of the dephosphorylated form of phosphorylase purified from rat liver and measurement of its activity in crude liver preparations. Biochim Biophys Acta, 1975. 410(1): p. 45-60.
147. Colomb, M. G., A. Cheruy, and P. V. Vignais, Nucleoside diphosphokinase from beef heart cytosol. I. Physical and kinetic properties. Biochemistry, 1972. 11(18): p. 3370-8.
148. Krebs, H. A., Equilibria in transamination systems. Biochem J, 1953. 54(1): p. 82-6.
149. Nisselbaum, J. S. and O. Bodansky, Kinetics and electrophoretic properties of the isozymes of aspartate aminotransferase from pig heart. J Biol Chem, 1966. 241(11): p. 2661-4.
150. Rakhmanova, T. I. and T. N. Popova, Regulation of 2-oxoglutarate metabolism in rat liver by NADP-isocitrate dehydrogenase and aspartate aminotransferase. Biochemistry-Moscow, 2006. 71(2): p. 211-217.
151. Palmieri, L., et al., Citrin and aralar1 are Ca2+-stimulated aspartate/glutamate transporters in mitochondria. Embo Journal, 2001. 20(18): p. 5060-5069.
152. Lanoue, K. F., et al., Kinetic-Properties of Aspartate Transport in Rat-Heart Mitochondrial Inner Membranes. Archives of Biochemistry and Biophysics, 1979. 195(2): p. 578-590.
153. Dierks, T. and R. Kramer, Asymmetric orientation of the reconstituted aspartate/glutamate carrier from mitochondria. Biochim Biophys Acta, 1988. 937(1): p. 112-26.
154. Indiveri, C., et al., Reaction mechanism of the reconstituted oxoglutarate carrier from bovine heart mitochondria. Eur J Biochem, 1991. 198(2): p. 339-47.
155. Euler, H., E. Adler, and G. Günther, Über die Komponenten der Dehydrasesysteme. XV.—Zur Kenntnis der Dehydrierung von a-Glycerin-phosphorsäure im Tierkörper, in Hoppe-Seyler's Zeitschrift für physiologische Chemie. 1937. p. 1.
156. Ostro, M. J. and T. P. Fondy, Isolation and characterization of multiple molecular forms of cytosolic NAD-linked glycerol-3-phosphate dehydrogenase from normal and neoplastic rabbit tissues. J Biol Chem, 1977. 252(15): p. 5575-83.
157. White, H. B. and N. O. Kaplan, Purification and Properties of 2 Types of Diphosphopyridine Nucleotide-Linked Glycerol 3-Phosphate Dehydrogenases from Chicken Breast Muscle and Chicken Liver. Journal of Biological Chemistry, 1969. 244(21): p. 6031-&.
158. Ponsot, E., et al., Mitochondrial tissue specificity of substrates utilization in rat cardiac and skeletal muscles. J Cell Physiol, 2005. 203(3): p. 479-86.
159. Glock, G. E. and P. Mclean, Further Studies on the Properties and Assay of Glucose 6-Phosphate Dehydrogenase and 6-Phosphogluconate Dehydrogenase of Rat Liver. Biochemical Journal, 1953. 55(3): p. 400-408.
160. Taketa, K. and B. M. Pogell, Effect of Palmityl Coenzyme a on Glucose 6-Phosphate Dehydrogenase and Other Enzymes. Journal of Biological Chemistry, 1966. 241(3): p. 720-&.
161. Corpas, F. J., et al., Kinetic-Properties of Hexose-Monophosphate Dehydrogenases 0.1. Isolation and Partial-Purification of Glucose-6-Phosphate-Dehydrogenase from Rat-Liver and Kidney Cortex. Life Sciences, 1994. 56(3): p. 179-189.
162. Bauer, H. P., et al., 6-Phosphogluconolactonase—Purification, Properties and Activities in Various Tissues. European Journal of Biochemistry, 1983. 133(1): p. 163-168.
163. Villet, R. H. and K. Dalziel, The nature of the carbon dioxide substrate and equilibrium constant of the 6-phosphogluconate dehydrogenase reaction. Biochem J, 1969. 115(4): p. 633-8.
164. Procsal, D. and D. Holten, Purification and Properties of Rat-Liver 6-Phosphogluconate Dehydrogenase—Activity at Normal in-Vivo Concentration of Coenzyme. Biochemistry, 1972. 11(7): p. 1310-&.
165. Weisz, K. S., P. J. Schofield, and M. R. Edwards, Human-Brain 6-Phosphogluconate Dehydrogenase—Purification and Kinetic-Properties. Journal of Neurochemistry, 1985. 44(2): p. 510-517.
166. Horecker, B. L. and J. Hurwitz, The purification of phosphoketopentoepimerase from Lactobacillus pentosus and the preparation of xylulose 5-phosphate. J Biol Chem, 1956. 223(2): p. 993-1008.
167. Akana, J., et al., D-ribulose 5-phosphate 3-epimerase: Functional and structural relationships to members of the ribulose-phosphate binding (beta/alpha)(8)-barrel superfamily. Biochemistry, 2006. 45(8): p. 2493-2503.
168. Tabachnick, M., et al., The oxidative pentose phosphate cycle. III. The interconversion of ribose 5-phosphate, ribulose 5-phosphate and xylulose 5-phosphate. Arch Biochem Biophys, 1958. 74(2): p. 315-25.
169. Kiely, M. E., A. L. Stuart, and T. Wood, Partial-Purification and Kinetic Properties of Ribose-5-Phosphate Ketol-Isomerase and Ribulose-5-Phosphate 3-Epimerase from Various Sources. Biochimica Et Biophysica Acta, 1973. 293(2): p. 534-541.
170. Venkataraman, R. and E. Racker, Mechanism of Action of Transaldolase 0.1. Crystallization and Properties of Yeast Enzyme. Journal of Biological Chemistry, 1961. 236(7): p. 1876-&.
171. Sprenger, G. A., et al., Transaldolase-B of Escherichia-Coli K-12—Cloning of Its Gene, Talb, and Characterization of the Enzyme from Recombinant Strains. Journal of Bacteriology, 1995. 177(20): p. 5930-5936.
172. Heinrich, P. C., H. P. Morris, and G. Weber, Behavior of Transaldolase (Ec-2.2.1.2) and Transketolase (Ec-2.2.1.1) Activities in Normal, Neoplastic, Differentiating, and Regenerating Liver. Cancer Research, 1976. 36(9): p. 3189-3197.
173. Datta, A. G. and E. Racker, Mechanism of action of transketolase. I. Properties of the crystalline yeast enzyme. J Biol Chem, 1961. 236: p. 617-23.
174. Paoletti, F., Purification and Properties of Transketolase from Fresh Rat-Liver. Archives of Biochemistry and Biophysics, 1983. 222(2): p. 489-496.
175. Takeuchi, T., K. Nishino, and Y. Itokawa, Purification and Characterization of, and Preparation of an Antibody to, Transketolase from Human Red-Blood-Cells. Biochimica Et Biophysica Acta, 1986. 872(1-2): p. 24-32.
176. Meshalkina, L. E., O. N. Solovjeva, and G. A. Kochetov, Interaction of Transketolase from Human Tissues with Substrates. Biochemistry-Moscow, 2011. 76(9): p. 1061-1064.
177. Novello, F. and P. Mclean, Pentose Phosphate Pathway of Glucose Metabolism—Measurement of Non-Oxidative Reactions of Cycle. Biochemical Journal, 1968. 107(6): p. 775-&.
178. Halperin, M. L., I. B. Fritz, and B. H. Robinson, Effects of Palmitoyl Coa on Citrate and Malate Transport by Rat-Liver Mitochondria. Proceedings of the National Academy of Sciences of the United States of America, 1972. 69(4): p. 1003-&.
179. Bisaccia, F., et al., Kinetic Characterization of the Reconstituted Tricarboxylate Carrier from Rat-Liver Mitochondria. Biochimica Et Biophysica Acta, 1990. 1019(3): p. 250-256.
180. Bisaccia, F., et al., Reaction-Mechanism of the Reconstituted Tricarboxylate Carrier from Rat-Liver Mitochondria. Biochimica Et Biophysica Acta, 1993. 1142(1-2): p. 139-145.
181. Potapova, I. A., et al., Phosphorylation of recombinant human ATP:citrate lyase by cAMP-dependent protein kinase abolishes homotropic allosteric regulation of the enzyme by citrate and increases the enzyme activity. Allosteric activation of ATP:citrate lyase by phosphorylated sugars. Biochemistry, 2000. 39(5): p. 1169-79.
182. Trumble, G. E., M. A. Smith, and W. W. Winder, Purification and characterization of rat skeletal muscle acetyl-CoA carboxylase. Eur J Biochem, 1995. 231(1): p. 192-8.
183. Bianchi, A., et al., Identification of an isozymic form of acetyl-CoA carboxylase. J Biol Chem, 1990. 265(3): p. 1502-9.
184. Jamil, H. and N. B. Madsen, Phosphorylation State of Acetyl-Coenzyme-a Carboxylase. 1. Linear Inverse Relationship to Activity Ratios at Different Citrate Concentrations. Journal of Biological Chemistry, 1987. 262(2): p. 630-637.
185. Zhou, D., et al., Expression, purification, and characterization of human malonyl-CoA decarboxylase. Protein Expr Purif, 2004. 34(2): p. 261-9.
186. Suematsu, N., et al., Molecular cloning and functional expression of rat liver cytosolic acetyl-CoA hydrolase. Eur J Biochem, 2001. 268(9): p. 2700-9.
187. Katano, T., et al., Competitive inhibition of AQP7-mediated glycerol transport by glycerol derivatives. Drug Metab Pharmacokinet, 2014. 29(4): p. 348-51.
188. Robinson, J. and E. A. Newsholme, Some properties of hepatic glycerol kinase and their relation to the control of glycerol utilization. Biochem J, 1969. 112(4): p. 455-64.
189. Yet, S. F., Y. K. Moon, and H. S. Sul, Purification and reconstitution of murine mitochondrial glycerol-3-phosphate acyltransferase. Functional expression in baculovirus-infected insect cells. Biochemistry, 1995. 34(22): p. 7303-10.
190. Yamashita, A., et al., Topology of acyltransferase motifs and substrate specificity and accessibility in 1-acyl-sn-glycero-3-phosphate acyltransferase 1. Biochim Biophys Acta, 2007. 1771(9): p. 1202-15.
191. Han, G. S. and G. M. Carman, Characterization of the human LPIN1-encoded phosphatidate phosphatase isoforms. J Biol Chem, 2010. 285(19): p. 14628-38.
192. Coleman, R. and R. M. Bell, Triacylglycerol synthesis in isolated fat cells. Studies on the microsomal diacylglycerol acyltransferase activity using ethanol-dispersed diacylglycerols. J Biol Chem, 1976. 251(15): p. 4537-43.
193. Hosaka, K., U. Schiele, and S. Numa, Diacylglycerol acyltransferase from rat liver microsomes. Separation and acyl-donor specificity. Eur J Biochem, 1977. 76(1): p. 113-8.
194. Ong, K. T., et al., Adipose Triglyceride Lipase Is a Major Hepatic Lipase That Regulates Triacylglycerol Turnover and Fatty Acid Signaling and Partitioning. Hepatology, 2011. 53(1): p. 116-126.
195. Osterlund, T., et al., Domain-structure analysis of recombinant rat hormone-sensitive lipase. Biochemical Journal, 1996. 319: p. 411-420.
196. Ikeda, Y., K. Okamura, and S. Fujii, Purification and Characterization of Rat-Liver Microsomal Monoacylglycerol Lipase in Comparison to Other Esterases. Biochimica Et Biophysica Acta, 1977. 488(1): p. 128-139.
197. Tucker, G. A. and A. P. Dawson, The kinetics of rat liver and heart mitochondrial beta-hydroxybutyrate dehydrogenase. Biochem J, 1979. 179(3): p. 579-81. 198. Fenselau, A. and K. Wallis, Substrate specificity and mechanism of action of acetoacetate coenzyme A transferase from rat heart. Biochemistry, 1974. 13(19): p. 3884-8.
199. Halestrap, A. P., Pyruvate and ketone-body transport across the mitochondrial membrane. Exchange properties, pH-dependence and mechanism of the carrier. Biochem J, 1978. 172(3): p. 377-87.
200. Broer, S., et al., Characterization of the high-affinity monocarboxylate transporter MCT2 in Xenopus laevis oocytes. Biochem J, 1999. 341 (Pt 3): p. 529-35.
201. Segawa, H., et al., Identification and functional characterization of a Na+-independent neutral amino acid transporter with broad substrate selectivity. J Biol Chem, 1999. 274(28): p. 19745-51.
202. Schadewaldt, P., Determination of branched-chain L-amino-acid aminotransferase activity in Methods in Enzymology. 2000, Elsevier. p. 23-32.
203. Ichihara, A. and E. Koyama, Transaminase of branched chain amino acids. I. Branched chain amino acids-alpha-ketoglutarate transaminase. J Biochem, 1966. 59(2): p. 160-9.
204. Schadewaldt, P. and F. Adelmeyer, Coupled enzymatic assay for estimation of branched-chain L-amino acid aminotransferase activity with 2-Oxo acid substrates. Anal Biochem, 1996. 238(1): p. 65-71.
205. Taylor, R. T., V. Shakespeare, and W. T. Jenkins, Branched chain amino acid aminotransferase. IV. Kinetics of the transamination reactions. J Biol Chem, 1970. 245(19): p. 4880-5.
206. Hutson, S. M. and T. R. Hall, Identification of the mitochondrial branched chain aminotransferase as a branched chain alpha-keto acid transport protein. J Biol Chem, 1993. 268(5): p. 3084-91.
207. Wynn, R. M., et al., Molecular mechanism for regulation of the human mitochondrial branched-chain alpha-ketoacid dehydrogenase complex by phosphorylation. Structure, 2004. 12(12): p. 2185-96.
208. Parker, P. J. and P. J. Randle, Branched chain 2-oxo-acid dehydrogenase complex of rat liver. FEBS Lett, 1978. 90(1): p. 183-6.
209. Paul, H. S. and S. A. Adibi, Activation of hepatic branched chain alpha-keto acid dehydrogenase by a skeletal muscle factor. J Biol Chem, 1982. 257(21): p. 12581-8.
210. Paxton, R., et al., Role of branched-chain 2-oxo acid dehydrogenase and pyruvate dehydrogenase in 2-oxobutyrate metabolism. Biochem J, 1986. 234(2): p. 295-303.
211. He, M., T. P. Burghardt, and J. Vockley, A novel approach to the characterization of substrate specificity in short/branched chain Acyl-CoA dehydrogenase. J Biol Chem, 2003. 278(39): p. 37974-86.
212. Shimomura, Y., et al., Purification and partial characterization of 3-hydroxyisobutyryl-coenzyme A hydrolase of rat liver. J Biol Chem, 1994. 269(19): p. 14248-53.
213. Rougraff, P. M., et al., Purification and characterization of 3-hydroxyisobutyrate dehydrogenase from rabbit liver. J Biol Chem, 1988. 263(1): p. 327-31.
214. Goodwin, G. W., et al., Purification and characterization of methylmalonate-semialdehyde dehydrogenase from rat liver. Identity to malonate-semialdehyde dehydrogenase. J Biol Chem, 1989. 264(25): p. 14965-71.
215. Luo, M. J., L. F. Mao, and H. Schulz, Short-chain 3-hydroxy-2-methylacyl-CoA dehydrogenase from rat liver: purification and characterization of a novel enzyme of isoleucine metabolism. Arch Biochem Biophys, 1995. 321(1): p. 214-20.
216. Haapalainen, A. M., et al., Crystallographic and kinetic studies of human mitochondrial acetoacetyl-CoA thiolase: the importance of potassium and chloride ions for its structure and function. Biochemistry, 2007. 46(14): p. 4305-21.
217. Finocchiaro, G., M. Ito, and K. Tanaka, Purification and properties of short chain acyl-CoA, medium chain acyl-CoA, and isovaleryl-CoA dehydrogenases from human liver. J Biol Chem, 1987. 262(17): p. 7982-9.
218. Chu, C. H. and D. Cheng, Expression, purification, characterization of human 3-methylcrotonyl-CoA carboxylase (MCCC). Protein Expr Purif, 2007. 53(2): p. 421-7.
219. Mack, M., et al., Biochemical characterization of human 3-methylglutaconyl-CoA hydratase and its role in leucine metabolism. FEBS J, 2006. 273(9): p. 2012-22.
220. Mack, M., et al., 3-Methylglutaconyl-CoA hydratase from Acinetobacter sp. Arch Microbiol, 2006. 185(4): p. 297-306.
221. Kaziro, Y., et al., Metabolism of propionic acid in animal tissues. VIII. Crystalline propionyl carboxylase. J Biol Chem, 1961. 236: p. 1917-23.
222. Kalousek, F., M. D. Darigo, and L. E. Rosenberg, Isolation and characterization of propionyl-CoA carboxylase from normal human liver. Evidence for a protomeric tetramer of nonidentical subunits. J Biol Chem, 1980. 255(1): p. 60-5.
223. Vlasie, M., S. Chowdhury, and R. Banerjee, Importance of the histidine ligand to coenzyme B12 in the reaction catalyzed by methylmalonyl-CoA mutase. J Biol Chem, 2002. 277(21): p. 18523-7.
224. Meyer, J. and P. M. Vignais, Kinetic study of glutamate transport in rat liver mitochondria. Biochim Biophys Acta, 1973. 325(3): p. 375-84.
225. Palmieri, F., et al., Mitochondrial metabolite carrier proteins: Purification, reconstitution, and transport studies. Mitochondrial Biogenesis and Genetics, Pt A, 1995. 260: p. 349-369.
226. Fahien, L. A. and E. Kmiotek, Regulation of glutamate dehydrogenase by palmitoyl-coenzyme A. Arch Biochem Biophys, 1981. 212(1): p. 247-53.
227. Lee, W. K., et al., Purification and characterization of glutamate dehydrogenase as another isoprotein binding to the membrane of rough endoplasmic reticulum. J Cell Biochem, 1999. 76(2): p. 244-53.
228. Engel, P. C. and S. S. Chen, A product-inhibition study of bovine liver glutamate dehydrogenase. Biochem J, 1975. 151(2): p. 305-18.
229. Chee, P. Y., J. L. Dahl, and L. A. Fahien, Purification and Properties of Rat-Brain Glutamate-Dehydrogenase. Journal of Neurochemistry, 1979. 33(1): p. 53-&.

TABLE 9
Metabolite concentrations (Table 9 uses separate reference numbering)

| | | | Mammalian | | Modeled | | |
|---|---|---|---|---|---|---|---|
| Metabolite | | Location | min [mM] | max [mM] | min [mM] | max [mM] | References |
| ACoA | Acetyl coenzyme A | cell | <0.01 | 0.11 | 0.0061 | 0.0673 | [1-7] |
| Acyl-CoA | Acyl coenzyme A | cell | <0.01 | 0.11 | 0.02 | 0.05 | [2, 4, 7] |
| CoAₘᵢₜₒ | Coenzyme A | mito | 0.05 | 2.26 | 0.0213 | 1.1477 | [8, 9] |
| NAD+ | Nicotinamide adenine dinucleotide | cell | 0.59 | 1.45 | 1.13 | 1.14 | [1, 10] |
| NAD+_{cyt} | Nicotinamide adenine dinucleotide | cyt | 0.51 | 1.39 | 1.13 | 1.13 | [11] |
| NAD+ₘᵢₜₒ | Nicotinamide adenine dinucleotide | mito | 0.19 | 0.87 | 0.026 | 0.05 | [9, 11] |
| NADH | Reduced nicotinamide adenine dinucleotide | cell | 0.02 | 0.36 | <0.01 | 0.0042 | [1, 12] |
| NADH_{cyt} | Reduced nicotinamide adenine dinucleotide | cyt | <0.01 | 0.01 | <0.01 | <0.01 | [11] |
| NADHₘᵢₜₒ | Reduced nicotinamide adenine dinucleotide | mito | 0.05 | 0.20 | <0.01 | 0.024 | [11] |
| P | Phosphate | cell | 2.25 | 20.50 | 11.37 | 21.7 | [2, 13-15] |
| P_{cyt} | Phosphate | cyt | 2.18 | 7.81 | 5.45 | 15.25 | [15, 16] |
| AMP | Adenosine monophosphate | cell | 0.07 | 2.62 | 0.02 | 3.89 | [2, 10, 17, 18] |
| ADP | Adenosine diphosphate | cell | 0.69 | 4.49 | 0.34 | 2.67 | [1, 2, 10, 17-19] |
| ADP_{cyt} | Adenosine diphosphate | cyt | 0.32 | 0.93 | 0.34 | 2.66 | [15] |
| ATP | Adenosine triphosphate | cell | 4.05 | 28.29 | 3.28 | 9.32 | [1, 2, 6, 12, 13, 17, 19-25] |
| GDP | Guanosine diphosphate | cell | 0.11 | 0.21 | 0.04 | 0.61 | [10] |
| GTP | Guanosine triphosphate | cell | 0.31 | 0.59 | 0.47 | 1.04 | [10] |
| Glc6P | Glucose 6-phosphate | cell | 0.10 | 0.93 | 0.05 | 0.34 | [2, 27] |
| Fru6P | Fructose-6-phosphate | cell | 0.03 | 0.23 | <0.01 | 0.08 | [2, 17, 27] |
| Fru26P2 | Fructose-2,6-bisphosphate | cell | <0.01 | 0.01 | <0.01 | 0.01 | [17] |
| Fru16P2 | Fructose-1,6-bisphosphate | cell | 0.01 | 0.13 | <0.01 | <0.01 | [2, 17, 26, 27] |
| DHAP | Dihydroxyacetone phosphate | cell | 0.01 | 0.14 | 0.01 | 0.037 | [2, 17] |
| GAP | Glyceraldehyde 3-phosphate | cell | 0.02 | 0.09 | <0.01 | 0.02 | [2, 27] |
| 13BPG | 1,3-Bisphosphoglycerate | cell | <0.01 | <0.01 | <0.01 | <0.01 | [26] |
| 3PG | 3-Phosphoglycerate | cell | 0.03 | 0.57 | 0.06 | 0.21 | [2, 26, 27] |
| 2PG | 2-Phosphoglycerate | cell | <0.01 | 0.03 | <0.01 | 0.036 | [2, 27] |
| PEP | Phosphoenolpyruvate | cell | 0.01 | 0.04 | 0.01 | 0.01 | [2, 26, 27] |
| Pyr | Pyruvate | cell | 0.01 | 6.00 | 0.02 | 1.5 | [1, 2, 6, 26, 27] |
| Gly3P | Glycerol 3-phosphate | cell | <0.01 | 0.25 | <0.01 | 0.09 | [2, 26, 27] |
| Glc1P | Glucose 1-phosphate | cell | 0.01 | 0.16 | <0.01 | 0.02 | [2, 26] |
| UDP-Glc | Uridine diphosphate glucose | cell | 0.26 | 0.72 | <0.01 | 2.1 | [2, 16] |
| Cit | Citrate | cell | 0.19 | 1.42 | 0.04 | 0.1 | [1, 2, 28] |
| IsoCit | Isocitrate | cell | 0.02 | 0.07 | <0.01 | <0.01 | [2, 28] |
| aKG | Alpha-ketoglutarate | cell | 0.05 | 0.29 | <0.01 | 0.05 | [1, 2, 28] |
| aKGₘᵢₜₒ | Alpha-ketoglutarate | mito | 0.13 | 0.21 | 0.02 | 0.15 | [9] |
| Suc | Succinate | cell | 0.26 | 4.42 | <0.01 | 0.06 | [1, 2, 6] |
| Fum | Fumarate | cell | 0.11 | 0.72 | 0.5 | 0.66 | [1, 2] |
| Mal | Malate | cell | 0.13 | 0.49 | 0.11 | 0.99 | [1, 2, 28] |
| OA | Oxaloacetate | cell | 0.02 | 0.05 | <0.01 | 0.04 | [2] |
| Asp | Aspartate | cell | 3.01 | 10.78 | <0.01 | 4.74 | [2, 28, 29] |
| Gln | Glutamine | cell | 10.38 | 26.24 | 10.33 | 10.33 | [30, 31] |
| Glu | Glutamate | cell | 4.63 | 18.72 | <0.01 | 1.09 | [2, 28, 29, 31] |
| IsoLeu | Isoleucine | cell | <0.01 | 0.23 | 0.18 | 0.2 | [29-31] |
| Leu | Leucine | cell | 0.07 | 0.37 | 0.33 | 0.39 | [29, 30] |
| Val | Valine | cell | 0.23 | 0.36 | 0.35 | 0.4 | [29] |
| Acyl-Carn | Acylcarnitine | cell | 0.01 | 0.94 | 0.03 | 0.14 | [9, 32] |
| Acyl-Carn_{cyt} | Acylcarnitine | cyt | 0.03 | 0.38 | 0.01 | 0.08 | [8] |
| Acyl-Carnₘᵢₜₒ | Acylcarnitine | mito | 0.00 | 0.22 | 0.08 | 0.44 | [8] |
| Carn | Carnitine | cell | 0.74 | 4.24 | 0.78 | 0.89 | [2, 14, 33] |
| Carn_{cyt} | Carnitine | cyt | 2.56 | 2.65 | 0.32 | 0.39 | [8] |
| Carnₘᵢₜₒ | Carnitine | mito | 1.84 | 2.03 | 2.66 | 3.02 | [8] |
| TAG | Triacylglycerol | cell | 3.34 | 9.78 | 1.4 | 2.77 | [2, 34] |
| DAG | Diacyl-glycerol | cell | 0.03 | 0.43 | 0.51 | 0.51 | [2, 34] |
| MalCoA | Malonyl coenzyme A | cell | <0.01 | 0.02 | <0.01 | 0.01 | [3, 5, 7, 35-37] |
| C4CoA | Butyryl coenzyme A | cell | <0.01 | 0.01 | <0.01 | <0.01 | [38] |
| C6CoA | Hexanoyl coenzyme A | cell | <0.01 | 0.01 | <0.01 | <0.01 | [39] |
| C8CoA | Octanoyl coenzyme A | cell | <0.01 | 0.01 | <0.01 | <0.01 | [39] |
| C10CoA | Decanoyl coenzyme A | cell | 0.01 | 0.02 | <0.01 | <0.01 | [39] |
| C14CoA | Myristoyl coenzyme A | cell | <0.01 | <0.01 | <0.01 | <0.01 | [4] |
| C16CoA | Palmitoyl coenzyme A | cell | <0.01 | 0.01 | <0.01 | <0.01 | [4] |

References
[1.] Sharma, N., et al., Regulation of pyruvate dehydrogenase activity and citric acid cycle intermediates during high cardiac power generation. J Physiol, 2005. 562(Pt 2): p. 593-603.
[2.] Taegtmeyer, H., et al., Assessing Cardiac Metabolism: A Scientific Statement From the American Heart Association. Circ Res, 2016. 118(10): p. 1659-701.
[3.] Saddik, M., et al., Acetyl-CoA carboxylase regulation of fatty acid oxidation in the heart. J Biol Chem, 1993. 268(34): p. 25836-45.
[4.] Maoz, D., et al., Immediate no-flow ischemia decreases rat heart nonesterified fatty acid and increases acyl-CoA species concentrations. Lipids, 2005. 40(11): p. 1149-54.
[5.] Stanley, W. C., et al., beta-Hydroxybutyrate inhibits myocardial fatty acid oxidation in vivo independent of changes in malonyl-CoA content. Am J Physiol Heart Circ Physiol, 2003. 285(4): p. H1626-31.
[6.] Sun, G., et al., Shotgun metabolomics approach for the analysis of negatively charged water-soluble cellular metabolites from mouse heart tissue. Anal Chem, 2007. 79(17): p. 6629-40.
[7.] Bedi, K. C., Jr., et al., Evidence for Intramyocardial Disruption of Lipid Metabolism and Increased Myocardial Ketone Utilization in Advanced Human Heart Failure. Circulation, 2016. 133(8): p. 706-16.
[8.] Idell-Wenger, J. A., L. W. Grotyohann, and J. R. Neely, Coenzyme A and carnitine distribution in normal and ischemic hearts. J Biol Chem, 1978. 253(12): p. 4310-8.
[9.] Russell, R. R., 3rd and H. Taegtmeyer, Coenzyme A sequestration in rat hearts oxidizing ketone bodies. J Clin Invest, 1992. 89(3): p. 968-73.
[10.] Kalsi, K. K., et al., Energetics and function of the failing human heart with dilated or hypertrophic cardiomyopathy. Eur J Clin Invest, 1999. 29(6): p. 469-77.
[11.] Kobayashi, K. and J. R. Neely, Control of maximum rates of glycolysis in rat cardiac muscle. Circ Res, 1979. 44(2): p. 166-75.
[12.] Wan, B., et al., Effects of cardiac work on electrical potential gradient across mitochondrial membrane in perfused rat hearts. Am J Physiol, 1993. 265(2 Pt 2): p. H453-60.
[13.] Chidsey, C. A., et al., Biochemical studies of energy production in the failing human heart. J Clin Invest, 1966. 45(1): p. 40-50.
[14.] Bowe, C., et al., Lipid intermediates in chronically volume-overloaded rat hearts. Effect of diffuse ischemia. Pflugers Arch, 1984. 402(3): p. 317-20.
[15.] Masuda, T., G. P. Dobson, and R. L. Veech, The Gibbs-Donnan near-equilibrium system of heart. J Biol Chem, 1990. 265(33): p. 20321-34.
[16.] Kashiwaya, Y., et al., Control of glucose utilization in working perfused rat heart. J Biol Chem, 1994. 269(41): p. 25502-14.
[17.] Narabayashi, H., J. W. Lawson, and K. Uyeda, Regulation of phosphofructokinase in perfused rat heart. Requirement for fructose 2,6-bisphosphate and a covalent modification. J Biol Chem, 1985. 260(17): p. 9750-8.
[18.] Kobayashi, K. and J. R. Neely, Mechanism of pyruvate dehydrogenase activation by increased cardiac work. J Mol Cell Cardiol, 1983. 15(6): p. 369-82.
[19.] Wischeler, B. S., E. R. Muller-Ruchholtz, and H. Reinauer, [Influence of heart work and substrate uptake on the regulation of pyruvate dehydrogenase activity in isolated guinea pig hearts (author's transl)]. Pflugers Arch, 1975. 355(1): p. 27-37.
[20.] El-Sharkawy, A. M., et al., Quantification of human high-energy phosphate metabolite concentrations at 3 T with partial volume and sensitivity corrections. Nmr in Biomedicine, 2013. 26(11): p. 1363-1371.
[21.] Weiss, R. G., G. Gerstenblith, and P. A. Bottomley, ATP flux through creatine kinase in the normal, stressed, and failing human heart. Proceedings of the National Academy of Sciences of the United States of America, 2005. 102(3): p. 808-813.
[22.] Smith, C. S., et al., Altered creatine kinase adenosine triphosphate kinetics in failing hypertrophied human myocardium. Circulation, 2006. 114(11): p. 1151-1158.
[23.] Bottomley, P. A., et al., Reduced Myocardial Creatine Kinase Flux in Human Myocardial Infarction An In Vivo Phosphorus Magnetic Resonance Spectroscopy Study. Circulation, 2009. 119(14): p. 1918-1924.
[24.] Meininger, M., et al., Concentrations of human cardiac phosphorus metabolites determined by SLOOP 31P NMR spectroscopy. Magn Reson Med, 1999. 41(4): p. 657-63.
[25.] Okada, M., et al., Influence of aging or left ventricular hypertrophy on the human heart: contents of phosphorus metabolites measured by 31P MRS. Magn Reson Med, 1998. 39(5): p. 772-82.
[26.] Cortassa, S., et al., From Metabolomics to Fluxomics: A Computational Procedure to Translate Metabolite Profiles into Metabolic Fluxes. Biophysical Journal, 2015. 108(1): p. 163-172.
[27.] Rovetto, M. J., W. F. Lamberton, and J. R. Neely, Mechanisms of glycolytic inhibition in ischemic rat hearts. Circ Res, 1975. 37(6): p. 742-51.
[28.] Randle, P. J., P. J. England, and R. M. Denton, Control of the tricarboxylate cycle and its interactions with glycolysis during acetate utilization in rat heart. Biochem J, 1970. 117(4): p. 677-95.
[29.] Scharff, R. and I. G. Wool, Effect of diabetes on the concentration of amino acids in plasma and heart muscle of rats. Biochem J, 1966. 99(1): p. 173-8.
[30.] Morgan, H. E., et al., Regulation of protein synthesis in heart muscle. I. Effect of amino acid levels on protein synthesis. J Biol Chem, 1971. 246(7): p. 2152-62.
[31.] Morgan, H. E., et al., Regulation of protein synthesis and degradation during in vitro cardiac work. Am J Physiol, 1980. 238(5): p. E431-42.
[32.] Whitmer, J. T., et al., Control of fatty acid metabolism in ischemic and hypoxic hearts. J Biol Chem, 1978. 253(12): p. 4305-9.
[33.] Cederbla. G, Lindsted. S, and K. Lundholm, Concentration of Carnitine in Human Muscle-Tissue. Clinica Chimica Acta, 1974. 53(3): p. 311-321.
[34.] Denton, R. M. and P. J. Randle, Hormonal control of lipid concentration in rat heart and gastrocnemius. Nature, 1965. 208(5009): p. 488.
[35.] McGarry, J. D., et al., Observations on the affinity for carnitine, and malonyl-CoA sensitivity, of carnitine palmitoyltransferase I in animal and human tissues. Demonstration of the presence of malonyl-CoA in non-hepatic tissues of the rat. Biochem J, 1983. 214(1): p. 21-8.
[36.] Minkler, P. E., et al., Quantification of malonyl-coenzyme A in tissue specimens by high-performance liquid chromatography/mass spectrometry. Anal Biochem, 2006. 352(1): p. 24-32.
[37.] Reszko, A. E., et al., Assay of the concentration and 13C-isotopic enrichment of malonyl-coenzyme A by gas chromatography-mass spectrometry. Anal Biochem, 2001. 298(1): p. 69-75.
[38.] Li, Q., et al., 4-Hydroxy-2(E)-nonenal (HNE) catabolismand formation of HNE adducts are modulated by beta oxidation of fatty acids in the isolated rat heart. Free Radic Biol Med, 2013. 58: p. 35-44.
[39.] Kasuya, F., et al., Analysis of medium-chain acyl-coenzyme A esters in mouse tissues by liquid chromatography-electrospray ionization mass spectrometry. Anal Biochem, 2004. 325(2): p. 196-205.

TABLE 10
Estimated Vₘₐₓ values

| | | | Estimated vₘₐₓ |
|---|---|---|---|
| Short Name | Long Name | EC/TCDB Number | value [μmol/g/h] |
| v_CD36 | fatty acid translocase | TCDB 4.C.1.1 | 4.20E+01 |
| v_acs1 | (Long-chain) acyl-coa synthetase 1 | EC 6.2.1.3 | 2.80E+00 |
| v_fatp1 | fatty acid transport protein 1 | TCDB 4.C.1.1 | 1.04E−01 |
| v_fatp4 | fatty acid transport protein 4 | TCDB 4.C.1.1 | 1.35E−01 |
| v_CPT1 | Carnitine O-palmitoyltransferase 1, liver isoform | EC 2.3.1.21 | 7.50E-02 |
| v_CACT | Carnitin-Acylcarnitin translocase | TCDB 2.A.29.8 | 5.00E+00 |
| v_CPT2 | Carnitine O-palmitoyltransferase 2, mitochondrial | EC 2.3.1.21 | 5.00E+01 |
| v_c4coa_scdh | Short chain acyl-coa dehydrogenase (c4) | EC 1.3.8.1 | 5.00E+00 |
| v_c6coa_mcdh | medium chain acyl-coa dehydrogenase (c6) | EC 1.3.8.7 | 5.25E+01 |
| v_c8coa_mcdh | medium chain acyl-coa dehydrogenase (c8) | EC 1.3.8.7 | 6.25E+01 |
| v_c10coa_mcdh | medium chain acyl-coa dehydrogenase (c10) | EC 1.3.8.7 | 1.20E+01 |
| v_c12coa_mcdh | medium chain acyl-coa dehydrogenase (c12) | EC 1.3.8.7 | 9.75E+00 |
| v_c10coa_lcdh | long chain acyl-coa dehydrogenase (c10) | EC 1.3.8.8 | 1.20E+00 |
| v_c12coa_lcdh | long chain acyl-coa dehydrogenase (c12) | EC 1.3.8.8 | 9.75E−01 |
| v_c14coa_lcdh | long chain acyl-coa dehydrogenase (c14) | EC 1.3.8.8 | 5.25E−01 |
| v_c16coa_lcdh | long chain acyl-coa dehydrogenase (c16) | EC 1.3.8.8 | 1.20E−01 |
| v_etf | ETF-FAD | — | 2.50E+02 |
| v_etfq | ETF-QO | — | 1.25E+03 |
| v_E_hyd_C4_CoA_MC | Enoyl-coa hydratase (Crontonase) (ec4) | EC 4.2.1.17 | 2.50E+03 |
| v_E_hyd_C6_CoA_MC | Enoyl-coa hydratase (Crontonase) (ec6) | EC 4.2.1.17 | 3.20E+04 |
| v_E_hyd_C8_CoA_MC | Enoyl-coa hydratase (Crontonase) (ec8) | EC 4.2.1.17 | 2.28E+04 |
| v_E_hyd_C10_CoA_MC | Enoyl-coa hydratase (Crontonase) (ec10) | EC 4.2.1.17 | 1.35E+04 |
| v_E_hyd_C12_CoA_MC | Enoyl-coa hydratase (Crontonase) (ec12) | EC 4.2.1.17 | 4.00E+03 |
| v_E_hyd_C14_CoA_MC | Enoyl-coa hydratase (Crontonase) (ec14) | EC 4.2.1.17 | 2.17E+03 |
| v_E_hyd_C16_CoA_MC | Enoyl-coa hydratase (Crontonase) (ec16) | EC 4.2.1.17 | 1.00E+03 |
| v_3HdH_C4_CoA | 3-hydroxyacyl-coa dehydrogenase (Ic4) | EC 1.1.1.35 | 5.75E+10 |
| v_3HdH_C6_CoA | 3-hydroxyacyl-coa dehydrogenase (Ic6) | EC 1.1.1.35 | 5.75E+10 |
| v_3HdH_C8_CoA | 3-hydroxyacyl-coa dehydrogenase (Ic8) | EC 1.1.1.35 | 5.75E+10 |
| v_3HdH_C10_CoA | 3-hydroxyacyl-coa dehydrogenase (Ic10) | EC 1.1.1.35 | 5.75E+10 |
| v_3HdH_C12_CoA | 3-hydroxyacyl-coa dehydrogenase (Ic12) | EC 1.1.1.35 | 5.75E+10 |
| v_3HdH_C14_CoA | 3-hydroxyacyl-coa dehydrogenase (Ic14) | EC 1.1.1.35 | 5.75E+10 |
| v_3HdH_C16_CoA | 3-hydroxyacyl-coa dehydrogenase (Ic16) | EC 1.1.1.35 | 5.75E+10 |
| v_3KT_C4_I | 3-ketoacyl-coa thiolase (kc4) | EC 2.3.1.16 | 2.50E+00 |
| v_3KT_C4_II | 3-ketoacyl-coa thiolase (kc4) | EC 2.3.1.16 | 2.50E+01 |
| v_3KT_C6 | 3-ketoacyl-coa thiolase (kc4) | EC 2.3.1.16 | 6.00E+00 |
| v_3KT_C8 | 3-ketoacyl-coa thiolase (kc4) | EC 2.3.1.16 | 5.50E+00 |
| v_3KT_C10 | 3-ketoacyl-coa thiolase (kc4) | EC 2.3.1.16 | 5.75E+00 |
| v_3KT_C12 | 3-ketoacyl-coa thiolase (kc4) | EC 2.3.1.16 | 5.25E+00 |
| v_3KT_C14 | 3-ketoacyl-coa thiolase (kc4) | EC 2.3.1.16 | 4.25E+00 |
| v_3KT_C16 | 3-ketoacyl-coa thiolase (kc4) | EC 2.3.1.16 | 2.50E+00 |
| v_pdhc | Pyruvate dehydrogenase complex | EC 1.2.4.1; EC 1.8.1.4; EC 2.3.1.12 | 1.00E−01 |
| v_cs | Citrate synthase, mitochondrial | EC 2.3.3.1 | 8.00E+01 |
| v_ac | Aconitase | EC 4.2.1.3 | 9.00E+04 |
| v_icdh_nad | NAD-dependent isocitrate dehydrogenase | EC 1.1.1.41 | 2.70E+01 |
| v_icdh_nadp | NADP-dependent isocitrate dehydrogenase | EC 1.1.1.42 | 4.50E+00 |
| v_akdhc | α-ketogluterate dehydrogenase | EC 1.2.4.2; EC 1.8.1.4; EC 2.3.1.61 | 4.50E+02 |
| v_succoas_atp | Succinyl-Coa Synthetase (ATP) | EC 6.2.1.4; EC 6.2.1.5; EC 6.2.1.5 | 2.25E+04 |
| v_succoas_gtp | Succinyl-Coa Synthetase (GTP) | EC 6.2.1.4; EC 6.2.1.4; EC 6.2.1.5 | 2.48E+03 |
| v_succdh | Succinate dehydrogenase | EC 1.3.5.1 | 9.00E+03 |
| v_fum | Fumerase | EC 4.2.1.2 | 1.80E+05 |
| v_mdh | Malate dehydrogenase, mitochondrial | EC 1.1.1.37 | 9.00E+04 |
| v_tdh | NAD(P) transhydrogenase, mitochondrial | EC 1.6.1.2 | 3.60E+03 |
| v_ATP_use | ATP usage | — | 1.20E−01 |
| v_Phos | Phosphate carrier protein, mitochondrial | TCDB 2.A.29.4.2 | 7.72E+10 |
| v_syn | F0F1 synthetase | EC 3.6.3.14 | 6.40E-06 |
| v_ex | ATP-ADP nucleotide exchanger | TCDB 2.A.29 | 7.20E-07 |
| v1 | Complex I | EC 1.6.5.3 | 6.72E-07 |
| v3 | Complex III | EC 1.10.2.2 | 2.40E-06 |
| v4 | Complex IV | EC 1.9.3.1 | 9.60E-04 |
| v_ak_cyt | Adenylate kinase | EC 2.7.4.3 | 8.00E+03 |
| v_02_diff | O2 diffusion | — | 8.00E+01 |
| v_ppase | Pyrophosphatase | EC 3.6.1.1 | 8.00E−02 |
| I_C_ED | Chloride electro diffusion | — | 8.00E−01 |
| IK_P | Sodium pump | — | 2.40E−05 |
| I_K_ED | Sodium electro diffusion | — | 8.00E−01 |
| I_Na_P | Potassium pump | — | 1.60E-04 |
| I_Na_ED | Sodium electro diffusion | | 8.00E−01 |
| I_H ED | Mitochondrial uncoupling protein | TC 2.A.29.24.1; TC 2.A.29.24.3; | 8.00E−01 |
| | | TC 2.A.29.3.2; TC 2.A.29.3.4; | |
| | | TC 2.A.29.3.5 | |
| v_gpi | Glucose-6-phosphate isomerase | EC 5.3.1.9 | 3.51E+03 |
| v_ald | Fructose-bisphosphate aldolase B | EC 4.1.2.13 | 1.17E+03 |
| v_tpi | Triosephosphate isomerase 1 | EC 5.3.1.1 | 1.17E+03 |
| v_gapdh | Glyceraldehydephosphate dehydrogenase | EC 1.2.1.12 | 1.76E+05 |
| v_pgk | Phosphyglyceratekinase (Pgk) | EC 2.7.2.3 | 5.85E+02 |
| v_pgm | 2-Phospho-D-glycerate 2,3 phosphomutase (Pgm) | EC 5.4.2.1 | 5.85E+05 |
| v_eno | 2-Phospho-D-glycerate hydrolase (Eno) | EC 4.2.1.11 | 1.17E+03 |
| v_glcT1 | Glucose transporter type 1 | TC 2.A.1.1 | 4.15E-04 |
| v_glcT4 | Glucose transporter type 4 | TC 2.A.1.1 | 6.31E−03 |
| v_hk1 | Hexokinase | EC 2.7.1.1 | 4.68E−02 |
| v_hk2 | Hexokinase | EC 2.7.1.1 | 4.68E−03 |
| v_fbp2 | Phosphofructokinase 2 (FBP2) | EC 2.7.1.105; EC 3.1.3.46; EC 3.1.3.46 | 4.68E−03 |
| v_pfk2 | Phosphofructokinase 2 (Pfk2) | EC 2.7.1.105; EC 3.1.3.46; EC 3.1.3.46 | 1.17E−02 |
| v_fbp1 | Fructose-1,6-bisphosphatase (Fbp1) | EC 3.1.3.11 | 1.17E+00 |
| v_pfk1 | Phosphofructokinase 1 (Pfk1) | EC 2.7.1.11 | 5.85E−01 |
| v_pk | Pyruvate kinase (Pk) | EC 2.7.1.40 | 5.85E−02 |
| v_pc | Pyruvate carboxylase; mitochondrial | EC 6.4.1.1 | 1.17E−02 |
| v_mal_pyrT | Malate-pyruvate antiport (MalPyrT) | — | 4.68E−01 |
| v_me_nadp | Identifier nicht gefunden | — | 1.17E+01 |
| v_ldh | Lactate dehydrogenase | EC 1.1.1.27 | 1.17E+05 |
| v_mdh_cyt | Malate dehydrogenase, cytoplasmic | EC 1.1.1.37 | 1.17E+03 |
| v_lacT | Lactate transport (LacT) | TCDB 2.A.1.13.1; TCDB 2.A.1.13.5; | 1.17E−02 |
| | | TCDB 2.A.1.13.6; TCDB | |
| | | 2.A.1.13.7; TCDB 2.A.1.13.9 | |
| v_pyrT | pyruvate transport (pyrT) | TCDB 2.A.1.13.1 | 1.17E−02 |
| v_pyrT_mito | Mitochondrial pyruvate transport | TCDB 2.A.1.13.1 | 1.17E+05 |
| v_ndk_cyt | Nudiki (cytosolic) | EC 2.7.4.6 | 1.17E+04 |
| v_ndk_mito | Nudiki (mitochondrial) | EC 2.7.4.6 | 1.17E+06 |
| v_ASAT_mito | Aspartate aminotransferase, mitochondrial | EC 2.6.1.1 | 1.50E+02 |
| v_ASAT_in | Aspartate aminotransferase, cytoplasmic | EC 2.6.1.1 | 1.50E+02 |
| v_asp_glu_T | aspartate -glutamate carrier | TCDB 2.A.29.14.1 | 2.25E−01 |
| v_mal_akg_T | Malate - α-ketogluterate carrier | TCDB 2.A.29.2.13 | 1.50E+06 |
| v_g3pdh_cyt | Glycerol-3-phosphate dehydrogenase (cytosolic) | EC 1.1.1.8 | 1.50E+06 |
| v_g3pdh_mito | Glycerol-3-phosphate dehydrogenase (mitochondrial) | EC 1.1.5.3 | 3.00E-10 |
| v_g6pd | Glucose-6-phosphate 1-dehydrogenase | EC 1.1.1.49 | 5.00E-06 |
| v_pglase | 6-phosphogluconolactonase | EC 3.1.1.31 | 3.00E−05 |
| v_pgdh | 6-phosphogluconate dehydrogenase; decarboxylating | EC 1.1.1.44 | 1.00E−01 |
| v_rpe | Ribulose-phosphate 3-epimerase | EC 5.1.3.1 | 5.00E−03 |
| v_rpi | Ribose-5-phosphate isomerase | EC 5.3.1.6 | 1.00E−02 |
| v_taldo | Transaldolase | EC 2.2.1.2 | 1.00E−02 |
| v_tkl1 | Transketolase 1 | EC 2.2.1.1 | 1.00E+02 |
| v_tkl2 | Transketolase 2 | EC 2.2.1.1 | 1.00E+00 |
| v_Cit_Mal | Citrate-malate exchanger | TCDB 2.A.29.7.2 | 3.00E−03 |
| v_Cit_Lys | ATP dependent citrate lyase | EC 2.3.3.8 | 1.00E−05 |
| v_ACC1 | Acetyl-CoA carboxylase 1 | EC 6.4.1.2 | 1.40E−05 |
| v_Mal_CoA_dc | Malonyl-CoA decarboxylase | EC 4.1.1.9 | 1.00E-06 |
| v_glycerol_uptake | Glycerol-uptake | | 7.50E−03 |
| v_glycerol_kinase | Glycerol kinase | EC 2.7.1.30 | 3.60E−03 |
| v_gpat | Glycerol-3-phosphate acyltransferase | EC 2.3.1.15 | 3.00E−03 |
| v_agpat | Acetyl glycerol-3-phosphate acyltransferase | EC 2.3.1.51 | 4.50E−03 |
| v_PAP | Phosphatidic acid phosphatase | EC 3.1.3.4 | 9.00E−03 |
| v_dgat | Diacylglycerol acyltransferase | EC 2.3.1.20 | 1.05E−02 |
| v_ld_syn | LD synthesis (tag) | — | 3.00E−03 |
| v_atgl | ATGL | EC 3.1.1.3 | 3.00E−04 |
| v_hsl | Hormone-sensitive lipase | EC 3.1.1.79 | 3.00E−04 |
| v_magl | Monoacylglycerol lipase | EC 3.1.1.23 | 3.00E−04 |
| v_g16pi | alpha-D-Glucose 1-phosphate 1,6-phosphomutase | EC 5.4.2.2 | 1.20E+04 |
| v_upgase | UTP:Glucose-1-phosphate uridylyltransferase (UPGase) | EC 2.7.7.9 | 9.00E+00 |
| v_ndkutp | Nudiki (cytosolic) (udp) | EC 2.7.4.6 | 6.00E+01 |
| v_gs | Glycogen synthase (GS) | EC 2.4.1.11 | 3.00E−04 |
| v_gp | Glycogen-phosphorylase (GP) | EC 2.4.1.1 | 1.80E−02 |
| v_acac_cytT | Acetoacetate export (MCT1/MCT2) | TCDB 2.A.1.13.1; TCDB 2.A.1.13.5; | 2.85E−02 |
| | | TCDB 2.A.1.13.6; TCDB | |
| | | 2.A.1.13.7; TCDB 2.A.1.13.9 | |
| v_bhbut_cytT | B-Hydroxy butyrate export (MCT1/MCT2) | TCDB 2.A.13.1 | 2.85E−02 |
| v_acac_mito_ex | Acetoacetate transport (mitochondrial) | TCDB 2.A. 13.1 | 8.55E−02 |
| v_bhbut_mito_ex | B-Hydroxy butyrate transport (mitochondrial) | TCDB 2.A.13.1 | 8.55E−02 |
| v_bHBDH | D-beta-hydroxybutyrate dehydrogenase, mitochondrial | EC 1.1.1.30 | 5.70E+03 |
| v_scot | Succinyl-CoA:3-ketoacid coenzyme A transferase 1, | EC 2.8.3.5 | 5.70E+01 |
| | mitochondrial | | |
| v_valT | valine transporter | — | 1.00E−01 |
| v_leuT | leucine transporter | — | 1.00E−01 |
| v_isoleuT | isoleucine transporter | — | 1.00E−01 |
| v_BCAAT_val | Branched-chain-amino-acid aminotransferase | EC 2.6.1.42 | 1.00E−01 |
| v_BCAAT_leu | Branched-chain-amino-acid aminotransferase | EC 2.6.1.42 | 1.00E−01 |
| v_BCAAT_isoleu | Branched-chain-amino-acid aminotransferase | EC 2.6.1.42 | 1.00E−01 |
| v_BCKADH_aKIVA | Branched-chain alpha-keto acid dehydrogenase | EC 1.2.4.4 | 1.00E−01 |
| v_BCKADH_aKICA | Branched-chain alpha-keto acid dehydrogenase | EC 1.2.4.4 | 1.00E−01 |
| v_BCKADH_KMeVA | Branched-chain alpha-keto acid dehydrogenase | EC 1.2.4.4 | 1.00E−01 |
| v_MBCoADH_IsoButCoA | 2-methylacyl-CoA dehydrogenase | EC 1.3.99.12 | 1.00E+01 |
| v_MBCoADH_MeButCoA | 2-methylacyl-CoA dehydrogenase | EC 1.3.99.12 | 1.00E+01 |
| v_ECoAH_MeAcrCoA | Enoyl-CoA hydratase, mitochondrial | EC 4.2.1.17 | 1.00E+03 |
| v_ECoAH_TigCoA | Enoyl-CoA hydratase, mitochondrial | EC 4.2.1.17 | 1.00E+01 |
| v_HibCDA_HibCoA | 3-hydroxyisobutyryl-CoA hydrolase, mitochondrial | EC 3.1.2.4 | 1.00E+01 |
| v_HibCDH_HBA | 3-hydroxyisobutyryl-CoA hydrolase, mitochondrial | EC 3.1.2.4 | 1.00E+01 |
| v_MMSDH_MMSALD | Methylmalonate-semialdehyde dehydrogenase, mitochondrial | EC 1.2.1.27 | 1.00E+01 |
| v_HMBCDH_MeHButCoA | Methylmalonate-semialdehyde dehydrogenase, mitochondria | EC 1.2.1.27 | 1.00E+01 |
| v_MAACT_MeAACoA | 3-ketoacyl-CoA thiolase, mitochondrial | EC 2.3.1.16 | 1.00E+01 |
| v_IVCoADH_isoValCoA | Isovaleryl-CoA dehydrogenase, mitochondrial | EC 1.3.8.4 | 1.00E+01 |
| v_MECCC_MeCroCoA | Methylcrotonoyl-CoA carboxylase, mitochondrial | EC 6.4.1.4 | 1.00E+01 |
| v_MEGCCH_MeGCCoA | Methylglutaconyl-CoA hydratase, mitochondrial | EC 4.2.1.18 | 1.00E+01 |
| v_HMGCL_HMeGCoA | Hydroxymethylglutaryl-CoA lyase, mitochondrial | EC 4.1.3.4 | 1.00E+01 |
| v_PCC_PropCoa | Propionyl-CoA carboxylase, mitochondrial | EC 6.1.4.3 | 1.00E+01 |
| v_MMCM_MeMalCoa | Methylmalonyl-CoA mutase, mitochondrial | EC 5.4.99.2 | 1.00E+01 |

1. A computer-implemented method for determining an individual metabolic cardiac profile of a subject comprising
a) providing a heart tissue sample from said subject,
b) quantifying proteins in said sample to obtain protein quantities of the heart tissue sample, and
c) applying information about the protein quantities from b) to a mathematical model.

2. The computer-implemented method according to claim 1, wherein said heart tissue sample is a left ventricle, a right ventricle, a septum, a left atrium, and/or a right atrium heart tissue sample obtained from said subject during a myocardium examination or cardiac surgery.

3. The computer-implemented method according to claim 1, wherein said method further comprises quantitatively determining metabolites in a plasma, blood, or serum sample from said subject, wherein said metabolites are selected from the group consisting of glucose, lactate, pyruvate, glycerol, fatty acids, glutamate, glutamine, leucin, isoleucine, valine, acetate, B-hydroxybutyrate, catecholamines, insulin and combinations thereof.

4. The computer-implemented method according to claim 1, wherein the method further comprises quantitatively determining of an individual cardiac parameter of the subject comprising heart rate, blood pressure, pressure-volume loops, and/or heart power.

5. The computer-implemented method according to claim 1, wherein the protein quantities of the heart tissue sample from the subject are determined using a protein quantification method selected from the group consisting of mass spectrometry, large scale mass spectrometry, immunoassay, Western blot, microfluidics/nanotechnology sensor, and aptamer capture assay, wherein said method comprises:
a) solubilizing the heart tissue sample to obtain a solubilized heart tissue sample,
b) extracting proteins from the solubilized heart tissue sample of a) according to the protein quantification method, wherein said proteins are optionally fragmented into peptides,
c) transferring said extracted proteins and/or peptides from b) to a device of said protein quantification method and identifying and quantifying the proteins and/or peptides in said sample,
wherein said protein quantification method provides a protein profile of said sample from the subject.

6. The computer-implemented method according to claim 5, wherein the protein profile, individual cardiac parameters and/or the metabolites of the subject are introduced into the mathematical model.

7. The computer-implemented method according to claim 1, wherein said individual cardiac metabolic profile comprises a substrate uptake rate, a myocardial ATP consumption, a myocardial ATP production reserve, a myocardial ATP production at a cardiac workload, and a myocardial ATP production at maximal workload, wherein the myocardial ATP production reserve is calculated as a difference between the myocardial ATP-production at said cardiac workload and the myocardial ATP production at said maximal workload.

8. The computer-implemented method according to claim 5, wherein the mathematical model of the individual metabolic cardiac profile of the subject comprises
inputing a cardiac kinetic model and providing metabolic parameters relating to the cardiac kinetic model, and/or
providing individual cardiac parameters at cardiac workload,
parametrizing said mathematical model to the heart tissue sample of said subject by calculating a maximal activity Vₘₐₓ of said subject, and
computing a cardiac energy expenditure profile of said subject at cardiac workload,
wherein said individual metabolic cardiac profile of said subject is optionally compared to a non-diseased subject at cardiac workload.

9. The computer-implemented method according to claim 8, wherein computing the maximal activity Vₘₐₓ for model parametrization for the heart tissue sample of the subject comprises
a) input of the protein profile of the subject, and
b) loading at least one reference data set, wherein said reference data set comprises a reference data set containing the quantities of data entries, wherein each data entry of the quantity contains at least one correlated compatible protein label and/or metabolite label, and
c) computing the maximal enzyme activity Vₘₐₓ of the subject, wherein Vₘₐₓ is calculated by the formula
$${V}_{max}^{subject} = {V}_{max}^{ref} \frac{{E}^{subject}}{{E}^{ref}}$$
by applying the protein quantities of the subject to E^{subject} and by applying Vₘₐₓ^{ref} and protein quantities to E^{ref} of any of the reference data sets.

10. The computer-implemented method according to claim 1, wherein said individual metabolic cardiac profile is calculated for a plurality of cardiac workloads, including rest, stress or cardiac pacing, wherein individual cardiac parameter including heart rate, blood pressure, heart power are determined at said cardiac workloads.

11. The computer-implemented method according to claim 1, wherein a plurality of said mathematical models are used in said computations for the heart at physiological state, including normal post-absorptive, post prandial, and fasted, and for the heart at pathological state, including ischemic or diabetic.

12. The computer-implemented method according to claim 1, and calculating, via the computer-implemented method, prognosis of a cardiovascular related disorder, an effect of a change in nutritional interventions, activity and/or therapeutic interventions on protein expression and on the time variation of a metabolic parameter in the heart tissue sample of the subject.

13. The computer-implemented method according to claim 1, and preventing, ascertaining, prognosing or treating, via the computer-implemented method, a cardiovascular related disorder or detecting a perturbation of a normal biological state of the heart from a subject.

14. The computer-implemented method according to claim 1, and, via the computer-implemented method,
(i) selecting a nutritional or a therapeutic intervention, and
(ii) evaluating or preventing a therapeutic intervention.

15. A computer program adapted to execute a mathematical modelling algorithm that will be performed by a computing device/module to produce outputs of given data provided as inputs according to claim 1, wherein said computer program, is written in a programming language selected from the group consisting of Fortran, C #, C/C++, High Level Shading Language, and Python.

16. The computer-implemented method according to claim 2, wherein said heart tissue sample is obtained during a cardiac catheter examination.

17. The computer-implemented method according to claim 3 wherein the metabolites are quantitively determined in the plasma sample from said subject.

18. The computer-implemented method according to claim 5, wherein the protein quantification method is large scale mass spectrometry, said proteins are fragmented into peptides, the extracted peptides from b) are transferred to a mass spectrometer and the peptides in said sample are quantified.

19. The computer program according to claim 15, said computer program is MATLAB.
